(19) United States
(12) Patent Application Publication
(10) Pub. No.: US 2022/0249484 A1
(43) Pub. Date: Aug. 11, 2022

(54) METHODS FOR PREDICTING RESPONSIVENESS OF CANCER TO FERROPTOSIS-INDUCING THERAPIES

(71) Applicant: Memorial Sloan Kettering Cancer Center, New York, NY (US)

(72) Inventor: Xuejun JIANG, New York, NY (US)

(73) Assignee: Memorial Sloan Kettering Cancer Center, New York, NY (US)

(21) Appl. No.: 17/611,805

(22) PCT Filed: May 15, 2020
(86) PCT No.: PCT/US2020/033203

§ 371 Date: Nov. 16, 2021

Related U.S. Application Data
(60) Provisional application No. 62/849,645, filed on May 17, 2019.

(51) Int. Cl. A61K 31/513; C12Q 1/6886; G01N 33/574; A61K 31/40; A61K 31/44; A61K 31/366; A61P 35/00

(52) U.S. Cl. CPC A61K 31/513; C12Q 1/6886; G01N 33/57496; A61K 31/40; A61K 31/44; G01N 2800/52; A61P 35/00; C12Q 2600/156; C12Q 2600/106; C12Q 2600/158; A61K 31/366

(57) ABSTRACT

The present disclosure relates generally to methods for determining whether a patient diagnosed with cancer will benefit from or is predicted to be responsive to treatment with a ferroptosis-inducing therapy. These methods are based on screening a cancer patient for mutations in cadherin and/or the Merlin-Hippo-YAP signaling pathway.

### CROSS-REFERENCE TO RELATED APPLICATIONS

This application is a U.S. National Stage Application under 35 U.S.C. § 371 of International Patent Application No. PCT/US2020/033203, filed on May 15, 2020, which claims the benefit of and priority to U.S. Provisional Patent Application No. 62/849,645, filed May 17, 2019, the entire contents of each of which are incorporated herein by reference.

### STATEMENT OF GOVERNMENT SUPPORT

This invention was made with government support under CA204232 awarded by the National Institutes of Health. The government has certain rights in the invention.

### TECHNICAL FIELD

The present technology relates to methods for determining whether a patient diagnosed with cancer will benefit from or is predicted to be responsive to treatment with a ferroptosis-inducing therapy. These methods are based on screening a cancer patient for mutations in cadherin and/or the Merlin-Hippo-YAP signaling pathway.

### SEQUENCE LISTING

The instant application contains a Sequence Listing which has been submitted electronically in ASCII format and is hereby incorporated by reference in its entirety. Said ASCII copy, created on Jun. 8, 2020, is named 115872-0847_SL.txt and is 79,378 bytes in size.

### BACKGROUND

The following description of the background of the present technology is provided simply as an aid in understanding the present technology and is not admitted to describe or constitute prior art to the present technology.

Ferroptosis is triggered by an inability of cellular antioxidant defenses to overcome the oxidative stress of metabolic activity, leading to a wave of iron-dependent cellular lipid peroxidation and, ultimately, cell death. Glutathione peroxidase-4 (GPX4), a glutathione-dependent enzyme catalyzing the clearance of lipid ROS, plays an essential role in preventing cells from ferroptosis. Inactivation of GPX4 renders the cell unable to detoxify lipid peroxides, by-products of cellular metabolism, which, when in excess, damage cellular membranes, and kill the cell via ferroptosis. As such, loss of GPX4 function, either by its direct inhibition or by depriving cystine/cysteine, a building block for its cofactor glutathione, can induce ferroptosis. A prominent role for ferroptosis in cancer is also emerging. Numerous types of therapy-resistant cancer cells, especially those with mesenchymal and de-differentiated characteristics, are more susceptible to ferroptosis. Knowledge of the molecular mechanisms underlying the sensitivity of mesenchymal cancer cells to ferroptosis are not clear. Because the induction of ferroptosis may be a promising therapeutic approach for killing such otherwise therapy-resistant, metastasis-prone cancer cells, there is an urgent need for developing reliable and accurate methods for predicting whether a cancer patient would be responsive to a ferroptosis-inducing therapy.

### SUMMARY OF THE PRESENT TECHNOLOGY

In one aspect, the present disclosure provides a method for selecting a cancer patient for treatment with a ferroptosis-inducing therapy comprising (a) detecting the presence of a mutation in at least one polynucleotide encoding one or more proteins selected from the group consisting of E-cadherin, N-cadherin, Merlin, Mst1, Mst2, Lats1, and Lats2 in a biological sample obtained from the cancer patient, wherein the mutation is a frameshift mutation, a missense mutation, a deletion, an insertion, a nonsense mutation, an inversion, or a translocation; and (b) administering to the cancer patient an effective amount of a ferroptosis-inducing agent. The mutation may be detected using any nucleic acid detection assay known in the art such as next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In some embodiments, the biological sample comprises genomic DNA, cDNA, RNA, and/or mRNA.

In one aspect, the present disclosure provides a method for treating a therapy-resistant, metastasis-prone cancer in a patient in need thereof comprising administering to the cancer patient an effective amount of a ferroptosis-inducing agent, wherein mRNA or polypeptide expression and/or activity levels of one or more of E-cadherin, N-cadherin, Merlin, Mst1, Mst2, Lats1, and Lats2 in a biological sample obtained from the patient are reduced compared to that observed in a control sample obtained from a healthy subject or a predetermined threshold. In another aspect, the present disclosure provides a method for treating a therapy-resistant, metastasis-prone cancer in a patient in need thereof comprising administering to the cancer patient an effective amount of a ferroptosis-inducing agent, wherein mRNA or polypeptide expression and/or activity levels of one or more of YAP, TAZ, TFRC, ACSL4, and TGF-β are elevated compared to that observed in a control sample obtained from a healthy subject or a predetermined threshold. The metastasis-prone cancer may be resistant to chemotherapy or radiation therapy. Additionally or alternatively, in some embodiments, the patient is diagnosed with or suffers from a cancer selected from the group consisting of mesothelioma, lung cancer, liver cancer, colon cancer, rectal cancer, and breast cancer.

Additionally or alternatively, in some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). In some embodiments, TFRC mRNA expression levels are detected using a forward primer comprising the sequence of SEQ ID NO: 36 and a reverse primer comprising the sequence of SEQ ID NO: 37 or a probe comprising the sequence of SEQ ID NO: 15, SEQ ID NO: 16, SEQ ID NO: 36, SEQ ID NO: 37, or any complement thereof. In certain embodiments, ACSL4 mRNA expression levels are detected using a forward primer comprising the sequence of SEQ ID NO: 34 and a reverse primer comprising the sequence of SEQ ID NO: 35 or a probe comprising the sequence of SEQ ID NO: 34, SEQ ID NO: 35, or any complement thereof. In other embodiments, Merlin mRNA expression levels are detected using a forward primer comprising the sequence of SEQ ID NO: 1 and a reverse primer comprising the sequence of SEQ ID NO: 2 or a probe comprising the sequence of SEQ ID NO: 1, SEQ ID NO: 2, SEQ ID NO: 7, SEQ ID NO: 8, SEQ ID NO: 9, SEQ ID NO: 10, or any complement thereof. In some embodiments, E-cadherin or N-cadherin mRNA expression levels are detected using a probe comprising the sequence of SEQ ID NO: 3, SEQ ID NO: 4, SEQ ID NO: 5, SEQ ID NO: 6, or any complement thereof. In certain embodiments, Lats1 or Lats2 mRNA expression levels are detected using a probe comprising the sequence of SEQ ID NO: 11, SEQ ID NO: 12, SEQ ID NO: 13, SEQ ID NO: 14, or any complement thereof.

Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any of the embodiments of methods disclosed herein, the ferroptosis-inducing agent is a class 1 ferroptosis inducer (system Xₑ⁻ inhibitor) or a class 2 ferroptosis inducer (glutathione peroxidase 4 (GPx4) inhibitor). Examples of ferroptosis-inducing agents include, but are not limited to, erastin, erastin derivatives (e.g., MEII, PE, AE, imidazole ketone erastin (IKE)), DPI2, BSO, SAS, lanperisone, SRS13-45, SRS13-60, RSL3, DPI7, DPI10, DPI12, DPI13, DPI17, DPI18, DPI19, ML160, sorafenib, and artemisinin derivatives. Additionally or alternatively, in some embodiments of the methods disclosed herein, the patient is human.

### BRIEF DESCRIPTION OF THE DRAWINGS

FIG. 1A shows an immunofluorescence staining demonstrating that cystine deprivation induced HCT116 human colon cancer cells to undergo a form of program cell death known as ferroptosis when cultured at low cell density, but the cells became resistant to ferroptosis when the culture density approached high confluence.

FIG. 1B shows a bar graph demonstrating that cystine deprivation-induced ferroptosis in HCT116 human colon cancer cells depended on the confluence of the cultured cells.

FIG. 1C shows a bar graph demonstrating that cystine deprivation-induced ferroptosis in HCT116 human colon cancer cells was associated with the production of lipid reactive oxygen species (ROS).

FIG. 1D shows a graph illustrating that cystine deprivation-induced ferroptosis was dependent on cell density in most human cancer epithelial cell lines tested, such as HepG2 (liver cancer), PC9 and H1650 (lung cancer), and MDA-MB-231 (breast cancer), but not BT474 (breast cancer), which was resistant to ferroptosis. In particular, the MDA-MB-231 cells were sensitive regardless of cell density, and H1650 cells were the most sensitive to ferroptosis.

FIG. 1E shows an immunofluorescence staining illustrating that inhibiting cystine import with erastin, an inhibitor of the cystine/glutamate antiporter, induced the human cancer epithelial cell lines of FIG. 1D to undergo ferroptosis in in vivo context, mimicked by culturing cells into 3D multicellular tumor spheroids.

FIG. 1F shows a bar graph demonstrating that inhibiting cystine import with erastin reduced the viability of the lung cancer cell line H1650, and the breast cancer cell line MDA-MB-231, when cultured into 3D multicellular tumor spheroids.

FIG. 1G shows an immunoblot illustrating the levels of E-cadherin, an important cell-cell adhesion molecule, in the tested human cancer epithelial cell lines. In particular, the ferroptosis-resistant cell line BT474 expressed the highest levels of E-cadherin; while the ferroptosis density-dependent hypersensitive cell line H1650 expressed low levels of E-cadherin and E-cadherin was undetectable in the density-independent ferroptosis sensitive cell line MDA-MB-231.

FIG. 111 shows an immunoblot illustrating the levels of E-cadherin in E-cadherin depleted HCT116 human colon cancer cells; and that N-cadherin expression was not induced in the absence of E-cadherin.

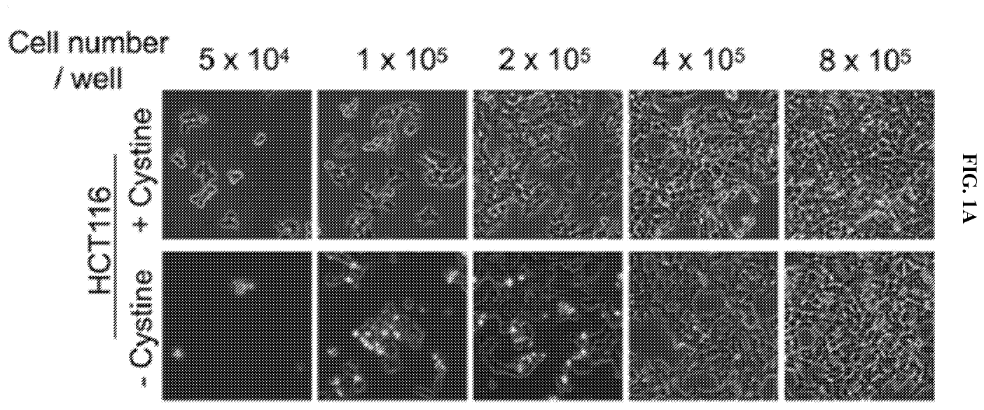

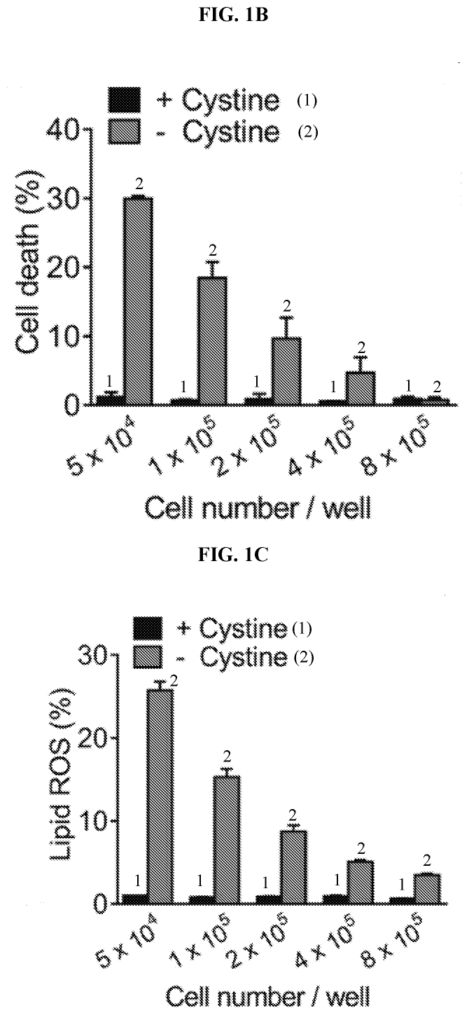

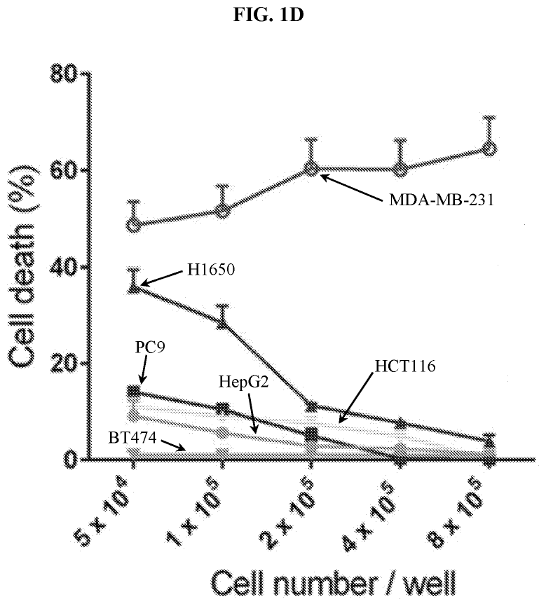

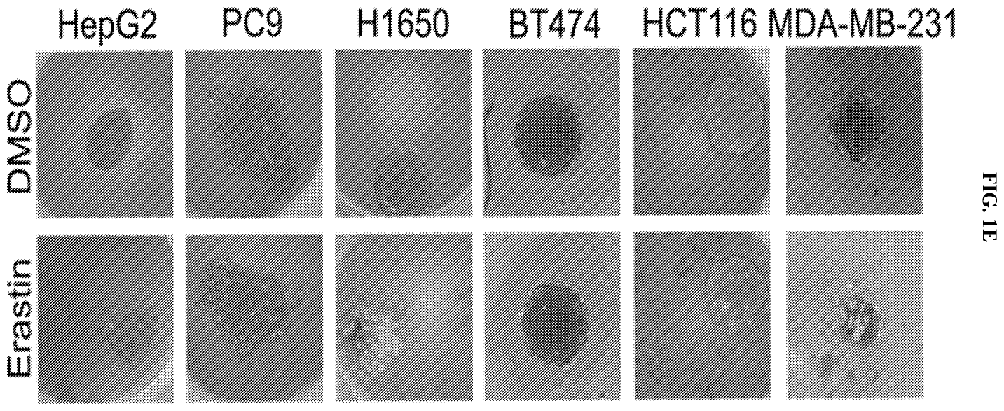

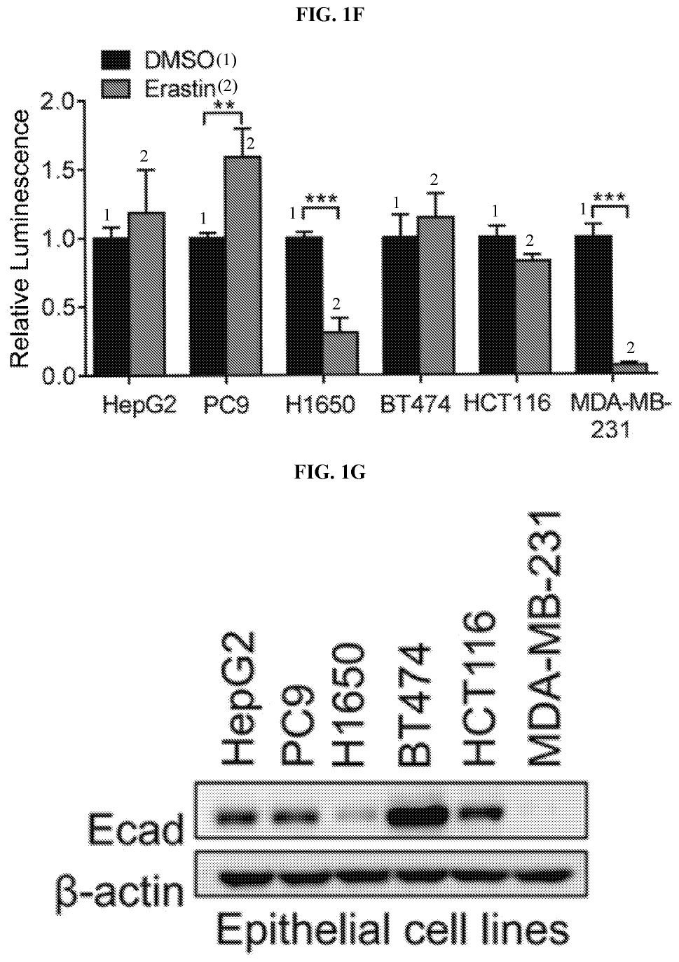

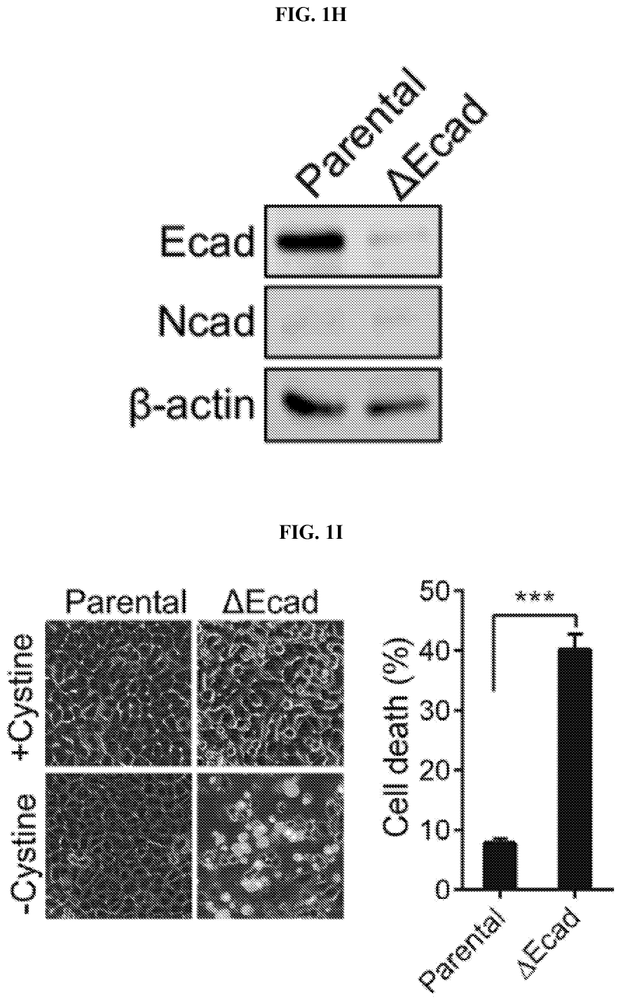

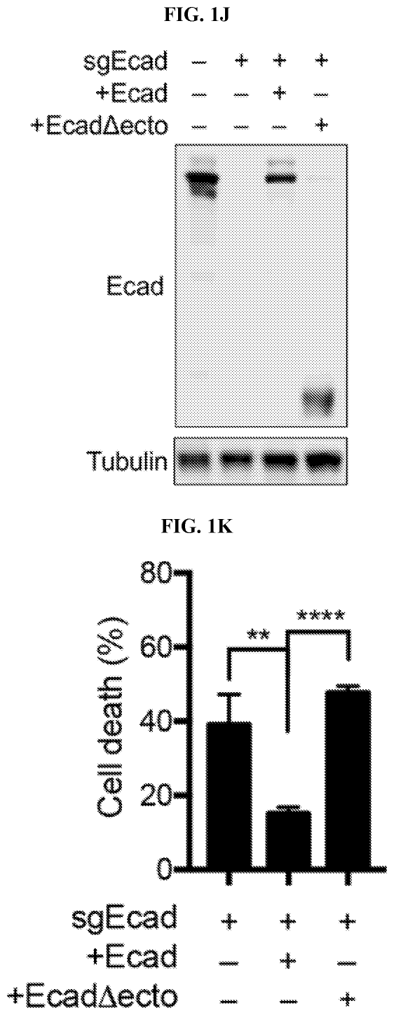

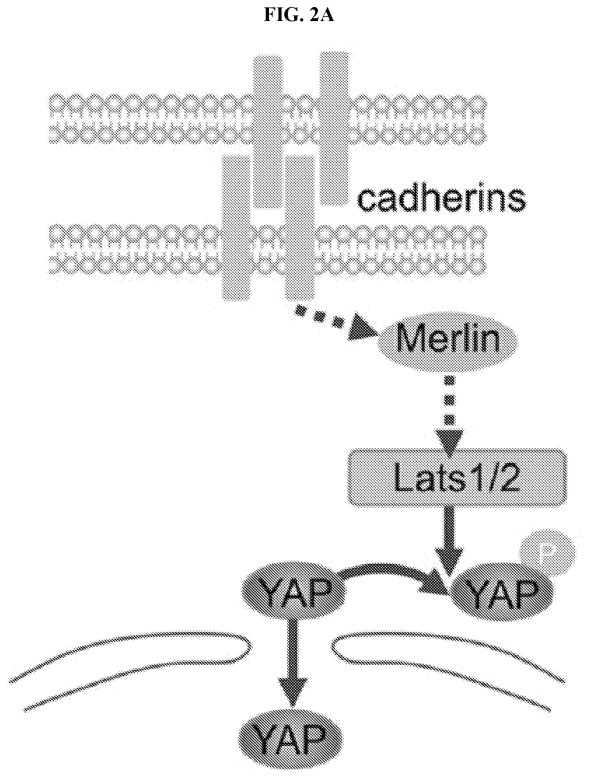

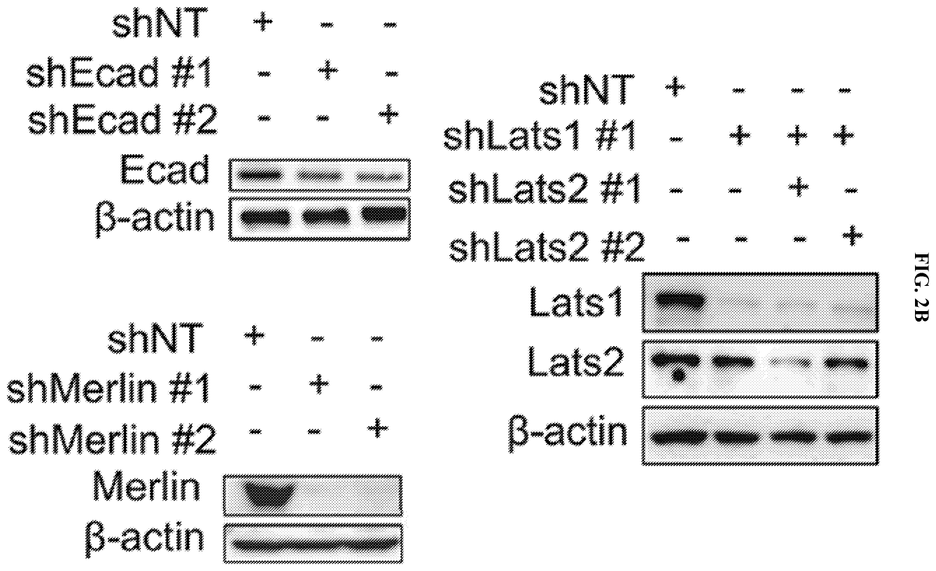

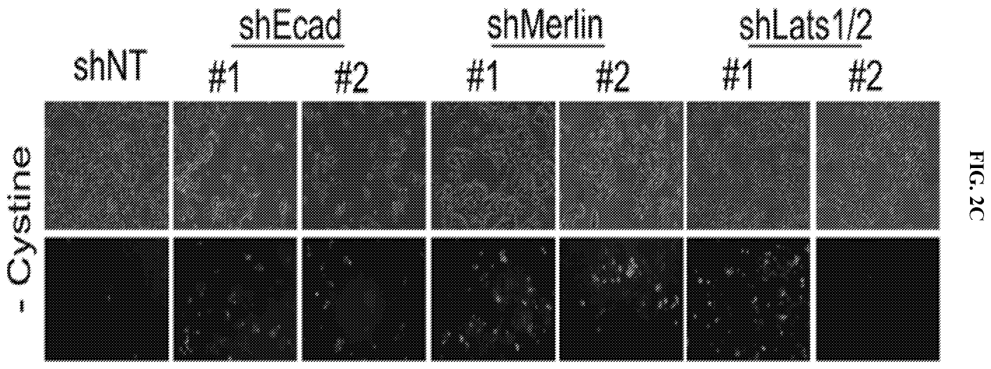

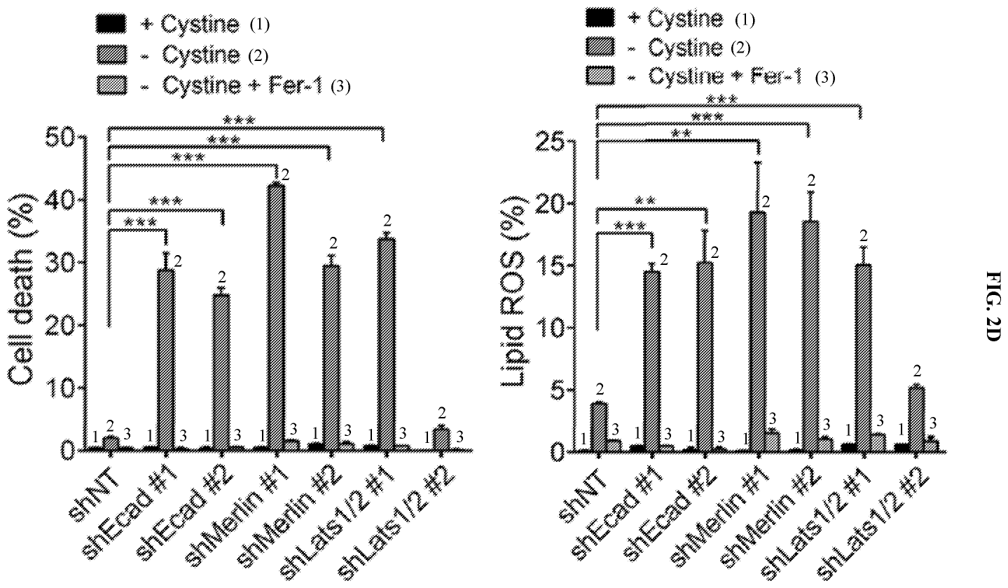

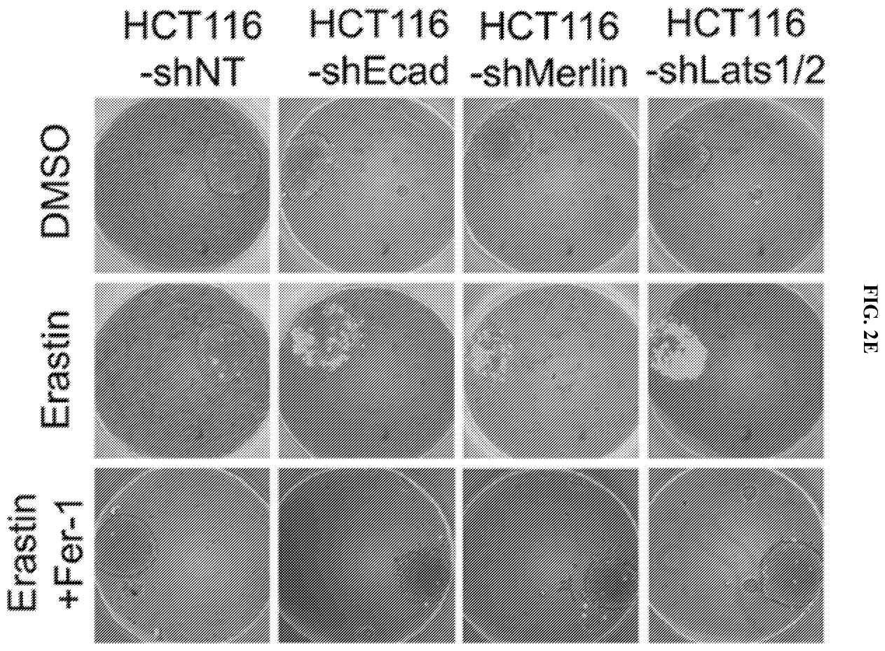

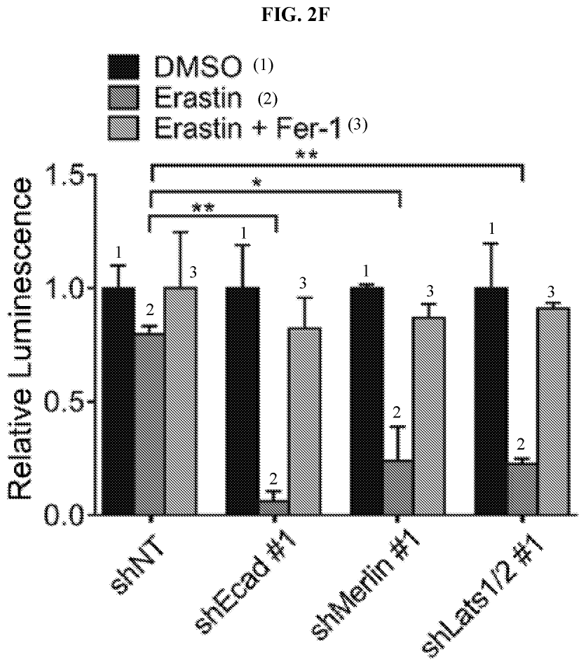

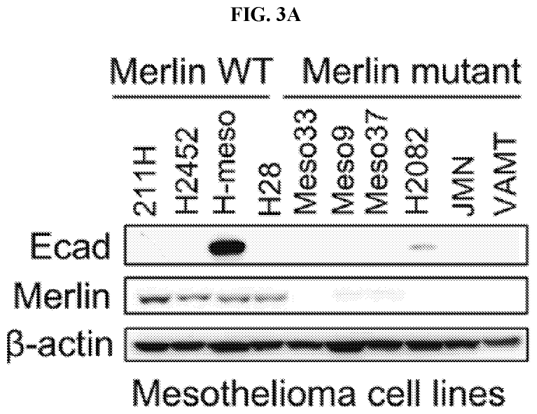

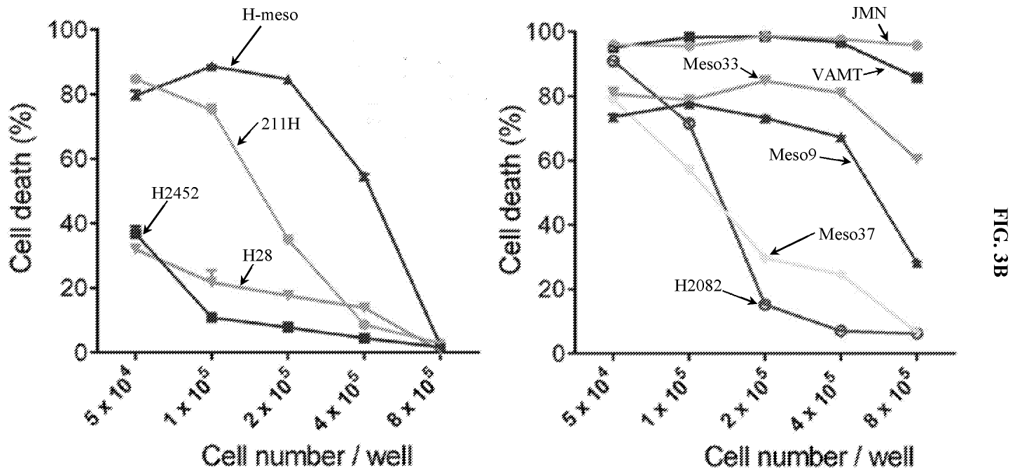

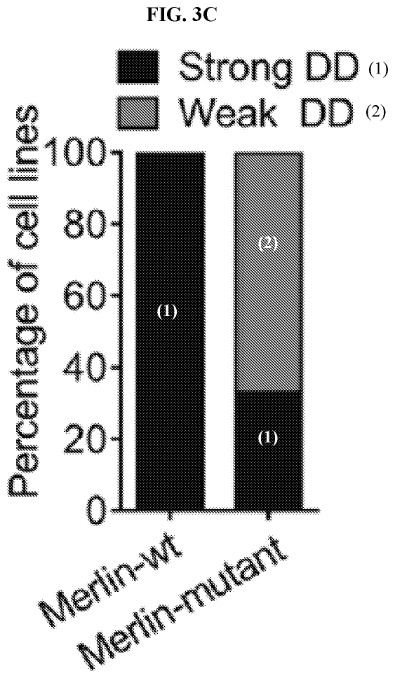

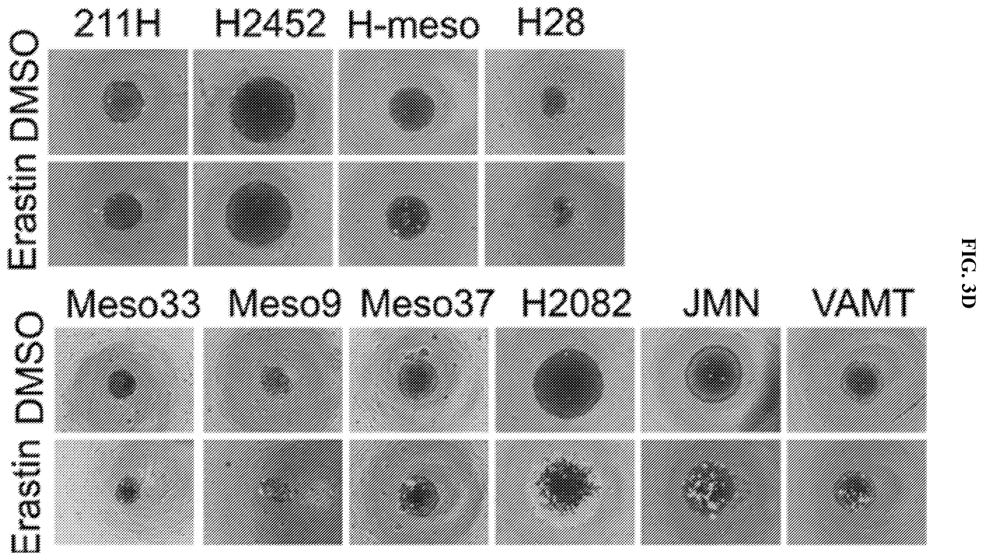

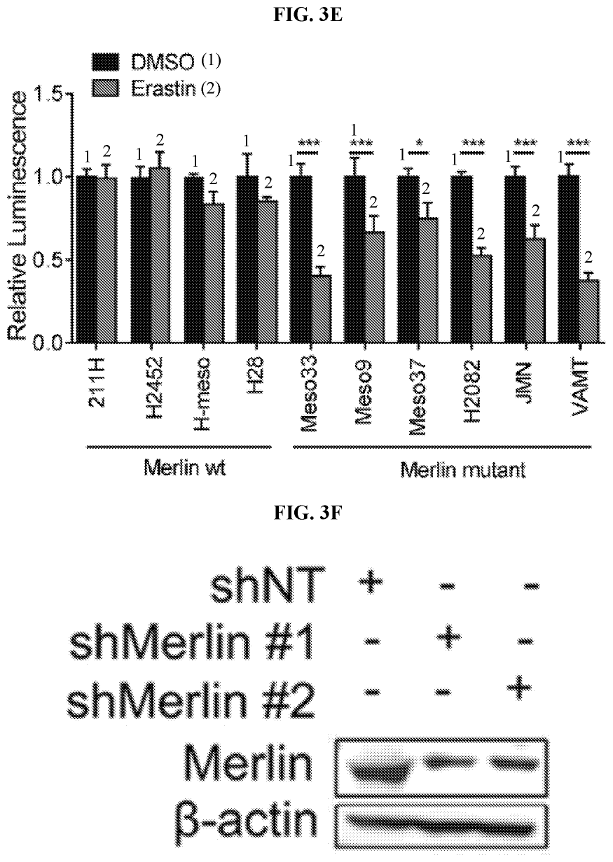

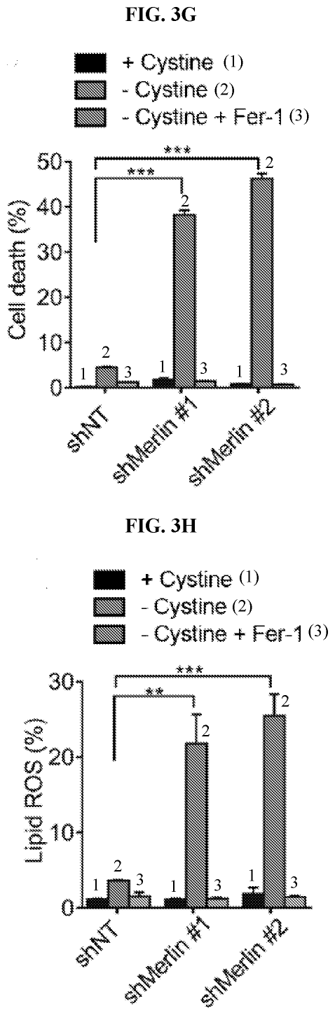

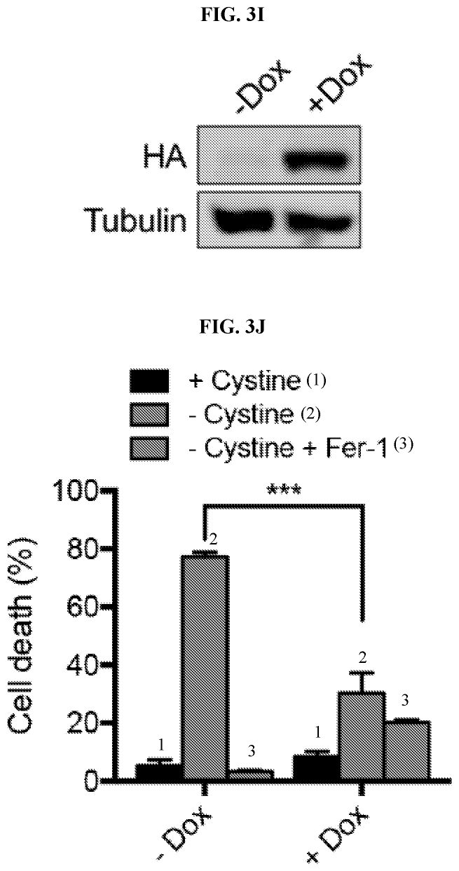

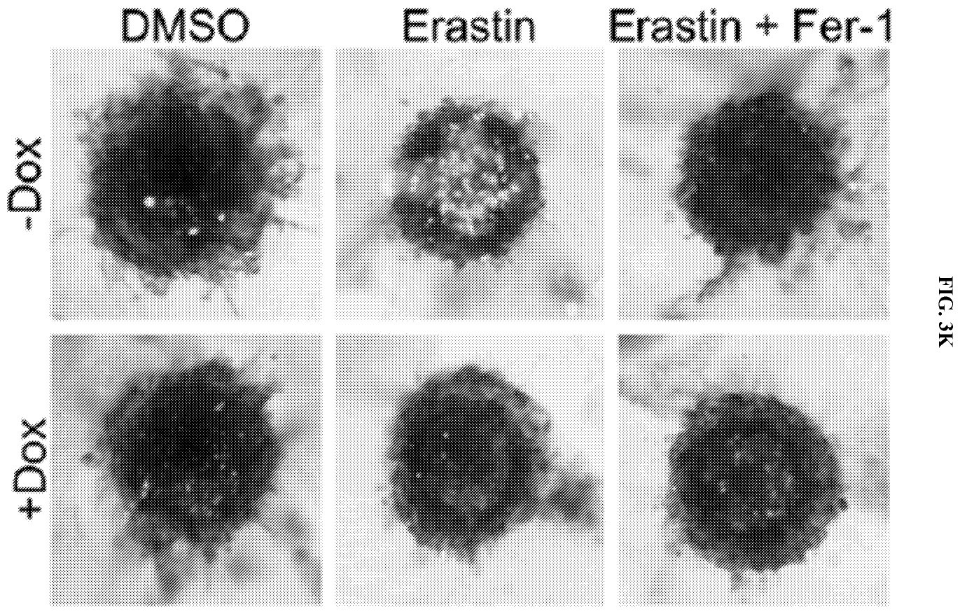

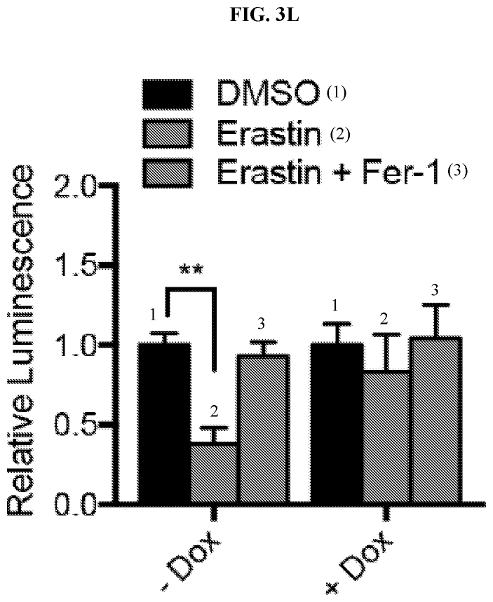

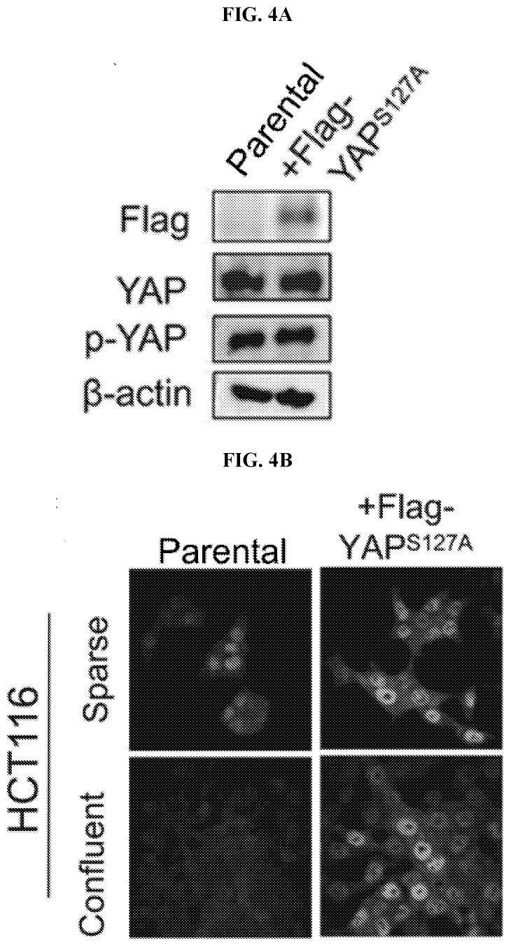

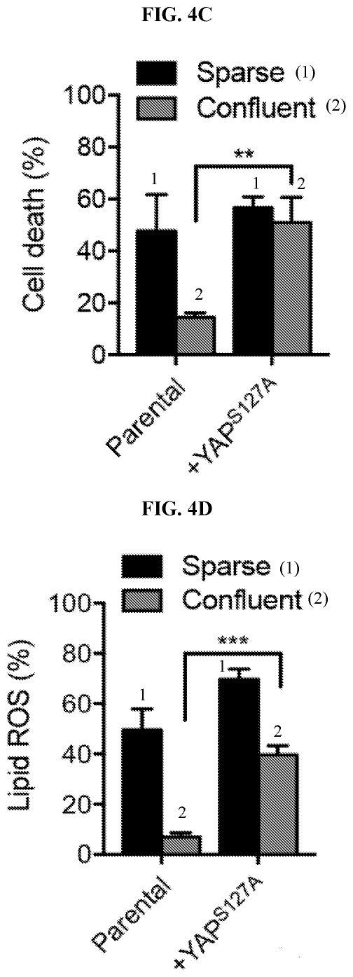

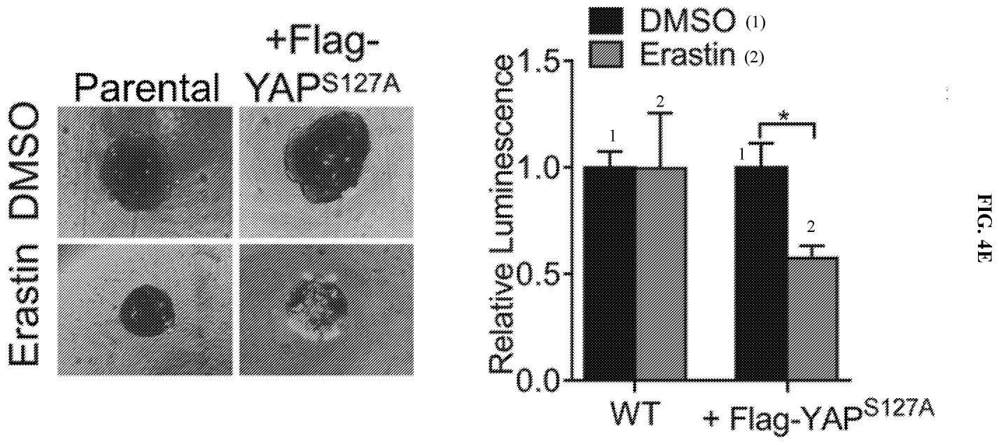

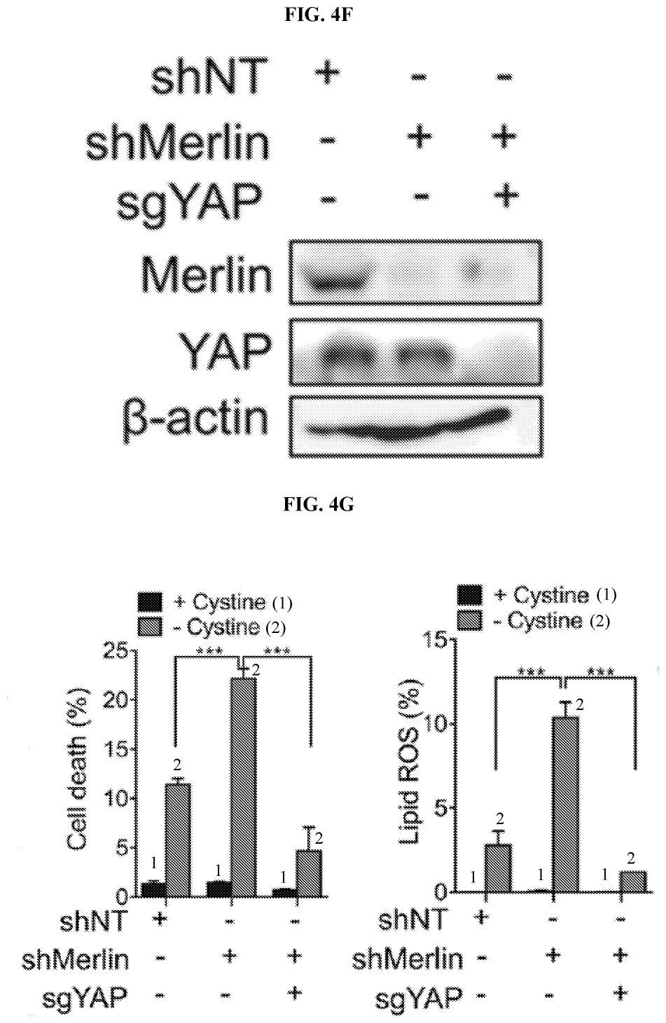

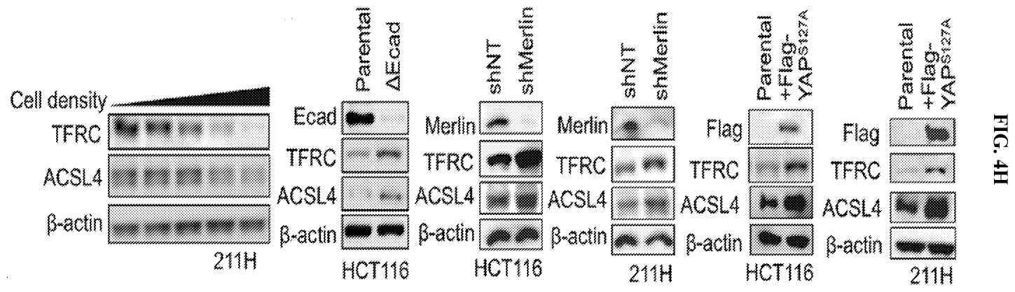

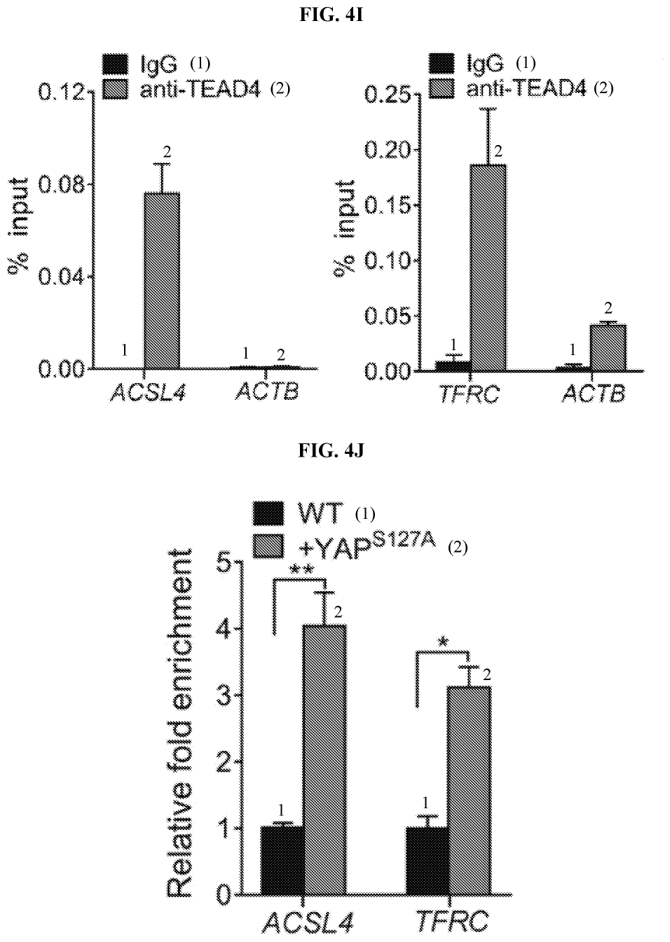

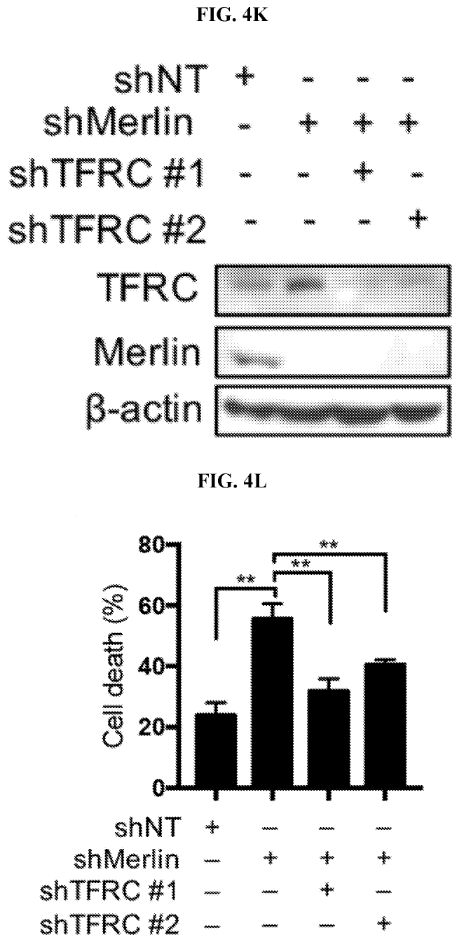

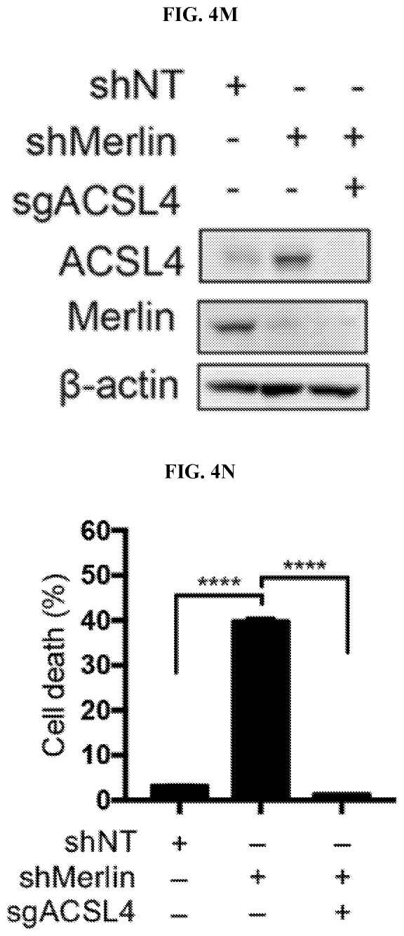

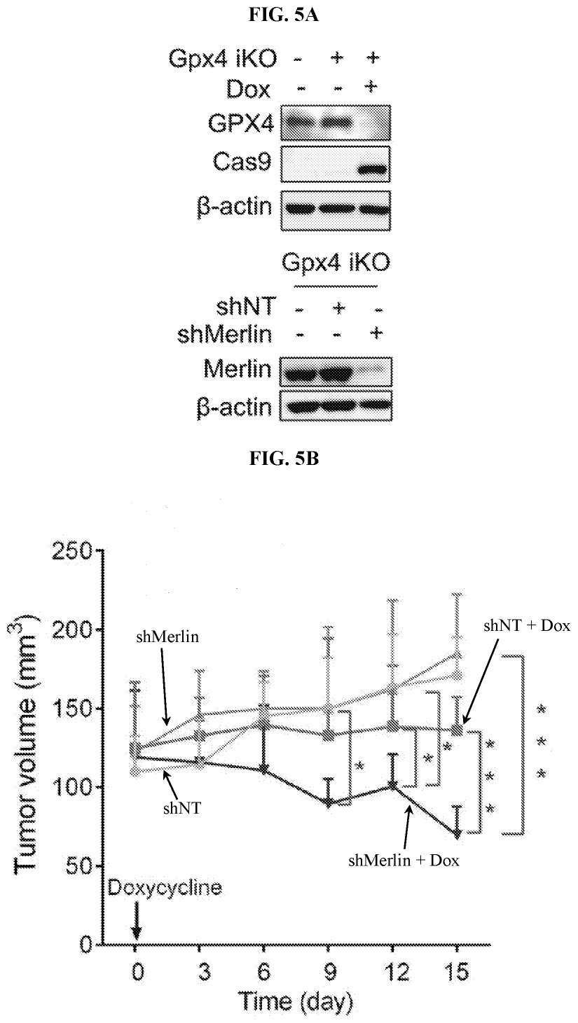

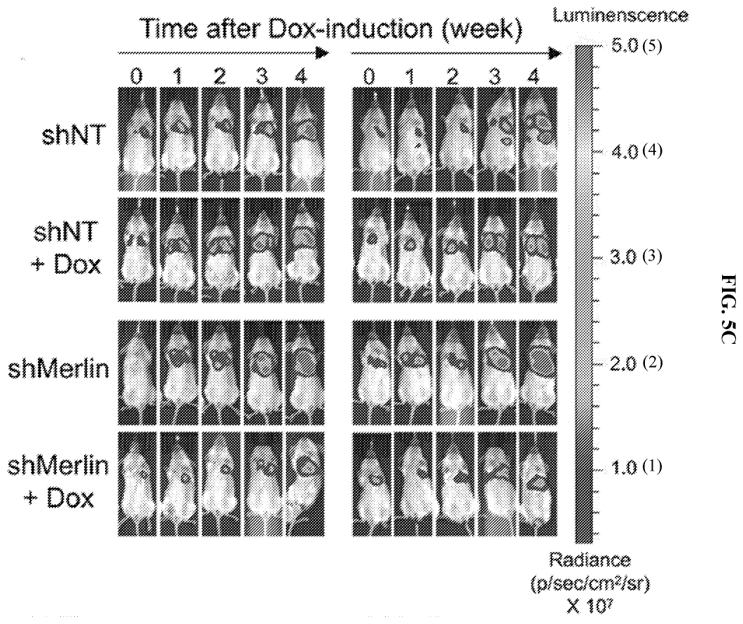

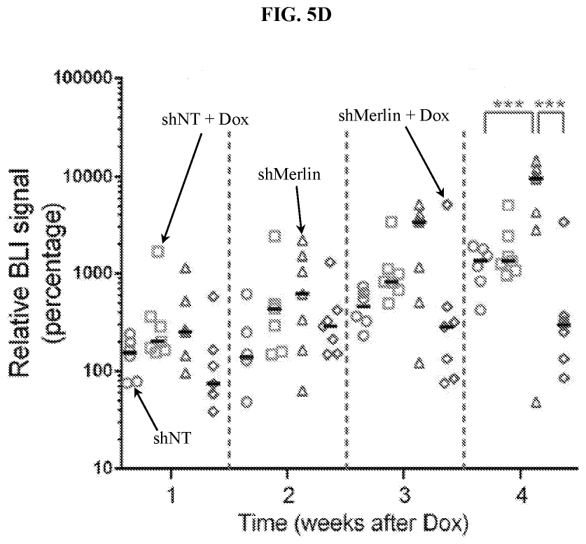

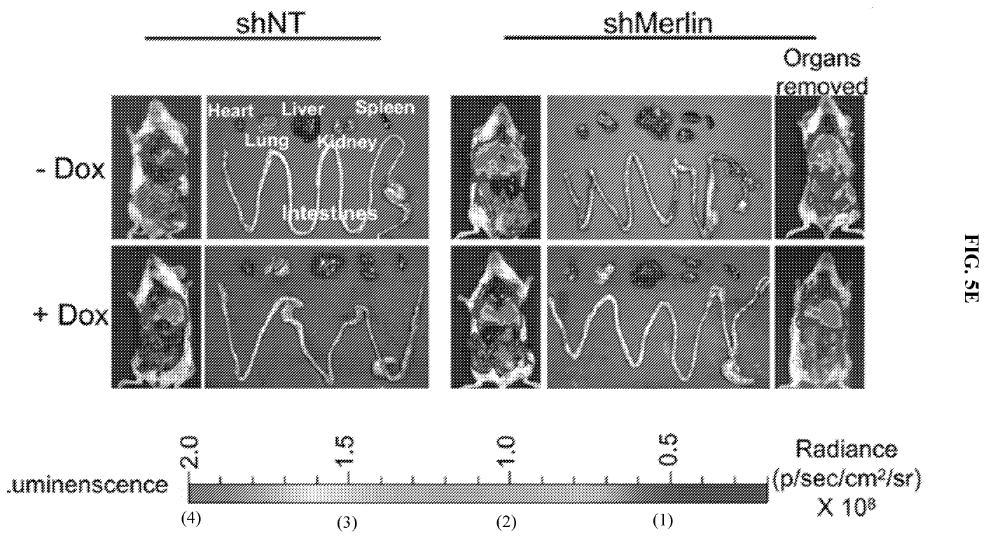

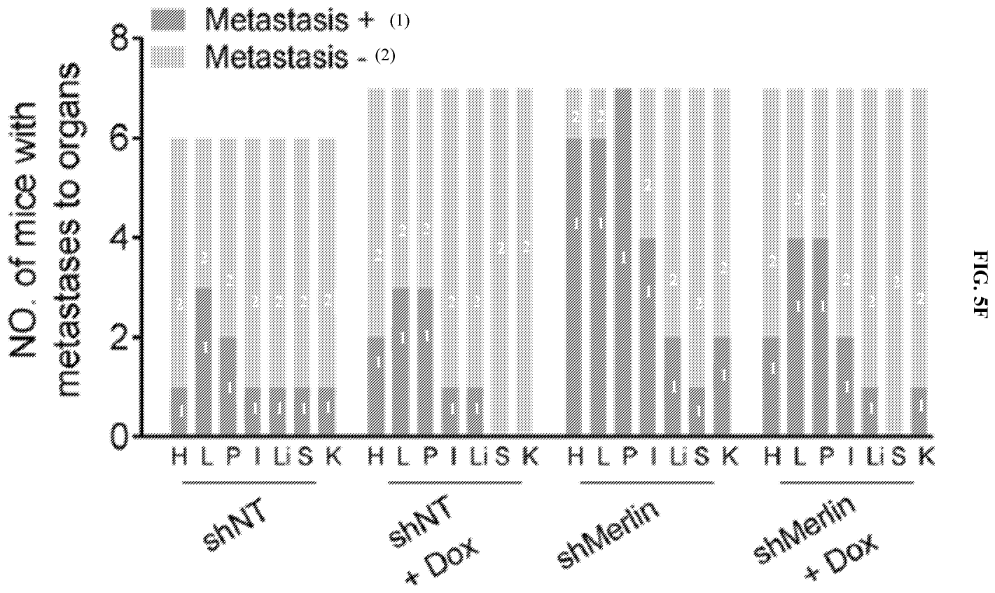

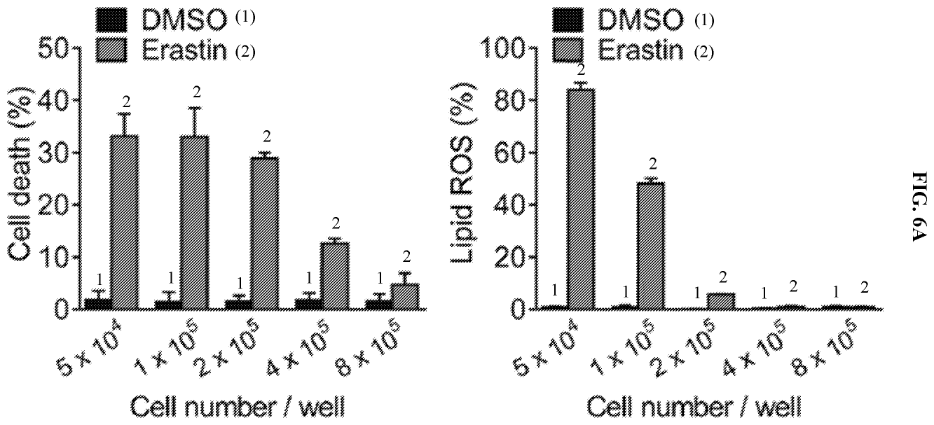

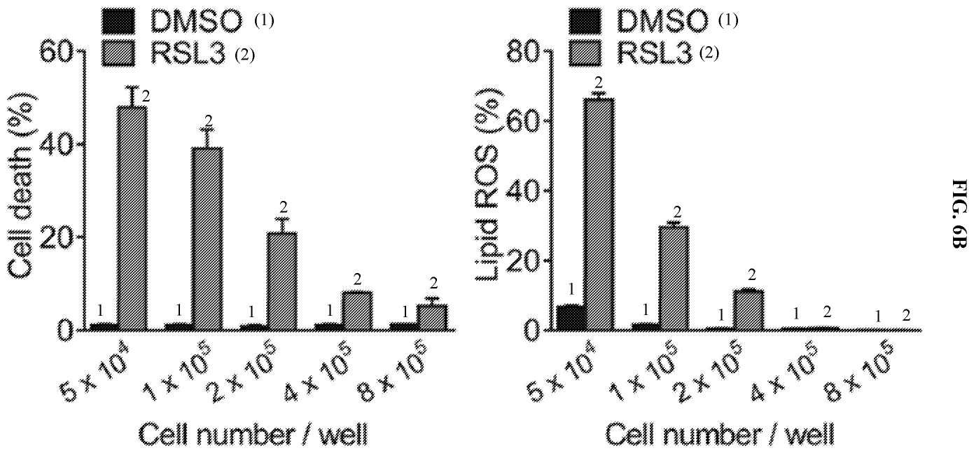

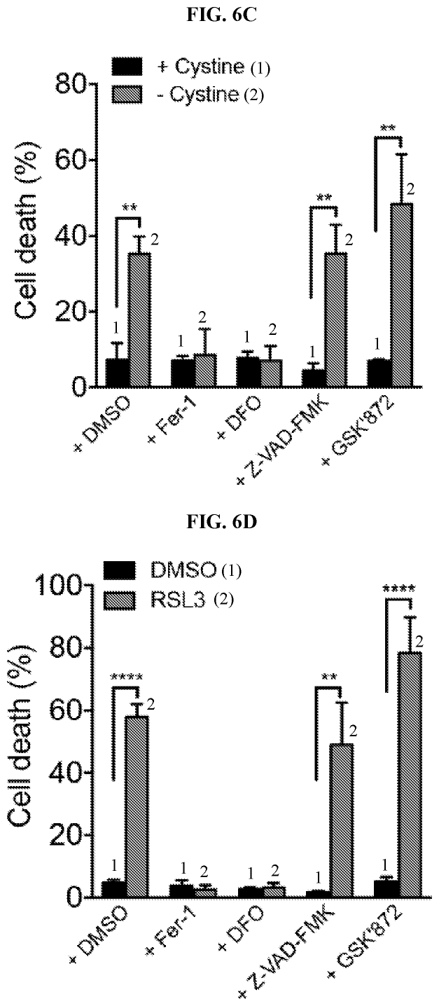

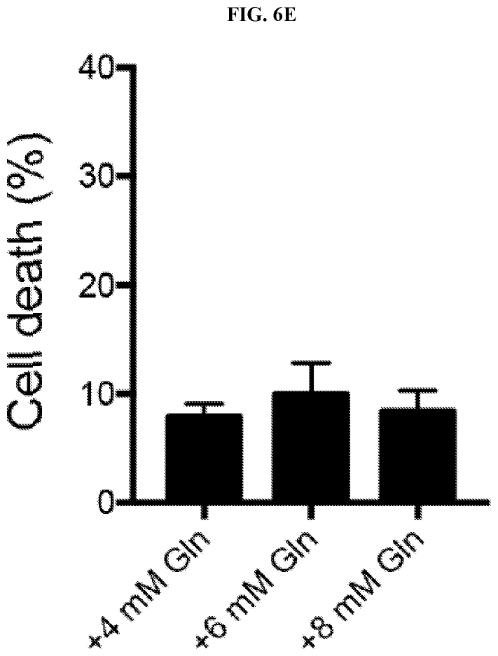

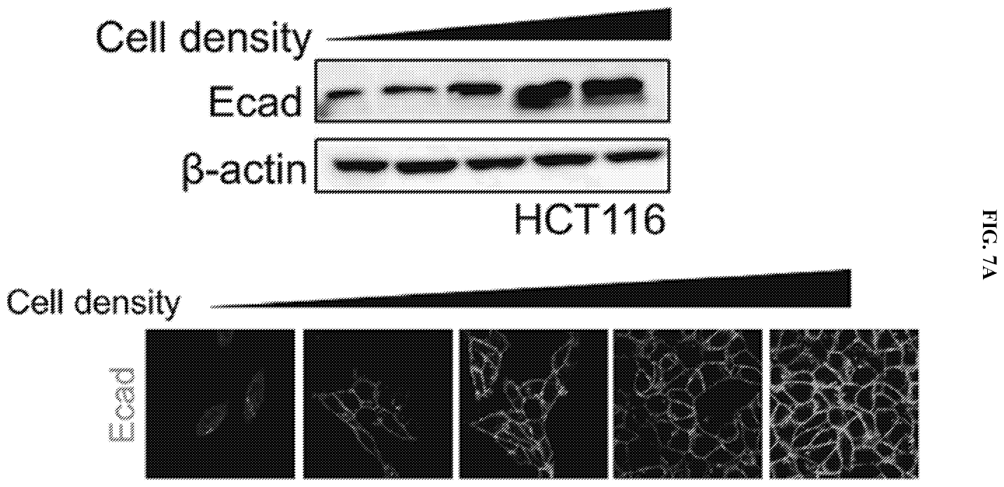

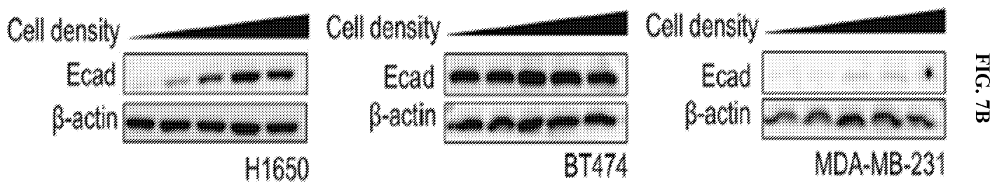

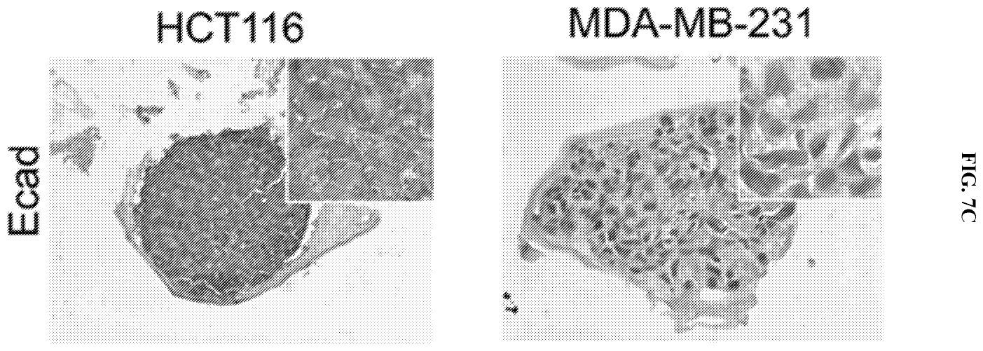

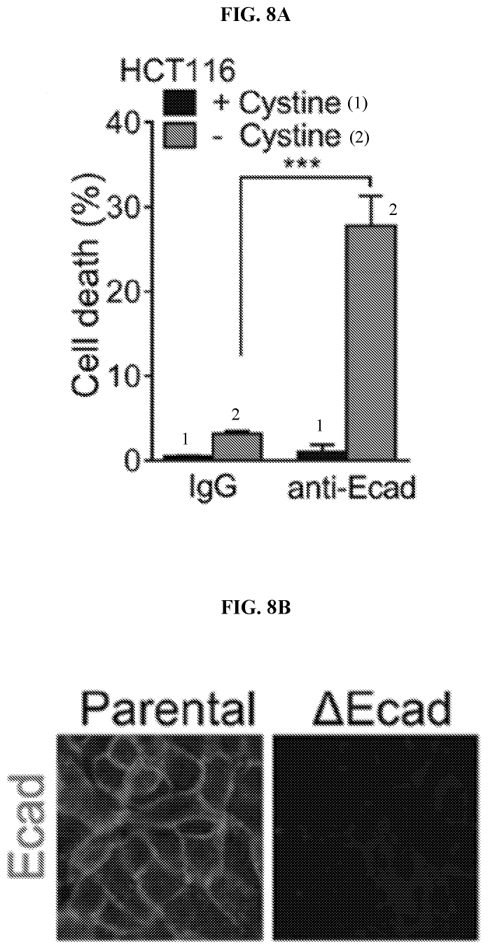

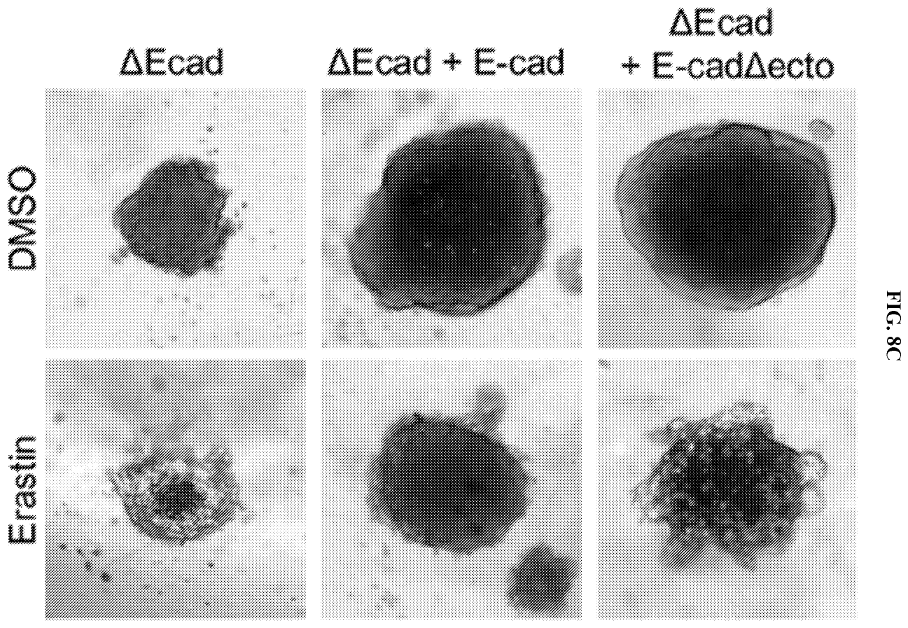

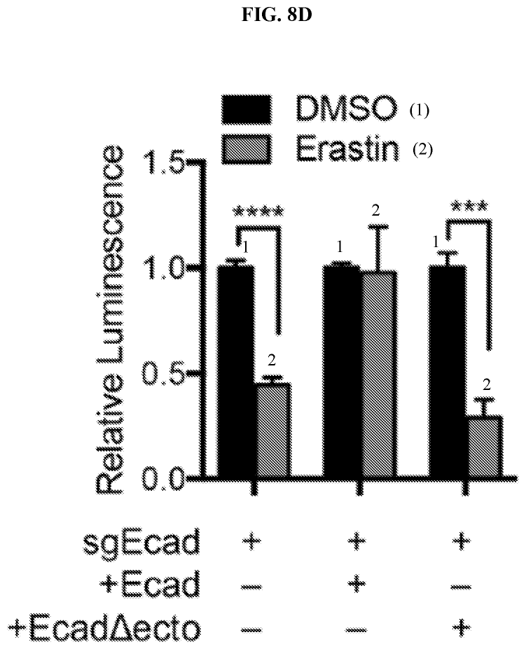

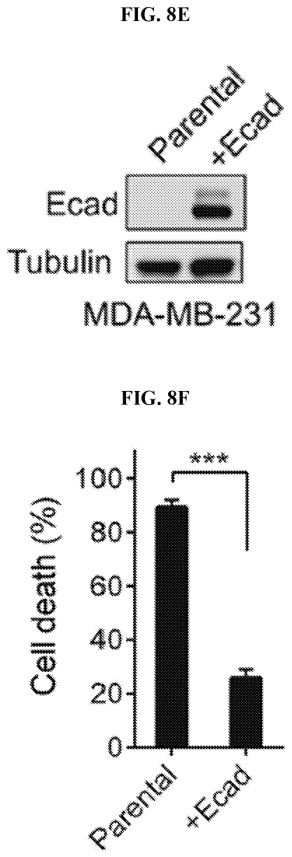

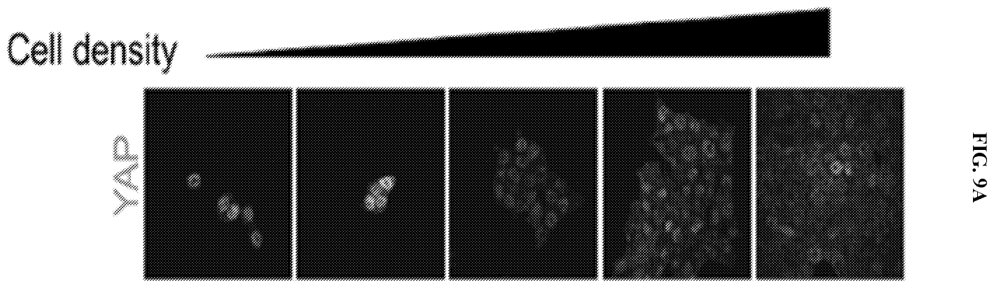

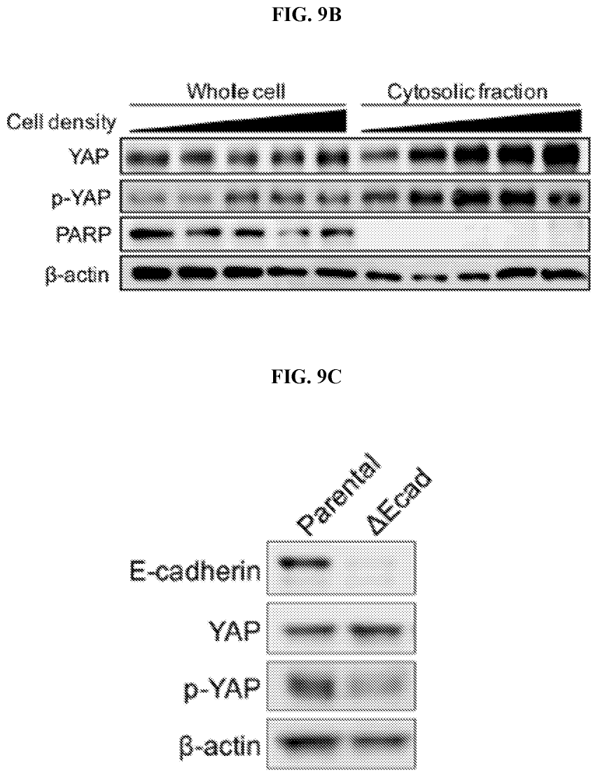

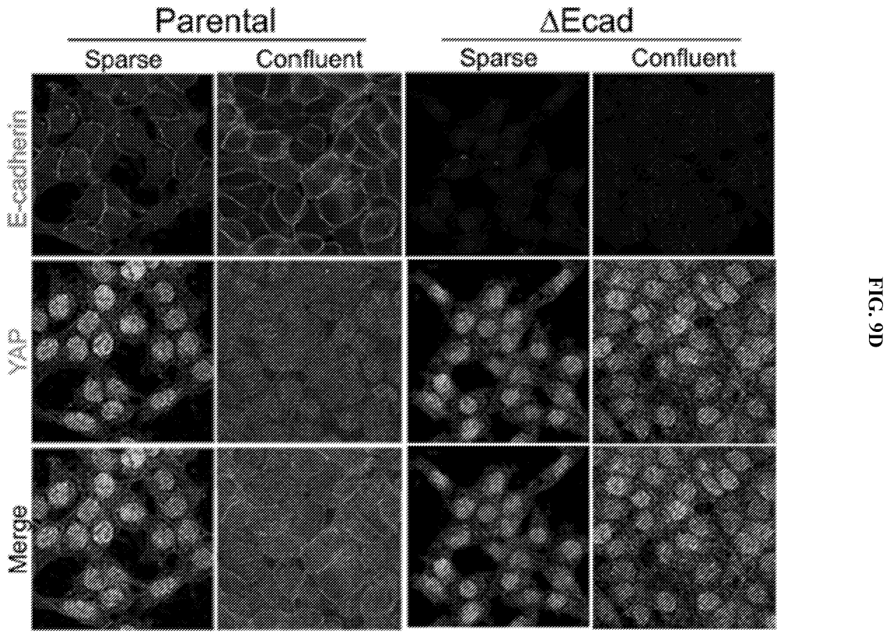

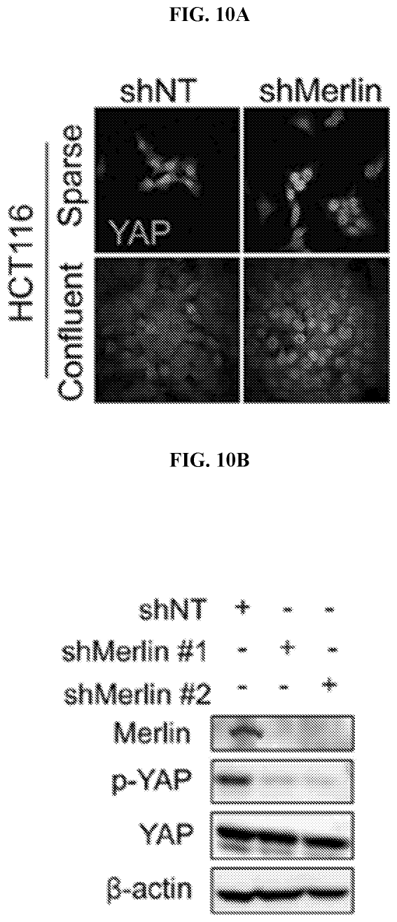

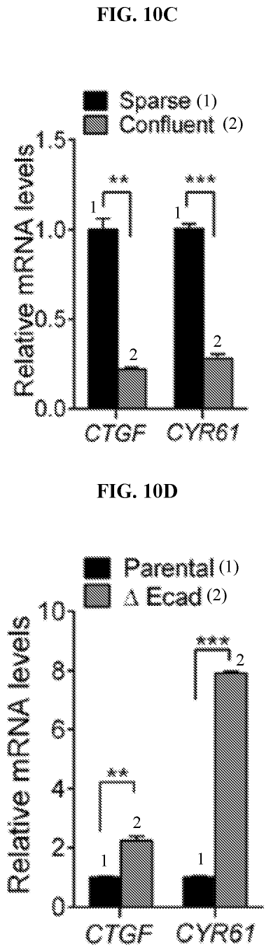

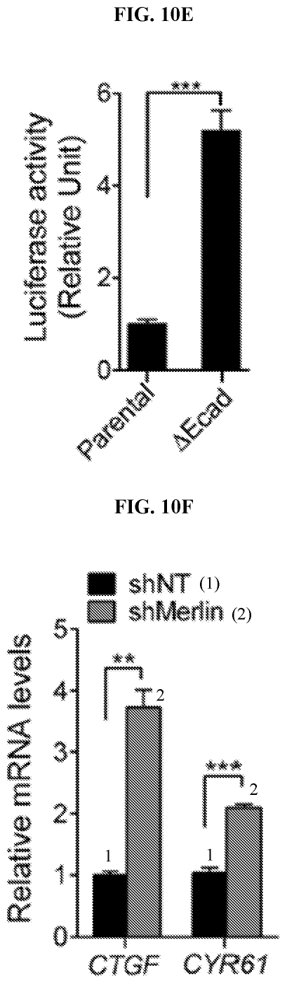

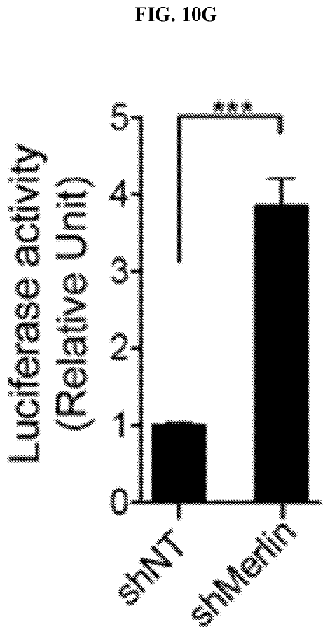

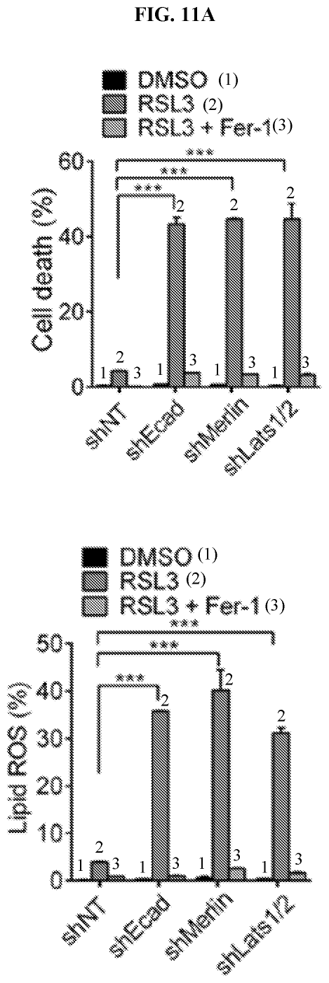

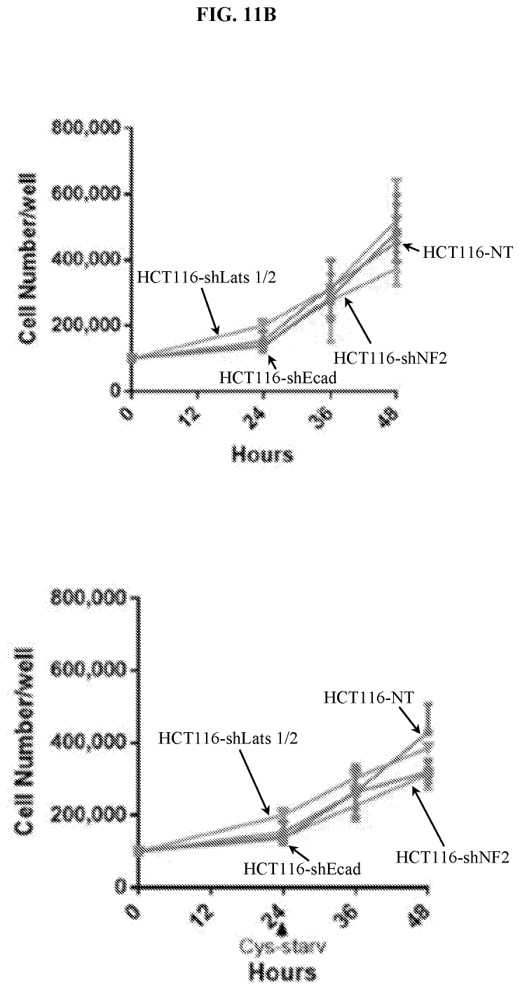

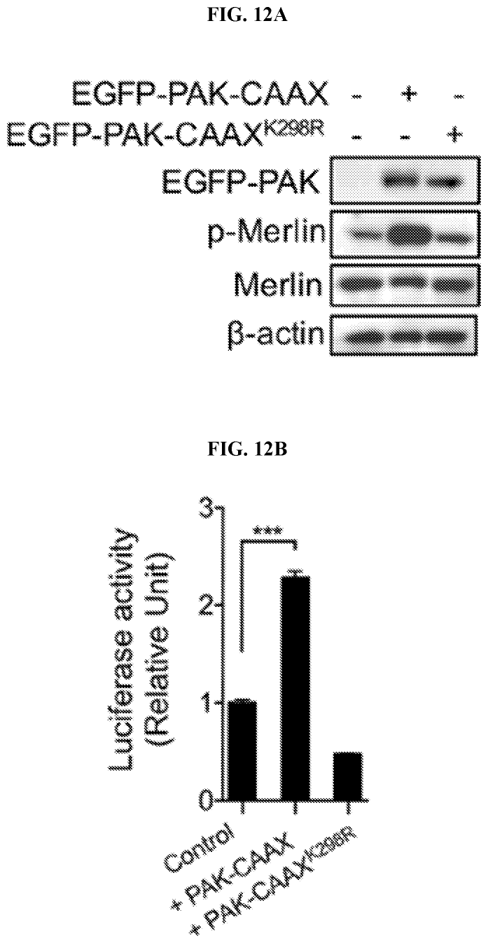

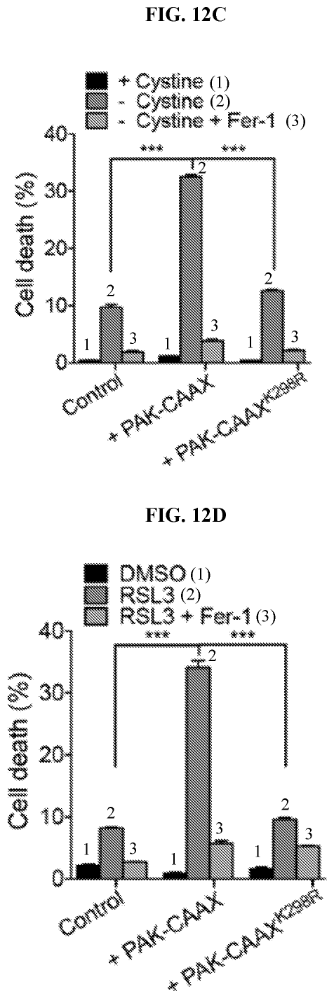

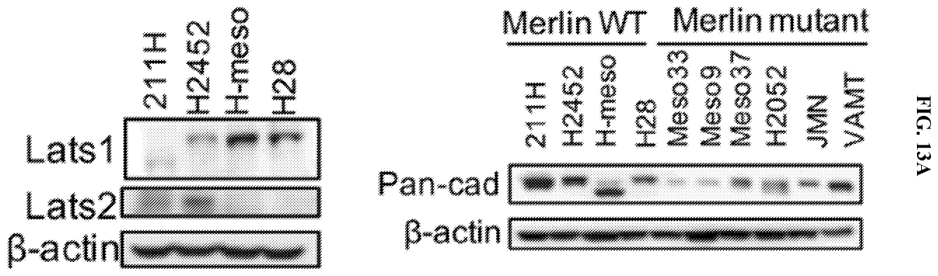

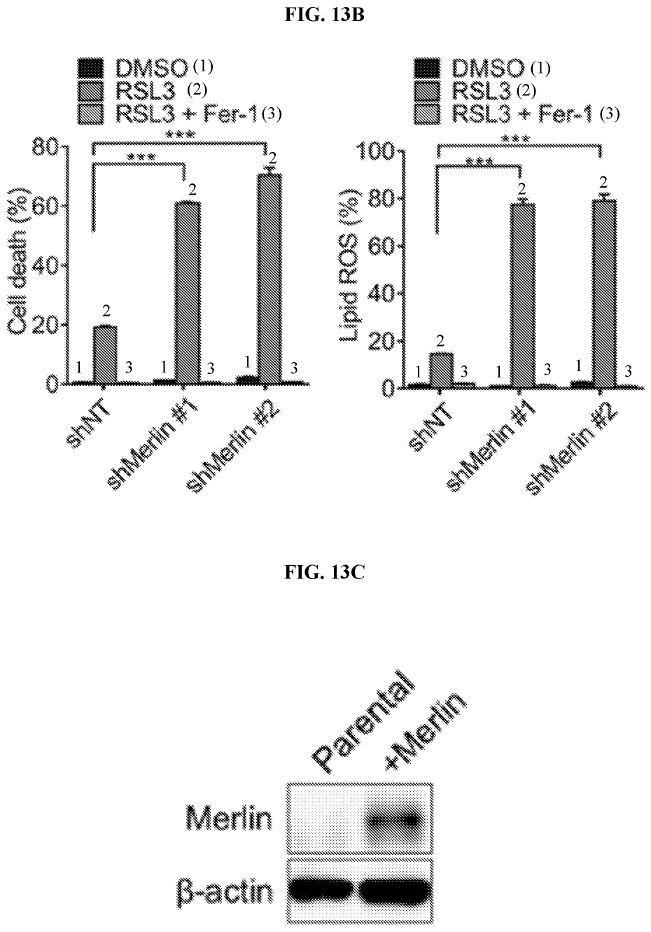

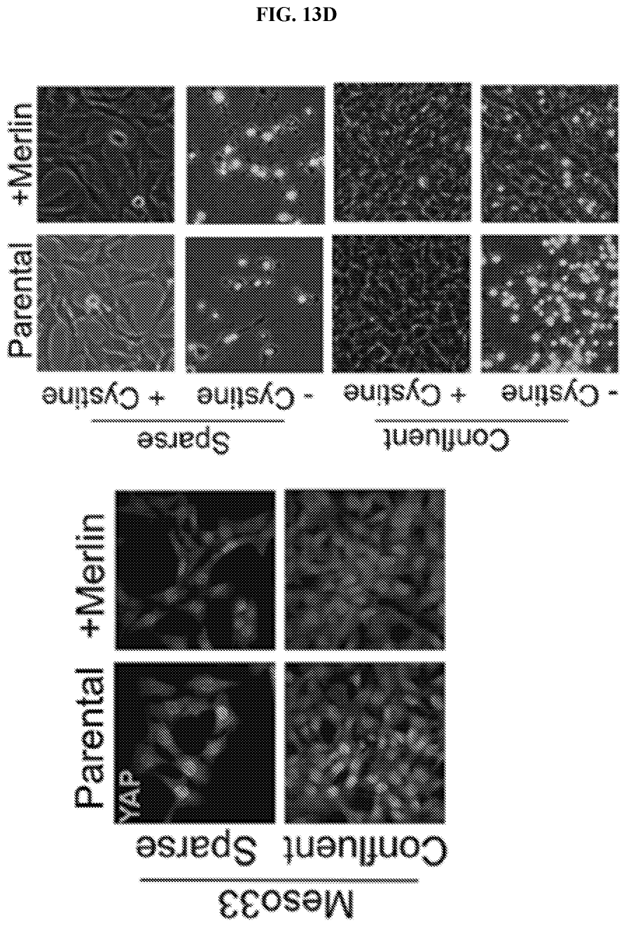

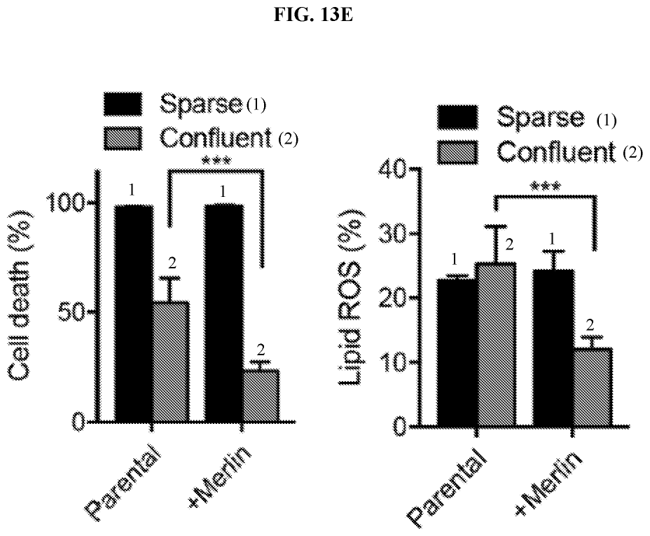

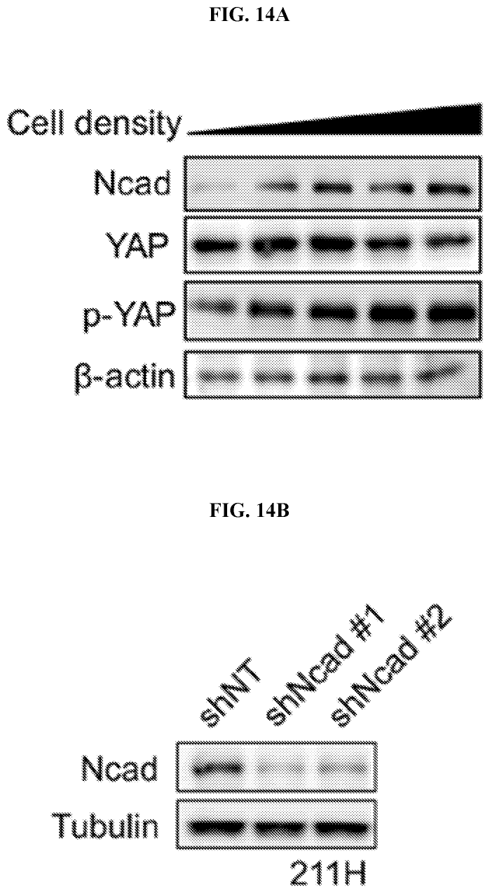

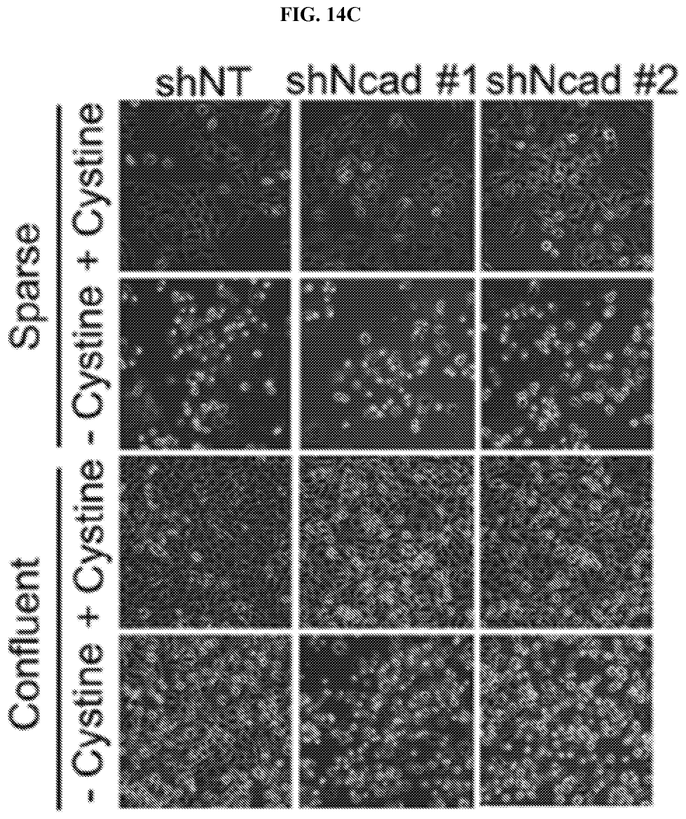

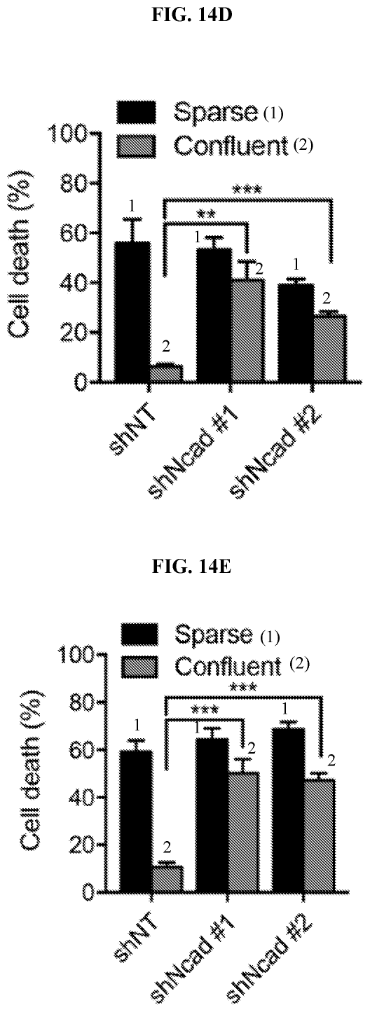

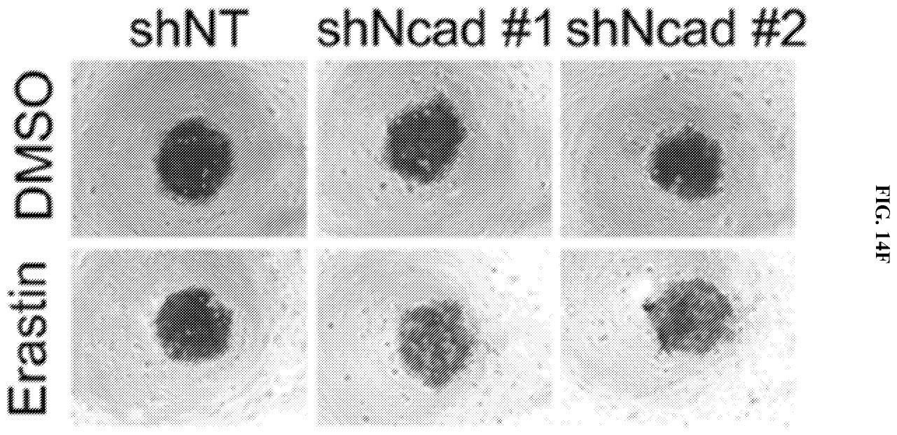

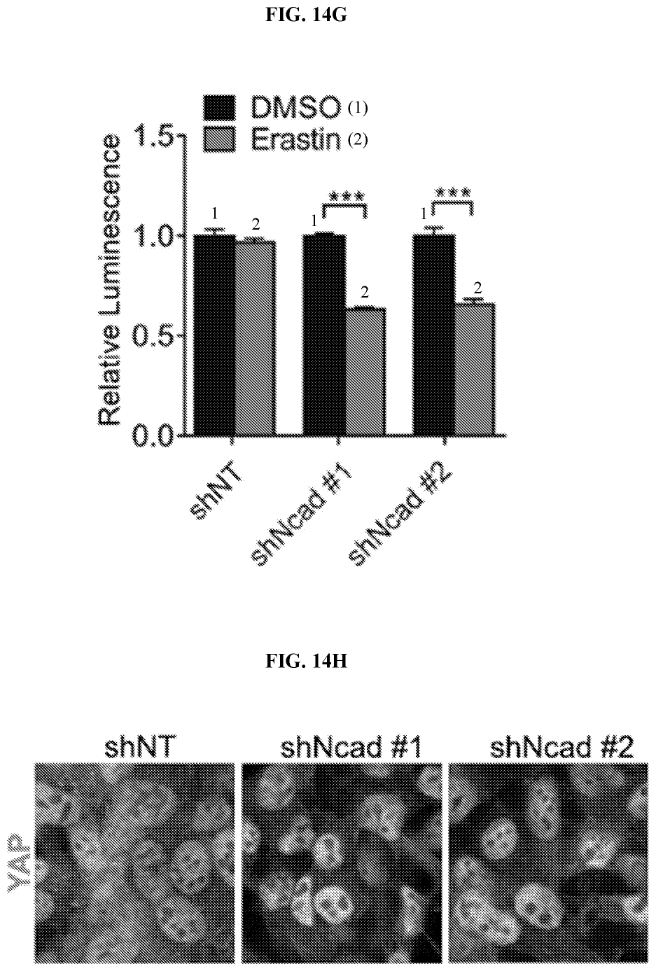

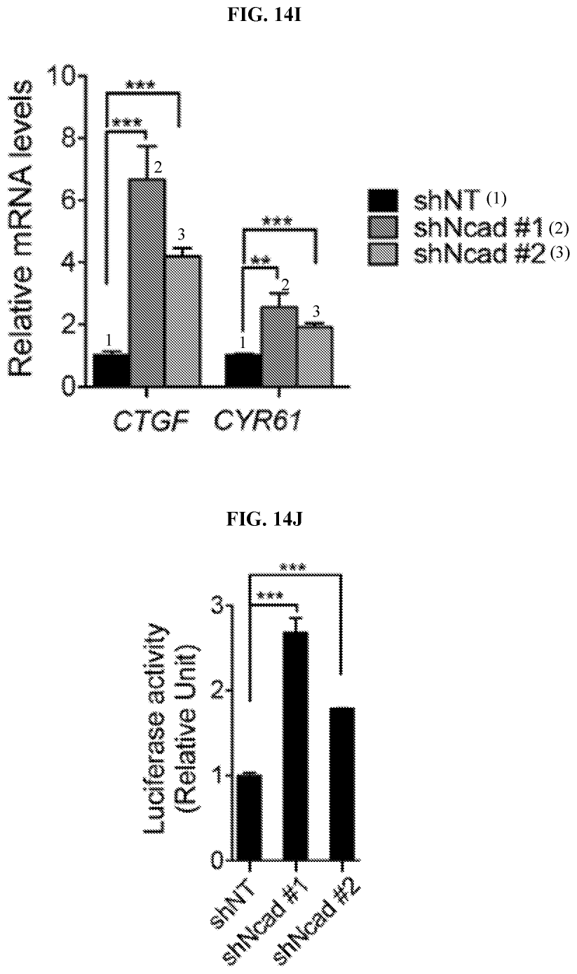

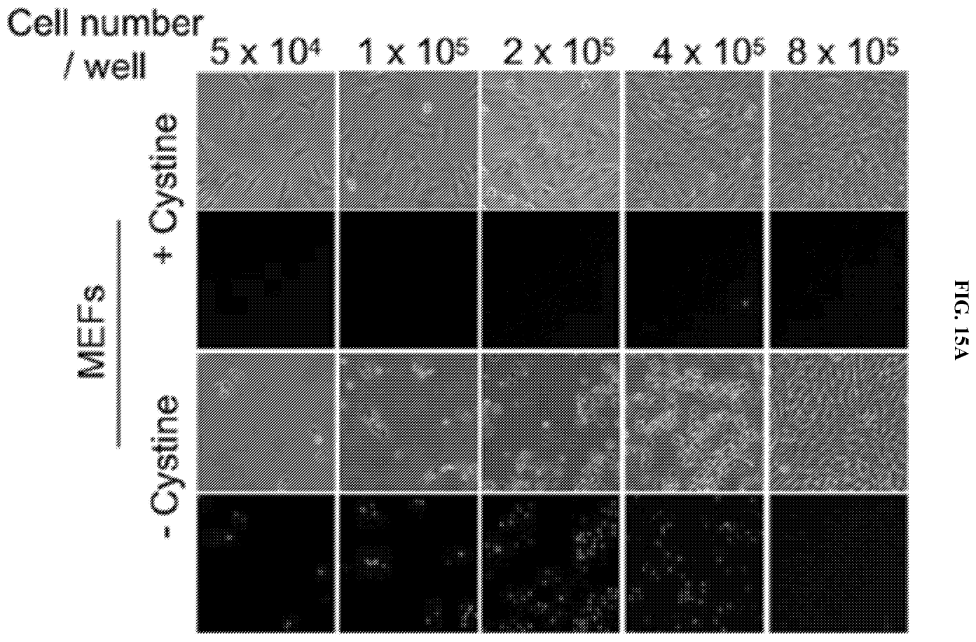

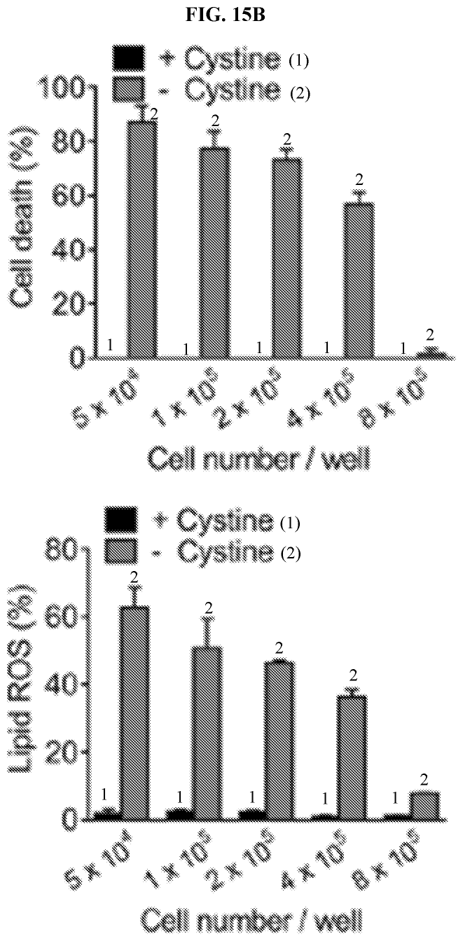

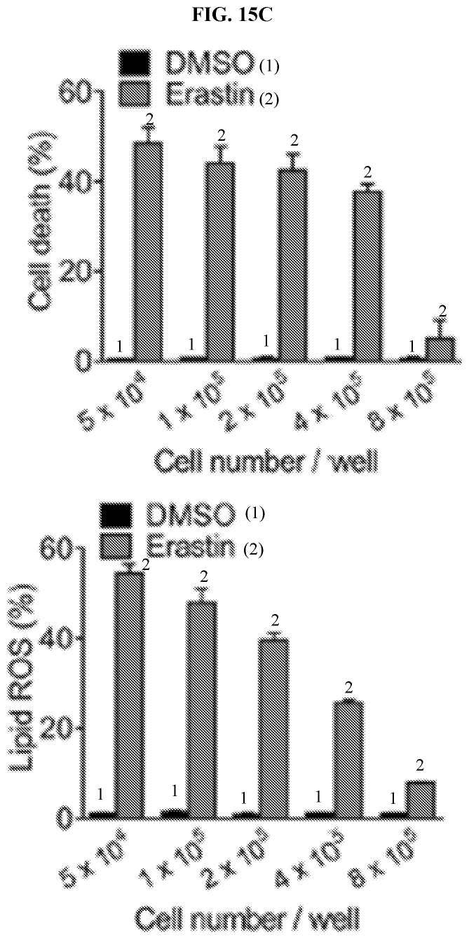

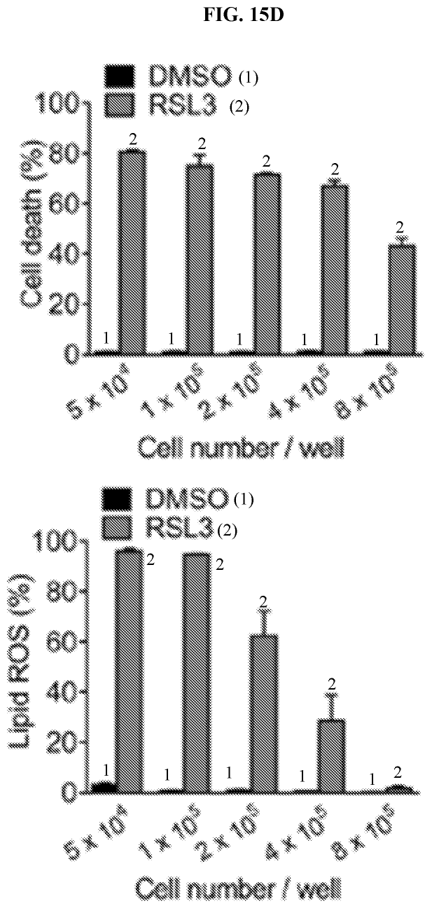

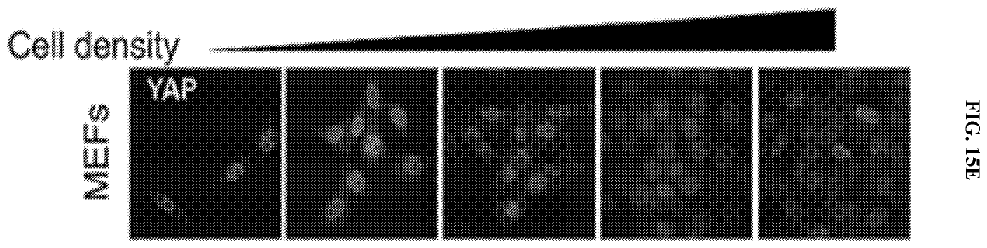

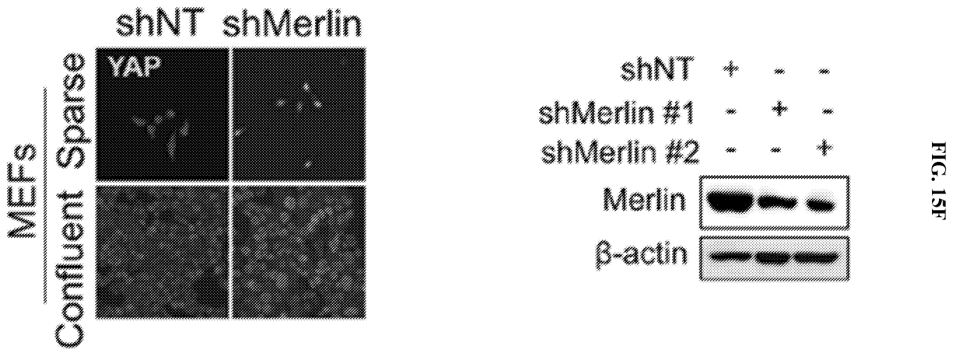

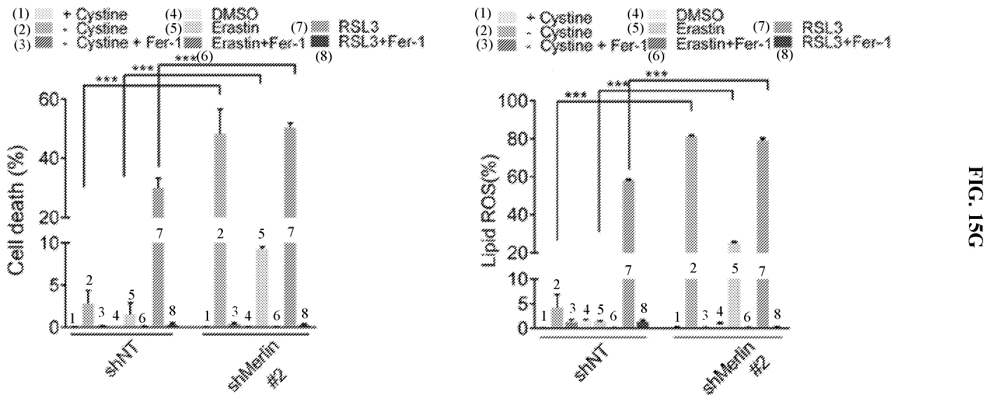

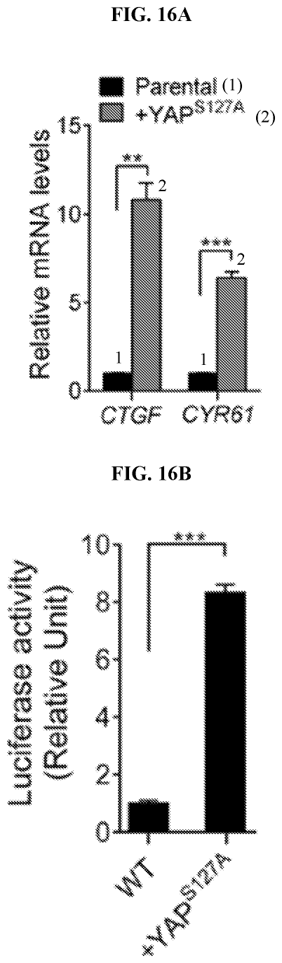

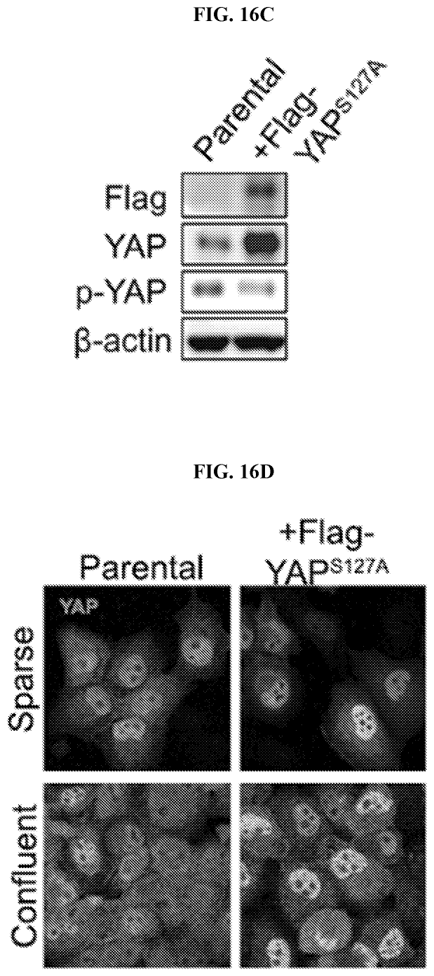

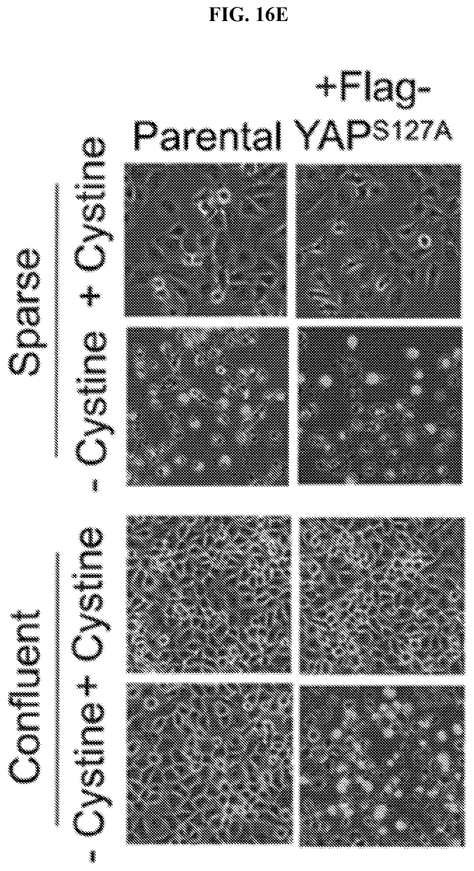

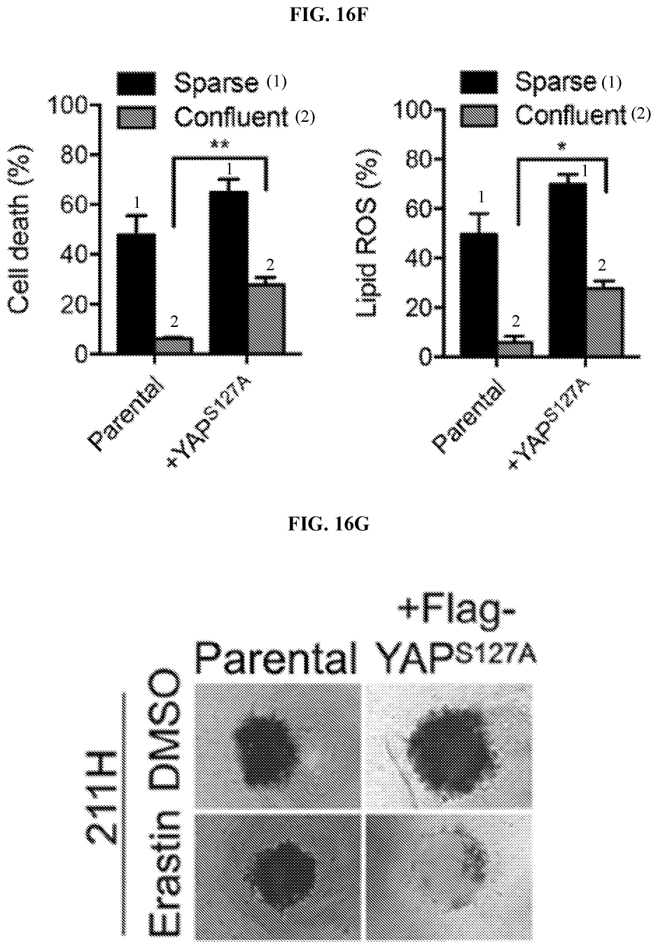

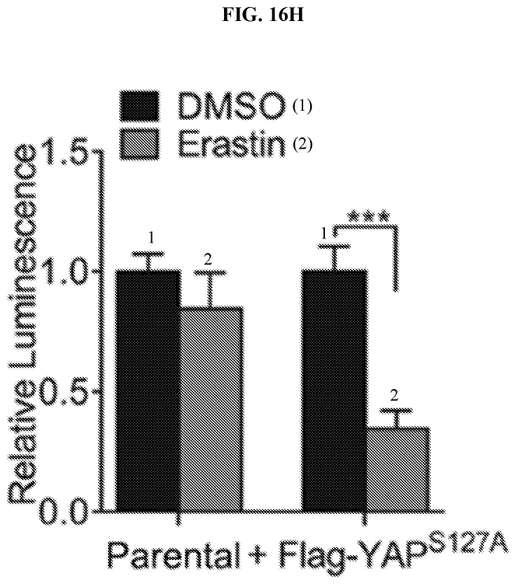

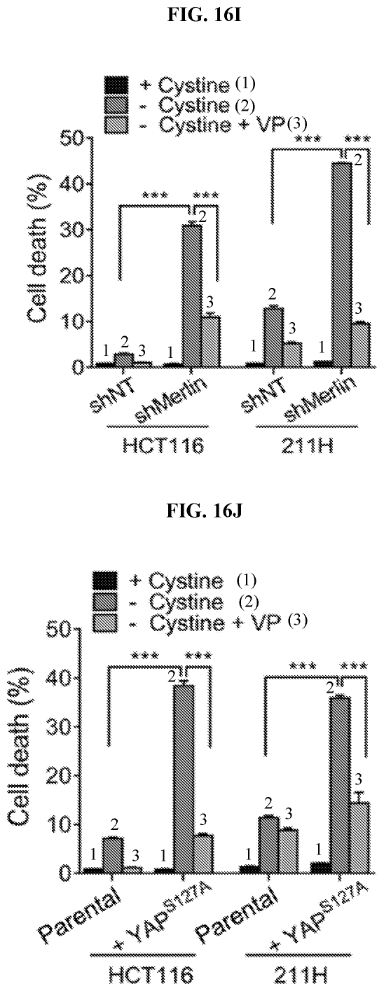

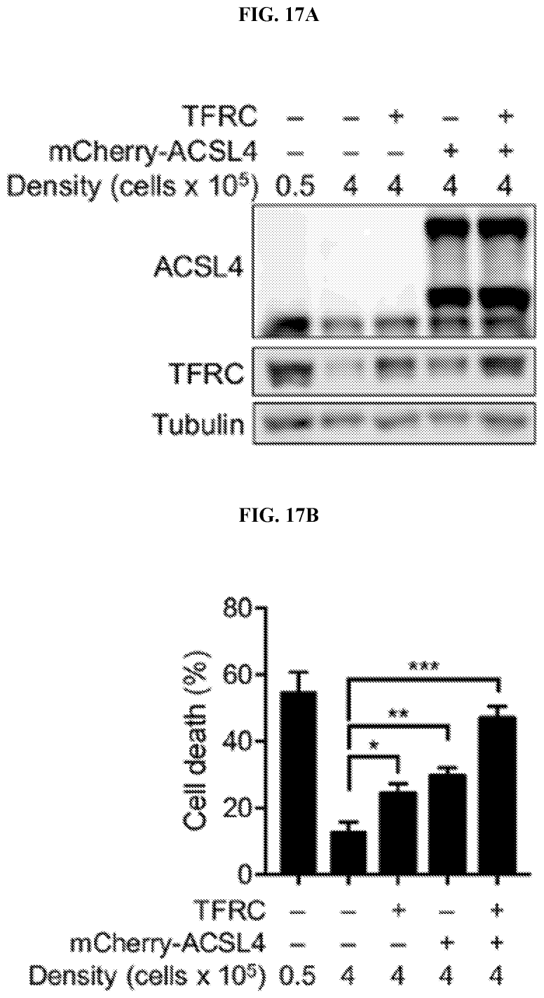

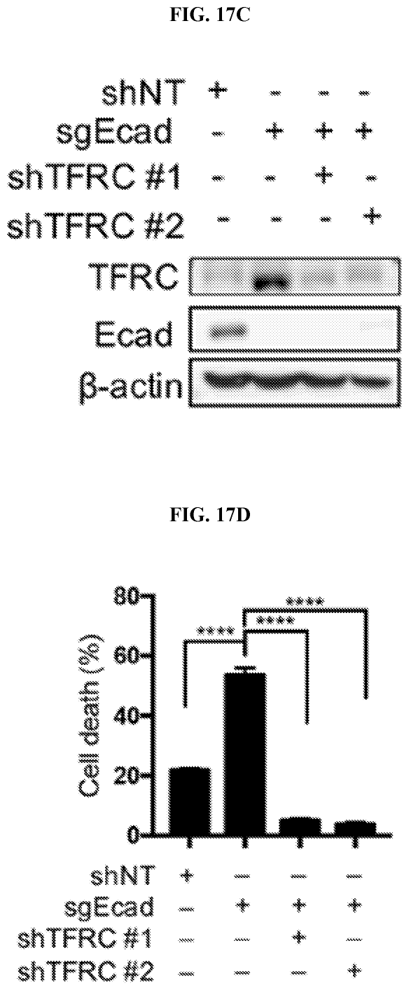

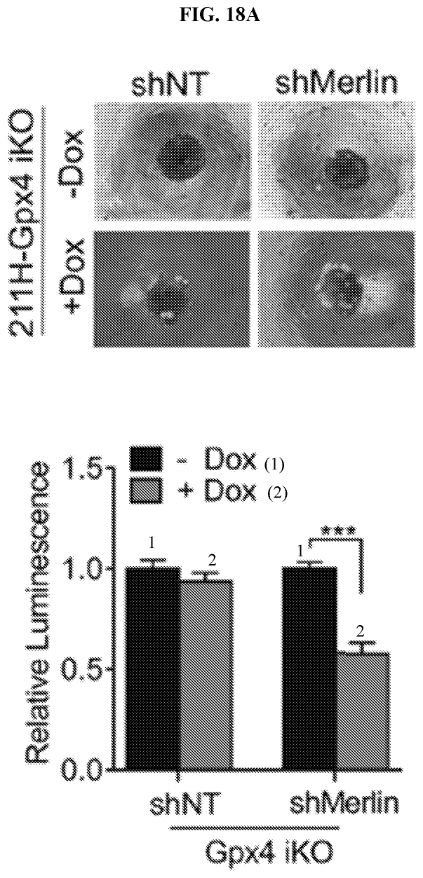

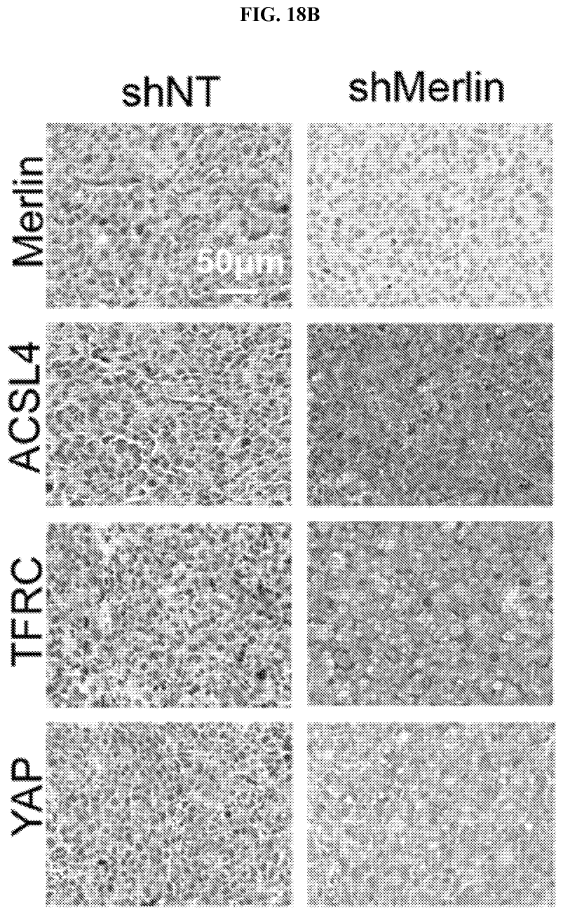

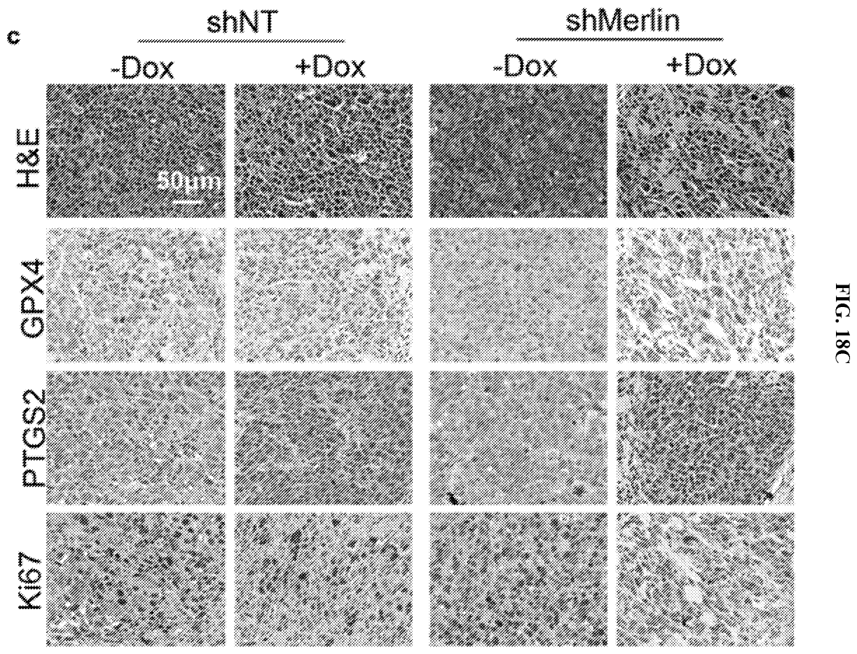

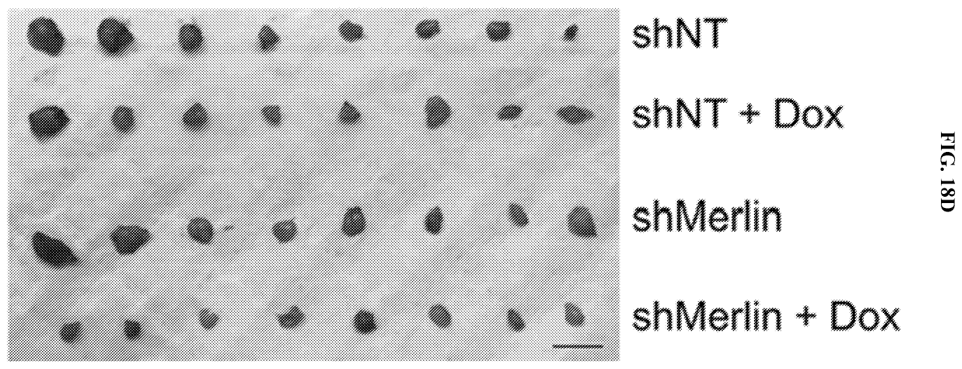

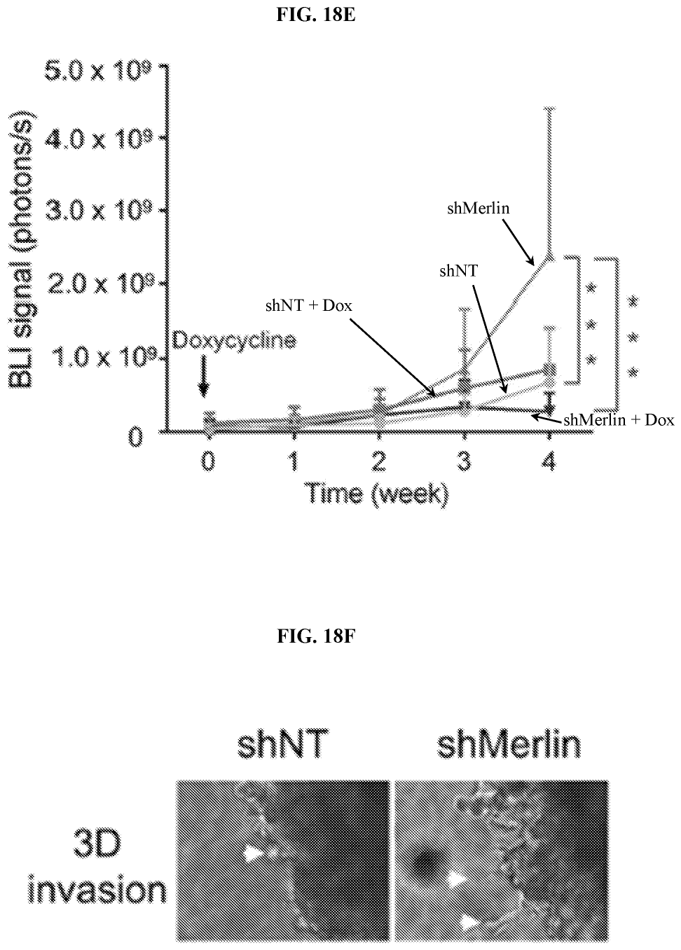

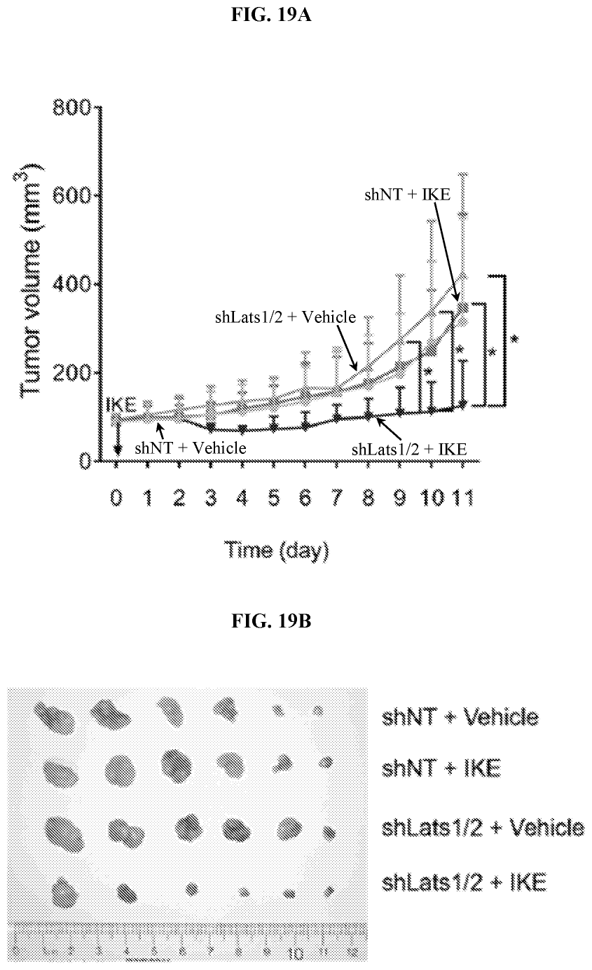

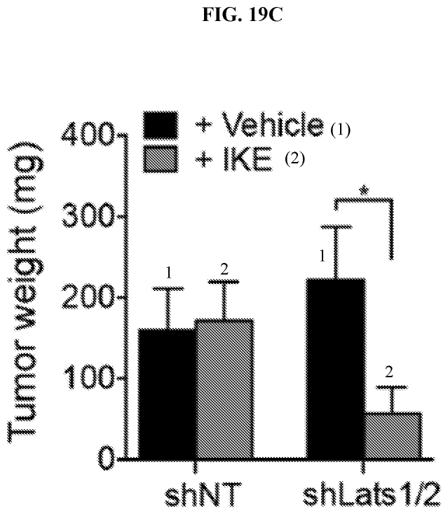

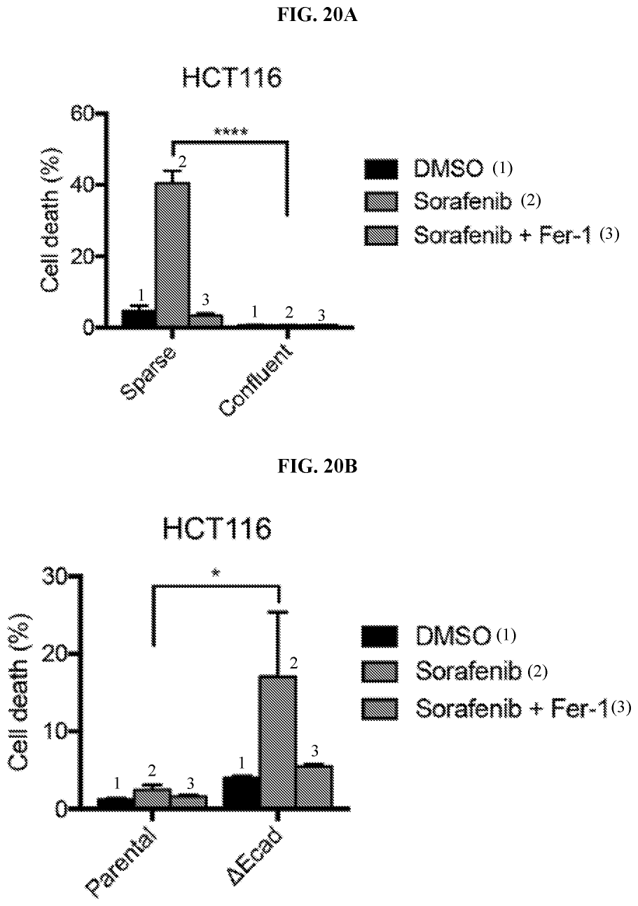

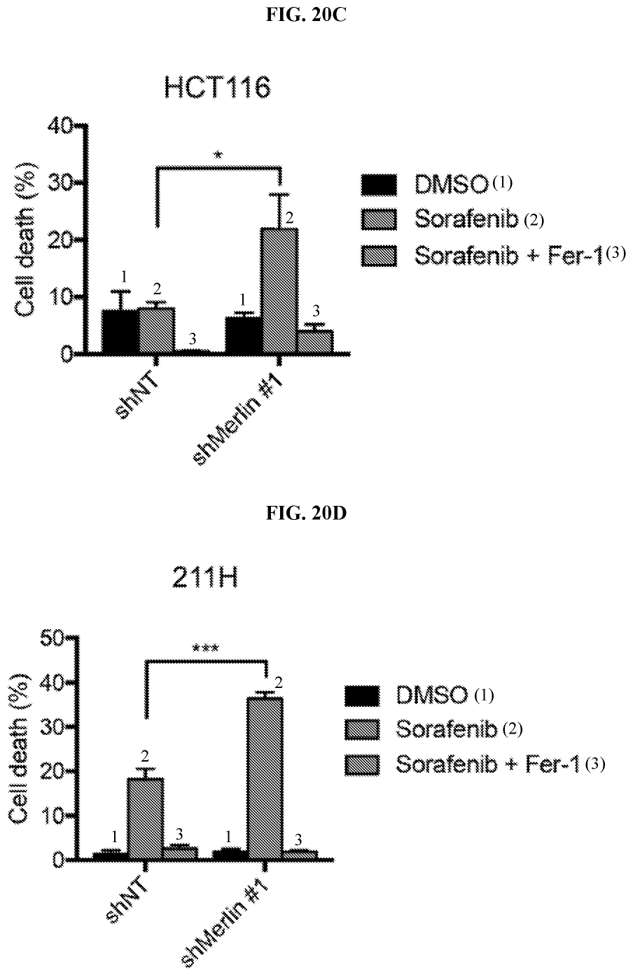

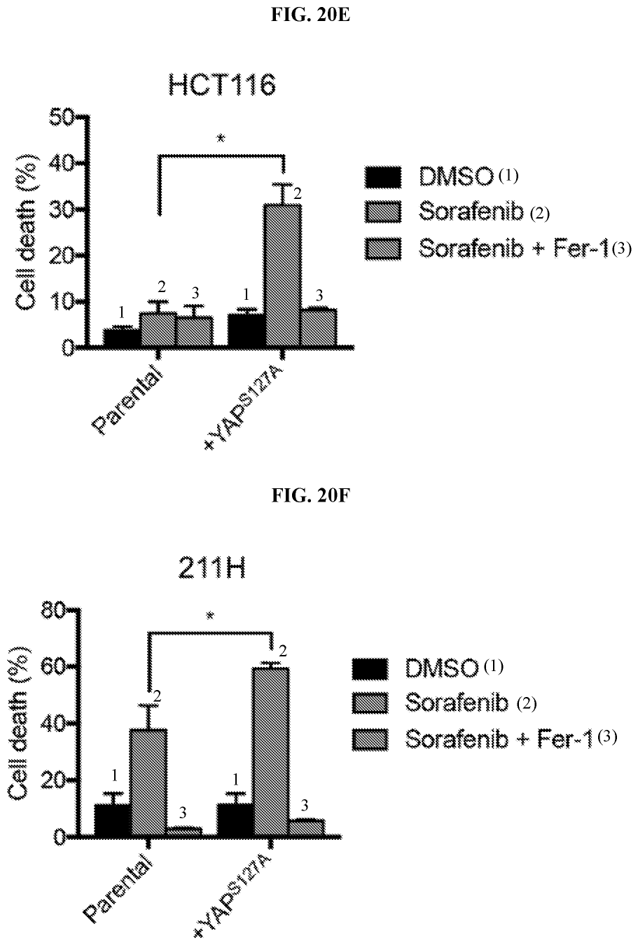

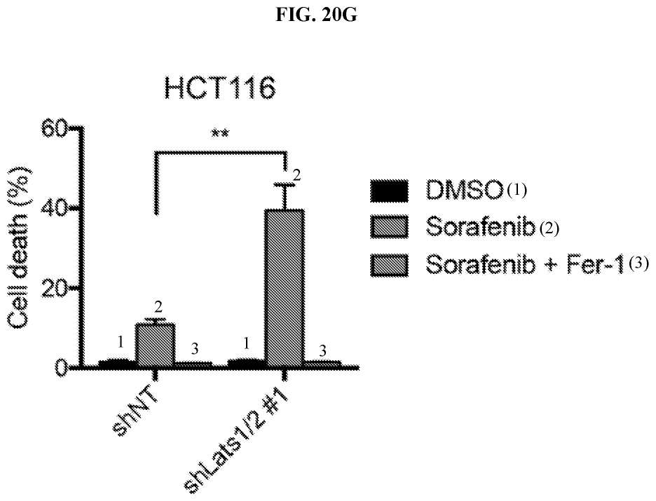

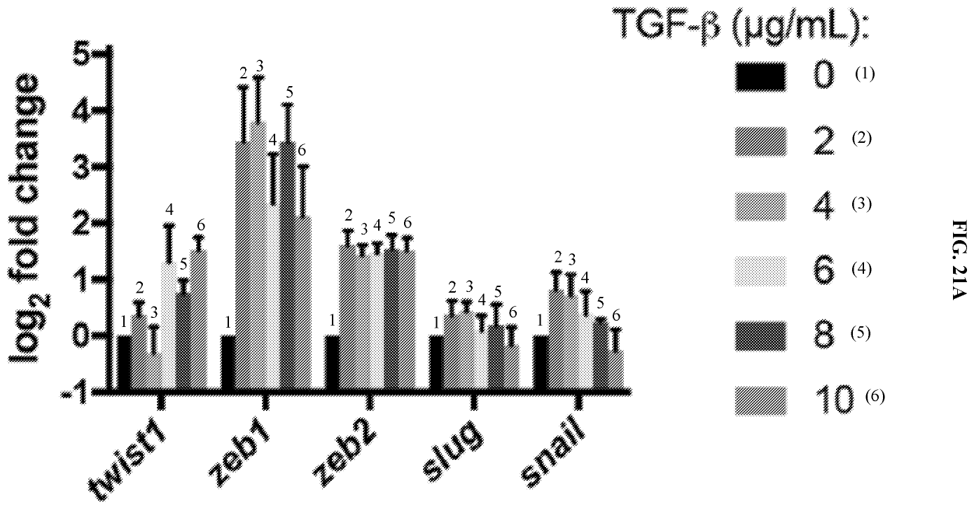

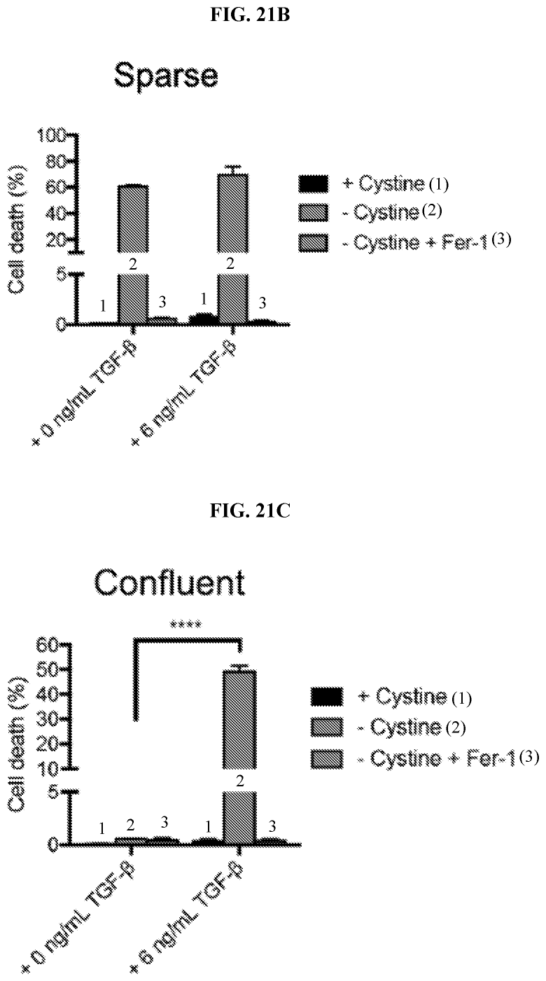

FIG. 1I shows an immunofluorescence and a bar graph illustrating that E-cadherin depleted HCT116 human colon cancer cells were hyper-sensitive to cystine-deprivation induced ferroptosis.

FIG. 1J shows an immunoblot confirming the expression of E-cadherin in cadherin depleted HCT116 cells rescued with either wild type E-cadherin or E-cadherin mutant lacking the ectodomain (EcadΔecto). The E-cadherin ectodomain is required for cadherin homomeric interaction.

FIG. 1K shows a bar graph illustrating that the re-expression of wild type E-cadherin but not the E-cadherin mutant lacking the ectodomain (EcadΔecto) in cadherin depleted HCT116 cells restored resistance to cystine deprivation induced ferroptosis.

FIG. 2A shows a schematic diagram illustrating the Cadherin-Hippo-YAP signaling pathway. In particular, E-cadherin signaling activates the tumor suppressor Merlin and a kinase cascade that activates Lats1/2, which phosphorylates the S127 residue of the pro-oncogenic transcription cofactor YAP. YAP phosphorylation decreases its nuclear localization and suppresses its function.

FIG. 2B shows an immunoblot illustrating the levels of RNAi knock-down of E-cadherin, Merlin, Lats 1, and Lats 2 in HCT116 cells.

FIG. 2C shows an immunofluorescence of cystine deprived ferroptosis of RNAi knock-down of E-cadherin, Merlin, Lats 1, and Lats 2 in confluent cultures of HCT116 cells.

FIG. 2D shows a bar graph illustrating that RNAi knock-down of E-cadherin, Merlin, Lats 1, and Lats 2 sensitized HCT116 cells to cystine-deprivation induced ferroptosis, and increased lipid Reactive Oxygen species (ROS) production upon cystine deprivation, both of which were inhibited by the ferroptosis inhibitor ferrostating (Fer-1).

FIG. 2E shows an immunofluorescence staining illustrating that RNAi knock-down of E-cadherin, Merlin, Lats 1, and Lats 2 in HCT116 cells enhanced erastin-induced ferroptosis in tumor spheroids generated from these cells; and that enhancement was inhibited by the ferroptosis inhibitor ferrostating (Fer-1).

FIG. 2F shows a bar graph demonstrating that inhibiting cystine import with erastin reduced the viability of tumor spheroids generated from RNAi knock-down of E-cadherin, Merlin, Lats 1, and Lats 2 in HCT116 cells; and that viability was rescued by inhibiting ferroptosis with ferrostating (Fer-1).

FIG. 3A shows an immunoblot demonstrating the expression of Merlin in four out of ten patient-derived malignant mesothelioma cell lines, and that some, but not all cells also expressed E-cadherin. In particular, the mesothelioma cell lines 211H, H2452, H-meso, and H28 expressed wild type Merlin, while merlin expression was undetectable in Meso33, Meso9, Meso37, H2082, JMN, and VAMT. E-cadherin was strongly expressed in H-meso and weakly expressed in H2082.

FIG. 3B shows a bar graph demonstrating that Merlin-wild type mesothelioma cell lines underwent cystine deprivation induced ferroptosis and were resistant to ferroptosis at high density; whereas some Merlin-mutant mesothelioma cell lines were hypersensitive to cystine deprivation induced ferroptosis even at high density.

FIG. 3C shows a bar graph demonstrating the percentage mesothelioma cell lines with a strong or weak response to density-dependent regulation of ferroptosis. In particular, 100 percent of Merlin-wild type and less than 40% of Merlin-mutant mesothelioma cell lines had a strong response.

FIG. 3D shows a fluorescence staining demonstrating that tumor spheroids generated from Merlin-wt mesothelioma cell lines were resistant to erastin-induced ferroptosis, whereas those generated from Merlin-mutant mesothelioma cell lines were sensitive.

FIG. 3E shows a bar graph demonstrating that tumor spheroids generated from Merlin-mutant mesothelioma cell lines, but not those generated from Merlin-wt mesothelioma cell lines had reduced viability following erastin-treatment.

FIG. 3F shows an immunoblot illustrating the levels of Merlin knockdown following Merlin RNAi treatment of a Merlin-wt mesothelioma cell line (MSTO-211H).

FIG. 3G shows a bar graph illustrating that highly confluent Merlin-wt MSTO-211H mesothelioma cells became sensitive to cystine deprivation induced ferroptosis following Merlin RNAi knockdown; and the effect was blocked by the ferroptosis inhibitor fer-1.

FIG. 311 shows a bar graph illustrating that the production of lipid reactive oxygen species was enhanced in highly confluent Merlin-wt MSTO-211H mesothelioma cells that were deprived of cystine and treated with Merlin RNAi; and that the effect was blocked by the ferroptosis inhibitor fer-1.

FIG. 31 shows an immunoblot illustrating the level of Merlin in Merlin-mutant Meso33 mesothelioma cells transfected with a doxycycline-inducible Merlin construct following doxycycline treatment.

FIG. 3J shows a bar graph illustrating that doxycycline-induced Merlin expression in Merlin-mutant Meso33 mesothelioma cells transfected with a doxycycline-inducible Merlin construct restored these cells ferroptosis resistance at high density; and that the effect was blocked by the ferroptosis inhibitor fer-1.

FIG. 3K shows a fluorescence staining illustrating that tumor spheroids generated from Merlin-mutant Meso33 mesothelioma cells expressing a doxycycline-inducible Merlin construct were resistant to erastin-induced ferroptosis following doxycycline treatment, and that the effect was blocked by the ferroptosis inhibitor fer-1.

FIG. 3L shows a bar graph illustrating that doxycycline induction of Merlin expression suppressed the erastin-induced cell death within tumor spheroids generated from Merlin-mutant Meso33 mesothelioma cells expressing a doxycycline-inducible Merlin construct.

FIG. 4A shows an immunoblot illustrating the levels of YAP^{S127}, YAP, and phospho-YAP in HCT116 human colon cancer cells expressing the YAP^{S127} mutant.

FIG. 4B shows an immunofluorescent staining illustrating the subcellular localization of YAP in HCT116 human colon cancer cells expressing the YAP^{S127} mutant. In particular, YAP was localized in the nucleus even when cells were cultured at a high density.

FIG. 4C shows a bar graph illustrating that HCT116 human colon cancer cells overexpressing the YAP^{S127} mutants were sensitive to cystine deprivation induced ferroptosis at high cell density.

FIG. 4D shows a bar graph illustrating the enhanced production of lipid reactive oxygen species in HCT116 human colon cancer cells ectopically expressing the YAP^{S127} mutants.

FIG. 4E shows an immunofluorescence staining illustrating that tumor spheroids generated from HCT116 human colon cancer cells overexpressing the YAP^{S127} mutant were sensitive to erastin-induced ferroptosis; and further shows a bar graph illustrating reduced cell viability within tumor spheroids generated from HCT116 human colon cancer cells overexpressing the YAP^{S127} mutant following erastin-induced ferroptosis.

FIG. 4F shows an immunoblot illustrating the levels of Merlin and YAP in YAP knockout and Merlin RNAi treated HCT116 human colon cancer cells.

FIG. 4G shows two bar graphs illustrating that YAP knockout abrogated Merlin RNAi-induced sensitization of HCT116 human colon cancer cells to cystine-deprivation induced ferroptosis. In particular, YAP knockout and Merlin RNAi treated HCT116 human colon cancer cells were resistant to cystine-deprivation induced ferroptosis, and these YAP-Merlin double mutant cells also produced low levels of lipid reactive oxygen compare to either single mutant.

FIG. 411 shows six immunoblots illustrating the expression levels of two YAP-TEAD gene targets, the transferrin receptor (TFRC) and the acyl-CoA synthase long chain family member 4 (ACSL4) in HCT116 human colon cancer cells and Merlin-wt MSTO-211H mesothelioma cells. In particular, the levels of TFRC and ACSL4 decreased with enhanced cell density; their levels also were upregulated in E-cadherin depleted cells, Merlin RNAi treated cells, and cells overexpressing the constitutively active YAP^{S127} mutant.

FIG. 41 shows a bar graph illustrating the quantification of a Chromatin Immunoprecipitation (ChIP) assay demonstrating the binding of the transcription factor TEAD4 to the promoters region of the acyl-CoA synthase long chain family member 4 (ACSL4) and the transferrin receptor (TFRC) in Merlin-wt MSTO-211H mesothelioma cells.

FIG. 4J shows a bar graph illustrating the quantification of a Chromatin Immunoprecipitation (ChIP) assay demonstrating that the binding of TEAD4 to the promoters region of the transferrin receptor (TFRC) and the acyl-CoA synthase long chain family member 4 (ACSL4) was stimulated by 3 to 4 fold in Merlin-wt MSTO-211H mesothelioma cells overexpressing the constitutively active YAP^{S127} mutant.

FIG. 4K shows an immunoblot illustrating the expression levels of Merlin and the transferrin receptor in Merlin RNAi treated and transferrin receptor depleted HCT116 human colon cancer cells.

FIG. 4L shows a bar graph illustrating that transferrin receptor RNAi abrogated the sensitivity of Merlin RNAi treated HCT116 human colon cancer cells to cystine-deprivation induced ferroptosis.

FIG. 4M shows an immunoblot illustrating the expression levels of Merlin and the acyl-CoA synthase long chain family member 4 (ACSL4) in Merlin RNAi and ACSL4 depleted HCT116 human colon cancer cells.

FIG. 4N shows a bar graph illustrating that acyl-CoA synthase long chain family member 4 (ACSL4) knockout abrogated the sensitivity of Merlin RNAi treated HCT116 human colon cancer cells to cystine-deprivation induced ferroptosis.

FIG. 5A shows the expression of GPX4, Cas9, and Merlin in Merlin-wt MSTO-211H mesothelioma cells expressing doxycycline-inducible CRISPR/Cas9-mediated GPX4 knockout (Gpx4-iKO) and Gpx4-iKO harboring control and Merlin RNAi.

FIG. 5B shows a graph illustrating the growth curve of xenograft tumors generated from Gpx4-iKO and Merlin RNAi-Gpx4-iKO cells subcutaneously injected in nude mice fed with normal or doxycycline diet. In particular, Merlin RNAi-Gpx4-iKO generated xenograft tumors receded upon Gpx4 knockout induction with doxycycline, while the growth of Gpx4-iKO generated xenograft tumors were statistically reduced.

FIG. 5C shows that representative bioluminescent imaging illustrating the growth of tumors generated from Gpx4-iKO and Merlin RNAi-Gpx4-iKO cells harboring a retroviral TK-GFP-Luciferase reporter in orthotopic intrapleural mouse model of mesothelioma in NOD/SCID mice. In particular, tumor generated from Merlin RNAi-Gpx4-iKO grew more aggressively than Gpx4-iKO tumors. However, Gpx4 knockout induction with doxycycline reduced the growth of Merlin RNAi-Gpx4-iKO tumors, but had no effect on the growth of Gpx4-iKO tumors.

FIG. 5D shows a dot plot quantifying the bioluminescent imaging signal from tumors generated from Gpx4-iKO and Merlin RNAi-Gpx4-iKO cells as described in FIG. 5C.

FIG. 5E shows the bioluminescent imaging signal of tumors in various organs before (whole mice) and after animals were sacrificed (excised organs). In particular, Gpx4-iKO tumors grew in the pleural cavity, attaching to the aortic arch, lung or thoracic muscles, whereas Merlin RNAi-Gpx4-iKO tumors metastasized to pericardium, peritoneum, abdominal organs including liver, intestine and distal lymph nodes. Doxycycline-induced GPX4 knockout mitigated the metastatic capability of Merlin RNAi-Gpx4-iKO tumors.

FIG. 5F shows a graph illustrating that the number of mice with tumor metastasis to excised organs were higher in Merlin RNAi-Gpx4-iKO tumors; and doxycycline-induced GPX4 knockout reduced the metastatic capability of Merlin RNAi-Gpx4-iKO tumors, but had no effect on Gpx4-iKO tumors. The excised organs includes: Heart (H), lung (L), peritoneum (P), intestine/mesenteric lymph nodes (I), liver (Li), spleen (S), kidneys (k).

FIG. 6A shows a bar graph demonstrating that inhibiting cystine import with erastin, an inhibitor of the cystine/glutamate antiporter, induces HCT116 human colon cancer cells to undergo ferroptosis in a cell density-dependent manner; and the induction of ferroptosis is dependent on the production of lipid reactive oxygen species (ROS).

FIG. 6B shows a bar graph demonstrating that inhibiting Glutathione Peroxidase-4, a glutathione-dependent enzyme catalyzing the clearance of lipid reactive oxygen species, with its covalent inhibitor RSL3, induces HCT116 human colon cancer cells to undergo ferroptosis in a cell density-dependent manner; and the induction of ferroptosis was dependent on the production of lipid reactive oxygen species (ROS).

FIG. 6C shows a bar graph demonstrating that low density cystine-starved HCT116 human colon cancer cells underwent death by ferroptosis, which was inhibited by the cell death inhibitor ferrostating (Fer-1) and DFO, but not death by apoptosis (Z-VAD-FMK) or necroptosis (GSK′872).

FIG. 6D shows a bar graph demonstrating that low density HCT116 human colon cancer cells treated with the covalent inhibitor of Glutathione Peroxidase-4 (RSL3), underwent death by ferroptosis, which was inhibited by the cell death inhibitor ferrostating (Fer-1) and DFO, but not death by apoptosis (Z-VAD-FMK) or necroptosis (GSK′872).

FIG. 6E shows a bar graph demonstrating that the resistance of high density HCT116 human colon cancer cells to cystine deprivation-induced ferroptosis was not caused by the depletion of nutrient, such glutamine.

FIG. 7A shows an immunoblot (top) and immunofluorescence (bottom) illustrating that the levels of E-cadherin in HCT116 human colon cancer cells increased with cell density.

FIG. 7B shows an immunoblot illustrating that the levels of E-cadherin in the ferroptosis density-dependent hypersensitive cell line H1650 increased with cell density; the ferroptosis-resistant cell line BT474 expressed high levels of E-cadherin at all densities; and the levels of E-cadherin was undetectable in the density-independent ferroptosis sensitive cell line MDA-MB-231.

FIG. 7C shows an immunohistochemistry staining illustrating a high expression of E-cadherin in tumor spheroids generated from HCT116 human colon cancer cells, but no E-cadherin expression was detected in tumor spheroids generated from MDA-MB-231 cells.

FIG. 8A shows a bar graph demonstrating that blocking E-cadherin-mediated cell-cell adhesion with an anti-E-cadherin antibody increased the sensitivity of high density cystine-deprived HCT116 human colon cancer cells to ferroptosis.

FIG. 8B shows an immunofluorescence staining illustrating that E-cadherin was depleted in E-cadherin HCT116 mutant cells generated by the CRISPR/Cas9 approach.

FIG. 8C shows that the re-expression of wild type E-cadherin, but not the E-cadherin mutant lacking the ectodomain (EcadΔecto) in E-cadherin depleted HCT116 human colon cancer cells restored resistance to cystine-deprivation-induced ferroptosis in tumor spheroids generated from the cells.

FIG. 8D shows a bar graph illustrating that the re-expression of wild type E-cadherin, but not the E-cadherin mutant lacking the ectodomain (EcadΔecto), in E-cadherin depleted HCT116 human colon cancer cells restored ferroptosis-mediated reduced cell viability within tumor spheroids generated from the cells.

FIG. 8E shows an immunoblot illustrating the ectopic expression of E-cadherin in E-cadherin-negative MDA-MB-231 cells.

FIG. 8F shows a bar graph illustrating that ectopic expression of E-cadherin in E-cadherin-negative MDA-MB-231 cells rendered the MDA-MB-231 cells resistant to cystine deprivation-induced ferroptosis at high density.

FIG. 9A shows an immunofluorescence staining illustrating that the nuclear localization of the pro-oncogenic transcription cofactor YAP decreased as the cell density of HCT116 human colon cancer cells increased.

FIG. 9B shows an immunoblot illustrating that the phosphorylation state of the pro-oncogenic transcription cofactor YAP increased as the cell density of the HCT116 human colon cancer cells increased, and that the cytosolic fraction of YAP and phospho-YAP increased with increased cell density.

FIG. 9C shows an immunoblot illustrating the levels of YAP and phospho-YAP in parental and E-cadherin-depleted HCT116 cells (ΔEcad).

FIG. 9D shows an immunofluorescence illustrating the levels of YAP, and E-cadherin in parental and E-cadherin-depleted HCT116 cells (ΔEcad) at low and high density.

FIG. 10A shows an immunofluorescence illustrating that Merlin RNAi induced the nuclear accumulation of YAP in HCT116 cells at high cell density.

FIG. 10B shows an immunoblot illustrating that Merlin RNAi decreased the levels of phospho-YAP in HCT116 cells and has no impact on the levels of YAP.

FIG. 10C shows a bar graph illustrating that low cell density increased YAP transcriptional activity, as measured by the relative mRNA levels of two canonical YAP targets CTGF and CYR61 in HCT116 cells.

FIG. 10D shows a bar graph illustrating that loss of E-cadherin (ΔEcad) increased YAP transcriptional activity as measured by the mRNA levels of two canonical YAP targets CTGF and CYR61.

FIG. 10E shows a bar graph illustrating that loss of E-cadherin (ΔEcad) increased YAP transcriptional activity captured with an 8×GTIIC-luciferase reporter assay that monitored YAP-TEAD transcriptional activity.

FIG. 10F shows a bar graph illustrating that Merlin RNAi increased YAP transcriptional activity as measured by the mRNA levels of two canonical YAP targets CTGF and CYR61.

FIG. 10G shows a bar graph illustrating that Merlin RNAi increased YAP transcriptional activity, which was captured with an 8×GTIIC-luciferase reporter assay that monitored YAP-TEAD transcriptional activity.

FIG. 11A shows a bar graph illustrating that RNAi knock-down of E-cadherin, Merlin, Lats 1, and Lats 2 in HCT116 cells enhanced RSL3-induced ferroptosis, and increased RSL3-induced lipid Reactive Oxygen species (ROS) production, both of which were inhibited by the ferroptosis inhibitor ferrostating (Fer-1).

FIG. 11B shows a cell growth graph illustrating that RNAi knock-down of E-cadherin, Merlin, Lats 1, and Lats 2 in HCT116 cells did not affect cell proliferation in the presence or absence of cystine.

FIG. 12A shows an immunoblot illustrating that expression of a constitutively active p21-Activated Kinase (PAK-CAAX) in HCT116 cells enhanced the phosphorylation of Merlin, but expression of an inactive mutant (PAK^{K298R}-CAAX) did not.

FIG. 12B shows a bar graph illustrating that constitutively active PAK-CAAX, but not the inactive PAK^{K298R}-CAAX mutant enhanced the transcriptional activity of YAP as measured with the 8×GTIIC-luciferase reporter assay that monitored YAP-TEAD transcriptional activity.

FIG. 12C shows a bar graph illustrating that constitutively active PAK-CAAX, but not the inactive PAK^{K298R}-CAAX mutant, enhanced cystine deprivation-induced ferroptosis, which was inhibited by the ferroptosis inhibitor Fer-1.

FIG. 12D shows a bar graph illustrating that constitutively active PAK-CAAX, but not the inactive PAK^{K298R}-CAAX mutant, enhanced RSL3-induced ferroptosis, which was inhibited by the ferroptosis inhibitor Fer-1.

FIG. 13A shows two western blot demonstrating the expression of a cadherin protein (Pan-cadherin) in ten patient-derived malignant mesothelioma cell lines, and the expression of lats1, or Lats2 four mesothelioma cell lines 211H, H2452, H-meso that expressed wild-type Merlin.

FIG. 13B shows a bar graph illustrating that highly confluent Merlin-wt MSTO-211H mesothelioma cells treated with Merlin RNAi became sensitive to RSL3-induced ferroptosis, and exhibited enhanced production of lipid reactive oxygen species; and that the effect was blocked by the ferroptosis inhibitor fer-1.

FIG. 13C shows an immunoblot and immunofluorescence staining illustrating the level of Merlin, and the subcellular localization of YAP in Merlin-mutant Meso33 mesothelioma cells reconstituted with wild-type Merlin. In particular, the subcellular localization of YAP was decreased in Merlin reconstituted highly confluent cells.

FIG. 13D shows an immunofluorescence staining illustrating that highly confluent Merlin-mutant Meso33 mesothelioma cells reconstituted with wild-type Merlin became resistant to cystine-deprivation induced ferroptosis.

FIG. 13E shows a bar graph illustrating the resistance of highly confluent Merlin-mutant Meso33 mesothelioma cells reconstituted with wild-type Merlin to cystine-deprivation induced ferroptosis, and that these cells also produced less lipid reactive oxygen species.

FIG. 14A shows an immunoblot illustrating that in Merlin-wt MSTO-11H mesothelioma cells, the levels of N-cadherin and phospho-YAP, but not YAP, increased in a cell density-dependent manner.

FIG. 14B shows an immunoblot illustrating the levels of N-cadherin expression following N-cadherin RNAi.

FIG. 14C shows an immunofluorescence demonstrating that N-cadherin RNAi sensitized Merlin-wt MSTO-11H mesothelioma cells to cystine-deprivation induced ferroptosis when cultured at high confluence.

FIG. 14D shows a bar graph quantifying the sensitization of highly confluent Merlin-wt MSTO-11H mesothelioma cells to cystine-deprivation induced ferroptosis following N-cadherin RNAi.

FIG. 14E shows a bar graph quantifying the sensitization of highly confluent Merlin-wt MSTO-11H mesothelioma cells to RSL3-induced ferroptosis following N-cadherin RNAi.

FIG. 14F shows an immunofluorescence staining illustrating that tumor spheroids generated from N-cadherin RNAi treated Merlin-wt MSTO-11H mesothelioma cells were sensitized to erastin-induced ferroptosis.

FIG. 14G shows a bar graph quantifying the reduced cell viability within tumor spheroids generated from N-cadherin RNAi treated Merlin-wt MSTO-11H mesothelioma cells following erastin-induced ferroptosis.

FIG. 1411 shows an immunofluorescence staining illustrating the subcellular localization of YAP in N-cadherin RNAi treated Merlin-wt MSTO-11H mesothelioma cells.

FIG. 141 shows a bar graph illustrating that N-cadherin RNAi increased YAP transcriptional activity as measured by the mRNA levels of two canonical YAP targets CTGF and CYR61 in N-cadherin RNAi treated Merlin-wt MSTO-11H mesothelioma cells.

FIG. 14J shows a bar graph illustrating that in N-cadherin RNAi treated Merlin-wt MSTO-11H mesothelioma cells, N-cadherin RNAi increased YAP transcriptional activity captured by a 8×GTIIC-luciferase reporter assay that monitored YAP-TEAD transcriptional activity.

FIG. 15A shows an immunofluorescent staining illustrating that MEFs, which are non-epithelial cell in origin, are also sensitive to cystine-deprivation induced ferroptosis when cultured at high confluence.

FIG. 15B shows two bar graphs illustrating that cystine deprivation-induced ferroptosis in MEF cells was cell density-dependent, and that ferroptosis was coupled with enhanced production of lipid reactive oxygen species.

FIG. 15C shows two bar graphs illustrating that Erastin-induced ferroptosis in MEF cells was cell density-dependent, and that ferroptosis was coupled with enhanced production of lipid reactive oxygen species.

FIG. 15D shows two bar graphs illustrating RSL3-induced ferroptosis in MEF cells was cell density-dependent, and that ferroptosis was coupled with enhanced production of lipid reactive oxygen species.

FIG. 15E shows an immunofluorescence staining that illustrates the increased exclusion of YAP from the nucleus with increasing cell density in MEF cells.

FIG. 15F shows an immunoblot and an immunofluorescence illustrating the levels of Merlin in MEF cells following Merlin RNAi (Left) and that Merlin RNAi enhanced YAP nuclear accumulation.

FIG. 15G shows a bar graph illustrating that in confluent MEF cells, Merlin RNAi increased cystine deprivation-, Erastin-, and RSL3-induced ferroptosis and the production of lipid reactive oxygen species; which were blocked by the inhibition of ferroptosis with Ferrostatin (fer-1).

FIG. 16A shows a bar graph illustrating that a YAP mutant with serine to alanine substitution at position 127 (YAP^{S127}) is constitutively active based on the enhanced mRNA levels of two canonical YAP targets CTGF and CYR61 in HCT116 human colon cancer cells ectopically expressing the YAP^{S127} mutants.

FIG. 16B shows a bar graph illustrating the quantification of the YAP-TEAD transcriptional activity in HCT116 human colon cancer cells ectopically expressing the YAP^{S127} mutants captured with the luciferase activity of an 8×GTIIC-luciferase reporter.

FIG. 16C shows an immunoblot illustrating the levels of YAP^{S127}, YAP, and phospho-YAP in Merlin-wt MSTO-211H mesothelioma cells expressing the YAP^{S127} mutant.

FIG. 16D shows an immunofluorescent staining illustrating the subcellular localization of YAP in Merlin-wt MSTO-211H mesothelioma cells expressing the YAP^{S127} mutant.

FIG. 16E shows an immunofluorescence staining and a bar graph illustrating that HCT116 human colon cancer cells ectopically expressing the YAP^{S127} mutants were sensitive to cystine deprivation induced ferroptosis even at high cell density.

FIG. 16F shows a bar graph illustrating the enhanced production of lipid reactive oxygen species in HCT116 human colon cancer cells ectopically expressing the YAP^{S127} mutants

FIG. 16G shows an immunofluorescence staining illustrating that tumor spheroids generated from Merlin-wt MSTO-211H mesothelioma cells overexpressing the YAP^{S127} mutant were sensitive to erastin-induced ferroptosis.

FIG. 1611 shows a bar graph illustrating reduced cell viability within tumor spheroids generated from Merlin-wt MSTO-211H mesothelioma cells overexpressing the YAP^{S127} mutant following erastin-induced ferroptosis.

FIG. 161 shows a bar graph illustrating that inhibiting the interaction of YAP with TEAD family of transcription factors with veterpofin (VP) blocked the sensitization of Merlin RNAi treated HCT116 human colon cancer cells and Merlin-wt MSTO-211H mesothelioma cells to cystine deprivation induced ferroptosis.

FIG. 16J shows a bar graph illustrating that inhibiting the interaction of YAP with TEAD family of transcription factors with veterpofin (VP) blocked the sensitization of YAP^{S127}-expressing HCT116 cells and Merlin-wt MSTO-211H cells to cystine-deprivation-induced ferroptosis.

FIG. 17A shows an immunoblot illustrating the expression levels of the transferrin receptor (TFRC) and the acyl-CoA synthase long chain family member 4 (ACSL4) in HCT116 cells overexpressing TFRC, ACSL4, or both.

FIG. 17B shows a bar graph illustrating that confluent HCT116 cells overexpressing TFRC or ACSL4 were partially sensitized to RSL3-induced ferroptosis, while co-expression of TFRC and ACSL4 enhanced RSL3-induced ferroptosis.

FIG. 17C shows an immunoblot illustrating the expression levels of the transferrin receptor (TFRC), and E-cadherin in HCT116 cells depleted of E-cadherin and TFRC.

FIG. 17D shows a bar graph illustrating that the transferrin receptor RNAi abrogated the sensitivity of E-cadherin depleted HCT116 human colon cancer cells to cystine-deprivation induced ferroptosis.

FIG. 18A shows an immunofluorescence staining and a bar graph illustrating that tumor spheroids generated from Merlin-wt MSTO-211H cells expressing Merlin RNAi-Gpx4-iKO were more sensitive to GPX4-induced ferroptosis and had reduced cell viability than those generated from Merlin-wt MSTO-211H cells expressing Gpx4-iKO. GPX4 was induced with doxycycline treatment, and GPX4-induced ferroptosis was tested

FIG. 18B shows an immunohistochemical staining of Merlin, ACSL4, TFR, and YAP in xenograft tumors generated from Gpx4-iKO and Merlin RNAi-Gpx4-iKO cells subcutaneously injected in nude mice fed with normal or doxycycline diet. Tumors were counter-stained with haematoxylin (blue). In particular, MerlinRNAi increased the levels of TFRC and ACSL4 as well as the nuclear accumulation of YAP.

FIG. 18C shows an haematoxylin and eosin (H&E) and an immunohistochemical staining of GPX4, PTGS2, and Ki67 in xenograft tumors generated from Gpx4-iKO and Merlin RNAi-Gpx4-iKO cells subcutaneously injected in nude mice fed with normal or doxycycline diet. Tumors were counter-stained with haematoxylin (blue). In particular, the levels of GPX4 were reduced in the tumors.

FIG. 18D shows images illustrating the growth of resected subcutaneous tumors generated from Gpx4-iKO and Merlin RNAi-Gpx4-iKO cells harboring a retroviral TK-GFP-Luciferase reporter and implanted in orthotopic intrapleural mouse model of mesothelioma in NOD/SCID mice.

FIG. 18E shows a graph illustrating the bioluminescent imaging signal from tumors generated from Gpx4-iKO and Merlin RNAi-Gpx4-iKO cells as described in FIG. 18D. In particular, tumor generated from Merlin RNAi-Gpx4-iKO grew more aggressively than Gpx4-iKO tumors. However, Gpx4 knockout induction with doxycycline reduced the growth of Merlin RNAi-Gpx4-iKO tumors, but had no effect on the growth of Gpx4-iKO tumors.

FIG. 18F shows an image illustrating the metastatic behavior of tumor spheroids generated from Merlin-wt MSTO-211H cells expressing Gpx4-iKO or Merlin RNAi-Gpx4-iKO grown in Matrigel. In particular, Merlin RNAi-Gpx4-iKO tumor spheroids extended more protrusions from the spheroid body into the Matrigel.

FIG. 19A shows a graph illustrating the growth curve of xenograft tumors generated from HCT116 human colon cancer cells expressing hairpins targeting Lats 1/2 subcutaneously injected in nude mice and treated with an erastin analog, imidazole ketone erastin (IKE). In particular, HCT116-derived xenograft tumors grew slowly or receded in response to IKE treatment when Lats1/2 were inhibited.

FIG. 19B shows an image of resected tumors illustrating the growth of xenograft tumors generated from HCT116 human colon cancer cells expressing hairpins targeting Lats 1/2 as described in FIG. 19A; and in response to IKE treatment, the size of tumors derived from Lats1/2 depleted HCT116 cells was reduced.

FIG. 19C shows a bar graph quantifying the mass of resected xenograft tumors generated from HCT116 human colon cancer cells expressing hairpins targeting Lats 1/2 as described in FIG. 19A, and illustrating that in response to IKE treatment, the growth of tumors derived from Lats1/2 depleted HCT116 cells was reduced.

FIG. 20A shows a bar graph illustrating that HCT116 human colon cancer cells were susceptible to sorafenib-induced ferroptosis, when cultured at low, but not at high density. Sorafenib is used for the treatment of hepatocellular and renal carcinoma, and can stabilize malignant mesothelioma.

FIG. 20B shows a bar graph illustrating that loss of E-cadherin sensitized confluent HCT116 human colon cancer cells to sorafenib-induced ferroptosis.

FIG. 20C shows a bar graph illustrating that Merlin RNAi sensitized confluent HCT116 human colon cancer cells to sorafenib-induced ferroptosis.

FIG. 20D shows a bar graph illustrating that Merlin RNAi sensitized confluent Merlin-wt MSTO-211H mesothelioma cells to sorafenib-induced ferroptosis.

FIG. 20E shows a bar graph illustrating that expression of the constitutively active YAP^{S127A} mutant sensitized confluent HCT116 human colon cancer cells to sorafenib-induced ferroptosis.

FIG. 20F shows a bar graph illustrating that expression of the constitutively active YAP^{S127A} mutant sensitized confluent Merlin-wt MSTO-211H mesothelioma cells to sorafenib-induced ferroptosis.

FIG. 20G shows a bar graph illustrating that Lats1/2 RNAi sensitized confluent HCT116 human colon cancer cells to sorafenib-induced ferroptosis.

FIG. 21A shows a bar graph illustrating the expression levels of Epithelial-Mesenchymal Transition-related genes in NF639 mouse mammary tumor cell line containing MMTV-neu treated with Tumor Growth Factor-β (TGFβ).

FIG. 21B shows a bar graph illustrating that TGFβ treatment changed the sensitivity of NF639 cells to cystine deprivation induced ferroptosis, when grown at low cell density.

FIG. 21C shows a bar graph illustrating that TGFβ treatment sensitized confluent NF639 cells to cystine deprivation induced ferroptosis.

### DETAILED DESCRIPTION

It is to be appreciated that certain aspects, modes, embodiments, variations and features of the present methods are described below in various levels of detail in order to provide a substantial understanding of the present technology.

In practicing the present methods, many conventional techniques in molecular biology, protein biochemistry, cell biology, microbiology and recombinant DNA are used. See, e.g., Sambrook and Russell eds. (2001) Molecular Cloning: A Laboratory Manual, 3rd edition; the series Ausubel et al., eds. (2007) Current Protocols in Molecular Biology; the series Methods in Enzymology (Academic Press, Inc., N.Y.); MacPherson et al., (1991) PCR 1: A Practical Approach (IRL Press at Oxford University Press); MacPherson et al., (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); and Herzenberg et al., eds (1996) Weir's Handbook of Experimental Immunology.

The present disclosure demonstrates that tumorigenic alterations of multiple components of the cadherin-Merlin-Hippo-YAP signaling axis (loss of function of Ecad or Merlin, and super-activation of YAP) all sensitize cancer cells to ferroptosis. Accordingly, such tumorigenic mutations may be useful as biomarkers to predict the responsiveness of cancer cells to the induction of ferroptosis. Indeed, analysis of mouse xenograft models for mesothelioma showed that Merlin-defective mesothelioma cells were more malignant and metastatic, but were also more susceptible to ferroptosis. As demonstrated by the Examples herein, cancer cell sensitivity to ferroptosis can be increased by specific mutations, thus suggesting that there is some selectivity of the ferroptosis-inducing agents toward cancer cells over normal tissue. Moreover, ferroptosis-inducing cancer therapy confers another significant benefit in overcoming cancer cell resistance to current treatments. Numerous tumorigenic alterations in the Ecad-Merlin-Hippo-YAP signaling axis not only predict malignancy and tendency of metastasis, but also make cancer cells highly resistant to chemotherapies and various targeted treatments. For example, YAP, frequently activated in liver cancer, can promote resistance to tyrosine kinase inhibitors through upregulated expression of the AXL tyrosine kinase; and Merlin mutation has been implicated in resistance of melanoma to BRAF inhibitors. Importantly, these genetic alterations may lead to the selection of ferroptosis induction as a viable therapeutic approach.

### Definitions

Unless defined otherwise, all technical and scientific terms used herein generally have the same meaning as commonly understood by one of ordinary skill in the art to which this technology belongs. As used in this specification and the appended claims, the singular forms “a”, “an” and “the” include plural referents unless the content clearly dictates otherwise. For example, reference to “a cell” includes a combination of two or more cells, and the like. Generally, the nomenclature used herein and the laboratory procedures in cell culture, molecular genetics, organic chemistry, analytical chemistry and nucleic acid chemistry and hybridization described below are those well-known and commonly employed in the art.

As used herein, the term “about” in reference to a number is generally taken to include numbers that fall within a range of 1%-5% in either direction (greater than or less than) of the number unless otherwise stated or otherwise evident from the context.

The term “adapter” refers to a short, chemically synthesized, nucleic acid sequence which can be used to ligate to the end of a nucleic acid sequence in order to facilitate attachment to another molecule. The adapter can be single-stranded or double-stranded. An adapter can incorporate a short (typically less than 50 base pairs) sequence useful for PCR amplification or sequencing.

As used herein, an “alteration” of a gene or gene product (e.g., a marker gene or gene product) refers to the presence of a mutation or mutations within the gene or gene product, e.g., a mutation, which affects the quantity or activity of the gene or gene product, as compared to the normal or wild-type gene. The genetic alteration can result in changes in the quantity, structure, and/or activity of the gene or gene product in a cancer tissue or cancer cell, as compared to its quantity, structure, and/or activity, in a normal or healthy tissue or cell (e.g., a control). For example, an alteration which is associated with cancer, or predictive of responsiveness to anti-cancer therapeutics, can have an altered nucleotide sequence (e.g., a mutation), amino acid sequence, chromosomal translocation, intra-chromosomal inversion, copy number, expression level, protein level, protein activity, in a cancer tissue or cancer cell, as compared to a normal, healthy tissue or cell. Exemplary mutations include, but are not limited to, point mutations (e.g., silent, missense, or nonsense), deletions, insertions, inversions, linking mutations, duplications, translocations, inter- and intra-chromosomal rearrangements. Mutations can be present in the coding or non-coding region of the gene.

As used herein, the terms “amplify” or “amplification” with respect to nucleic acid sequences, refer to methods that increase the representation of a population of nucleic acid sequences in a sample. Nucleic acid amplification methods are well known to the skilled artisan and include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), recombinase-polymerase amplification (RPA)(TwistDx, Cambridge, UK), transcription mediated amplification, signal mediated amplification of RNA technology, loop-mediated isothermal amplification of DNA, helicase-dependent amplification, single primer isothermal amplification, and self-sustained sequence replication (3 SR), including multiplex versions or combinations thereof. Copies of a particular nucleic acid sequence generated in vitro in an amplification reaction are called “amplicons” or “amplification products.”

The terms “cancer” or “tumor” are used interchangeably and refer to the presence of cells possessing characteristics typical of cancer-causing cells, such as uncontrolled proliferation, immortality, metastatic potential, rapid growth and proliferation rate, and certain characteristic morphological features. Cancer cells are often in the form of a tumor, but such cells can exist alone within an animal, or can be a non-tumorigenic cancer cell. As used herein, the term “cancer” includes premalignant, as well as malignant cancers.

The terms “complementary” or “complementarity” as used herein with reference to polynucleotides (i.e., a sequence of nucleotides such as an oligonucleotide or a target nucleic acid) refer to the base-pairing rules. The complement of a nucleic acid sequence as used herein refers to an oligonucleotide which, when aligned with the nucleic acid sequence such that the 5′ end of one sequence is paired with the 3′ end of the other, is in “antiparallel association.” For example, the sequence “5′-A-G-T-3′” is complementary to the sequence “3′-T-C-A-5.” Certain bases not commonly found in naturally-occurring nucleic acids may be included in the nucleic acids described herein. These include, for example, inosine, 7-deazaguanine, Locked Nucleic Acids (LNA), and Peptide Nucleic Acids (PNA). Complementarity need not be perfect; stable duplexes may contain mismatched base pairs, degenerative, or unmatched bases. Those skilled in the art of nucleic acid technology can determine duplex stability empirically considering a number of variables including, for example, the length of the oligonucleotide, base composition and sequence of the oligonucleotide, ionic strength and incidence of mismatched base pairs. A complement sequence can also be an RNA sequence complementary to the DNA sequence or its complement sequence, and can also be a cDNA.

As used herein, a “control” is an alternative sample used in an experiment for comparison purpose. A control can be “positive” or “negative.” A “control nucleic acid sample” or “reference nucleic acid sample” as used herein, refers to nucleic acid molecules from a control or reference sample. In certain embodiments, the reference or control nucleic acid sample is a wild type or a non-mutated DNA or RNA sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

“Detecting” as used herein refers to determining the presence of a mutation or alteration in a nucleic acid of interest in a sample. Detection does not require the method to provide 100% sensitivity. Analysis of nucleic acid markers can be performed using techniques known in the art including, but not limited to, sequence analysis, and electrophoretic analysis. Non-limiting examples of sequence analysis include Maxam-Gilbert sequencing, Sanger sequencing, capillary array DNA sequencing, thermal cycle sequencing (Sears et al., Biotechniques, 13:626-633 (1992)), solid-phase sequencing (Zimmerman et al., Methods Mol. Cell Biol, 3:39-42 (1992)), sequencing with mass spectrometry such as matrix-assisted laser desorption/ionization time-of-flight mass spectrometry (MALDI-TOF/MS; Fu et al., Nat. Biotechnol, 16:381-384 (1998)), and sequencing by hybridization. Chee et al., Science, 274:610-614 (1996); Drmanac et al., Science, 260:1649-1652 (1993); Drmanac et al., Nat. Biotechnol, 16:54-58 (1998). Non-limiting examples of electrophoretic analysis include slab gel electrophoresis such as agarose or polyacrylamide gel electrophoresis, capillary electrophoresis, and denaturing gradient gel electrophoresis. Additionally, next generation sequencing methods can be performed using commercially available kits and instruments from companies such as the Life Technologies/Ion Torrent PGM or Proton, the Illumina HiSEQ or MiSEQ, and the Roche/454 next generation sequencing system.

“Detectable label” as used herein refers to a molecule or a compound or a group of molecules or a group of compounds used to identify a nucleic acid or protein of interest. In some embodiments, the detectable label may be detected directly. In other embodiments, the detectable label may be a part of a binding pair, which can then be subsequently detected. Signals from the detectable label may be detected by various means and will depend on the nature of the detectable label. Detectable labels may be isotopes, fluorescent moieties, colored substances, and the like. Examples of means to detect detectable labels include but are not limited to spectroscopic, photochemical, biochemical, immunochemical, electromagnetic, radiochemical, or chemical means, such as fluorescence, chemifluorescence, or chemiluminescence, or any other appropriate means.

As used herein, the term “effective amount” refers to a quantity sufficient to achieve a desired therapeutic and/or prophylactic effect, e.g., an amount which results in the prevention of, or a decrease in a disease or disorder or one or more signs or symptoms associated with a disease or disorder. In the context of therapeutic or prophylactic applications, the amount of a composition administered to the subject will depend on the degree, type, and severity of the disease and on the characteristics of the individual, such as general health, age, sex, body weight and tolerance to drugs. The skilled artisan will be able to determine appropriate dosages depending on these and other factors. The compositions can also be administered in combination with one or more additional therapeutic compounds. In the methods described herein, the therapeutic compounds may be administered to a subject having one or more signs or symptoms of a disease or disorder. As used herein, a “therapeutically effective amount” of a compound refers to compound levels in which the physiological effects of a disease or disorder are, at a minimum, ameliorated.

“Gene” as used herein refers to a DNA sequence that comprises regulatory and coding sequences necessary for the production of an RNA, which may have a non-coding function (e.g., a ribosomal or transfer RNA) or which may include a polypeptide or a polypeptide precursor. The RNA or polypeptide may be encoded by a full length coding sequence or by any portion of the coding sequence so long as the desired activity or function is retained. Although a sequence of the nucleic acids may be shown in the form of DNA, a person of ordinary skill in the art recognizes that the corresponding RNA sequence will have a similar sequence with the thymine being replaced by uracil, i.e., “T” is replaced with “U.”

The term “hybridize” as used herein refers to a process where two substantially complementary nucleic acid strands (at least about 65% complementary over a stretch of at least 14 to 25 nucleotides, at least about 75%, or at least about 90% complementary) anneal to each other under appropriately stringent conditions to form a duplex or heteroduplex through formation of hydrogen bonds between complementary base pairs. Hybridizations are typically and preferably conducted with probe-length nucleic acid molecules, preferably 15-100 nucleotides in length, more preferably 18-50 nucleotides in length. Nucleic acid hybridization techniques are well known in the art. See, e.g., Sambrook, et al., 1989, Molecular Cloning: A Laboratory Manual, Second Edition, Cold Spring Harbor Press, Plainview, N.Y. Hybridization and the strength of hybridization (i.e., the strength of the association between the nucleic acids) is influenced by such factors as the degree of complementarity between the nucleic acids, stringency of the conditions involved, and the thermal melting point (Tₘ) of the formed hybrid. Those skilled in the art understand how to estimate and adjust the stringency of hybridization conditions such that sequences having at least a desired level of complementarity will stably hybridize, while those having lower complementarity will not. For examples of hybridization conditions and parameters, see, e.g., Sambrook, et al., 1989, Molecular Cloning: A Laboratory Manual, Second Edition, Cold Spring Harbor Press, Plainview, N.Y.; Ausubel, F. M. et al. 1994, Current Protocols in Molecular Biology, John Wiley & Sons, Secaucus, N.J. In some embodiments, specific hybridization occurs under stringent hybridization conditions. An oligonucleotide or polynucleotide (e.g., a probe or a primer) that is specific for a target nucleic acid will “hybridize” to the target nucleic acid under suitable conditions.

As used herein, the terms “individual”, “patient”, or “subject” are used interchangeably and refer to an individual organism, a vertebrate, a mammal, or a human. In a preferred embodiment, the individual, patient or subject is a human.

As used herein, the term “library” refers to a collection of nucleic acid sequences, e.g., a collection of nucleic acids derived from whole genomic, subgenomic fragments, cDNA, cDNA fragments, RNA, RNA fragments, or a combination thereof. In one embodiment, a portion or all of the library nucleic acid sequences comprises an adapter sequence. The adapter sequence can be located at one or both ends. The adapter sequence can be useful, e.g., for a sequencing method (e.g., an NGS method), for amplification, for reverse transcription, or for cloning into a vector.

The library can comprise a collection of nucleic acid sequences, e.g., a target nucleic acid sequence (e.g., a tumor nucleic acid sequence), a reference nucleic acid sequence, or a combination thereof. In some embodiments, the nucleic acid sequences of the library can be derived from a single subject. In other embodiments, a library can comprise nucleic acid sequences from more than one subject (e.g., 2, 3, 4, 5, 6, 7, 8, 9, 10, 20, 30 or more subjects). In some embodiments, two or more libraries from different subjects can be combined to form a library having nucleic acid sequences from more than one subject.

A “library nucleic acid sequence” refers to a nucleic acid molecule, e.g., a DNA, RNA, or a combination thereof, that is a member of a library. Typically, a library nucleic acid sequence is a DNA molecule, e.g., genomic DNA or cDNA. In some embodiments, a library nucleic acid sequence is fragmented, e.g., sheared or enzymatically prepared, genomic DNA. In certain embodiments, the library nucleic acid sequences comprise sequence from a subject and sequence not derived from the subject, e.g., adapter sequence, a primer sequence, or other sequences that allow for identification, e.g., “barcode” sequences.

The term “multiplex PCR” as used herein refers to amplification of two or more PCR products or amplicons which are each primed using a distinct primer pair.

“Next-generation sequencing or NGS” as used herein, refers to any sequencing method that determines the nucleotide sequence of either individual nucleic acid molecules (e.g., in single molecule sequencing) or clonally expanded proxies for individual nucleic acid molecules in a high throughput parallel fashion (e.g., greater than 10³, 10⁴, 10⁵ or more molecules are sequenced simultaneously). In one embodiment, the relative abundance of the nucleic acid species in the library can be estimated by counting the relative number of occurrences of their cognate sequences in the data generated by the sequencing experiment. Next generation sequencing methods are known in the art, and are described, e.g., in Metzker, M. Nature Biotechnology Reviews 11:31-46 (2010).

As used herein, “oligonucleotide” refers to a molecule that has a sequence of nucleic acid bases on a backbone comprised mainly of identical monomer units at defined intervals. The bases are arranged on the backbone in such a way that they can bind with a nucleic acid having a sequence of bases that are complementary to the bases of the oligonucleotide. The most common oligonucleotides have a backbone of sugar phosphate units. A distinction may be made between oligodeoxyribonucleotides that do not have a hydroxyl group at the 2′ position and oligoribonucleotides that have a hydroxyl group at the 2′ position. Oligonucleotides may also include derivatives, in which the hydrogen of the hydroxyl group is replaced with organic groups, e.g., an allyl group. Oligonucleotides of the method which function as primers or probes are generally at least about 10-15 nucleotides long and more preferably at least about 15 to 25 nucleotides long, although shorter or longer oligonucleotides may be used in the method. The exact size will depend on many factors, which in turn depend on the ultimate function or use of the oligonucleotide. The oligonucleotide may be generated in any manner, including, for example, chemical synthesis, DNA replication, restriction endonuclease digestion of plasmids or phage DNA, reverse transcription, PCR, or a combination thereof. The oligonucleotide may be modified e.g., by addition of a methyl group, a biotin or digoxigenin moiety, a fluorescent tag or by using radioactive nucleotides.

As used herein, the term “primer” refers to an oligonucleotide, which is capable of acting as a point of initiation of nucleic acid sequence synthesis when placed under conditions in which synthesis of a primer extension product which is complementary to a target nucleic acid strand is induced, i.e., in the presence of different nucleotide triphosphates and a polymerase in an appropriate buffer (“buffer” includes pH, ionic strength, cofactors etc.) and at a suitable temperature. One or more of the nucleotides of the primer can be modified for instance by addition of a methyl group, a biotin or digoxigenin moiety, a fluorescent tag or by using radioactive nucleotides. A primer sequence need not reflect the exact sequence of the template. For example, a non-complementary nucleotide fragment may be attached to the 5′ end of the primer, with the remainder of the primer sequence being substantially complementary to the strand. The term primer as used herein includes all forms of primers that may be synthesized including peptide nucleic acid primers, locked nucleic acid primers, phosphorothioate modified primers, labeled primers, and the like. The term “forward primer” as used herein means a primer that anneals to the anti-sense strand of dsDNA. A “reverse primer” anneals to the sense-strand of dsDNA.

As used herein, “primer pair” refers to a forward and reverse primer pair (i.e., a left and right primer pair) that can be used together to amplify a given region of a nucleic acid of interest.

“Probe” as used herein refers to nucleic acid that interacts with a target nucleic acid via hybridization. A probe may be fully complementary to a target nucleic acid sequence or partially complementary. The level of complementarity will depend on many factors based, in general, on the function of the probe. A probe or probes can be used, for example to detect the presence or absence of a mutation in a nucleic acid sequence by virtue of the sequence characteristics of the target. Probes can be labeled or unlabeled, or modified in any of a number of ways well known in the art. A probe may specifically hybridize to a target nucleic acid. Probes may be DNA, RNA or a RNA/DNA hybrid. Probes may be oligonucleotides, artificial chromosomes, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may comprise modified nucleobases, modified sugar moieties, and modified internucleotide linkages. A probe may be used to detect the presence or absence of a target nucleic acid. Probes are typically at least about 10, 15, 20, 25, 30, 35, 40, 50, 60, 75, 100 nucleotides or more in length.

As used herein, a “sample” refers to a substance that is being assayed for the presence of a mutation in a nucleic acid of interest. Processing methods to release or otherwise make available a nucleic acid for detection are well known in the art and may include steps of nucleic acid manipulation. A biological sample may be a body fluid or a tissue sample. In some cases, a biological sample may consist of or comprise blood, plasma, sera, urine, feces, epidermal sample, vaginal sample, skin sample, cheek swab, sperm, amniotic fluid, cultured cells, bone marrow sample, tumor biopsies, aspirate and/or chorionic villi, cultured cells, and the like. Fresh, fixed or frozen tissues may also be used. In one embodiment, the sample is preserved as a frozen sample or as formaldehyde- or paraformaldehyde-fixed paraffin-embedded (FFPE) tissue preparation. For example, the sample can be embedded in a matrix, e.g., an FFPE block or a frozen sample. Whole blood samples of about 0.5 to 5 ml collected with EDTA, ACD or heparin as anti-coagulant are suitable.

The term “sensitivity,” as used herein in reference to the methods of the present technology, is a measure of the ability of a method to detect a preselected sequence variant in a heterogeneous population of sequences. A method has a sensitivity of S % for variants of F % if, given a sample in which the preselected sequence variant is present as at least F % of the sequences in the sample, the method can detect the preselected sequence at a preselected confidence of C %, S % of the time. By way of example, a method has a sensitivity of 90% for variants of 5% if, given a sample in which the preselected variant sequence is present as at least 5% of the sequences in the sample, the method can detect the preselected sequence at a preselected confidence of 99%, 9 out of 10 times (F=5%; C=99%; S=90%).

The term “specific” as used herein in reference to an oligonucleotide primer means that the nucleotide sequence of the primer has at least 12 bases of sequence identity with a portion of the nucleic acid to be amplified when the oligonucleotide and the nucleic acid are aligned. An oligonucleotide primer that is specific for a nucleic acid is one that, under the stringent hybridization or washing conditions, is capable of hybridizing to the target of interest and not substantially hybridizing to nucleic acids which are not of interest. Higher levels of sequence identity are preferred and include at least 75%, at least 80%, at least 85%, at least 90%, at least 95% and more preferably at least 98% sequence identity.

“Specificity,” as used herein, is a measure of the ability of a method to distinguish a truly occurring preselected sequence variant from sequencing artifacts or other closely related sequences. It is the ability to avoid false positive detections. False positive detections can arise from errors introduced into the sequence of interest during sample preparation, sequencing error, or inadvertent sequencing of closely related sequences like pseudo-genes or members of a gene family. A method has a specificity of X % if, when applied to a sample set of N_{Total} sequences, in which X_{True} sequences are truly variant and X_{Not true} are not truly variant, the method selects at least X % of the not truly variant as not variant. E.g., a method has a specificity of 90% if, when applied to a sample set of 1,000 sequences, in which 500 sequences are truly variant and 500 are not truly variant, the method selects 90% of the 500 not truly variant sequences as not variant. Exemplary specificities include 90, 95, 98, and 99%.

The term “stringent hybridization conditions” as used herein refers to hybridization conditions at least as stringent as the following: hybridization in 50% formamide, 5×SSC, 50 mM NaH₂PO₄, pH 6.8, 0.5% SDS, 0.1 mg/mL sonicated salmon sperm DNA, and 5× Denhart's solution at 42° C. overnight; washing with 2×SSC, 0.1% SDS at 45° C.; and washing with 0.2×SSC, 0.1% SDS at 45° C. In another example, stringent hybridization conditions should not allow for hybridization of two nucleic acids which differ over a stretch of 20 contiguous nucleotides by more than two bases.

As used herein, the terms “target sequence” and “target nucleic acid sequence” refer to a specific nucleic acid sequence to be detected and/or quantified in the sample to be analyzed.

“Treating” or “treatment” as used herein covers the treatment of a disease or disorder described herein, in a subject, such as a human, and includes: (i) inhibiting a disease or disorder, i.e., arresting its development; (ii) relieving a disease or disorder, i.e., causing regression of the disorder; (iii) slowing progression of the disorder; and/or (iv) inhibiting, relieving, or slowing progression of one or more symptoms of the disease or disorder. In some embodiments, treatment means that the symptoms associated with the disease are, e.g., alleviated, reduced, cured, or placed in a state of remission.

It is also to be appreciated that the various modes of treatment of disorders as described herein are intended to mean “substantial,” which includes total but also less than total treatment, and wherein some biologically or medically relevant result is achieved. The treatment may be a continuous prolonged treatment for a chronic disease or a single, or few time administrations for the treatment of an acute condition.

Methods for Detecting Polynucleotides Associated with Increased Susceptibility to Ferroptosis

Polynucleotides associated with increased susceptibility to ferroptosis may be detected by a variety of methods known in the art. Non-limiting examples of detection methods are described below. The detection assays in the methods of the present technology may include purified or isolated DNA (genomic or cDNA), RNA or protein or the detection step may be performed directly from a biological sample without the need for further DNA, RNA or protein purification/isolation.

Nucleic Acid Amplification and/or Detection

Polynucleotides associated with increased susceptibility to ferroptosis can be detected by the use of nucleic acid amplification techniques that are well known in the art. The starting material may be genomic DNA, cDNA, RNA or mRNA. Nucleic acid amplification can be linear or exponential. Specific variants or mutations may be detected by the use of amplification methods with the aid of oligonucleotide primers or probes designed to interact with or hybridize to a particular target sequence in a specific manner, thus amplifying only the target variant.

Non-limiting examples of nucleic acid amplification techniques include polymerase chain reaction (PCR), real-time quantitative PCR (qPCR), digital PCR (dPCR), reverse transcriptase polymerase chain reaction (RT-PCR), nested PCR, ligase chain reaction (see Abravaya, K. et al., Nucleic Acids Res. (1995), 23:675-682), branched DNA signal amplification (see Urdea, M. S. et al., AIDS (1993), 7(suppl 2):S11-S14), amplifiable RNA reporters, Q-beta replication, transcription-based amplification, boomerang DNA amplification, strand displacement activation, cycling probe technology, isothermal nucleic acid sequence based amplification (NASBA) (see Kievits, T. et al., J Virological Methods (1991), 35:273-286), Invader Technology, next-generation sequencing technology or other sequence replication assays or signal amplification assays.

Primers: Oligonucleotide primers for use in amplification methods can be designed according to general guidance well known in the art as described herein, as well as with specific requirements as described herein for each step of the particular methods described. In some embodiments, oligonucleotide primers for cDNA synthesis and PCR are 10 to 100 nucleotides in length, preferably between about 15 and about 60 nucleotides in length, more preferably 25 and about 50 nucleotides in length, and most preferably between about 25 and about 40 nucleotides in length.

Tₘ of a polynucleotide affects its hybridization to another polynucleotide (e.g., the annealing of an oligonucleotide primer to a template polynucleotide). In certain embodiments of the disclosed methods, the oligonucleotide primer used in various steps selectively hybridizes to a target template or polynucleotides derived from the target template (i.e., first and second strand cDNAs and amplified products). Typically, selective hybridization occurs when two polynucleotide sequences are substantially complementary (at least about 65% complementary over a stretch of at least 14 to 25 nucleotides, preferably at least about 75%, more preferably at least about 90% complementary). See Kanehisa, M., Polynucleotides Res. (1984), 12:203, incorporated herein by reference. As a result, it is expected that a certain degree of mismatch at the priming site is tolerated. Such mismatch may be small, such as a mono-, di- or tri-nucleotide. In certain embodiments, 100% complementarity exists.

Probes: Probes are capable of hybridizing to at least a portion of the nucleic acid of interest or a reference nucleic acid (i.e., wild-type sequence). Probes may be an oligonucleotide, artificial chromosome, fragmented artificial chromosome, genomic nucleic acid, fragmented genomic nucleic acid, RNA, recombinant nucleic acid, fragmented recombinant nucleic acid, peptide nucleic acid (PNA), locked nucleic acid, oligomer of cyclic heterocycles, or conjugates of nucleic acid. Probes may be used for detecting and/or capturing/purifying a nucleic acid of interest.

Typically, probes can be about 10 nucleotides, about 20 nucleotides, about 25 nucleotides, about 30 nucleotides, about 35 nucleotides, about 40 nucleotides, about 50 nucleotides, about 60 nucleotides, about 75 nucleotides, or about 100 nucleotides long. However, longer probes are possible. Longer probes can be about 200 nucleotides, about 300 nucleotides, about 400 nucleotides, about 500 nucleotides, about 750 nucleotides, about 1,000 nucleotides, about 1,500 nucleotides, about 2,000 nucleotides, about 2,500 nucleotides, about 3,000 nucleotides, about 3,500 nucleotides, about 4,000 nucleotides, about 5,000 nucleotides, about 7,500 nucleotides, or about 10,000 nucleotides long.

Probes may also include a detectable label or a plurality of detectable labels. The detectable label associated with the probe can generate a detectable signal directly. Additionally, the detectable label associated with the probe can be detected indirectly using a reagent, wherein the reagent includes a detectable label, and binds to the label associated with the probe.

In some embodiments, detectably labeled probes can be used in hybridization assays including, but not limited to Northern blots, Southern blots, microarray, dot or slot blots, and in situ hybridization assays such as fluorescent in situ hybridization (FISH) to detect a target nucleic acid sequence within a biological sample. Certain embodiments may employ hybridization methods for measuring expression of a polynucleotide gene product, such as mRNA. Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davis, PNAS. 80: 1194 (1983).

Detectably labeled probes can also be used to monitor the amplification of a target nucleic acid sequence. In some embodiments, detectably labeled probes present in an amplification reaction are suitable for monitoring the amount of amplicon(s) produced as a function of time. Examples of such probes include, but are not limited to, the 5′-exonuclease assay (TAQMAN® probes described herein (see also U.S. Pat. No. 5,538,848) various stem-loop molecular beacons (see for example, U.S. Pat. Nos. 6,103,476 and 5,925,517 and Tyagi and Kramer, 1996, Nature Biotechnology 14:303-308), stemless or linear beacons (see, e.g., WO 99/21881), PNA Molecular Beacons™ (see, e.g., U.S. Pat. Nos. 6,355,421 and 6,593,091), linear PNA beacons (see, for example, Kubista et al., 2001, SPIE 4264:53-58), non-FRET probes (see, for example, U.S. Pat. No. 6,150,097), Sunrise®/Amplifluor™ probes (U.S. Pat. No. 6,548,250), stem-loop and duplex Scorpion probes (Solinas et al., 2001, Nucleic Acids Research 29:E96 and U.S. Pat. No. 6,589,743), bulge loop probes (U.S. Pat. No. 6,590,091), pseudo knot probes (U.S. Pat. No. 6,589,250), cyclicons (U.S. Pat. No. 6,383,752), MGB Eclipse™ probe (Epoch Biosciences), hairpin probes (U.S. Pat. No. 6,596,490), peptide nucleic acid (PNA) light-up probes, self-assembled nanoparticle probes, and ferrocene-modified probes described, for example, in U.S. Pat. No. 6,485,901; Mhlanga et al., 2001, Methods 25:463-471; Whitcombe et al., 1999, Nature Biotechnology. 17:804-807; Isacsson et al., 2000, Molecular Cell Probes. 14:321-328; Svanvik et al., 2000, Anal Biochem. 281:26-35; Wolffs et al., 2001, Biotechniques 766:769-771; Tsourkas et al., 2002, Nucleic Acids Research. 30:4208-4215; Riccelli et al., 2002, Nucleic Acids Research 30:4088-4093; Zhang et al., 2002 Shanghai. 34:329-332; Maxwell et al., 2002, J. Am. Chem. Soc. 124:9606-9612; Broude et al., 2002, Trends Biotechnol. 20:249-56; Huang et al., 2002, Chem. Res. Toxicol. 15:118-126; and Yu et al., 2001, J. Am. Chem. Soc 14:11155-11161.

In some embodiments, the detectable label is a fluorophore. Suitable fluorescent moieties include but are not limited to the following fluorophores working individually or in combination: 4-acetamido-4′-isothiocyanatostilbene-2,2′disulfonic acid; acridine and derivatives: acridine, acridine isothiocyanate; Alexa Fluors: Alexa Fluor® 350, Alexa Fluor® 488, Alexa Fluor® 546, Alexa Fluor® 555, Alexa Fluor® 568, Alexa Fluor® 594, Alexa Fluor® 647 (Molecular Probes); 5-(2-aminoethyl)aminonaphthalene-1-sulfonic acid (EDANS); 4-amino-N-[3-vinylsulfonyl)phenyl]naphthalimide-3,5 disulfonate (Lucifer Yellow VS); N-(4-anilino-1-naphthyl)maleimide; anthranilamide; Black Hole Quencher™ (BHQ™) dyes (biosearch Technologies); BODIPY dyes: BODIPY® R-6G, BOPIPY® 530/550, BODIPY® FL; Brilliant Yellow; coumarin and derivatives: coumarin, 7-amino-4-methylcoumarin (AMC, Coumarin 120),7-amino-4-trifluoromethylcouluarin (Coumarin 151); Cy2®, Cy3®, Cy3.5®, Cy5®, Cy5.5®; cyanosine; 4′,6-diaminidino-2-phenylindole (DAPI); 5′, 5″-dibromopyrogallol-sulfonephthalein (Bromopyrogallol Red); 7-diethylamino-3-(4′-isothiocyanatophenyl)-4-methylcoumarin; diethylenetriamine pentaacetate; 4,4′-diisothiocyanatodihydro-stilbene-2,2′-disulfonic acid; 4,4′-diisothiocyanatostilbene-2,2′-disulfonic acid; 5-[dimethylamino]naphthalene-1-sulfonyl chloride (DNS, dansyl chloride); 4-(4′-dimethylaminophenylazo)benzoic acid (DABCYL); 4-dimethylaminophenylazophenyl-4′-isothiocyanate (DABITC); Eclipse™ (Epoch Biosciences Inc.); eosin and derivatives: eosin, eosin isothiocyanate; erythrosin and derivatives: erythrosin B, erythrosin isothiocyanate; ethidium; fluorescein and derivatives: 5-carboxyfluorescein (FAM), 5-(4,6-dichlorotriazin-2-yl)amino fluorescein (DTAF), 2′,7′-dimethoxy-4′5′-dichloro-6-carboxyfluorescein (JOE), fluorescein, fluorescein isothiocyanate (FITC), hexachloro-6-carboxyfluorescein (HEX), QFITC (XRITC), tetrachlorofluorescem (TET); fiuorescamine; IR144; IR1446; lanthamide phosphors; Malachite Green isothiocyanate; 4-methylumbelliferone; ortho cresolphthalein; nitrotyrosine; pararosaniline; Phenol Red; B-phycoerythrin, R-phycoerythrin; allophycocyanin; o-phthaldialdehyde; Oregon Green®; propidium iodide; pyrene and derivatives: pyrene, pyrene butyrate, succinimidyl 1-pyrene butyrate; QSY® 7; QSY® 9; QSY® 21; QSY® 35 (Molecular Probes); Reactive Red 4 (Cibacron®Brilliant Red 3B-A); rhodamine and derivatives: 6-carboxy-X-rhodamine (ROX), 6-carboxyrhodamine (R6G), lissamine rhodamine B sulfonyl chloride, rhodamine (Rhod), rhodamine B, rhodamine 123, rhodamine green, rhodamine X isothiocyanate, riboflavin, rosolic acid, sulforhodamine B, sulforhodamine 101, sulfonyl chloride derivative of sulforhodamine 101 (Texas Red); terbium chelate derivatives; N,N,N′,N′-tetramethyl-6-carboxyrhodamine (TAIVIRA); tetramethyl rhodamine; tetramethyl rhodamine isothiocyanate (TRITC); and VIC®. Detector probes can also comprise sulfonate derivatives of fluorescenin dyes with S03 instead of the carboxylate group, phosphoramidite forms of fluorescein, phosphoramidite forms of CY 5 (commercially available for example from Amersham).

Detectably labeled probes can also include quenchers, including without limitation black hole quenchers (Biosearch), Iowa Black (IDT), QSY quencher (Molecular Probes), and Dabsyl and Dabcel sulfonate/carboxylate Quenchers (Epoch).

Detectably labeled probes can also include two probes, wherein for example a fluorophore is on one probe, and a quencher is on the other probe, wherein hybridization of the two probes together on a target quenches the signal, or wherein hybridization on the target alters the signal signature via a change in fluorescence.

In some embodiments, interchelating labels such as ethidium bromide, SYBR® Green I (Molecular Probes), and PicoGreen® (Molecular Probes) are used, thereby allowing visualization in real-time, or at the end point, of an amplification product in the absence of a detector probe. In some embodiments, real-time visualization may involve the use of both an intercalating detector probe and a sequence-based detector probe. In some embodiments, the detector probe is at least partially quenched when not hybridized to a complementary sequence in the amplification reaction, and is at least partially unquenched when hybridized to a complementary sequence in the amplification reaction.

In some embodiments, the amount of probe that gives a fluorescent signal in response to an excited light typically relates to the amount of nucleic acid produced in the amplification reaction. Thus, in some embodiments, the amount of fluorescent signal is related to the amount of product created in the amplification reaction. In such embodiments, one can therefore measure the amount of amplification product by measuring the intensity of the fluorescent signal from the fluorescent indicator.

Primers or probes may be designed to selectively hybridize to any portion of a nucleic acid sequence encoding a polypeptide selected from among E-cadherin, N-cadherin, Merlin, Mst1, Mst2, Lats1, Lats2, YAP, TAZ, TFRC, ACSL4, and TGF-β. Exemplary nucleic acid sequences of the human orthologs of these genes are provided below:

```
Homo sapiens cadherin 1 (CDH1), transcript variant 1, mRNA (NCBI Reference
Sequence: NM_004360.5)
(SEQ ID NO: 38)
      1 agtggcgtcg gaactgcaaa gcacctgtga gcttgcggaa gtcagttcag actccagccc
    61 gctccagccc ggcccgaccc gaccgcaccc ggcgcctgcc ctcgctcggc gtccccggcc
  121 agccatgggc ccttggagcc gcagcctctc ggcgctgctg ctgctgctgc aggtctcctc
  181 ttggctctgc caggagccgg agccctgcca ccctggcttt gacgccgaga gctacacgtt
  241 cacggtgccc cggcgccacc tggagagagg ccgcgtcctg ggcagagtga attttgaaga
  301 ttgcaccggt cgacaaagga cagcctattt ttccctcgac acccgattca aagtgggcac
  361 agatggtgtg attacagtca aaaggcctct acggtttcat aacccacaga tccatttctt
  421 ggtctacgcc tgggactcca cctacagaaa gttttccacc aaagtcacgc tgaatacagt
  481 ggggcaccac caccgccccc cgccccatca ggcctccgtt tctggaatcc aagcagaatt
  541 gctcacattt cccaactcct ctcctggcct cagaagacag aagagagact gggttattcc
  601 tcccatcagc tgcccagaaa atgaaaaagg cccatttcct aaaaacctgg ttcagatcaa
  661 atccaacaaa gacaaagaag gcaaggtttt ctacagcatc actggccaag gagctgacac
  721 accccctgtt ggtgtcttta ttattgaaag agaaacagga tggctgaagg tgacagagcc
  781 tctggataga gaacgcattg ccacatacac tctcttctct cacgctgtgt catccaacgg
  841 gaatgcagtt gaggatccaa tggagatttt gatcacggta accgatcaga atgacaacaa
  901 gcccgaattc acccaggagg tctttaaggg gtctgtcatg gaaggtgctc ttccaggaac
  961 ctctgtgatg gaggtcacag ccacagacgc ggacgatgat gtgaacacct acaatgccgc
1021 catcgcttac accatcctca gccaagatcc tgagctccct gacaaaaata tgttcaccat
1081 taacaggaac acaggagtca tcagtgtggt caccactggg ctggaccgag agagtttccc
1141 tacgtatacc ctggtggttc aagctgctga ccttcaaggt gaggggttaa gcacaacagc
1201 aacagctgtg atcacagtca ctgacaccaa cgataatcct ccgatcttca atcccaccac
1261 gtacaagggt caggtgcctg agaacgaggc taacgtcgta atcaccacac tgaaagtgac
1321 tgatgctgat gcccccaata ccccagcgtg ggaggctgta tacaccatat tgaatgatga
1381 tggtggacaa tttgtcgtca ccacaaatcc agtgaacaac gatggcattt tgaaaacagc
1441 aaagggcttg gattttgagg ccaagcagca gtacattcta cacgtagcag tgacgaatgt
1501 ggtacctttt gaggtctctc tcaccacctc cacagccacc gtcaccgtgg atgtgctgga
1561 tgtgaatgaa gcccccatct ttgtgcctcc tgaaaagaga gtggaagtgt ccgaggactt
1621 tggcgtgggc caggaaatca catcctacac tgcccaggag ccagacacat ttatggaaca
1681 gaaaataaca tatcggattt ggagagacac tgccaactgg ctggagatta atccggacac
1741 tggtgccatt tccactcggg ctgagctgga cagggaggat tttgagcacg tgaagaacag
1801 cacgtacaca gccctaatca tagctacaga caatggttct ccagttgcta ctggaacagg
1861 gacacttctg ctgatcctgt ctgatgtgaa tgacaacgcc cccataccag aacctcgaac
1921 tatattcttc tgtgagagga atccaaagcc tcaggtcata aacatcattg atgcagacct
1981 tcctcccaat acatctccct tcacagcaga actaacacac ggggcgagtg ccaactggac
2041 cattcagtac aacgacccaa cccaagaatc tatcattttg aagccaaaga tggccttaga
2101 ggtgggtgac tacaaaatca atctcaagct catggataac cagaataaag accaagtgac
2161 caccttagag gtcagcgtgt gtgactgtga aggggccgct ggcgtctgta ggaaggcaca
2221 gcctgtcgaa gcaggattgc aaattcctgc cattctgggg attcttggag gaattcttgc
2281 tttgctaatt ctgattctgc tgctcttgct gtttcttcgg aggagagcgg tggtcaaaga
2341 gcccttactg cccccagagg atgacacccg ggacaacgtt tattactatg atgaagaagg
2401 aggcggagaa gaggaccagg actttgactt gagccagctg cacaggggcc tggacgctcg
2461 gcctgaagtg actcgtaacg acgttgcacc aaccctcatg agtgtccccc ggtatcttcc
2521 ccgccctgcc aatcccgatg aaattggaaa ttttattgat gaaaatctga aagcggctga
2581 tactgacccc acagccccgc cttatgattc tctgctcgtg tttgactatg aaggaagcgg
2641 ttccgaagct gctagtctga gctccctgaa ctcctcagag tcagacaaag accaggacta
2701 tgactacttg aacgaatggg gcaatcgctt caagaagctg gctgacatgt acggaggcgg
2761 cgaggacgac taggggactc gagagaggcg ggccccagac ccatgtgctg ggaaatgcag
2821 aaatcacgtt gctggtggtt tttcagctcc cttcccttga gatgagtttc tggggaaaaa
2881 aaagagactg gttagtgatg cagttagtat agctttatac tctctccact ttatagctct
2941 aataagtttg tgttagaaaa gtttcgactt atttcttaaa gctttttttt ttttcccatc
3001 actctttaca tggtggtgat gtccaaaaga tacccaaatt ttaatattcc agaagaacaa
3061 ctttagcatc agaaggttca cccagcacct tgcagatttt cttaaggaat tttgtctcac
3121 ttttaaaaag aaggggagaa gtcagctact ctagttctgt tgttttgtgt atataatttt
3181 ttaaaaaaaa tttgtgtgct tctgctcatt actacactgg tgtgtccctc tgcctttttt
3241 ttttttttaa gacagggtct cattctatcg gccaggctgg agtgcagtgg tgcaatcaca
3301 gctcactgca gccttgtcct cccaggctca agctatcctt gcacctcagc ctcccaagta
3361 gctgggacca caggcatgca ccactacgca tgactaattt tttaaatatt tgagacgggg
3421 tctccctgtg ttacccaggc tggtctcaaa ctcctgggct caagtgatcc tcccatcttg
3481 gcctcccaga gtattgggat tacagacatg agccactgca cctgcccagc tccccaactc
3541 cctgccattt tttaagagac agtttcgctc catcgcccag gcctgggatg cagtgatgtg
3601 atcatagctc actgtaacct caaactctgg ggctcaagca gttctcccac cagcctcctt
3661 tttatttttt tgtacagatg gggtcttgct atgttgccca agctggtctt aaactcctgg
3721 cctcaagcaa tccttctgcc ttggcccccc aaagtgctgg gattgtgggc atgagctgct
3781 gtgcccagcc tccatgtttt aatatcaact ctcactcctg aattcagttg ctttgcccaa
3841 gataggagtt ctctgatgca gaaattattg ggctctttta gggtaagaag tttgtgtctt
3901 tgtctggcca catcttgact aggtattgtc tactctgaag acctttaatg gcttccctct
3961 ttcatctcct gagtatgtaa cttgcaatgg gcagctatcc agtgacttgt tctgagtaag
4021 tgtgttcatt aatgtttatt tagctctgaa gcaagagtga tatactccag gacttagaat
4081 agtgcctaaa gtgctgcagc caaagacaga gcggaactat gaaaagtggg cttggagatg
4141 gcaggagagc ttgtcattga gcctggcaat ttagcaaact gatgctgagg atgattgagg
4201 tgggtctacc tcatctctga aaattctgga aggaatggag gagtctcaac atgtgtttct
4261 gacacaagat ccgtggtttg tactcaaagc ccagaatccc caagtgcctg cttttgatga
4321 tgtctacaga aaatgctggc tgagctgaac acatttgccc aattccaggt gtgcacagaa
4381 aaccgagaat attcaaaatt ccaaattttt ttcttaggag caagaagaaa atgtggccct
4441 aaagggggtt agttgagggg tagggggtag tgaggatctt gatttggatc tctttttatt
4501 taaatgtgaa tttcaacttt tgacaatcaa agaaaagact tttgttgaaa tagctttact
4561 gtttctcaag tgttttggag aaaaaaatca accctgcaat cactttttgg aattgtcttg
4621 atttttcggc agttcaagct atatcgaata tagttctgtg tagagaatgt cactgtagtt
4681 ttgagtgtat acatgtgtgg gtgctgataa ttgtgtattt tctttggggg tggaaaagga
4741 aaacaattca agctgagaaa agtattctca aagatgcatt tttataaatt ttattaaaca
4801 attttgttaa a
Homo sapiens cadherin 2 (CDH2), transcript variant 1, mRNA (NCBI Reference
Sequence: NM_001792.5)
(SEQ ID NO: 39)
      1 gccgtttctc cgcgccgctg ttggtgctgc cgctgcctcc tcctcctccg ccgccgccgc
    61 cgccgccgcc gcctcctccg gctcttcgct cggcccctct ccgcctccat gtgccggata
  121 gcgggagcgc tgcggaccct gctgccgctg ctggcggccc tgcttcaggc gtctgtagag
  181 gcttctggtg aaatcgcatt atgcaagact ggatttcctg aagatgttta cagtgcagtc
  241 ttatcgaagg atgtgcatga aggacagcct cttctcaatg tgaagtttag caactgcaat
  301 ggaaaaagaa aagtacaata tgagagcagt gagcctgcag attttaaggt ggatgaagat
  361 ggcatggtgt atgccgtgag aagctttcca ctctcttctg agcatgccaa gttcctgata
  421 tatgcccaag acaaagagac ccaggaaaag tggcaagtgg cagtaaaatt gagcctgaag
  481 ccaaccttaa ctgaggagtc agtgaaggag tcagcagaag ttgaagaaat agtgttccca
  541 agacaattca gtaagcacag tggccaccta caaaggcaga agagagactg ggtcatccct
  601 ccaatcaact tgccagaaaa ctccagggga ccttttcctc aagagcttgt caggatcagg
  661 tctgatagag ataaaaacct ttcactgcgg tacagtgtaa ctgggccagg agctgaccag
  721 cctccaactg gtatcttcat tatcaacccc atctcgggtc agctgtcggt gacaaagccc
  781 ctggatcgcg agcagatagc ccggtttcat ttgagggcac atgcagtaga tattaatgga
  841 aatcaagtgg agaaccccat tgacattgtc atcaatgtta ttgacatgaa tgacaacaga
  901 cctgagttct tacaccaggt ttggaatggg acagttcctg agggatcaaa gcctggaaca
  961 tatgtgatga ccgtaacagc aattgatgct gacgatccca atgccctcaa tgggatgttg
1021 aggtacagaa tcgtgtctca ggctccaagc accccttcac ccaacatgtt tacaatcaac
1081 aatgagactg gtgacatcat cacagtggca gctggacttg atcgagaaaa agtgcaacag
1141 tatacgttaa taattcaagc tacagacatg gaaggcaatc ccacatatgg cctttcaaac
1201 acagccacgg ccgtcatcac agtgacagat gtcaatgaca atcctccaga gtttactgcc
1261 atgacgtttt atggtgaagt tcctgagaac agggtagaca tcatagtagc taatctaact
1321 gtgaccgata aggatcaacc ccatacacca gcctggaacg cagtgtacag aatcagtggc
1381 ggagatccta ctggacggtt cgccatccag accgacccaa acagcaacga cgggttagtc
1441 accgtggtca aaccaatcga ctttgaaaca aataggatgt ttgtccttac tgttgctgca
1501 gaaaatcaag tgccattagc caagggaatt cagcaccccc ctcagtcaac tgcaaccgtg
1561 tctgttacag ttattgacgt aaatgaaaac ccttattttg cccccaatcc taagatcatt
1621 cgccaagaag aagggcttca tgccggtacc atgttgacaa cattcactgc tcaggaccca
1681 gatcgatata tgcagcaaaa tattagatac actaaattat ctgatcctgc caattggcta
1741 aaaatagatc ctgtgaatgg acaaataact acaattgctg ttttggaccg agaatcacca
1801 aatgtgaaaa acaatatata taatgctact ttccttgctt ctgacaatgg aattcctcct
1861 atgagtggaa caggaacgct gcagatctat ttacttgata ttaatgacaa tgcccctcaa
1921 gtgttacctc aagaggcaga gacttgcgaa actccagacc ccaattcaat taatattaca
1981 gcacttgatt atgacattga tccaaatgct ggaccatttg cttttgatct tcctttatct
2041 ccagtgacta ttaagagaaa ttggaccatc actcggctta atggtgattt tgctcagctt
2101 aatttaaaga taaaatttct tgaagctggt atctatgaag ttcccatcat aatcacagat
2161 tcgggtaatc ctcccaaatc aaatatttcc atcctgcgtg tgaaggtttg ccagtgtgac
2221 tccaacgggg actgcacaga tgtggacagg attgtgggtg cggggcttgg caccggtgcc
2281 atcattgcca tcctgctctg catcatcatc ctgcttatcc ttgtgctgat gtttgtggta
2341 tggatgaaac gccgggataa agaacgccag gccaaacaac ttttaattga tccagaagat
2401 gatgtaagag ataatatttt aaaatatgat gaagaaggtg gaggagaaga agaccaggac
2461 tatgacttga gccagctgca gcagcctgac actgtggagc ctgatgccat caagcctgtg
2521 ggaatccgac gaatggatga aagacccatc cacgccgagc cccagtatcc ggtccgatct
2581 gcagccccac accctggaga cattggggac ttcattaatg agggccttaa agcggctgac
2641 aatgacccca cagctccacc atatgactcc ctgttagtgt ttgactatga aggcagtggc
2701 tccactgctg ggtccttgag ctcccttaat tcctcaagta gtggtggtga gcaggactat
2761 gattacctga acgactgggg gccacggttc aagaaacttg ctgacatgta tggtggaggt
2821 gatgactgaa cttcagggtg aacttggttt ttggacaagt acaaacaatt tcaactgata
2881 ttcccaaaaa gcattcagaa gctaggcttt aactttgtag tctactagca cagtgcttgc
2941 tggaggcttt ggcataggct gcaaaccaat ttgggctcag agggaatatc agtgatccat
3001 actgtttgga aaaacactga gctcagttac acttgaattt tacagtacag aagcactggg
3061 attttatgtg cctttttgta cctttttcag attggaatta gttttctgtt taaggcttta
3121 atggtactga tttctgaaac gataagtaaa agacaaaata ttttgtggtg ggagcagtaa
3181 gttaaaccat gatatgcttc aacacgcttt tgttacattg catttgcttt tattaaaata
3241 caaaattaaa caaacaaaaa aactcatgga gcgattttat tatcttgggg gatgagacca
3301 tgagattgga aaatgtacat tacttctagt tttagacttt agtttgtttt tttttttttc
3361 actaaaatct taaaacttac tcagctggtt gcaaataaag ggagttttca tatcaccaat
3421 ttgtagcaaa attgaatttt ttcataaact agaatgttag acacattttg gtcttaatcc
3481 atgtacactt ttttatttct gtatttttcc acttcactgt aaaaatagta tgtgtacata
3541 atgttttatt ggcatagtct atggagaagt gcagaaactt cagaacatgt gtatgtatta
3601 tttggactat ggattcaggt tttttgcatg tttatatctt tcgttatgga taaagtattt
3661 acaaaacagt gacatttgat tcaattgttg agctgtagtt agaatactca atttttaatt
3721 tttttaattt ttttattttt tattttcttt ttggtttggg gagggagaaa agttcttagc
3781 acaaatgttt tacataattt gtaccaaaaa aaaaaaaaaa ggaaaggaaa gaaaggggtg
3841 gcctgacact ggtggcacta ctaagtgtgt gtttttttaa aaaaaaaatg gaaaaaaaaa
3901 agcttttaaa ctggagagac ttctgacaac agctttgcct ctgtattgtg taccagaata
3961 taaatgatac acctctgacc ccagcgttct gaataaaatg ctaattttgg atctgg
Homo sapiens neurofibromin 2 (NF2), transcript variant 1, mRNA (NCBI
Reference Sequence: NM_000268.3)
(SEQ ID NO: 40)
      1 tgcgcttccc gcgggcgcgc ggagtgagga cggtgacagc cacgcgcgcg cgtacgcgcc
    61 cgatgcagcg cggccccgtg accctagtcg gccgctgaga ggcgcgcgga gtctgggccg
  121 ctgccgtcta ggggtcccgt cccgaggcgt ccccggcatc tccggcccga atcccggagt
  181 gccgggtcgc gcctgcaccg aaggtcccgg ctcctgtgcc ctccctgcag ccgtcagggc
  241 ccgtccccca actccccttt ccgctcaggc agggtcctcg cggcccatgc tggccgctgg
  301 ggacccgcgc agcccagacc gttcccgggc cgggcagccg gccaccatgg tggccctgag
  361 gcctgtgcag caactccagg ggggctaaag ggctcagagt gcaggccgtg gggcgcgagg
  421 gtcccgggcc tgagccccgc gccatggccg gggccatcgc ttcccgcatg agcttcagct
  481 ctctcaagag gaagcaaccc aagacgttca ccgtgaggat cgtcaccatg gacgccgaga
  541 tggagttcaa ttgcgagatg aagtggaaag ggaaggacct ctttgatttg gtgtgccgga
  601 ctctggggct ccgagaaacc tggttctttg gactgcagta cacaatcaag gacacagtgg
  661 cctggctcaa aatggacaag aaggtactgg atcatgatgt ttcaaaggaa gaaccagtca
  721 cctttcactt cttggccaaa ttttatcctg agaatgctga agaggagctg gttcaggaga
  781 tcacacaaca tttattcttc ttacaggtaa agaagcagat tttagatgaa aagatctact
  841 gccctcctga ggcttctgtg ctcctggctt cttacgccgt ccaggccaag tatggtgact
  901 acgaccccag tgttcacaag cggggatttt tggcccaaga ggaattgctt ccaaaaaggg
  961 taataaatct gtatcagatg actccggaaa tgtgggagga gagaattact gcttggtacg
1021 cagagcaccg aggccgagcc agggatgaag ctgaaatgga atatctgaag atagctcagg
1081 acctggagat gtacggtgtg aactactttg caatccggaa taaaaagggc acagagctgc
1141 tgcttggagt ggatgccctg gggcttcaca tttatgaccc tgagaacaga ctgaccccca
1201 agatctcctt cccgtggaat gaaatccgaa acatctcgta cagtgacaag gagtttacta
1261 ttaaaccact ggataagaaa attgatgtct tcaagtttaa ctcctcaaag cttcgtgtta
1321 ataagctgat tctccagcta tgtatcggga accatgatct atttatgagg agaaggaaag
1381 ccgattcttt ggaagttcag cagatgaaag cccaggccag ggaggagaag gctagaaagc
1441 agatggagcg gcagcgcctc gctcgagaga agcagatgag ggaggaggct gaacgcacga
1501 gggatgagtt ggagaggagg ctgctgcaga tgaaagaaga agcaacaatg gccaacgaag
1561 cactgatgcg gtctgaggag acagctgacc tgttggctga aaaggcccag atcaccgagg
1621 aggaggcaaa acttctggcc cagaaggccg cagaggctga gcaggaaatg cagcgcatca
1681 aggccacagc gattcgcacg gaggaggaga agcgcctgat ggagcagaag gtgctggaag
1741 ccgaggtgct ggcactgaag atggctgagg agtcagagag gagggccaaa gaggcagatc
1801 agctgaagca ggacctgcag gaagcacgcg aggcggagcg aagagccaag cagaagctcc
1861 tggagattgc caccaagccc acgtacccgc ccatgaaccc aattccagca ccgttgcctc
1921 ctgacatacc aagcttcaac ctcattggtg acagcctgtc tttcgacttc aaagatactg
1981 acatgaagcg gctttccatg gagatagaga aagaaaaagt ggaatacatg gaaaagagca
2041 agcatctgca ggagcagctc aatgaactca agacagaaat cgaggccttg aaactgaaag
2101 agagggagac agctctggat attctgcaca atgagaactc cgacaggggt ggcagcagca
2161 agcacaatac cattaaaaag ctcaccttgc agagcgccaa gtcccgagtg gccttctttg
2221 aagagctcta gcaggtgacc cagccacccc aggacctgcc acttctcctg ctaccgggac
2281 cgcgggatgg accagatatc aagagagcca tccataggga gctggctggg ggtttccgtg
2341 ggagctccag aactttcccc agctgagtga agagcccagc ccctcttatg tgcaattgcc
2401 ttgaactacg accctgtaga gatttctctc atggcgttct agttctctga cctgagtctt
2461 tgttttaaga agtatttgtc ttcctttgtc taatgtggga ttcctgactc ccttcgtcca
2521 aggcaccggt gtgtgtgtgt cttgcactcc agagctgacc tccaccgccc agcctgggaa
2581 gtcattgtag ggagtgagac actgaagccc tgagaagcca gtgccatcat ccccaccccg
2641 cccagggttc cggaacattc attcccccac cggtgaggac ctggcatgca gcgaagcagc
2701 ccagcccggc ggatcccagg ccagcacgcc tgccggcttc tcatcgtcag ggagcccgcc
2761 cagagctcgt gacgagcaag tgctgggtcc ccgccaggca ccccgaggcg gcgctctggc
2821 tggcagctgg tggggaatag gcagggcagc tgtggctggg gagagacttt aggcagaagc
2881 tgtgatgcag gctgactgcc agccgagggg ctgggtagtg ccgtgcggga gctgatggta
2941 cagggcactc gctgtccccc tccggccacc ctagaccagg gtccgagagg caggcaggag
3001 ccactcatgt cttccccatt gcccgacgcc catagacgct ccttcctgtg tggggctggg
3061 gtactccctg gtcgtactgc agtcagcacc cgtaacccgg ctatgaccag ggatctgtaa
3121 gccctgtggc tccacaggtg ctgcttctca ctggcccaga ctctgagctc caccggccca
3181 gtctgcacgg cccatctgct tcaccttccc tcccagccac gtgccagtgg ccacagccca
3241 cttcccaacc cactgttgta cccaggcctc actttgctgt tgcccttgtc cctcttcggg
3301 ccctgaattt tctgttccct gggggccagc cagggccctt tgtgcccctc ccagcacagg
3361 cctgatgcag gtgtccactc acaggtggcg ctcacctagg ctgtcacagg acccacctcc
3421 atgccaggca acagagggcc acagaaccac ccccacggct cactccttgg tctggggcca
3481 ccttcttgcc ctttcttttt tttttttttt ttttttttcc gagatggagt ctctctctgt
3541 cacccaggtg ggagtgtggt ggcacaatct cggctcactg caacctccac ctcctgagtt
3601 caagcaattc tcctgcctca gcctcccaag tagctgggac tacaggcatg caccaccaca
3661 cttggccaat gttctgtatt tttaataggg acggggtttt gccatgttag ctaggctggt
3721 ctcaaactca cacctgggat tacaggcatg agccactgca cccagcccct tcttgccctt
3781 tcttttctcc atggctgatg ctgctgtggc cagccagggc ccttgagatc cttccagttt
3841 ggctgttatg caaagcaggt gatttgtctt aatcagataa aagatagagg ctatgggggc
3901 ctcaagattt ttggagagca gaggtggtct ctggcaattc catctggttt tgagaaactt
3961 agcagctcac agagcacaga gatcctgcct tcttcctact atcaggctga cctaatgggg
4021 ttgggctgct cggcaactgc ttgggtcacc ttgccccaag gaaaccagcc ctgggtgcca
4081 cccagccact tagggtctac agggtgggac tccagaccta gagcgtaagt atggatgttg
4141 tggccctgtg tcttcctagt gtgacccagc caggagcgga agcttcaggc gtttgtaaag
4201 tgaggtctgg ctctgcctcc tccgtttttt ttttttttct gtttctgttt ctgttttttt
4261 tttgagatgg agtctcgctc tgtcgcccag gctggagtgc agtgtcacga tctcagctca
4321 ctgcaacctc cgcctcccag gtacaagaga ttctcctgcc tcagcctccc gagtagctgg
4381 gactacaggc gtgtgccacc atgcctggct aatttttgta tttttagtag agatggggtt
4441 tcgccatgtt agccagactg gtctcgaact cctgacctca ggtgatcctc ccaccccggc
4501 ttcccaaagt tctgggatta taggcgtgag ccaccatgcc cggtctcttc tcagtcttga
4561 agcccatccc tggatttcca ccaggagtta ctttcctcct gacctgtaaa tttgttcttt
4621 aacaatggct gcaggtggga gcatatggtg gtttataaaa acgctgtcgg gctttgtttc
4681 cttctttagc tgccgtgtct acttctgaag tctgggaagt gccaagccac gcggcctcaa
4741 gggagctggc tggtgtttga gctgtggcag aagcacctgg ggctccaggg agcaggctgg
4801 gaactgcagg accttgctca gccaggagca cttccccctc cttgaggcag gaatactgag
4861 gtgcctcccc acagatggag aaggtggaga ggaggatggg cctcaggagc atctcaagcc
4921 ccagtagcag gagaaagaaa gaaagagatg cctggttttc acagactggt tcctgtggct
4981 gggatgactg catccttttt tttttttttt tgagacggag tttttgcctt tgtcgcccag
5041 gctggagtgc aatggcgtga tctcggctca ccgcaacctc cgcctcccgg attcaagcaa
5101 ttctcctgcc tcagcctccc gagtagctgg gattacaggc acgcacctcc acgtccggct
5161 aattttgtat ttttagtgga gacggggttt ctccatgtcg gtcaggctgg tctcgaactc
5221 ccgacctcag gtgatctgcc cacctcggcc tcccaaagtg ctgggattac aggcatgagc
5281 caccgcgctt ggccagactg cgtccttttt aagcgaacat tttagggcct gggagtttgt
5341 caagtaagga agtctcaagc ccaaagagca gcgtcctgac catggtggtt tcattacgag
5401 cccttctgct ggctctcagg cagaagcccc acagcaccgg gaccattcat gaggtcactg
5461 cccagctcat gatgtccgtg aggctgtcct tttggccagt agccgtgtgc agctgtgtgg
5521 cacagatggc ttcgttcatc ctgatcaagg ccccacctca gccacagcag tccccccaac
5581 ctgtgttgtc caccctatta ttcatgtacc tgccaggccc tgctagatag caccccgtgg
5641 cattacataa cacttcatga gtggctgtgt cttgtaattt tggggacagg tttctctctt
5701 tccctctctt ttttttgtca aaagcccaga gactgacaac cagctgcagt gtctaagtgt
5761 tcctcactga cagggtgggg cctcaccacc cctggaggga gcagcgttgg cagggagaca
5821 gcctggccca gtgaccctgg gcccaagcca gcccctccag ggctttcagg gaagcgccat
5881 ccattttcaa agatgtcaaa cgtcacttct tcctgtaggg cccgagtcct gcctcctatc
5941 agggccagat catagaaggc tattttctat tctggggaac gattataact taaatgattg
6001 ttttaataaa aattctaagc tggaaaataa aaaaaaaaaa aaaaaa
Homo sapiens macrophage stimulating 1 (MST1), transcript variant 1, mRNA
(NCBI Reference Sequence: NM_020998.3)
(SEQ ID NO: 41)
      1 cagcaggccc tgagctgacg tgtggagcca gagccaccca atcccgtagg gacaggtttc
    61 acaacttccc ggatggggct gtggtgggtc acagtgcagc ctccagccag aaggatgggg
  121 tggctcccac tcctgctgct tctgactcaa tgcttagggg tccctgggca gcgctcgcca
  181 ttgaatgact tccaagtgct ccggggcaca gagctacagc acctgctaca tgcggtggtg
  241 cccgggcctt ggcaggagga tgtggcagat gctgaagagt gtgctggtcg ctgtgggccc
  301 ttaatggact gccgggcctt ccactacaac gtgagcagcc atggttgcca actgctgcca
  361 tggactcaac actcgcccca cacgaggctg cggcgttctg ggcgctgtga cctcttccag
  421 aagaaagact acgtacggac ctgcatcatg aacaatgggg ttgggtaccg gggcaccatg
  481 gccacgaccg tgggtggcct gccctgccag gcttggagcc acaagttccc aaatgatcac
  541 aagtacacgc ccactctccg gaatggcctg gaagagaact tctgccgtaa ccctgatggc
  601 gaccccggag gtccttggtg ctacacaaca gaccctgctg tgcgcttcca gagctgcggc
  661 atcaaatcct gccgggaggc cgcgtgtgtc tggtgcaatg gcgaggaata ccgcggcgcg
  721 gtagaccgca cggagtcagg gcgcgagtgc cagcgctggg atcttcagca cccgcaccag
  781 caccccttcg agccgggcaa gttcctcgac caaggtctgg acgacaacta ttgccggaat
  841 cctgacggct ccgagcggcc atggtgctac actacggatc cgcagatcga gcgagagttc
  901 tgtgacctcc cccgctgcgg gtccgaggca cagccccgcc aagaggccac aactgtcagc
  961 tgcttccgcg ggaagggtga gggctaccgg ggcacagcca ataccaccac tgcgggcgta
1021 ccttgccagc gttgggacgc gcaaatcccg catcagcacc gatttacgcc agaaaaatac
1081 gcgtgcaaag accttcggga gaacttctgc cggaaccccg acggctcaga ggcgccctgg
1141 tgcttcacac tgcggcccgg catgcgcgcg gccttttgct accagatccg gcgttgtaca
1201 gacgacgtgc ggccccagga ctgctaccac ggcgcagggg agcagtaccg cggcacggtc
1261 agcaagaccc gcaagggtgt ccagtgccag cgctggtccg ctgagacgcc gcacaagccg
1321 cagttcacgt ttacctccga accgcatgca caactggagg agaacttctg ccggaaccca
1381 gatggggata gccatgggcc ctggtgctac acgatggacc caaggacccc attcgactac
1441 tgtgccctgc gacgctgcgc tgatgaccag ccgccatcaa tcctggaccc cccagaccag
1501 gtgcagtttg agaagtgtgg caagagggtg gatcggctgg atcagcggcg ttccaagctg
1561 cgcgtggttg ggggccatcc gggcaactca ccctggacag tcagcttgcg gaatcggcag
1621 ggccagcatt tctgcggggg gtctctagtg aaggagcagt ggatactgac tgcccggcag
1681 tgcttctcct cctgccatat gcctctcacg ggctatgagg tatggttggg caccctgttc
1741 cagaacccac agcatggaga gccaagccta cagcgggtcc cagtagccaa gatggtgtgt
1801 gggccctcag gctcccagct tgtcctgctc aagctggaga gatctgtgac cctgaaccag
1861 cgtgtggccc tgatctgcct gccccctgaa tggtatgtgg tgcctccagg gaccaagtgt
1921 gagattgcag gctggggtga gaccaaaggt acgggtaatg acacagtcct aaatgtggcc
1981 ttgctgaatg tcatctccaa ccaggagtgt aacatcaagc accgaggacg tgtgcgggag
2041 agtgagatgt gcactgaggg actgttggcc cctgtggggg cctgtgaggg tgactacggg
2101 ggcccacttg cctgctttac ccacaactgc tgggtcctgg aaggaattat aatccccaac
2161 cgagtatgcg caaggtcccg ctggccagct gtcttcacgc gtgtctctgt gtttgtggac
2221 tggattcaca aggtcatgag actgggttag gcccagcctt gatgccatat gccttgggga
2281 ggacaaaact tcttgtcaga cataaagcca tgtttcctct ttatgcctgt aaaaaaaaaa
2341 aaaaaaaa
Homo sapiens serine/threonine kinase 3 (STK3)NIST-2, transcript variant 1,
mRNA (NCBI Reference Sequence: NM_006281.4)
(SEQ ID NO: 42)
      1 agatccgccg cggagttacg ggaaagttgg tccgagttcc cagagtttcc ctctgtggtg
    61 ccctaggctc ggccggccgg tgccccggct cctttcctcc tttcggcctt cgccgtccac
  121 caggtccctc tctctgtccc cggccgccat ggagcagccg ccggcgccta agagtaaact
  181 aaaaaagctg agtgaagaca gtttgactaa gcagcctgaa gaagtttttg atgtattaga
  241 gaagcttgga gaagggtctt atggaagtgt atttaaagca atacacaagg aatccggtca
  301 agttgtcgca attaaacaag tacctgttga atcagatctt caggaaataa tcaaagaaat
  361 ttccataatg cagcaatgtg acagcccata tgttgtaaag tactatggca gttattttaa
  421 gaatacagac ctctggattg ttatggagta ctgtggcgct ggctctgtct cagacataat
  481 tagattacga aacaagacat taatagaaga tgaaattgca accattctta aatctacatt
  541 gaaaggacta gaatatttgc actttatgag aaaaatacac agagatataa aagctggaaa
  601 tattctcctc aatacagaag gacatgcaaa attggcagat tttggagtgg ctggtcagtt
  661 aacagataca atggcaaaac gcaatactgt aataggaact ccattttgga tggctcctga
  721 ggtgattcaa gaaataggct ataactgtgt ggccgacatc tggtcccttg gcattacttc
  781 tatagaaatg gctgaaggaa aacctcctta tgctgatata catccaatga gggctatttt
  841 tatgattccc acaaatccac caccaacatt cagaaagcca gaactttggt ccgatgattt
  901 caccgatttt gttaaaaagt gtttggtgaa gaatcctgag cagagagcta ctgcaacaca
  961 acttttacag catcctttta tcaagaatgc caaacctgta tcaatattaa gagacctgat
1021 cacagaagct atggagatca aagctaaaag acatgaggaa cagcaacgag aattggaaga
1081 ggaagaagaa aattcggatg aagatgagct ggattcccac accatggtga agactagtgt
1141 ggagagtgtg ggcaccatgc gggccacaag cacgatgagt gaaggggccc agaccatgat
1201 tgaacataat agcacgatgt tggaatccga cttggggacc atggtgataa acagtgagga
1261 tgaggaagaa gaagatggaa ctatgaaaag aaatgcaacc tcaccacaag tacaaagacc
1321 atctttcatg gactactttg ataagcaaga cttcaagaat aagagtcacg aaaactgtaa
1381 tcagaacatg catgaaccct tccctatgtc caaaaacgtt tttcctgata actggaaagt
1441 tcctcaagat ggagactttg actttttgaa aaatctaagt ttagaagaac tacagatgcg
1501 gttaaaagca ctggacccca tgatggaacg ggagatagaa gaacttcgtc agagatacac
1561 tgcgaaaaga cagcccattc tggatgcgat ggatgcaaag aaaagaaggc agcaaaactt
1621 ttgagtctaa tttcctctct gtttttaact attctggaga ccaagaaacc actaggaatt
1681 gaaggaatat ttggatattt ttaatcctaa gattttgccc tacaattagg cagaggtcaa
1741 aaagtgacaa tggtacatgc ccaggtaaat tcccaaaagg cagaattgac agttgtatct
1801 gctgtgcatt cactctaaga tgaggagaac aaaagaagtg tattctcttg ttctgtcagc
1861 tgcataccag taataaaact gttatgaaat ggattttcaa ggtctctaaa ccttgaaaat
1921 ccaaagctat tgttgcattg tacagcactg aagggcttta tgttacaata ttctttattc
1981 ctatctagta tactaggcta tttattgtat ccccttaggt aaacttattt atttatgcta
2041 ttttgctttg tttcattttt taaggacaag atcaggatag ctttggtgaa ggtagggtca
2101 tattaatatg atgataatgt gcaaccaatt tatactttct gcagggagct atggggtaca
2161 ttccttgatt tccaggatag tttttcaaat aggaaagcaa taatggcagt agttctcaaa
2221 tgggctaggc cttttttata ttgaagcaat aattccattt ttaccctttg aaattttgtt
2281 tttttgattt ttgatgtttg gtacaaatag aactatatat atttaggtaa aatagatcta
2341 tcgtgtttaa aaccaaagaa atcaatggaa cccttgcaca aaaaagtgtg ataaatattt
2401 ttaaataaaa acttaataca aatgtaattt gttaatattg tttcatgttt tatgtgtaga
2461 tctaatagct gaactgattc aaactgtaat aagctcatca atttcatttc tatgaaaatg
2521 tgctctgttg tcacaggatg tttctgttga ttttattcat ttcctgggaa ttggtaaaca
2581 tcatgttcct gatgataacc cagtagcaaa aacatttgta ctgagtggta caagccttgg
2641 ggactgaaaa aaaaaagatt aaaaccatta aaaagaaact catttttacg ctgaatgaac
2701 atttatatga ttgcattggg accagtcatt tcctaagcta catatggcca tcttgacagt
2761 gttttttctt ttgtgtgttt aattattatg tgtaaatcat aaagacaaat aaatttcact
2821 gtgccaccca gca
Homo sapiens large tumor suppressor kinase 1 (LATS1), transcript variant 1,
mRNA (NCBI Reference Sequence: NM_004690.4)
(SEQ ID NO: 43)
      1 agcggagtgc ggcggcggcg acactgagtg gaaggcaaaa tggcggcggc ggcggcggtg
    61 gcctggtgtt aaggggagag ccaggtcctc acgacccctg ggacgggccg cgctggcccg
  121 cggcagcccc cccgttcgtc tccccgctct gccccaccag ggatacttgg ggttgctggg
  181 acggactctg gccgcctcag cgtccgccct caggcccgtg gccgctgtcc aggagctctg
  241 ctctcccctc cagagttaat tatttatatt gtaaagaatt ttaacagtcc tggggacttc
  301 cttgaaggat cattttcact tttgctcaga agaaagctct ggatctatca aataaagaag
  361 tccttcgtgt gggctacata tatagatgtt ttcatgaaga ggagtgaaaa gccagaagga
  421 tatagacaaa tgaggcctaa gacctttcct gccagtaact atactgtcag tagccggcaa
  481 atgttacaag aaattcggga atcccttagg aatttatcta aaccatctga tgctgctaag
  541 gctgagcata acatgagtaa aatgtcaacc gaagatcctc gacaagtcag aaatccaccc
  601 aaatttggga cgcatcataa agccttgcag gaaattcgaa actctctgct tccatttgca
  661 aatgaaacaa attcttctcg gagtacttca gaagttaatc cacaaatgct tcaagacttg
  721 caagctgctg gatttgatga ggatatggtt atacaagctc ttcagaaaac taacaacaga
  781 agtatagaag cagcaattga attcattagt aaaatgagtt accaagatcc tcgacgagag
  841 cagatggctg cagcagctgc cagacctatt aatgccagca tgaaaccagg gaatgtgcag
  901 caatcagtta accgcaaaca gagctggaaa ggttctaaag aatccttagt tcctcagagg
  961 catggcccgc cactaggaga aagtgtggcc tatcattctg agagtcccaa ctcacagaca
1021 gatgtaggaa gacctttgtc tggatctggt atatcagcat ttgttcaagc tcaccctagc
1081 aacggacaga gagtgaaccc cccaccacca cctcaagtaa ggagtgttac tcctccacca
1141 cctccaagag gccagactcc ccctccaaga ggtacaactc cacctccccc ttcatgggaa
1201 ccaaactctc aaacaaagcg ctattctgga aacatggaat acgtaatctc ccgaatctct
1261 cctgtcccac ctggggcatg gcaagagggc tatcctccac cacctctcaa cacttccccc
1321 atgaatcctc ctaatcaagg acagagaggc attagttctg ttcctgttgg cagacaacca
1381 atcatcatgc agagttctag caaatttaac tttccatcag ggagacctgg aatgcagaat
1441 ggtactggac aaactgattt catgatacac caaaatgttg tccctgctgg cactgtgaat
1501 cggcagccac cacctccata tcctctgaca gcagctaatg gacaaagccc ttctgcttta
1561 caaacagggg gatctgctgc tccttcgtca tatacaaatg gaagtattcc tcagtctatg
1621 atggtgccaa acagaaatag tcataacatg gaactatata acattagtgt acctggactg
1681 caaacaaatt ggcctcagtc atcttctgct ccagcccagt catccccgag cagtgggcat
1741 gaaatcccta catggcaacc taacatacca gtgaggtcaa attcttttaa taacccatta
1801 ggaaatagag caagtcactc tgctaattct cagccttctg ctacaacagt cactgcaatt
1861 acaccagctc ctattcaaca gcctgtgaaa agtatgcgtg tattaaaacc agagctacag
1921 actgctttag cacctacaca cccttcttgg ataccacagc caattcaaac tgttcaaccc
1981 agtccttttc ctgagggaac cgcttcaaat gtgactgtga tgccacctgt tgctgaagct
2041 ccaaactatc aaggaccacc accaccctac ccaaaacatc tgctgcacca aaacccatct
2101 gttcctccat acgagtcaat cagtaagcct agcaaagagg atcagccaag cttgcccaag
2161 gaagatgaga gtgaaaagag ttatgaaaat gttgatagtg gggataaaga aaagaaacag
2221 attacaactt cacctattac tgttaggaaa aacaagaaag atgaagagcg aagggaatct
2281 cgtattcaaa gttattctcc tcaagcattt aaattcttta tggagcaaca tgtagaaaat
2341 gtactcaaat ctcatcagca gcgtctacat cgtaaaaaac aattagagaa tgaaatgatg
2401 cgggttggat tatctcaaga tgcccaggat caaatgagaa agatgctttg ccaaaaagaa
2461 tctaattaca tccgtcttaa aagggctaaa atggacaagt ctatgtttgt gaagataaag
2521 acactaggaa taggagcatt tggtgaagtc tgtctagcaa gaaaagtaga tactaaggct
2581 ttgtatgcaa caaaaactct tcgaaagaaa gatgttcttc ttcgaaatca agtcgctcat
2641 gttaaggctg agagagatat cctggctgaa gctgacaatg aatgggtagt tcgtctatat
2701 tattcattcc aagataagga caatttatac tttgtaatgg actacattcc tgggggtgat
2761 atgatgagcc tattaattag aatgggcatc tttccagaaa gtctggcacg attctacata
2821 gcagaactta cctgtgcagt tgaaagtgtt cataaaatgg gttttattca tagagatatt
2881 aaacctgata atattttgat tgatcgtgat ggtcatatta aattgactga ctttggcctc
2941 tgcactggct tcagatggac acacgattct aagtactatc agagtggtga ccatccacgg
3001 caagatagca tggatttcag taatgaatgg ggggatccct caagctgtcg atgtggagac
3061 agactgaagc cattagagcg gagagctgca cgccagcacc agcgatgtct agcacattct
3121 ttggttggga ctcccaatta tattgcacct gaagtgttgc tacgaacagg atacacacag
3181 ttgtgtgatt ggtggagtgt tggtgttatt ctttttgaaa tgttggtggg acaacctcct
3241 ttcttggcac aaacaccatt agaaacacaa atgaaggtta tcaactggca aacatctctt
3301 cacattccac cacaagctaa actcagtcct gaagcttctg atcttattat taaactttgc
3361 cgaggacccg aagatcgctt aggcaagaat ggtgctgatg aaataaaagc tcatccattt
3421 tttaaaacaa ttgacttctc cagtgacctg agacagcagt ctgcttcata cattcctaaa
3481 atcacacacc caacagatac atcaaatttt gatcctgttg atcctgataa attatggagt
3541 gatgataacg aggaagaaaa tgtaaatgac actctcaatg gatggtataa aaatggaaag
3601 catcctgaac atgcattcta tgaatttacc ttccgaaggt tttttgatga caatggctac
3661 ccatataatt atccgaagcc tattgaatat gaatacatta attcacaagg ctcagagcag
3721 cagtcggatg aagatgatca aaacacaggc tcagagatta aaaatcgcga tctagtatat
3781 gtttaacaca ctagtaaata aatgtaatga ggatttgtaa aagggcctga aatgcgaggt
3841 gttttgaggt tctgagagta aaattatgca aatatgacag agctatatat gtgtgctctg
3901 tgtacaatat tttattttcc taaattatgg gaaatccttt taaaatgtta atttattcca
3961 gccgtttaaa tcagtattta gaaaaaaatt gttataagga aagtaaatta tgaactgaat
4021 attatagtca gttcttggta cttaaagtac ttaaaataag tagtgctttg tttaaaagga
4081 gaaacctggt atctatttgt atatatgcta aataatttta aaatacaaga gtttttgaaa
4141 tttttttgaa agacagtttt agttttatct tgctttaacc aaatatgaaa cataccccct
4201 attttacaga gctctttttt cccctcataa ccttgttttt ggtagaaaat aagctagaga
4261 aattaagcca tcgtgttggt gagtgttcct aggctaatga taatctgtat aattcacatc
4321 ctgaaactaa ggaatacagg gttgaaaaaa tattaatatg tttgtcagaa ggaaaaataa
4381 tgcatttatc ttccccccca ccccccgccc catggaatat ttaatctatt taatcttctt
4441 gcatttattt ctcaagaatt actggcttta aaagaagcca aagcactact agcttttttt
4501 ccatattggt atttttgatg ctgcttccaa ttttaaaagg gaacaaagct gccataaatc
4561 gaaatgttca atactaaaag ctaaaatatt tctcaccatc ctaagcagat aattatttta
4621 attttcatat acttttcctg tatagtaact attttgatta tatcatcaat gttacctgtt
4681 tcctctttca gaacagtgct gcatatacag attgttattg gcaaaggaaa atctggctat
4741 ctggcaatat tttacctaag cgcagattaa ttggtgaaaa aattaactct taagatggcc
4801 attaataatt aggaaagttt acagagtggt cttagtagaa aattcaagtc ctcctaattt
4861 atttaaggtt caataatgcg ttcaacatgc ctgttatgta taacgcttag gttctaagga
4921 agattaaggt ttcataccaa aatacatgta gcttatcttt taggaagggg aaaaaggctc
4981 cattttgacc atagtaaaat ttgtgttgtg ttttatttcc ttttcttaag ctccactgat
5041 aagggattgt ttttatcaaa agttactatt tgtagattgg aggcataatt ttagtgattt
5101 tcatactttt agctttcttc gcataaaagc taattgaaac cgtatatgta gtaaaattaa
5161 aggcagagct gttgcagttg aattggagag ttagggcaaa gaacacttat tagcccacac
5221 ttcccacctt tctacaggtg gtcctttcag agctcagcct gaaaacccac tactgtgtta
5281 tcgtgcgtct tttggggtta gtggttcttt tgagaatctg aaggaagctg tggactcttc
5341 ctagaaaaaa aaaccacaca tacacataca atgttgcatg cagtttcaag ggattttgga
5401 catattgaaa cctatcacag gctgtaggtt atggacctct gtgccatgag aaaattgata
5461 cattaaacta agaactttgt ttttaactta ccaatcacta ctcagcacat cttatataag
5521 ctgataattt gtgatggaaa aggtctgtag catgtgatat aaggtgacct tatgaatgcc
5581 tctcttgctg gtacattaag ttgttttaat atatcatttg gaggggactg aaatgttagg
5641 ctcattacaa gcttgataca gaaatatttc tgaaggattt ctaatcagaa ttgtaaaaca
5701 atgtgctatc atgaaatcgc agtcttcacc tcatggttca tggaacattt ggttagtccc
5761 ataaaatcct atgcaaaaca aagtagttca agaattttta ggtgggtagt cacatttata
5821 aggtattcct cttactcttt gggctttttc agtctgattt atttaaattt tcatttagtt
5881 gttttacttt tggactaagg tgcaatacag tagaagataa ctttgttaca tttatgttgt
5941 aggaaaacta aggtgctgtc tcctccccct tcccttccca caaaatctgt attcccccta
6001 ttgctgaaat gtaacagaca ctacaaattt tgtattcttt ttttgttttt tgttttgaga
6061 cagggtctca ctctgtcacc caggctggag ggcagtggcg cttcacagct cactgcatcc
6121 tcaaccttgg gggctcacgc agtcctcccg cctcagcctc ccaagtagct gggcatgcgc
6181 caccaagccc agctaatttt tgtatcttta gtagagatgg ggtttcgcca tgttgcccag
6241 gttggtgtgg aattcctggg ctccagttat atgcccacct cagcctccca aagtgctggg
6301 attacagacg tgacccaccg cgcctggcgc aaatatgtat tcttttaaaa tttcctctga
6361 tactataagc tttttgcatt tatctgaagc agtatacatg cctttggtat cagcaatttt
6421 aacagtttgg atatacttat cagctatctt attccaaaac tacatctact tcttccagta
6481 tagaatctgg tgcttcctga ccaaaaagat gagaaaaaca atgttaaaaa tatagatgct
6541 ttccattgaa atggagtgaa aacattggtt ctatatgttt tcttttaaaa taattttctt
6601 attaaaaact tgctgtcttt attatactta ccctttttat gcatatcaat agtatttata
6661 agatgtgttc tataattatg taattgtaga tactgttatg cattgtccag tgacatcata
6721 aggcaggccc tactgctgta tcttttctac cttcttattt gtaatagaaa ctatagaatg
6781 tatgactaaa aagtcacttt gagattgact tttttaaaaa gttattacct tctgctgttg
6841 caaagtgcaa aactgtgagt ggaattgttt tattctgact taatgtgtta gaaattagag
6901 aatacagtgg gaggattttt agacattgct gctgctgtta cccaaggtat tttagataaa
6961 aaatttttaa taaacatccc tttggtattt aaagtggaac atttagcctg ttcattttaa
7021 tctaaagcaa aaagtaattt gggtcaaaat attggtatat ttgtaaagcg ccttaatata
7081 tccctttgtg gaaggcacta cacagtttac ttttatattg tattgtgtat ataagtattt
7141 tgtattaaaa ttgaatcagt ggcaacatta aagttttata aaatcatgct ttgttagaaa
7201 aagaattaca gctttgcaat ataactaatt gtttcgcata attctgaatg taatagatat
7261 gaataatcag cctgtgtttt taatgaactt atttgtattt tcccaatcat tttctctagt
7321 gtaatgtttg ctgggataat aaaaaaaatt caaatctttc aa
Homo sapiens large tumor suppressor kinase 2 (LATS2), mRNA (NCBI
Reference Sequence: NM_014572.3)
(SEQ ID NO: 44)
      1 gacgcccgtg gaatgccaac aatgtagcga atgtcccact tgggtctgcg ctttggaacc
    61 gcggcgtgag cgccccggga agatggagca gtcgccgtcc acgccaccgc cgccgcccgg
  121 ggctcccccg tccctgcggg gccagcagca gctccagcca ccagtgcccg gtctcccggc
  181 gcgagaggcc cgggagccgc cggccaggac gcccccgagg gtgtagaccg cgcccctgga
  241 gagagtgata atcttcaaaa tgaagacttt ggaaaatttt aggttctcta taggaactac
  301 aaaaatggaa ggaaagaaca ttttcaaaag gaaattattt tgaaagtatg tttacaacaa
  361 actgatacta ttgacagttt ttttttttaa ataataaaac actttaagaa gattgtattt
  421 atggtaaaag gaaactggac taacaatgag gccaaagact tttcctgcca cgacttattc
  481 tggaaatagc cggcagcgac tgcaagagat tcgtgagggg ttaaaacagc catccaagtc
  541 ttcggttcag gggctacccg caggaccaaa cagtgacact tccctggatg ccaaagtcct
  601 ggggagcaaa gatgccacca ggcagcagca gcagatgaga gccaccccaa agttcggacc
  661 ttatcagaaa gccttgaggg aaatcagata ttccttgttg ccttttgcta atgaatcggg
  721 cacctctgca gctgcagaag tgaaccggca aatgctgcag gaactggtga acgcaggatg
  781 cgaccaggag atggctggcc gagctctcaa gcagactggc agcaggagca tcgaggccgc
  841 cctggagtac atcagcaaga tgggctacct ggacccgagg aatgagcaga ttgtgcgggt
  901 cattaagcag acctccccag gaaaggggct catgccaacc ccagtgacgc ggaggcccag
  961 cttcgaagga accggcgatt cgtttgcgtc ctaccaccag ctgagcggta ccccctacga
1021 gggcccaagc ttcggcgctg acggccccac ggcgctggag gagatgccgc ggccgtacgt
1081 ggactacctt ttccccggag tcggccccca cgggcccggc caccagcacc agcacccacc
1141 caagggctac ggtgccagcg tagaggcagc aggggcacac ttcccgctgc agggcgcgca
1201 ctacgggcgg ccgcacctgc tggtgcctgg ggaacccctg ggctacggag tgcagcgcag
1261 cccctccttc cagagcaaga cgccgccgga gaccgggggt tacgccagcc tgcccacgaa
1321 gggccaggga ggaccgccag gcgccggcct cgctttccca ccccctgccg ccgggctcta
1381 cgtgccgcac ccacaccaca agcaggccgg tcccgcggcc caccagctgc atgtgctggg
1441 ctcccgcagc caggtgttcg ccagcgacag ccccccgcag agcctgctca ctccctcgcg
1501 gaacagcctc aacgtggacc tgtatgaatt gggcagcacc tccgtccagc agtggccggc
1561 tgccaccctg gcccgccggg actccctgca gaagccgggc ctggaggcgc cgccgcgcgc
1621 gcacgtggcc ttccggcctg actgcccagt gcccagcagg accaactcct tcaacagcca
1681 ccagccgcgg cccggtccgc ctggcaaggc cgagccctcc ctgcccgccc ccaacaccgt
1741 gacggctgtc acggccgcgc acatcttgca cccggtgaag agcgtgcgtg tgctgaggcc
1801 ggagccgcag acggctgtgg ggccctcgca ccccgcctgg gtgcccgcgc ctgccccggc
1861 ccccgccccc gcccccgccc cggctgcgga gggcttggac gccaaggagg agcatgccct
1921 ggcgctgggc ggcgcaggcg ccttcccgct ggacgtggag tacggaggcc cagaccggag
1981 gtgcccgcct ccgccctacc cgaagcacct gctgctgcgc agcaagtcgg agcagtacga
2041 cctggacagc ctgtgcgcag gcatggagca gagcctccgt gcgggcccca acgagcccga
2101 gggcggcgac aagagccgca aaagcgccaa gggggacaaa ggcggaaagg ataaaaagca
2161 gattcagacc tctcccgttc ccgtccgcaa aaacagcaga gacgaagaga agagagagtc
2221 acgcatcaag agctactcgc catacgcctt taagttcttc atggagcagc acgtggagaa
2281 tgtcatcaaa acctaccagc agaaggttaa ccggaggctg cagctggagc aagaaatggc
2341 caaagctgga ctctgtgaag ctgagcagga gcagatgcgg aagatcctct accagaaaga
2401 gtctaattac aacaggttaa agagggccaa gatggacaag tctatgtttg tcaagatcaa
2461 aaccctgggg atcggtgcct ttggagaagt gtgccttgct tgtaaggtgg acactcacgc
2521 cctgtacgcc atgaagaccc taaggaaaaa ggatgtcctg aaccggaatc aggtggccca
2581 cgtcaaggcc gagagggaca tcctggccga ggcagacaat gagtgggtgg tcaaactcta
2641 ctactccttc caagacaaag acagcctgta ctttgtgatg gactacatcc ctggtgggga
2701 catgatgagc ctgctgatcc ggatggaggt cttccctgag cacctggccc ggttctacat
2761 cgcagagctg actttggcca ttgagagtgt ccacaagatg ggcttcatcc accgagacat
2821 caagcctgat aacattttga tagatctgga tggtcacatt aaactcacag atttcggcct
2881 ctgcactggg ttcaggtgga ctcacaattc caaatattac cagaaaggga gccatgtcag
2941 acaggacagc atggagccca gcgacctctg ggatgatgtg tctaactgtc ggtgtgggga
3001 caggctgaag accctagagc agagggcgcg gaagcagcac cagaggtgcc tggcacattc
3061 actggtgggg actccaaact acatcgcacc cgaggtgctc ctccgcaaag ggtacactca
3121 actctgtgac tggtggagtg ttggagtgat tctcttcgag atgctggtgg ggcagccgcc
3181 ctttttggca cctactccca cagaaaccca gctgaaggtg atcaactggg agaacacgct
3241 ccacattcca gcccaggtga agctgagccc tgaggccagg gacctcatca ccaagctgtg
3301 ctgctccgca gaccaccgcc tggggcggaa tggggccgat gacctgaagg cccacccctt
3361 cttcagcgcc attgacttct ccagtgacat ccggaagcag ccagccccct acgttcccac
3421 catcagccac cccatggaca cctcgaattt cgaccccgta gatgaagaaa gcccttggaa
3481 cgatgccagc gaaggtagca ccaaggcctg ggacacactc acctcgccca ataacaagca
3541 tcctgagcac gcattttacg aattcacctt ccgaaggttc tttgatgaca atggctaccc
3601 ctttcgatgc ccaaagcctt caggagcaga agcttcacag gctgagagct cagatttaga
3661 aagctctgat ctggtggatc agactgaagg ctgccagcct gtgtacgtgt agatgggggc
3721 caggcacccc caccactcgc tgcctcccag gtcagggtcc cggagccggt gccctcacag
3781 gccaataggg aagccgaggg ctgttttgtt ttaaattagt ccgtcgatta cttcacttga
3841 aattctgctc ttcaccaaga aaacccaaac aggacacttt tgaaaacagg actcagcatc
3901 gctttcaata ggcttttcag gaccttcact gcattaaaac aatatttttg aaaatttagt
3961 acagtttaga aagagcactt attttgttta tatccatttt ttcttactaa attataggga
4021 ttaactttga caaatcatgc tgctgttatt ttctacattt gtattttatc catagcactt
4081 attcacattt aggaaaagac ataaaaactg aagaacattg atgagaaatc tctgtgcaat
4141 aatgtaaaaa aaaaaaaaga taacactctg ctcaatgtca cggagaccat tttatccaca
4201 caatggtttt tgttttttat tttttcccat gtttcaaaat tgtgatataa tgatataatg
4261 ttaaaagctg ctttttttgg ctttttgcat atctagtata ataggaagtg tgagcaaggt
4321 gatgatgtgg ctgtgatttc cgacgtctgg tgtgtggaga gtactgcatg agcagagttc
4381 ttctattata aaattaccat atcttgccat tcacagcagg tcctgtgaat acgtttttac
4441 tgagtgtctt taaatgaggt gttctagaca gtgtgctgat aatgtattgt gcgggtgacc
4501 tcttcgctat gattgtatct cttactgttt tgttaaagaa atgcagatgt gtaactgaga
4561 agtgatttgt gtgtgtgtct tggttgtgat tggattcttt gggggggggg aactgaaaca
4621 tttgtcatat actgaactta tatacatcaa aagggattaa tacagcgatg ccaaaaagtt
4681 taatcacgga cacatgtccg tttctgtagt ccgtatgctc tttcattctt ggtagagctg
4741 gtatgtggaa tgccatacct ctgaccctac tacttacctt tttactgaca gactgcccac
4801 actgaaagct tcagtgaatg ttcttagtcc tgttttcttc tgttactgtc aggaaactga
4861 gtgatctaat ggttctctca cttttttttt gttcttttag tgtactttga agtatcaaat
4921 cttaacttgg tttaaacaat acatattcct aacctttgta aaaaagcaaa gattcttcaa
4981 aatgacattg aaataaaaag taagccatac gtattttctt agaagtatag atgtatgtgc
5041 gtgtatacac acacacacac acacacagag ataaacacaa tattccttat ttcaaattag
5101 tatgattcct atttaaagtg atttatattt gagtaaaaag ttcaattctt ttttgctttt
5161 taaaaaatct gatgcttcat aattttcatt atattattcc acatattttt ccttgaagtt
5221 cttagcataa tgtatccatt acttagtata tatctaggca acaacactta gaagtttatc
5281 agtgtttaaa ctaaaaaaat aaagattcct gtgtactggt ttacatttgt gtgagtggca
5341 tactcaagtc tgctgtgcct gtcgtcgtga ctgtcagtat tctcgctatt ttatagtcgt
5401 gccatgttgt tactcacagc gctctgacat actttcatgt ggtaggttct ttctcaggaa
5461 ctcagtttaa ctattattta ttgatatatc attacctttg aaaagcttct actggcacaa
5521 tttattatta aaattttgaa tccaaa
Homo sapiens Yes associated protein 1 (YAP1), transcript variant 9, mRNA
(NCBI Reference Sequence: NM_001282101.1)
(SEQ ID NO: 45)
      1 gccgccgcca gggaaaagaa agggaggaag gaaggaacaa gaaaaggaaa taaagagaaa
    61 ggggaggcgg ggaaaggcaa cgagctgtcc ggcctccgtc aagggagttg gagggaaaaa
  121 gttctcaggc gccgcaggtc cgagtgcctc gcagcccctc ccgaggcgca gccgccagac
  181 cagtggagcc ggggcgcagg gcgggggcgg aggcgccggg gcgggggatg cggggccgcg
  241 gcgcagcccc ccggccctga gagcgaggac agcgccgccc ggcccgcagc cgtcgccgct
  301 tctccacctc ggcccgtgga gccggggcgt ccgggcgtag ccctcgctcg cctgggtcag
  361 ggggtgcgcg tcgggggagg cagaagccat ggatcccggg cagcagccgc cgcctcaacc
  421 ggccccccag ggccaagggc agccgccttc gcagcccccg caggggcagg gcccgccgtc
  481 cggacccggg caaccggcac ccgcggcgac ccaggcggcg ccgcaggcac cccccgccgg
  541 gcatcagatc gtgcacgtcc gcggggactc ggagaccgac ctggaggcgc tcttcaacgc
  601 cgtcatgaac cccaagacgg ccaacgtgcc ccagaccgtg cccatgaggc tccggaagct
  661 gcccgactcc ttcttcaagc cgccggagcc caaatcccac tcccgacagg ccagtactga
  721 tgcaggcact gcaggagccc tgactccaca gcatgttcga gctcattcct ctccagcttc
  781 tctgcagttg ggagctgttt ctcctgggac actgaccccc actggagtag tctctggccc
  841 agcagctaca cccacagctc agcatcttcg acagtcttct tttgagatac ctgatgatgt
  901 acctctgcca gcaggttggg agatggcaaa gacatcttct ggtcagagat acttcttaaa
  961 tcacatcgat cagacaacaa catggcagga ccccaggaag gccatgctgt cccagatgaa
1021 cgtcacagcc cccaccagtc caccagtgca gcagaatatg atgaactcgg cttcaggtcc
1081 tcttcctgat ggatgggaac aagccatgac tcaggatgga gaaatttact atataaacca
1141 taagaacaag accacctctt ggctagaccc aaggcttgac cctcgttttg ccatgaacca
1201 gagaatcagt cagagtgctc cagtgaaaca gccaccaccc ctggctcccc agagcccaca
1261 gggaggcgtc atgggtggca gcaactccaa ccagcagcaa cagatgcgac tgcagcaact
1321 gcagatggag aaggagaggc tgcggctgaa acagcaagaa ctgcttcggc aggtgaggcc
1381 acaggcaatg cggaatatca atcccagcac agcaaattct ccaaaatgtc aggagttagc
1441 cctgcgtagc cagttaccaa cactggagca ggatggtggg actcaaaatc cagtgtcttc
1501 tcccgggatg tctcaggaat tgagaacaat gacgaccaat agctcagatc ctttccttaa
1561 cagtggcacc tatcactctc gagatgagag tacagacagt ggactaagca tgagcagcta
1621 cagtgtccct cgaaccccag atgacttcct gaacagtgtg gatgagatgg atacaggtga
1681 tactatcaac caaagcaccc tgccctcaca gcagaaccgt ttcccagact accttgaagc
1741 cattcctggg acaaatgtgg accttggaac actggaagga gatggaatga acatagaagg
1801 agaggagctg atgccaagtc tgcaggaagc tttgagttct gacatcctta atgacatgga
1861 gtctgttttg gctgccacca agctagataa agaaagcttt cttacatggt tatagagccc
1921 tcaggcagac tgaattctaa atctgtgaag gatctaagga gacacatgca ccggaaattt
1981 ccataagcca gttgcagttt tcaggctaat acagaaaaag atgaacaaac gtccagcaag
2041 atactttaat cctctatttt gctcttcctt gtccattgct gctgttaatg tattgctgac
2101 ctctttcaca gttggctcta aagaatcaaa agaaaaaaac tttttatttc ttttgctatt
2161 aaaactactg ttcattttgg gggctggggg aagtgagcct gtttggatga tggatgccat
2221 tccttttgcc cagttaaatg ttcaccaatc attttaacta aatactcaga cttagaagtc
2281 agatgcttca tgtcacagca tttagtttgt tcaacagttg tttcttcagc ttcctttgtc
2341 cagtggaaaa acatgattta ctggtctgac aagccaaaaa tgttatatct gatattaaat
2401 acttaatgct gatttgaaga gatagctgaa accaaggctg aagactgttt tactttcagt
2461 attttctttt cctcctagtg ctatcattag tcacataatg accttgattt tattttagga
2521 gcttataagg catgagacaa tttccatata aatatattaa ttattgccac atactctaat
2581 atagattttg gtggataatt ttgtgggtgt gcattttgtt ctgttttgtt gggttttttg
2641 ttttttttgt ttttggcagg gtcggtgggg gggttggttg gttggttggt tttgtcggaa
2701 cctaggcaaa tgaccatatt agtgaatctg ttaatagttg tagcttggga tggttattgt
2761 agttgttttg gtaaaatctt catttcctgg ttttttttac caccttattt aaatctcgat
2821 tatctgctct ctcttttata tacatacaca cacccaaaca taacatttat aatagtgtgg
2881 tagtggaatg tatccttttt taggtttccc tgctttccag ttaattttta aaatggtagc
2941 gctttgtatg catttagaat acatgactag tagtttatat ttcactggta gtttaaatct
3001 ggttggggca gtctgcagat gtttgaagta gtttagtgtt ctagaaagag ctattactgt
3061 ggatagtgcc taggggagtg ctccacgccc tctgggcata cggtagatat tatctgatga
3121 attggaaagg agcaaaccag aaatggcttt attttctccc ttggactaat ttttaagtct
3181 cgattggaat tcagtgagta ggttcataat gtgcatgaca gaaataagct ttatagtggt
3241 ttaccttcat ttagctttgg aagttttctt tgccttagtt ttggaagtaa attctagttt
3301 gtagttctca tttgtaatga acacattaac gactagatta aaatattgcc ttcaagattg
3361 ttcttactta caagacttgc tcctacttct atgctgaaaa ttgaccctgg atagaatact
3421 ataaggtttt gagttagctg gaaaagtgat cagattaata aatgtatatt ggtagttgaa
3481 tttagcaaag aaatagagat aatcatgatt atacctttat ttttacagga agagatgatg
3541 taactagagt atgtgtctac aggagtaata atggtttcca aagagtattt tttaaaggaa
3601 caaaacgagc atgaattaac tcttcaatat aagctatgaa gtaatagttg gttgtgaatt
3661 aaagtggcac cagctagcac ctctgtgttt taagggtctt tcaatgtttc tagaataagc
3721 ccttattttc aagggttcat aacaggcata aaatctcttc tcctggcaaa agctgctatg
3781 aaaagcctca gcttgggaag atagattttt ttccccccaa ttacaaaatc taagtatttt
3841 ggcccttcaa tttggaggag ggcaaaagtt ggaagtaaga agttttattt taagtacttt
3901 cagtgctcaa aaaaatgcaa tcactgtgtt gtatataata gttcataggt tgatcactca
3961 taataattga ctctaaggct tttattaaga aaacagcaga aagattaaat cttgaattaa
4021 gtctgggggg aaatggccac tgcagatgga gttttagagt agtaatgaaa ttctacctag
4081 aatgcaaaat tgggtatatg aattacatag catgttgttg ggattttttt taatgtgcag
4141 aagatcaaag ctacttggaa ggagtgccta taatttgcca gtagccacag attaagatta
4201 tatcttatat atcagcagat tagctttagc ttagggggag ggtgggaaag tttggggggg
4261 gggttgtgaa gatttagggg gaccttgata gagaacttta taaacttctt tctctttaat
4321 aaagacttgt cttacaccgt gctgccatta aaggcagctg ttctagagtt tcagtcacct
4381 aagtacaccc acaaaacaat atgaatatgg agatcttcct ttacccctca actttaattt
4441 gcccagttat acctcagtgt tgtagcagta ctgtgatacc tggcacagtg ctttgatctt
4501 acgatgccct ctgtactgac ctgaaggaga cctaagagtc ctttcccttt ttgagtttga
4561 atcatagcct tgatgtggtc tcttgtttta tgtccttgtt cctaatgtaa aagtgcttaa
4621 ctgcttcttg gttgtattgg gtagcattgg gataagattt taactgggta ttcttgaatt
4681 gcttttacaa taaaccaatt ttataatctt taaatttatc aactttttac atttgtgtta
4741 ttttcagtca gggcttctta gatctactta tggttgatgg agcacattga tttggagttt
4801 cagatcttcc aaagcactat ttgttgtaat aacttttcta aatgtagtgc ctttaaagga
4861 aaaatgaaca cagggaagtg actttgctac aaataatgtt gctgtgttaa gtattcatat
4921 taaatacatg ccttctatat ggaacatggc agaaagactg aaaaataaca gtaattaatt
4981 gtgtaattca gaattcatac caatcagtgt tgaaactcaa acattgcaaa agtgggtggc
5041 aatattcagt gcttaacact tttctagcgt tggtacatct gagaaatgag tgctcaggtg
5101 gattttatcc tcgcaagcat gttgttataa gaattgtggg tgtgcctatc ataacaattg
5161 ttttctgtat cttgaaaaag tattctccac attttaaatg ttttatatta gagaattctt
5221 taatgcacac ttgtcaaata tatatatata gtaccaatgt taccttttta ttttttgttt
5281 tagatgtaag agcatgctca tatgttaggt acttacataa attgttacat tattttttct
5341 tatgtaatac ctttttgttt gtttatgtgg ttcaaatata ttctttcctt aaactcttaa
5401 aaaaaaaa
Homo sapiens transferrin receptor (TFRC), transcript variant 1, mRNA (NCBI
Reference Sequence: NM_003234.3)
(SEQ ID NO: 46)
      1 agagcgtcgg gatatcgggt ggcggctcgg gacggaggac gcgctagtgt gagtgcgggc
    61 ttctagaact acaccgaccc tcgtgtcctc ccttcatcct gcggggctgg ctggagcggc
  121 cgctccggtg ctgtccagca gccataggga gccgcacggg gagcgggaaa gcggtcgcgg
  181 ccccaggcgg ggcggccggg atggagcggg gccgcgagcc tgtggggaag gggctgtggc
  241 ggcgcctcga gcggctgcag gttcttctgt gtggcagttc agaatgatgg atcaagctag
  301 atcagcattc tctaacttgt ttggtggaga accattgtca tatacccggt tcagcctggc
  361 tcggcaagta gatggcgata acagtcatgt ggagatgaaa cttgctgtag atgaagaaga
  421 aaatgctgac aataacacaa aggccaatgt cacaaaacca aaaaggtgta gtggaagtat
  481 ctgctatggg actattgctg tgatcgtctt tttcttgatt ggatttatga ttggctactt
  541 gggctattgt aaaggggtag aaccaaaaac tgagtgtgag agactggcag gaaccgagtc
  601 tccagtgagg gaggagccag gagaggactt ccctgcagca cgtcgcttat attgggatga
  661 cctgaagaga aagttgtcgg agaaactgga cagcacagac ttcaccggca ccatcaagct
  721 gctgaatgaa aattcatatg tccctcgtga ggctggatct caaaaagatg aaaatcttgc
  781 gttgtatgtt gaaaatcaat ttcgtgaatt taaactcagc aaagtctggc gtgatcaaca
  841 ttttgttaag attcaggtca aagacagcgc tcaaaactcg gtgatcatag ttgataagaa
  901 cggtagactt gtttacctgg tggagaatcc tgggggttat gtggcgtata gtaaggctgc
  961 aacagttact ggtaaactgg tccatgctaa ttttggtact aaaaaagatt ttgaggattt
1021 atacactcct gtgaatggat ctatagtgat tgtcagagca gggaaaatca cctttgcaga
1081 aaaggttgca aatgctgaaa gcttaaatgc aattggtgtg ttgatataca tggaccagac
1141 taaatttccc attgttaacg cagaactttc attctttgga catgctcatc tggggacagg
1201 tgacccttac acacctggat tcccttcctt caatcacact cagtttccac catctcggtc
1261 atcaggattg cctaatatac ctgtccagac aatctccaga gctgctgcag aaaagctgtt
1321 tgggaatatg gaaggagact gtccctctga ctggaaaaca gactctacat gtaggatggt
1381 aacctcagaa agcaagaatg tgaagctcac tgtgagcaat gtgctgaaag agataaaaat
1441 tcttaacatc tttggagtta ttaaaggctt tgtagaacca gatcactatg ttgtagttgg
1501 ggcccagaga gatgcatggg gccctggagc tgcaaaatcc ggtgtaggca cagctctcct
1561 attgaaactt gcccagatgt tctcagatat ggtcttaaaa gatgggtttc agcccagcag
1621 aagcattatc tttgccagtt ggagtgctgg agactttgga tcggttggtg ccactgaatg
1681 gctagaggga tacctttcgt ccctgcattt aaaggctttc acttatatta atctggataa
1741 agcggttctt ggtaccagca acttcaaggt ttctgccagc ccactgttgt atacgcttat
1801 tgagaaaaca atgcaaaatg tgaagcatcc ggttactggg caatttctat atcaggacag
1861 caactgggcc agcaaagttg agaaactcac tttagacaat gctgctttcc ctttccttgc
1921 atattctgga atcccagcag tttctttctg tttttgcgag gacacagatt atccttattt
1981 gggtaccacc atggacacct ataaggaact gattgagagg attcctgagt tgaacaaagt
2041 ggcacgagca gctgcagagg tcgctggtca gttcgtgatt aaactaaccc atgatgttga
2101 attgaacctg gactatgaga ggtacaacag ccaactgctt tcatttgtga gggatctgaa
2161 ccaatacaga gcagacataa aggaaatggg cctgagttta cagtggctgt attctgctcg
2221 tggagacttc ttccgtgcta cttccagact aacaacagat ttcgggaatg ctgagaaaac
2281 agacagattt gtcatgaaga aactcaatga tcgtgtcatg agagtggagt atcacttcct
2341 ctctccctac gtatctccaa aagagtctcc tttccgacat gtcttctggg gctccggctc
2401 tcacacgctg ccagctttac tggagaactt gaaactgcgt aaacaaaata acggtgcttt
2461 taatgaaacg ctgttcagaa accagttggc tctagctact tggactattc agggagctgc
2521 aaatgccctc tctggtgacg tttgggacat tgacaatgag ttttaaatgt gatacccata
2581 gcttccatga gaacagcagg gtagtctggt ttctagactt gtgctgatcg tgctaaattt
2641 tcagtagggc tacaaaacct gatgttaaaa ttccatccca tcatcttggt actactagat
2701 gtctttaggc agcagctttt aatacagggt agataacctg tacttcaagt taaagtgaat
2761 aaccacttaa aaaatgtcca tgatggaata ttcccctatc tctagaattt taagtgcttt
2821 gtaatgggaa ctgcctcttt cctgttgttg ttaatgaaaa tgtcagaaac cagttatgtg
2881 aatgatctct ctgaatccta agggctggtc tctgctgaag gttgtaagtg gtcgcttact
2941 ttgagtgatc ctccaacttc atttgatgct aaataggaga taccaggttg aaagaccttc
3001 tccaaatgag atctaagcct ttccataagg aatgtagctg gtttcctcat tcctgaaaga
3061 aacagttaac tttcagaaga gatgggcttg ttttcttgcc aatgaggtct gaaatggagg
3121 tccttctgct ggataaaatg aggttcaact gttgattgca ggaataaggc cttaatatgt
3181 taacctcagt gtcatttatg aaaagagggg accagaagcc aaagacttag tatattttct
3241 tttcctctgt cccttccccc ataagcctcc atttagttct ttgttatttt tgtttcttcc
3301 aaagcacatt gaaagagaac cagtttcagg tgtttagttg cagactcagt ttgtcagact
3361 ttaaagaata atatgctgcc aaattttggc caaagtgtta atcttagggg agagctttct
3421 gtccttttgg cactgagata tttattgttt atttatcagt gacagagttc actataaatg
3481 gtgttttttt aatagaatat aattatcgga agcagtgcct tccataatta tgacagttat
3541 actgtcggtt ttttttaaat aaaagcagca tctgctaata aaacccaaca gatactggaa
3601 gttttgcatt tatggtcaac acttaagggt tttagaaaac agccgtcagc caaatgtaat
3661 tgaataaagt tgaagctaag atttagagat gaattaaatt taattagggg ttgctaagaa
3721 gcgagcactg accagataag aatgctggtt ttcctaaatg cagtgaattg tgaccaagtt
3781 ataaatcaat gtcacttaaa ggctgtggta gtactcctgc aaaattttat agctcagttt
3841 atccaaggtg taactctaat tcccattttg caaaatttcc agtacctttg tcacaatcct
3901 aacacattat cgggagcagt gtcttccata atgtataaag aacaaggtag tttttaccta
3961 ccacagtgtc tgtatcggag acagtgatct ccatatgtta cactaagggt gtaagtaatt
4021 atcgggaaca gtgtttccca taattttctt catgcaatga catcttcaaa gcttgaagat
4081 cgttagtatc taacatgtat cccaactcct ataattccct atcttttagt tttagttgca
4141 gaaacatttt gtggtcatta agcattgggt gggtaaattc aaccactgta aaatgaaatt
4201 actacaaaat ttgaaattta gcttgggttt ttgttacctt tatggtttct ccaggtcctc
4261 tacttaatga gatagtagca tacatttata atgtttgcta ttgacaagtc attttaactt
4321 tatcacatta tttgcatgtt acctcctata aacttagtgc ggacaagttt taatccagaa
4381 ttgacctttt gacttaaagc agagggactt tgtatagaag gtttgggggc tgtggggaag
4441 gagagtcccc tgaaggtctg acacgtctgc ctacccattc gtggtgatca attaaatgta
4501 ggtatgaata agttcgaagc tccgtgagtg aaccatcatt ataaacgtga tgatcagctg
4561 tttgtcatag ggcagttgga aacggcctcc tagggaaaag ttcatagggt ctcttcaggt
4621 tcttagtgtc acttacctag atttacagcc tcacttgaat gtgtcactac tcacagtctc
4681 tttaatcttc agttttatct ttaatctcct cttttatctt ggactgacat ttagcgtagc
4741 taagtgaaaa ggtcatagct gagattcctg gttcgggtgt tacgcacacg tacttaaatg
4801 aaagcatgtg gcatgttcat cgtataacac aatatgaata cagggcatgc attttgcagc
4861 agtgagtctc ttcagaaaac ccttttctac agttagggtt gagttacttc ctatcaagcc
4921 agtacgtgct aacaggctca atattcctga atgaaatatc agactagtga caagctcctg
4981 gtcttgagat gtcttctcgt taaggagatg ggccttttgg aggtaaagga taaaatgaat
5041 gagttctgtc atgattcact attctagaac ttgcatgacc tttactgtgt tagctctttg
5101 aatgttcttg aaattttaga ctttctttgt aaacaaatga tatgtcctta tcattgtata
5161 aaagctgtta tgtgcaacag tgtggagatt ccttgtctga tttaataaaa tacttaaaca
5221 ctgaaaaaaa aaaa
Homo sapiens acyl-CoA synthetase long chain family member 4 (ACSL4),
transcript variant 1, mRNA (NCBI Reference Sequence: NM_004458.2)
(SEQ ID NO: 47)
      1 gcgattcggc tggctctgcc acaccaccgc gcgcccccgc tccgcccgcc cctccgggcg
    61 cgtcttttcc gggctcgcgc tgagtcccgc ctccgccggc tgtccgggtg cgcgcgcgcc
  121 gctgcggctt tttctctggc ctccgccgcg cgctcctcct cgtcccagcg ctagcgggca
  181 cgcggttcct ttttgcgagc tttccgagtg ccaggcgccg gccggctgcg aagacgcggt
  241 gggccgcccc tccgattgaa atcacagaag atattcgtgt tcttcttaag agaaaaagag
  301 gacatttaaa aacgctatgg caaagagaat aaaagctaag cccacttcag acaaacctgg
  361 aagtccatat cgctctgtca cacacttcga ctcactagct gtaatagaca tccctggagc
  421 agatactctg gataaattat ttgaccatgc tgtatccaag tttgggaaga aggacagcct
  481 tgggaccagg gaaatcctaa gtgaagaaaa tgaaatgcag ccaaatggaa aagtttttaa
  541 gaagttaatt cttgggaatt ataaatggat gaactatctt gaagtgaatc gcagagtgaa
  601 taactttggt agtggactca ctgcactggg actaaaacca aagaacacca ttgccatctt
  661 ctgtgagacc agggccgaat ggatgattgc agcacagacc tgctttaagt acaactttcc
  721 tcttgtgact ttatatgcca cacttggcaa agaagcagta gttcatgggc taaatgaatc
  781 tgaggcttcc tatctgatta ccagtgttga acttctggaa agtaaactta agactgcatt
  841 gttagatatc agttgtgtta aacatatcat ttatgtggac aataaggcta tcaataaagc
  901 agagtaccct gaaggatttg agattcacag catgcaatca gtagaagagt tgggatctaa
  961 cccagaaaac ttgggcattc ctccaagtag accaacgcct tcagacatgg ccattgttat
1021 gtatactagt ggttctactg gccgacctaa gggagtgatg atgcatcata gcaatttgat
1081 agctggaatg acaggccagt gtgaaagaat acctggactg ggaccgaagg acacatatat
1141 tggctacttg cctttggctc atgtgctaga actgacagca gagatatctt gctttaccta
1201 tggctgcagg attggatatt cttctccgct tacactctct gaccagtcca gcaaaattaa
1261 aaaaggaagc aaaggagact gtactgtact gaagcccaca cttatggctg ctgttccgga
1321 aatcatggat agaatttata agaatgttat gagcaaagtc caagagatga attatattca
1381 gaaaactctg ttcaagatag ggtatgatta caaattggaa cagatcaaaa agggatatga
1441 tgcacctctt tgcaatctgt tactgtttaa aaaggtcaag gccctgctgg gagggaatgt
1501 ccgcatgatg ctgtctggag gggccccgct atctcctcag acacaccgat tcatgaatgt
1561 ctgcttctgc tgcccaattg gccagggtta tggactgaca gaatcatgtg gtgctgggac
1621 agttactgaa gtaactgact atactactgg cagagttgga gcacctctta tttgctgtga
1681 aattaagcta aaagactggc aagaaggcgg ttatacaatt aatgacaagc caaaccccag
1741 aggtgaaatc gtaattggtg gacagaacat ctccatggga tattttaaaa atgaagagaa
1801 aacagcagaa gattattctg tggatgaaaa tggacaaagg tggttttgca ctggtgatat
1861 tggagaattc catcccgatg gatgtttaca gattatagat cgtaagaaag atctagtgaa
1921 gttacaagca ggagagtatg tatctcttgg gaaagtagaa gctgcactga agaattgtcc
1981 acttattgac aacatctgtg cttttgccaa aagtgatcag tcctatgtga tcagttttgt
2041 ggttcctaac cagaaaaggt tgacactttt ggcacaacag aaaggggtag aaggaacttg
2101 ggttgatatc tgcaataatc ctgctatgga agctgaaata ctgaaagaaa ttcgagaagc
2161 tgcaaatgcc atgaaattgg agcgatttga aattccaatc aaggttcgat taagcccaga
2221 gccatggacc cctgaaactg gtttggtaac tgatgctttc aaactgaaaa ggaaggagct
2281 gaggaaccat tacctcaaag acattgaacg aatgtatggg ggcaaataaa atgttgttgt
2341 cttattgaca gttgtgcagg aggtagcctg gtggttttca acctctagaa ttttaagcct
2401 ttgttgaact gttagaatgt aaggtatatc attctaaaga tagagtaaaa agaaaacaaa
2461 accaaaagtt attaaaattg ttgtccggtt tactttaact tagttttgca tagttctagt
2521 gcagctgaaa ttgaaaagtt atttcccttt agctgtgtta ttatagagca gaaattctgt
2581 ttttaaaaat tagcctaaga tatacttgtt tttgtaaaga aaaatattta atgttgaaca
2641 aaataaattg gagttggagt agaatgtagt ttgaggaaat ttgcagcttc caatgcctct
2701 tgtcttccta tttcagaagt ttaaatatta agcatgacag aaaatatgta ttaacactac
2761 tcaaagcaaa agtgctgcag ggctttaaaa ttctcttcca accatttatc ttgaaggaaa
2821 aattcaatag taatataata cacaaaatca aataatacct tagaaggtat taagattata
2881 attgttgcat aggttagata tagagtcatt gtaatgttgt gaataattac agtgcctaaa
2941 ataagaatag aacaacatat acaacaccaa aaaatatcta gtaatatatt taaagggaaa
3001 ttgagctgct ttttttgaaa ctttgagatc taaaaataac tgtaattatt tgaatgacta
3061 agaggaaagt acattttttg aaatgctgaa aattgccttt ctgtgtttat tcaaactgaa
3121 aagctgagac caagagcaag gaaggtaaaa agttaacagg caaacatttt ctcttagaaa
3181 aggtgataaa atcataagta tttggaatta gaacccttgc acagcactga acctgggaaa
3241 gagatttaaa ctctgaattt atctttgata acagggattg attttaaaat gtacatgtat
3301 taaattacat ttgtaattta aggtctgttt gctgttgctg attttattct tgatcagtag
3361 tttgcatttc agaaagcctt tcattttgct ttaattttag caaagcgggt tataatgaat
3421 gacttcccca atatcttgct tgaacttaca ggtgattaac ttggatgagt tttgggaagt
3481 taaagggaag aaaacactgt tatcattttt tcctgtttgg gaagagctta gaaactggaa
3541 atactagatt tgggagaagg gcagagttac ttgataaggg actgatgttt gtgcagtaac
3601 ttgggagtgt ggtttctttt tgaatcttta attaaaacct gggattatat atccctgata
3661 aatattcaca cttgaaccat agttactgta aaatgcaaaa aatcttaata ctgttattct
3721 ttgcactttt tcttaatcat tttttatata tatgcatata tatatgtgtg tgtgtgtgtt
3781 gcttatgttg ttttgtacag atgtgggcca ccattgcaac aaaatacatt ctttttgctc
3841 taaaatattt atgaagaaaa tacttaaatg ttatgtatat ggtggtaata agggaaaaat
3901 caagtattat aaacaagaat gaaggttttt gtaaagattt ctgttcagcg ttttgcaagg
3961 taaaatttta ggcaagtttt ccctgaagtt atgtgtatgt gagtattctc attcttccca
4021 acttgccttt gaagagtgaa ataccattat tatcaagtag actactgttc agcttttatt
4081 cctgccctgc tgtttatccc ttaagaatga gtttcttaga cttttccaat atgtgatttt
4141 ttttcccatt tagaatggtg attttaaatg tgtgagtgca tgtactatct tatctcagat
4201 atttgcaccc ccaatctgcc cccaactccc aaaagctaga acactgccaa ctgatctgtt
4261 ataggtcctt tagaaacaca taattaacac ttaaggttgg gtgctgctaa ttctttgcaa
4321 aaatccaaat attgttaagg gaccagggag atgccactac cccttgattt tccatctaaa
4381 aatatacatg tttatgtaaa caaatctttc catatccata gtgacttttc aagtatttaa
4441 gcctaaagat tttgatctca catttttata cctgtttaaa ttgctcacag ttattacata
4501 cacatcagcc atcaactaaa gttgtacttt aaaaatttac tacaatatgt acatttctaa
4561 gtcaaacact tgtgactttt gctttaattc catgaatgtt cctgcctcct tgatatttgt
4621 atttattctt tttttctcta gagtagaggt ataattgtgt gatatttcag aaatacagat
4681 aaatgattca aaaagtcaca gttaaggaga atcatgtttc tttgatcatg aataactgat
4741 tagtaagtct tgcctatatt ttcctgatag catatgacaa atgtttctaa ggtaacaaga
4801 tgagaacaga taaagattgt gtggtgtttt ggatttggag agaaatattt taatttttaa
4861 atgcagttac aaattataat gtattcatat ttgtactttc tgttaaaatg catgattgca
4921 gaattgttta gattttgtgt ttattcttga tgaaaagctt tgtttgttct tgtttttaag
4981 tttgcactca aatcttaaga aataaatcca cccatgttat caaaaaaaaa aaaaaaaaa
Homo sapiens transforming growth factor beta 1 (TGFB1), mRNA (NCBI
Reference Sequence: NM_000660.6)
(SEQ ID NO: 48)
      1 acctccctcc gcggagcagc cagacagcga gggccccggc cgggggcagg ggggacgccc
    61 cgtccggggc acccccccgg ctctgagccg cccgcggggc cggcctcggc ccggagcgga
  121 ggaaggagtc gccgaggagc agcctgaggc cccagagtct gagacgagcc gccgccgccc
  181 ccgccactgc ggggaggagg gggaggagga gcgggaggag ggacgagctg gtcgggagaa
  241 gaggaaaaaa acttttgaga cttttccgtt gccgctggga gccggaggcg cggggacctc
  301 ttggcgcgac gctgccccgc gaggaggcag gacttgggga ccccagaccg cctccctttg
  361 ccgccgggga cgcttgctcc ctccctgccc cctacacggc gtccctcagg cgcccccatt
  421 ccggaccagc cctcgggagt cgccgacccg gcctcccgca aagacttttc cccagacctc
  481 gggcgcaccc cctgcacgcc gccttcatcc ccggcctgtc tcctgagccc ccgcgcatcc
  541 tagacccttt ctcctccagg agacggatct ctctccgacc tgccacagat cccctattca
  601 agaccaccca ccttctggta ccagatcgcg cccatctagg ttatttccgt gggatactga
  661 gacacccccg gtccaagcct cccctccacc actgcgccct tctccctgag gacctcagct
  721 ttccctcgag gccctcctac cttttgccgg gagaccccca gcccctgcag gggcggggcc
  781 tccccaccac accagccctg ttcgcgctct cggcagtgcc ggggggcgcc gcctccccca
  841 tgccgccctc cgggctgcgg ctgctgccgc tgctgctacc gctgctgtgg ctactggtgc
  901 tgacgcctgg ccggccggcc gcgggactat ccacctgcaa gactatcgac atggagctgg
  961 tgaagcggaa gcgcatcgag gccatccgcg gccagatcct gtccaagctg cggctcgcca
1021 gccccccgag ccagggggag gtgccgcccg gcccgctgcc cgaggccgtg ctcgccctgt
1081 acaacagcac ccgcgaccgg gtggccgggg agagtgcaga accggagccc gagcctgagg
1141 ccgactacta cgccaaggag gtcacccgcg tgctaatggt ggaaacccac aacgaaatct
1201 atgacaagtt caagcagagt acacacagca tatatatgtt cttcaacaca tcagagctcc
1261 gagaagcggt acctgaaccc gtgttgctct cccgggcaga gctgcgtctg ctgaggctca
1321 agttaaaagt ggagcagcac gtggagctgt accagaaata cagcaacaat tcctggcgat
1381 acctcagcaa ccggctgctg gcacccagcg actcgccaga gtggttatct tttgatgtca
1441 ccggagttgt gcggcagtgg ttgagccgtg gaggggaaat tgagggcttt cgccttagcg
1501 cccactgctc ctgtgacagc agggataaca cactgcaagt ggacatcaac gggttcacta
1561 ccggccgccg aggtgacctg gccaccattc atggcatgaa ccggcctttc ctgcttctca
1621 tggccacccc gctggagagg gcccagcatc tgcaaagctc ccggcaccgc cgagccctgg
1681 acaccaacta ttgcttcagc tccacggaga agaactgctg cgtgcggcag ctgtacattg
1741 acttccgcaa ggacctcggc tggaagtgga tccacgagcc caagggctac catgccaact
1801 tctgcctcgg gccctgcccc tacatttgga gcctggacac gcagtacagc aaggtcctgg
1861 ccctgtacaa ccagcataac ccgggcgcct cggcggcgcc gtgctgcgtg ccgcaggcgc
1921 tggagccgct gcccatcgtg tactacgtgg gccgcaagcc caaggtggag cagctgtcca
1981 acatgatcgt gcgctcctgc aagtgcagct gaggtcccgc cccgccccgc cccgccccgg
2041 caggcccggc cccaccccgc cccgcccccg ctgccttgcc catgggggct gtatttaagg
2101 acacccgtgc cccaagccca cctggggccc cattaaagat ggagagagga ctgcggatct
2161 ctgtgtcatt gggcgcctgc ctggggtctc catccctgac gttcccccac tcccactccc
2221 tctctctccc tctctgcctc ctcctgcctg tctgcactat tcctttgccc ggcatcaagg
2281 cacaggggac cagtggggaa cactactgta gttagatcta tttattgagc accttgggca
2341 ctgttgaagt gccttacatt aatgaactca ttcagtcacc atagcaacac tctgagatgc
2401 agggactctg ataacaccca ttttaaaggt gaggaaacaa gcccagagag gttaagggag
2461 gagttcctgc ccaccaggaa cctgctttag tgggggatag tgaagaagac aataaaagat
2521 agtagttcag gccaggcggg gtggctcacg cctgtaatcc tagcactttt gggaggcaga
2581 gatgggagga ttacttgaat ccaggcattt gagaccagcc tgggtaacat agtgagaccc
2641 tatctctaca aaacactttt aaaaaatgta cacctgtggt cccagctact ctggaggcta
2701 aggtgggagg atcacttgat cctgggaggt caaggctgca g
Homo sapiens tafazzin (TAZ), transcript variant 1, mRNA (NCBI Reference
Sequence: NM_000116.5)
(SEQ ID NO: 49)
      1 gctttccggc ggttgcaccg ggccggggtg ccagcgcccg ccttcccgtt tcctcccgtt
    61 ccgcagcgcg cccacggcct gtgaccccgg cgaccgctcc ccagtgacga gagagcgggg
  121 ccgggcgctg ctccggcctg acctgcgaag ggacctcggt ccagtcccct gttgcgccgc
  181 gcccccgtcc gtccgtgcgc gggccagtca ggggccagtg tctcgagcgg tcgaggtcgc
  241 agacctagag gcgccccaca ggccggcccg gggcgctggg agcgccggcc gcgggccggg
  301 tggggatgcc tctgcacgtg aagtggccgt tccccgcggt gccgccgctc acctggaccc
  361 tggccagcag cgtcgtcatg ggcttggtgg gcacctacag ctgcttctgg accaagtaca
  421 tgaaccacct gaccgtgcac aacagggagg tgctgtacga gctcatcgag aagcgaggcc
  481 cggccacgcc cctcatcacc gtgtccaatc accagtcctg catggacgac cctcatctct
  541 gggggatcct gaaactccgc cacatctgga acctgaagtt gatgcgttgg acccctgcag
  601 ctgcagacat ctgcttcacc aaggagctac actcccactt cttcagcttg ggcaagtgtg
  661 tgcctgtgtg ccgaggagca gaatttttcc aagcagagaa tgaggggaaa ggtgttctag
  721 acacaggcag gcacatgcca ggtgctggaa aaagaagaga gaaaggagat ggcgtctacc
  781 agaaggggat ggacttcatt ttggagaagc tcaaccatgg ggactgggtg catatcttcc
  841 cagaagggaa agtgaacatg agttccgaat tcctgcgttt caagtgggga atcgggcgcc
  901 tgattgctga gtgtcatctc aaccccatca tcctgcccct gtggcatgtc ggaatgaatg
  961 acgtccttcc taacagtccg ccctacttcc cccgctttgg acagaaaatc actgtgctga
1021 tcgggaagcc cttcagtgcc ctgcctgtac tcgagcggct ccgggcggag aacaagtcgg
1081 ctgtggagat gcggaaagcc ctgacggact tcattcaaga ggaattccag catctgaaga
1141 ctcaggcaga gcagctccac aaccacctcc agcctgggag ataggccttg cttgctgcct
1201 tctggattct tggcccgcac agagctgggg ctgagggatg gactgatgct tttagctcaa
1261 acgtggcttt tagacagatt tgttcataga ccctctcaag tgccctctcc gagctggtag
1321 gcattccagc tcctccgtgc ttcctcagtt acacaaagga cctcagctgc ttctcccact
1381 tggccaagca gggaggaaga agcttaggca gggctctctt tccttcttgc cttcagatgt
1441 tctctcccag gggctggctt caggagggag catagaaggc aggtgagcaa ccagttggct
1501 aggggagcag ggggcccacc agagctgtgg agaggggacc ctaagactcc tcggcctggc
1561 tcctacccac cgcccttgcc gaaccaggag ctgctcacta cctcctcagg gatggccgtt
1621 ggccacgtct tccttctgcc tgagcttccc ccccaccaca ggccctttcc tcaggcaagg
1681 tctggcctca ggtgggccgc aggcgggaaa agcagccctt ggccagaagt caagcccagc
1741 cacgtggagc ctagagtgag ggcctgaggt ctggctgctt gcccccatgc tggcgccaac
1801 aacttctcca tcctttctgc ctctcaacat cacttgaatc ctagggcctg ggttttcatg
1861 tttttgaaac agaaccataa agcatatgtg ttggcttgtt gtaaaa
```

Primers or probes can be designed so that they hybridize under stringent conditions to mutant nucleotide sequences of at least one of E-cadherin, N-cadherin, Merlin, Mst1, Mst2, Lats1, or Lats2, but not to the respective wild-type nucleotide sequences. Primers or probes can also be prepared that are complementary and specific for the wild-type nucleotide sequence of at least one of E-cadherin, N-cadherin, Merlin, Mst1, Mst2, Lats1, or Lats2, but not to any one of the corresponding mutant nucleotide sequences. In some embodiments, the mutant nucleotide sequences of at least one of E-cadherin, N-cadherin, Merlin, Mst1, Mst2, Lats1, or Lats2 may be a frameshift mutation, a missense mutation, a deletion, an insertion, a nonsense mutation, an inversion, or a translocation. Alternatively, primers or probes can be designed so that they selectively hybridize to YAP, TAZ, TFRC, ACSL4, or TGF-β.

In some embodiments, detection can occur through any of a variety of mobility dependent analytical techniques based on the differential rates of migration between different nucleic acid sequences. Exemplary mobility-dependent analysis techniques include electrophoresis, chromatography, mass spectroscopy, sedimentation, for example, gradient centrifugation, field-flow fractionation, multi-stage extraction techniques, and the like. In some embodiments, mobility probes can be hybridized to amplification products, and the identity of the target nucleic acid sequence determined via a mobility dependent analysis technique of the eluted mobility probes, as described in Published PCT Applications WO04/46344 and WO01/92579. In some embodiments, detection can be achieved by various microarrays and related software such as the Applied Biosystems Array System with the Applied Biosystems 1700 Chemiluminescent Microarray Analyzer and other commercially available array systems available from Affymetrix, Agilent, Illumina, and Amersham Biosciences, among others (see also Gerry et al., J. Mol. Biol. 292:251-62, 1999; De Bellis et al., Minerva Biotec 14:247-52, 2002; and Stears et al., Nat. Med. 9:14045, including supplements, 2003).

It is also understood that detection can comprise reporter groups that are incorporated into the reaction products, either as part of labeled primers or due to the incorporation of labeled dNTPs during an amplification, or attached to reaction products, for example but not limited to, via hybridization tag complements comprising reporter groups or via linker arms that are integral or attached to reaction products. In some embodiments, unlabeled reaction products may be detected using mass spectrometry.

### NGS Platforms

In some embodiments, high throughput, massively parallel sequencing employs sequencing-by-synthesis with reversible dye terminators. In other embodiments, sequencing is performed via sequencing-by-ligation. In yet other embodiments, sequencing is single molecule sequencing. Examples of Next Generation Sequencing techniques include, but are not limited to pyrosequencing, Reversible dye-terminator sequencing, SOLiD sequencing, Ion semiconductor sequencing, Helioscope single molecule sequencing etc.

The Ion Torrent™ (Life Technologies, Carlsbad, Calif.) amplicon sequencing system employs a flow-based approach that detects pH changes caused by the release of hydrogen ions during incorporation of unmodified nucleotides in DNA replication. For use with this system, a sequencing library is initially produced by generating DNA fragments flanked by sequencing adapters. In some embodiments, these fragments can be clonally amplified on particles by emulsion PCR. The particles with the amplified template are then placed in a silicon semiconductor sequencing chip. During replication, the chip is flooded with one nucleotide after another, and if a nucleotide complements the DNA molecule in a particular microwell of the chip, then it will be incorporated. A proton is naturally released when a nucleotide is incorporated by the polymerase in the DNA molecule, resulting in a detectable local change of pH. The pH of the solution then changes in that well and is detected by the ion sensor. If homopolymer repeats are present in the template sequence, multiple nucleotides will be incorporated in a single cycle. This leads to a corresponding number of released hydrogens and a proportionally higher electronic signal.

The 454TM GS FLX™ sequencing system (Roche, Germany), employs a light-based detection methodology in a large-scale parallel pyrosequencing system. Pyrosequencing uses DNA polymerization, adding one nucleotide species at a time and detecting and quantifying the number of nucleotides added to a given location through the light emitted by the release of attached pyrophosphates. For use with the 454™ system, adapter-ligated DNA fragments are fixed to small DNA-capture beads in a water-in-oil emulsion and amplified by PCR (emulsion PCR). Each DNA-bound bead is placed into a well on a picotiter plate and sequencing reagents are delivered across the wells of the plate. The four DNA nucleotides are added sequentially in a fixed order across the picotiter plate device during a sequencing run. During the nucleotide flow, millions of copies of DNA bound to each of the beads are sequenced in parallel. When a nucleotide complementary to the template strand is added to a well, the nucleotide is incorporated onto the existing DNA strand, generating a light signal that is recorded by a CCD camera in the instrument.

Sequencing technology based on reversible dye-terminators: DNA molecules are first attached to primers on a slide and amplified so that local clonal colonies are formed. Four types of reversible terminator bases (RT-bases) are added, and non-incorporated nucleotides are washed away. Unlike pyrosequencing, the DNA can only be extended one nucleotide at a time. A camera takes images of the fluorescently labeled nucleotides, then the dye along with the terminal 3′ blocker is chemically removed from the DNA, allowing the next cycle.

Helicos's single-molecule sequencing uses DNA fragments with added polyA tail adapters, which are attached to the flow cell surface. At each cycle, DNA polymerase and a single species of fluorescently labeled nucleotide are added, resulting in template-dependent extension of the surface-immobilized primer-template duplexes. The reads are performed by the Helioscope sequencer. After acquisition of images tiling the full array, chemical cleavage and release of the fluorescent label permits the subsequent cycle of extension and imaging.

Sequencing by synthesis (SBS), like the “old style” dye-termination electrophoretic sequencing, relies on incorporation of nucleotides by a DNA polymerase to determine the base sequence. A DNA library with affixed adapters is denatured into single strands and grafted to a flow cell, followed by bridge amplification to form a high-density array of spots onto a glass chip. Reversible terminator methods use reversible versions of dye-terminators, adding one nucleotide at a time, detecting fluorescence at each position by repeated removal of the blocking group to allow polymerization of another nucleotide. The signal of nucleotide incorporation can vary with fluorescently labeled nucleotides, phosphate-driven light reactions and hydrogen ion sensing having all been used. Examples of SBS platforms include Illumina GA and HiSeq 2000. The MiSeq® personal sequencing system (Illumina, Inc.) also employs sequencing by synthesis with reversible terminator chemistry.

In contrast to the sequencing by synthesis method, the sequencing by ligation method uses a DNA ligase to determine the target sequence. This sequencing method relies on enzymatic ligation of oligonucleotides that are adjacent through local complementarity on a template DNA strand. This technology employs a partition of all possible oligonucleotides of a fixed length, labeled according to the sequenced position. Oligonucleotides are annealed and ligated and the preferential ligation by DNA ligase for matching sequences results in a dinucleotide encoded color space signal at that position (through the release of a fluorescently labeled probe that corresponds to a known nucleotide at a known position along the oligo). This method is primarily used by Life Technologies' SOLiD™ sequencers. Before sequencing, the DNA is amplified by emulsion PCR. The resulting beads, each containing only copies of the same DNA molecule, are deposited on a solid planar substrate.

SMRT™ sequencing is based on the sequencing by synthesis approach. The DNA is synthesized in zero-mode wave-guides (ZMWs)-small well-like containers with the capturing tools located at the bottom of the well. The sequencing is performed with use of unmodified polymerase (attached to the ZMW bottom) and fluorescently labeled nucleotides flowing freely in the solution. The wells are constructed in a way that only the fluorescence occurring at the bottom of the well is detected. The fluorescent label is detached from the nucleotide at its incorporation into the DNA strand, leaving an unmodified DNA strand.

### Methods for Treatment Selection

In one aspect, the present disclosure provides a method for selecting a cancer patient for treatment with a ferroptosis-inducing therapy comprising (a) detecting the presence of a mutation in at least one polynucleotide encoding one or more proteins selected from the group consisting of E-cadherin, N-cadherin, Merlin, Mst1, Mst2, Lats1, and Lats2 in a biological sample obtained from the cancer patient, wherein the mutation is a frameshift mutation, a missense mutation, a deletion, an insertion, a nonsense mutation, an inversion, or a translocation; and (b) administering to the cancer patient an effective amount of a ferroptosis-inducing agent. The mutation may be detected using any nucleic acid detection assay known in the art such as next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH). In some embodiments, the biological sample comprises genomic DNA, cDNA, RNA, and/or mRNA.

In one aspect, the present disclosure provides a method for treating a therapy-resistant, metastasis-prone cancer in a patient in need thereof comprising administering to the cancer patient an effective amount of a ferroptosis-inducing agent, wherein mRNA or polypeptide expression and/or activity levels of one or more of E-cadherin, N-cadherin, Merlin, Mst1, Mst2, Lats1, and Lats2 in a biological sample obtained from the patient are reduced compared to that observed in a control sample obtained from a healthy subject or a predetermined threshold. In another aspect, the present disclosure provides a method for treating a therapy-resistant, metastasis-prone cancer in a patient in need thereof comprising administering to the cancer patient an effective amount of a ferroptosis-inducing agent, wherein mRNA or polypeptide expression and/or activity levels of one or more of YAP, TAZ, TFRC, ACSL4, and TGF-β are elevated compared to that observed in a control sample obtained from a healthy subject or a predetermined threshold. The metastasis-prone cancer may be resistant to chemotherapy or radiation therapy. Additionally or alternatively, in some embodiments, the patient is diagnosed with or suffers from a cancer selected from the group consisting of mesothelioma, lung cancer, liver cancer, colon cancer, rectal cancer, and breast cancer.

Additionally or alternatively, in some embodiments, mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH). In some embodiments, TFRC mRNA expression levels are detected using a forward primer comprising the sequence of SEQ ID NO: 36 and a reverse primer comprising the sequence of SEQ ID NO: 37 or a probe comprising the sequence of SEQ ID NO: 15, SEQ ID NO: 16, SEQ ID NO: 36, SEQ ID NO: 37, or any complement thereof. In certain embodiments, ACSL4 mRNA expression levels are detected using a forward primer comprising the sequence of SEQ ID NO: 34 and a reverse primer comprising the sequence of SEQ ID NO: 35 or a probe comprising the sequence of SEQ ID NO: 34, SEQ ID NO: 35, or any complement thereof. In other embodiments, Merlin mRNA expression levels are detected using a forward primer comprising the sequence of SEQ ID NO: 1 and a reverse primer comprising the sequence of SEQ ID NO: 2 or a probe comprising the sequence of SEQ ID NO: 1, SEQ ID NO: 2, SEQ ID NO: 7, SEQ ID NO: 8, SEQ ID NO: 9, SEQ ID NO: 10, or any complement thereof. In some embodiments, E-cadherin or N-cadherin mRNA expression levels are detected using a probe comprising the sequence of SEQ ID NO: 3, SEQ ID NO: 4, SEQ ID NO: 5, SEQ ID NO: 6, or any complement thereof. In certain embodiments, Lats1 or Lats2 mRNA expression levels are detected using a probe comprising the sequence of SEQ ID NO: 11, SEQ ID NO: 12, SEQ ID NO: 13, SEQ ID NO: 14, or any complement thereof.

Additionally or alternatively, in some embodiments, polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

In any of the embodiments of methods disclosed herein, the ferroptosis-inducing agent is a class 1 ferroptosis inducer (system X_{c}⁻ inhibitor) or a class 2 ferroptosis inducer (glutathione peroxidase 4 (GPx4) inhibitor). Examples of ferroptosis-inducing agents include, but are not limited to, erastin, erastin derivatives (e.g., MEII, PE, AE, imidazole ketone erastin (IKE)), DPI2, BSO, SAS, lanperisone, SRS13-45, SRS13-60, RSL3, DPI7, DPI10, DPI12, DPI13, DPI17, DPI18, DPI19, ML160, sorafenib, and artemisinin derivatives. Additionally or alternatively, in some embodiments of the methods disclosed herein, the patient is human.

### EXAMPLES

The present technology is further illustrated by the following Examples, which should not be construed as limiting in any way. The examples herein are provided to illustrate advantages of the present technology and to further assist a person of ordinary skill in the art with preparing or using the compositions and systems of the present technology. The examples should in no way be construed as limiting the scope of the present technology, as defined by the appended claims. The examples can include or incorporate any of the variations, aspects, or embodiments of the present technology described above. The variations, aspects, or embodiments described above may also further each include or incorporate the variations of any or all other variations, aspects or embodiments of the present technology.

### Example 1: Experimental Materials and Methods

Cell culture. Mouse Embryonic Fibroblasts (MEFs) and human epithelial tumor cells including HCT116, HepG2, PC9, H1650, BT474 and MDA-MB-231 were cultured in Dulbecco's Modified Eagle Medium (DMEM) containing 10% fetal calf serum (FCS), 2 mM L-glutamine, 100 units/ml penicillin and 100 μg/ml streptomycin. Human mesothelioma cell lines were cultured as previously described (Lopez-Lago et al., Mol. Cell. Biol. 29:4235-4249 (2009)).

Generation of three-dimensional spheroids. Spheroids were generated by plating tumor cells at 10³/well into U-bottom Ultra Low Adherence (ULA) 96-well plates (Corning, Tewksbury, Mass., USA). Optimal three-dimensional structures were achieved by centrifugation at 600 g for 5 min followed by addition of 2.5% Matrigel (Corning). Plates were incubated for 72 h at 37° C., 5% CO₂, 95% humidity for formation of a single spheroid of cells. Spheroids were then treated with erastin in fresh medium containing Matrigel for the indicated time.

Induction and inhibition of ferroptosis. To induce ferroptosis, cells with different density were seeded in 6-well plates. For cystine-starvation experiments, cells were washed with PBS twice and then cultured in cystine-free medium in the presence of 10% (v/v) dialyzed FBS for the indicated time. The ferroptosis inducing compounds erastin and RSL3 and the ferroptosis inhibitor Ferrostatin-1 were purchased from Sigma-Aldrich (St. Louis, Mo., USA).

Measurement of cell death, cell viability and lipid peroxidation. Cell death was analyzed by propidium iodide (Invitrogen, Waltham, Mass., USA) or SYTOX Green (Invitrogen) staining followed by microscopy or flow cytometry. For 3D spheroids, cell viability was determined using the CellTiter-Glo® 3D Cell Viability Assay (Promega, Madison, Wis., USA) according to the manufacturer's instructions. Viability was calculated by normalizing ATP levels to spheroids treated with normal full media. To analyze lipid peroxidation, cells were stained with 5 μM BODIPY-C11 (Invitrogen) for 30 minutes at 37° C. followed by flow cytometric analysis.

Immunoblotting. Nuclear and non-nuclear (membranes and cytosol) fractions were prepared as previously described. Proteins in the cell lysate were resolved on 8% or 15% SDS-PAGE gels and transferred to a nitrocellulose membrane. Membranes were incubated in 5% skim milk for 1 hour at room temperature and then with primary antibodies diluted in blocking buffer at 4° C. overnight. The following primary antibodies were used: rabbit anti-GPX4 (Abcam, Cambridge, Mass., USA), mouse anti-E-cadherin (Abcam), rabbit anti-N-cadherin, mouse anti-β-actin (Sigma-Aldrich), rabbit anti-Merlin (Cell Signaling, Danvers, Mass., USA), rabbit anti-phospho-Merlin (Cell Signaling), rabbit anti-Lats1 (Cell Signaling), rabbit anti-Lats2 (Cell Signaling), rabbit anti-YAP (Cell Signaling), rabbit anti-phospho-YAP (Ser127) (Cell Signaling), rabbit anti-TFRC (Cell Signaling), rabbit anti-ACSL4 (Santa Cruz Biotechnology, Dallas, Tex., USA), mouse anti-Cas9 (Cell Signaling), mouse anti-Flag (Sigma-Aldrich), mouse anti-HA (Sigma-Aldrich), rabbit anti-GFP (Invitrogen). Goat anti-mouse IgG (Thermo Fisher) or donkey anti-rabbit IgG (Invitrogen) conjugated to horseradish peroxidase and an Amersham Imager 600 (GE Healthcare Life Sciences, Marlborough, Mass., USA) were used for detection. Representative blots of at least two independent experiments are shown. After three washes, the membranes were incubated with goat anti-mouse HRP-conjugated antibody or donkey anti-rabbit HRP-conjugated antibody at room temperature for 1 hour and subjected to chemiluminescence using Clarity™ Western ECL Substrate (Bio-Rad, Hercules, Calif., USA).

Plasmids and cloning. pWZL Blast mouse E-cadherin and pWZL Blast DN E-cadherin were from the Weinberg Lab (Addgene plasmids #18804 and 18800, respectively). pRK5-Flag-HA-Merlin was from the Giancotti Lab (Addgene plasmid #27104). 8×GTIIC-luciferase was from the Piccolo lab (Addgene plasmid #34615). mCherry-TFR-20 was from the Davidson lab (Addgene plasmid #55144). pQCXIH-Flag-YAP-S127A was from the Guan Lab (Addgene plasmid #33092). pBABE-Flag-HA-Merlin was generated by subcloning using primers ACTGTTAATTAACATGGACTACAAAGACGATGACG (SEQ ID NO: 1) and ATGAGAGAATTCCAAGCTTCTGCAGGTCGACTC (SEQ ID NO: 2), digested by Pad and EcoRI FastDigest restriction enzymes (Thermo Fisher), and ligated into the empty pBABE-puro backbone using T4 ligase (NEB, Ipswich, Mass., USA). FUW-tetO-Flag-HA-Merlin was created by digesting pRK5-Flag-HA-Merlin with EcoRI and XbaI and was ligated into the FUW-tetO-MCS vector from the Piccolo lab (Addgene plasmid #84008). FUW-m2rtTA was from the Jaenisch lab (Addgene plasmid #20342).

Gene silencing and expression. Lentiviral vectors encoding shRNAs targeting human E-cadherin, human N-cadherin, human and mouse Merlin, human Lats1 and Lats2, and human TFRC were generated. See Table 1. Lentiviruses were produced by the co-transfection of the lentiviral vector with the Delta-VPR envelope and CMV VSV-G packaging plasmids into 293T cells using PEI. Media was changed 12 hours after transfection. The supernatant was collected 48 hours after transfection and passed through a 0.45 μm filter to eliminate cells. Cells were incubated with infectious particles in the presence of 4 μg/ml polybrene (Sigma-Aldrich) overnight and cells were given fresh complete medium. After 48 hours, cells were placed under the appropriate antibiotic selection.

TABLE 1
shRNA target sequences.

| | | SEQ | |
|---|---|---|---|
| | Target | ID | Target |
| shRNA | gene | NO: | sequence |
| shEcad #1 | Human CDH1 | 3 | GCAGAAATTAT |
| | | | TGGGCTCTTT |
| shEcad #2 | Human CDH1 | 4 | CCAGTGAACAA |
| | | | CGATGGCATT |
| shNcad #1 | Human CDH2 | 5 | CCAGTGACTAT |
| | | | TAAGAGAAAT |
| shNcad #2 | Human CDH2 | 6 | CGCATTATGCA |
| | | | AGACTGGATT |
| shMerlin #1 (h) | Human NF2 | 7 | GAAGCAACCCA |
| | | | AGACGTTCAC |
| shMerlin #2 (h) | Human NF2 | 8 | TAGTTCTCTGA |
| | | | CCTGAGTCTT |
| shMerlin #1 (m) | Mouse NF2 | 9 | CGAGCGTACAA |
| | | | GAGATGAGTT |
| shMerlin #2 (m) | Mouse NF2 | 10 | GOAGOAAGCAT |
| | | | AATACCATTA |
| shLats1 #1 | Human LATS1 | 11 | CAAGTCAGAAA |
| | | | TCCACCCAAA |
| shLats1 #2 | Human LATS1 | 12 | GATTACAACTT |
| | | | CACCTATTAC |
| shLats2 #1 | Human LATS2 | 13 | CCGTCGATTAC |
| | | | TTCACTFGAA |
| shLats2 #2 | Human LATS2 | 14 | GCCATGAAGAC |
| | | | CCTAAGGAAA |
| shTFRC #1 | Human TFRC | 15 | CCCAACAGATA |
| | | | CTGGAAGTTT |
| shTFRC #2 | Human TFRC | 16 | GCTGGTCAGTT |
| | | | CGTGATTAAA |

Generation of constitutive and inducible CRISPR/Cas9-mediated gene knockouts. E-cadherin, YAP, and ACSL4 depleted cells were generated with CRISPR/Cas9-mediated knockout system. HCT116 cells were transfected with a human E-cadherin CRISPR/Cas9 KO plasmid (sc-400031), and HCT116-shMerlin cells were transfected with a human YAP CRISPR/Cas9 KO plasmid (sc-400040 or a human ACSL4 CRISPR/Cas9 KO plasmid (sc-401649), all purchased from Santa Cruz Biotechnology. Target sequence was a pool of three different gRNA plasmids located within the coding DNA sequence fused to Streptococcus pyogenes Cas9, and GFP. Single GFP⁺ cells were sorted using a BD FACSAria II cytometer (BD Biosciences, Franklin Lakes, N.J., USA) to 96-well plate and single-cell clones were tested by Western blotting.

The lentiviral doxycycline-inducible FLAG-Cas9 vector pCW-Cas9 and pLX-sgRNA were from Eric Lander & David Sabatini (Addgene plasmids #50661 and 50662, respectively) (Wang et al., Science 343:80-84 (2014)). Guide RNA sequence CACGCCCGATACGCTGAGTG (SEQ ID NO: 17) was used to target human Gpx4. To construct the lentiviral sgRNA vector for Gpx4, a pair of oligonucleotides (Forward and Reverse) was annealed, phosphorylated and ligated into pLX-sgRNA. Lentiviral particles containing the sgRNA or Cas9 vectors were produced by co-transfection of the vectors with the Delta-VPR envelope and CMV VSV-G packaging plasmids into 293T cells using PEI. Media was changed 12 hours after transfection and supernatant was collected 48 hours after transfection. MSTO-211H cells in 6-well tissue culture plates were infected in pCW-Cas9 virus-containing supernatant containing 4 μg/mL of polybrene. 24 hours after infection, virus was removed, and cells were selected with 2 μg/ml puromycin. Single clones were screened for inducible Cas9 expression. 2 μg/ml doxycycline was added to the culture media for 3 days. Single clones with Cas9 expression were infected with Gpx4 gRNA virus-containing supernatant containing 8 μg/ml polybrene. Twenty-four hours after infection, virus was removed, and cells were selected with 10 μg/ml blasticidin. Single clones with doxycycline-inducible Cas9 expression and Gpx4 knockout were amplified for further experiments, named Gpx4 iKO MSTO-211H cells.

ChIP assay. Cells were crosslinked in 0.75% formaldehyde for 15 min, then glycine was added to a final concentration of 125 mM for 5 min. After wash with cold PBS, cells were collected in PBS and sonicated on an ultrasonic homogenizer for 10 min at 20% power on ice to shear DNA to an average fragment size of 200-1000 bp. Fifty μL of each sonicated sample was removed to determine DNA concentration and fragment size. Cell lysates were incubated overnight with 20 μL Magna ChIP™ Protein A+G Magnetic Beads (EMD Millipore, Burlington, Mass., USA) and 10 μg ChIP grade TEAD4 antibody (Abcam) at 4° C. Beads were collected, washed and treated with Proteinase K for 2 h at 60° C. and RNase for 1 h at 37° C. DNA was purified with a PCR purification kit (Qiagen, Germantown, Md., USA). DNA fragments were assessed by qRT-PCR using the primer sequences listed in Table 2. Samples were normalized to input DNA.

RNA extraction and qRT-PCR. RNA was extracted using the TRIzol reagent (Invitrogen). 20% chloroform was added to each sample, vortexed briefly, and incubated at room temperature for 15 min. Samples were then centrifuged at high speed at 4° C. for 15 min. The aqueous phase was moved to a new tube and an equal volume of isopropanol was added. Samples were incubated at room temperature for 10 min, followed by centrifugation at high speed at 4° C. for 10 min. Pellets were washed in 95% ethanol, dried, and resuspended in nuclease-free water. cDNA was synthesized using iScript™ cDNA Synthesis Kit according to the manufacturer's instructions (Bio-Rad). qRT-PCR was performed with IQ™ SYBR® Green Supermix (Bio-Rad) in a CFX Connect Real-Time PCR Detection System (Bio-Rad). The sequences of primers used are listed in Table 2.

TABLE 2
Primer sets for real-time RT-PCR and ChIP.

| Assay | Gene | Forward | Reverse |
|---|---|---|---|
| qRT-PCR | CTGF | CCAAT | TGGTG |
| | | GACAA | CAGCC |
| | | CGCCT | AGAAA |
| | | CCTG | GCTC |
| | | (SEQ ID | (SEQ ID |
| | | NO: 18) | NO: 19) |
| qRT-PCR | CYR61 | AGCCT | TTCTT |
| | | CGCAT | TCACA |
| | | CCTAT | AGGCG |
| | | ACAAC | GCACT |
| | | C | C |
| | | (SEQ ID | (SEQ ID |
| | | NO: 20) | NO: 21) |
| qRT-PCR | Twist1 | AGATG | GACTG |
| | | GCAAG | TCCAT |
| | | CTGCA | TTTCT |
| | | GCTAT | CCTTC |
| | | G | TCTG |
| | | (SEQ ID | (SEQ ID |
| | | NO: 22) | NO: 23) |
| qRT-PCR | Zeb1 | AGGTG | TGGAG |
| | | TAAGC | TTTGT |
| | | GCAGA | CTTCA |
| | | AAGCA | TCATC |
| | | G | G |
| | | (SEQ ID | (SEQ ID |
| | | NO: 24) | NO: 25) |
| qRT-PCR | Zeh2 | CGCTT | CTTGC |
| | | GACAT | CACAC |
| | | CGCTG | TCCGT |
| | | AAGGA | GCACT |
| | | (SEQ ID | (SEQ ID |
| | | NO: 26) | NO: 27) |
| qRT-PCR | Slug | CTTTC | ACAGC |
| | | TCTTG | AGCCA |
| | | CCCTC | GACTC |
| | | ACTGC | CTCAT |
| | | (SEQ ID | (SEQ ID |
| | | NO: 28) | NO: 29) |
| qRT-PCR | Snail | CATCC | CTTCT |
| | | GAGTG | CTAGG |
| | | GGTTT | CCCTG |
| | | GGAGG | GCTG |
| | | (SEQ ID | (SEQ ID |
| | | NO: 30) | NO: 31) |
| qRT-PCR | GAPDH | CCATG | CAGGG |
| | | TTCGT | GTGCT |
| | | CATGG | AAGCA |
| | | GTGTG | GTTGG |
| | | (SEQ ID | (SEQ ID |
| | | NO: 32) | NO: 33) |
| Real Time | ACSL4- | GTGCT | ACATT |
| ChIP | primer 1 | GTACC | GCCAC |
| | | TAAAT | AAGAG |
| | | GCAAC | TACAA |
| | | (SEQ ID | (SEQ ID |
| | | NO: 34) | NO: 35) |
| Real Time | TFR1- | ACGAG | CTCAG |
| ChIP | primer 1 | GGTCG | GAAGT |
| | | GTGTA | GACGC |
| | | GTTCT | ACAGC |
| | | (SEQ ID | (SEQ ID |
| | | NO: 36) | NO: 37) |
| Cloning | NF2 | ACTGT | ATGAG |
| | | TAATT | AGAAT |
| | | AACAT | TCCAA |
| | | GGACT | GCTTC |
| | | ACAAA | TGCAG |
| | | GACGA | GTCGA |
| | | TGACG | CTC |
| | | (SEQ ID | (SEQ ID |
| | | NO: 1) | NO: 2) |

In vivo xenograft mouse Study. Gpx4 iKO MSTO-211H cells were infected with lentiviral vectors encoding shRNAs targeting human Merlin or shNT (GeneCopoeia, Rockville, Md., USA). The resulting cells were called shNT-Gpx4 iKO MSTO-211H cells and shMerlin-Gpx4 iKO MSTO-211H cells. Six- to eight-week-old female athymic nu/nu mice were purchased from Envigo (East Millstone, N.J., USA). For s.c. tumor models, mice were injected in the right flank with 1×10⁷ shNT-GPX4 iKO MSTO-211H cells or shMerlin-GPX4 iKO MSTO-211H cells resuspended in 150 μL Matrigel. Tumors were measured with calipers every 3 days. When tumors reached a mean volume of 100 mm3, mice with similarly sized tumors were grouped into four treatment groups. For control or knockout cohorts, mice were given intraperitoneal (i.p.) injections of 0.9% sterile saline or Doxycycline (100 mg/kg body weight) for two days. At the same time, mice were provided with either a normal diet or Doxycycline diet for control or knockout cohorts, respectively. At the end of the study, mice were euthanized with CO₂ and tumors were taken for immunohistochemical staining. Results are presented as mean tumor volume±SD.

For shLats1/2 s.c. tumor models, female athymic nu/nu mice aged 6 to 8 weeks were injected in the right flank with 2×10⁶ shNT HCT116 cells or shLats1/2 HCT116 cells. Tumors were measured with calipers every day. When tumors reached a mean volume of 90 mm³, mice were randomized into four groups and treated with vehicle (65% D5W (5% dextrose in water), 5% Tween-80, 30% PEG-400) or 50 mg/kg IKE (65% D5W (5% dextrose in water), 5% Tween-80, 30% PEG-400) via IP injection once a day. At the end of the study, mice were euthanized with CO₂ and tumors were taken for measurement of weight.

Orthotopic pleural mesothelioma animal model. ShNT-Gpx4 iKO MSTO-211H cells and shMerlin-Gpx4 iKO MSTO-211H cells were infected with retroviral TK-GFP-Luciferase reporter vector (TGL). To develop the orthotopic mouse model of pleural mesothelioma, female NOD/SCID mice (Envigo, Somerset, N.J.) aged 6 to 8 weeks were used. Mice were anesthetized using inhaled isoflurane and oxygen. Intrapleural injection of 2×10⁶ shNT-Gpx4 iKO-TGL MSTO-211H cells or shMerlin-Gpx4 iKO-TGL MSTO-211H cells in 100 μl of serum-free medium via a left thoracic incision was performed to establish the orthotopic mesothelioma tumor model. Tumor growth was monitored by weekly bioluminescence imaging (BLI) for luciferase and mice were monitored daily for survival. Nod/Scid mice bearing tumors were anesthetized using isoflurane and injected i.p. with 50 mg/kg D-luciferin (Molecular Probes, Carlsbad, Calif., USA). BLI was measured with 18 filters (500-840 nm) in an IVIS Spectrum (PerkinElmer, Waltham, Mass., USA) 10 min after injection. During image acquisition, mice were maintained on isoflurane via nose cone. Bioluminescence images were acquired using an IVIS Spectrum. BLI signal was reported as total flux (photons per second), which represents the average of ventral and dorsal flux. At the end-point of the study, the animals were injected with D-luciferase and sacrificed 10 min later. Organs were exposed and BLI was measured. After organs were excised, BLI images were taken again as described.

Immunohistochemistry. Formalin-fixed, paraffin-embedded specimens were collected, and a routine H&E slide was first evaluated. Immunohistochemical staining was done on 5 μm-thick paraffin-embedded sections using mouse anti-Merlin (Abcam), rabbit anti-GPX4 (Abcam), rabbit anti-PTGS2 (Cell Signaling), mouse anti-Ki67 (Cell Signaling), rabbit anti-ACSL4 (Thermo Fisher), rabbit anti-TFRC (Abcam), and rabbit anti-YAP (Cell Signaling) antibodies with a standard avidin-biotin HRP detection system according to the instructions of the manufacturer (anti-mouse/rabbit HRP-DAB Cell & Tissue Staining Kit, R&D Systems Minneapolis, Minn.). Tissues were counterstained with haematoxylin, dehydrated, and mounted. In all cases, antigen retrieval was done with the BD Retrievagen Antigen Retrieval Systems as per instructions of the manufacturer.

Tumor spheroid invasion assay. Spheroids were generated as described in 200 μl complete growth medium and cultured for 72 h after cell seeding. The ULA 96-well plates containing 3-day old spheroids were placed on ice. 100 μl per well of growth medium was removed from the spheroid plates. Using ice-cold tips, 100 μl of Matrigel was transferred to each well and mixed gently with medium, avoiding disturbance of the spheroids. Plates were placed in an incubator at 37° C. to allow the Matrigel to solidify. One hour later, 100 μl/well of complete growth medium was added. Images for each tumor spheroid were taken 48 h later.

Statistical analysis. All statistical analyses were performed using GraphPad Prism 6.0 Software. Data are presented as mean±SD from 3 independent experiments. P-values were calculated with Student's unpaired t test (* P<0.05, ** P<0.01, *** P<0.001).

### Example 2: E-Cadherin-Mediated Intercellular Interaction Suppresses Ferroptosis

Cell Density-Dependent Inhibition of Ferroptosis.

To characterize in detail how cellular metabolism impacts ferroptosis, ingredients of culture medium or cell number in culture were altered. Cells became more resistant to ferroptosis when they approached to high confluence in culture.

HCT116 cells were seeded into 6-well plates at 5×10⁴-8×10⁵ cells per well as indicated. After 24 h, cells were treated with normal media (+Cystine) or cystine-free media (-Cystine) for 30 h and stained with SYTOX Green to show cell death and SYTOX Green staining were quantified by flow cytometry, and phase-contrast and fluorescent images were taken. To quantify lipid peroxidation, cells were stained with 2 μM C11-BODIPY followed by flow cytometry.

To test erastin-induced ferroptosis, HCT116 cells with different cell density were treated with 30 μM erastin and quantification of cell death by propidium iodide (PI) staining followed by flow cytometry (after 30 h of erastin treatment). Lipid ROS production was assessed by C11-BODIPY staining followed by flow cytometry (after 24 h of erastin treatment). To test RSL3-induced ferroptosis, HCT116 cells were cultured with indicated cell density and treated with 5 μM RSL3 for 24 h (cell death) or 16 h (lipid ROS).

FIG. 1A shows cystine starvation-induced ferroptosis in HCT116 cells cultured at different cell densities. Cells became more resistant to ferroptosis when they approached to high confluence in culture. When different numbers of HCT116 human colon cancer cells were seeded in culture dishes, the higher cell number conferred significant resistance to ferroptosis and associated lipid peroxidation when exposed to three commonly used inducers of ferroptosis: cystine starvation (FIGS. 1A-1C), inhibition of cystine import by erastin, an inhibitor of the cystine/glutamate antiporter system Xc− (FIG. 6A), and inhibition of GPX4 by its covalent inhibitor RSL3 (FIG. 6B). In contrast, sparse HCT116 cells underwent death by ferroptosis.

To confirm that sparse HCT116 cells were dying from ferroptosis but not another cell death modality, various cell death inhibitors were tested: ferrostatin-1 (Fer-1) and DFO, which inhibit ferroptosis, Z-VAD-FMK, a pan-caspase inhibitor, and GSK'872, a selective RIPK3 inhibitor that blocks necroptosis. HCT116 cells were seeded at a density of 5×10⁴ cells/well and grown for 24 hours. Cells were washed in PBS and cultured in cystine-free media with the indicated treatments for 30 h. Fer-1: Ferrostatin-1, 1 μM; DFO: iron chelator, 50 μg/mL; Z-VAD-FMK: pan-caspase inhibitor, 20 μM; GSK'872: RIPK3 inhibitor, 10 μM. HCT116 cells were seeded at a density of 5×10⁴ cells/well and grown for 24 hours, then treated with 5 μM RSL3 and the indicated cell death inhibitors for 24 hours before measuring cell death. These experiments confirmed that cells underwent ferroptosis, instead of other modes of cell death, under these conditions (FIGS. 6C-6D). Similar cell density-dependent ferroptosis was previously observed by Seiler et al., Cell Metab. 8: 237-248 (2008) and Schneider et al., Neoplasia 12:254-263 (2010). In Seiler et al., mouse embryonic fibroblasts (MEFs) harboring two floxed GPX4 alleles survived and grew when seeded at high density, but upon passage at lower density they died rapidly. In Schneider et al., GPX4-deficient MEFs formed 3D tumor spheroids as well as wild-type MEFs by an unknown mechanism.

To explore if such cell density-dependent regulation is a general property of ferroptosis, a panel of human epithelial cancer cell lines were tested, including HepG2 (liver cancer), PC9 and H1650 (lung cancer), BT474 and MDA-MB-231 (breast cancer), in addition to HCT116 (colon cancer). The panel of 6 epithelial cancer cell lines were seeded at indicated cell densities and were treated with cystine-free media for 30 h. Cell death was assessed by flow cytometry following PI staining. As shown in FIGS. 1D-1F, most cell lines showed cell density dependence upon induction by cystine starvation. Two exceptions were noted: MDA-MB-231 cells were always sensitive to ferroptosis, whereas BT474 cells were always resistant to ferroptosis, regardless of tested cell confluence. Among the cell lines showing cell density-dependent regulation, H1650 cells were most sensitive to ferroptosis.

To better mimic the in vivo context, these human epithelial cancer cell lines were cultured into 3D multicellular tumor spheroids. Spheroids generated from the indicated cancer cell lines were cultured for 72 h and treated with 15 μM erastin for 30 h. Cell death was measured by staining cells with SYTOX Green, and cell viability within spheroids was determined by measuring cellular ATP levels. Consistent with the results from 2D cell culture analysis, a prominent erastin-induced cell death response was observed in spheroids formed by MDA-MB-231 cells and H1650 cells (FIG. 1E). In addition, erastin treatment significantly reduced cell viability in MDA-MB-231 and H1650 spheroids (FIG. 1F).

Glutamine replenishment was conducted to ensure that density dependent cell death was not caused by the depletion of nutrients. Glutamine is required for cysteine deprivation-induced ferroptosis. HCT116 cells were seeded at a density of 8×10⁵ cells/well, grown for 24 hours, and treated with cystine-free media containing the indicated amounts of glutamine for 30 h. However, replenishing glutamine to the cultured cells failed to restore cell death (FIG. 6E).

E-Cadherin Expression is Regulated by Cell Density in Cell Lines that are Subject to Density-Dependent Regulation of Ferroptosis.

Cells tend to forge cell-cell contact with higher cell confluence, and E-cadherin (Ecad) is an important mediator of intercellular contact in epithelial cells. To investigate the involvement of Ecad in cell density-regulated ferroptosis, the protein levels of Ecad in indicated cancer cell lines were analyzed by Western blot (FIG. 1G; FIG. 7A). Ecad expression at different cell densities was also measured by Western blot and immunofluorescence (FIGS. 7A-7B). β-actin was blotted as a control. Ecad expression was detectable in all these cancer cell lines (FIG. 1G). The expression of Ecad, as well as its enrichment at regions of cell-cell contact, increased along with increasing cell density in density-dependent cells, such as HCT116 and H1650 cells (FIGS. 7A-7B).

However, E-cadherin expression in MDA-MB-231 cells, which are sensitive to ferroptosis independent of cell density, was undetectable (FIG. 1G). H1650 cells, which are sensitive to ferroptosis in the 3D spheroid test (FIG. 1F), expressed a relatively low level of Ecad (FIG. 1G). BT474 cells, which are resistant to ferroptosis regardless of cell confluence, tend to express high Ecad even at low cell density (FIG. 7B). To assess the expression of Ecad in an in vivo context, tumor spheroids were generated from HCT116 or MDA-MB-231 cells and were fixed, sectioned, and stained for Ecad expression by immunohistochemistry. Strong expression of Ecad was detected in spheroids generated from HCT116 cells, but not those generated from MDA-MB-231 cells (FIG. 7C). These results suggest that Ecad may be responsible for the observed cell density regulation of ferroptosis via mediating cell-cell contact.

Intercellular Contact Mediated by E-Cadherin Suppresses Ferroptosis.

To determine whether Ecad plays a causative role in cell density regulation of ferroptosis, the effect of inhibiting Ecad with respect to cell death induced by ferroptosis was tested. HCT116 cells were treated with either α-IgG or α-Ecad antibody that blocks Ecad homotypic intercellular dimerization. The cells were then subjected to cystine starvation for 30 h. Cell death was measured by PI staining coupled with flow cytometry. As shown in FIG. 8A, an anti-Ecad antibody that blocks Ecad intercellular dimerization increased the sensitivity of high-density cells to ferroptosis, and reversed their resistance to cystine deprivation induction ferroptosis (FIG. 8A; FIG. 1B).

Additionally, Ecad knockout HCT116 cells were generated using the CRISPR/Cas9 approach. Expression of Ecad and N-cadherin (Ncad) were assayed by Western blot and immunofluorescence (FIG. 1H; FIG. 8B). Cell death was measured as described above. Ecad-depleted (ΔEcad) cells were more sensitive to cystine deprivation-induced ferroptosis at high cell confluence when compared to parental HCT116 cells (FIGS. 1H-1I). Ecad depletion did not induce a compensatory expression of N-cadherin (Ncad) in HCT116 cells (FIG. 11I). To assess whether the suppression required full-length Ecad function, wild type or mutant Ecad lacking the ectodomain (EcadΔecto) were reconstituted into ΔEcad cells; and expression was confirmed by Western blot (FIG. 1J). The Ecad ectodomain is required for Ecad homotypic intercellular dimerization. The ΔEcad cells and the two reconstituted cell lines were then treated with cystine-free media for 30 hours and cell death was measured by flow cytometry. As shown in FIG. 1K, re-expression of full-length Ecad, but not a truncated mutant missing the ectodomain, restored resistance to ferroptosis in ΔEcad HCT116 cells.

To assess the role of Ecad ectodomain in vivo, HCT116 ΔEcad, and ΔEcad reconstituted with either Ecad or EcadΔecto were formed into spheroids and grown for 72 h, at which point they were treated with 15 μM erastin. After 30 h, spheroids were stained with SYTOX Green and imaged, and viability was measured by an ATP assay. The same results shown in FIG. 1K were observed in in vivo context (FIGS. 8C-8D). To assess whether ectopic expression of Ecad in ferroptosis resistant tumor cell line can be suppressed by Ecad, Ecad was ectopically expressed in Ecad-null MDA-MB-231 cells, and expression was confirmed by western blot (FIG. 8E). High density parental MDA-MB-231 cells and cells ectopically expressing Ecad were then subjected to cystine starvation for 18 hours, followed by cell death measurement. As shown in FIG. 8F, ectopic expression of Ecad in Ecad-negative MDA-MB-231 cells rendered the cells more resistant to cystine starvation at high cell confluence.

Taken together, Ecad negatively regulates ferroptosis by mediating cell-cell contact through intercellular homomeric interaction. Accordingly, these results demonstrate that the methods of the present technology are useful for selecting a cancer patient for treatment with a ferroptosis-inducing therapy.

### Example 3: Activation of the Hippo Signaling Pathway Suppresses Ferroptosis

Cell Density and E-Cadherin Regulate Activity of the Hippo Pathway.

Ecad-mediated intercellular interaction can signal to the intracellular Hippo pathway, which regulates a plethora of biological events, including cell proliferation and organ size control. As shown in FIG. 2A, the Hippo pathway involves multiple players, such as the tumor suppressor Merlin and a kinase cascade comprising Mst1/2 and Lats1/2. Lats1/2 suppresses the function of the pro-oncogenic transcription cofactor YAP by inducing its nuclear exclusion through phosphorylation at its S127 residue.

To determine the role of this pathway in density-dependent ferroptosis, YAP localization in HCT116 cells cultured at different cell densities was assessed by immunofluorescence. The levels of phosphorylated YAP (p-YAP) and YAP in whole cell or cytosolic fractions of these HCT116 cells were analyzed by Western blot. PARP was used as a nuclear protein marker. As shown in FIGS. 9A-9B, increased phosphorylation and decreased nuclear localization of YAP were observed as HCT116 cells grew more confluent.

The role of Ecad and Merlin on cell density-regulation of YAP phosphorylation and nuclear localization was determined by analyzing the levels of Ecad, YAP and phospho-YAP (S127) in parental and Ecad-knockout (ΔEcad) HCT116 cells by Western blot (FIG. 9C) and immunofluorescence (FIG. 9D). Merlin depletion was generated using RNAi technology. Expression levels of Merlin, phosphorylated-YAP, YAP and β-actin in HCT116 expressing control RNAi (shNT) and Merlin RNAi (shMerlin cells) were analyzed by Western blot (FIG. 10B), and subcellular localization of YAP was assessed by immunofluorescence (FIG. 10A). As shown in (FIGS. 9C-9D and FIGS. 10A-10B), Ecad knockout or Merlin RNAi both abolished cell density-regulated nuclear exclusion of YAP.

Knockout of E-Cadherin or Knockdown of Merlin Alters Activity of the Downstream Hippo Target, YAP.

To confirm that YAP is functionally activated at high cell density, the transcriptional levels of two canonical YAP targets, CTGF and CYR61 were measured by qPCR (FIGS. 10C-10D and 10F). HCT116 cells were seeded at 1×10⁵ (sparse) or 8×10⁵ (confluent) and grown for 24 hours. RNA was then purified and mRNA of the canonical YAP targets, CTGF and CYR61, was measured by qPCR. HCT116 and ΔEcad cells were also plated at high density and transcription levels of CTGF and CYR61 were measured by qPCR. To measure the impact of Merlin knockout on YAP activity, HCT116 cells with non-targeting hairpins or hairpins targeting Merlin were plated at high density and transcription levels of CTGF and CYR61 were measured by qPCR.

The activity of YAP was also assessed using an 8×GTIIC-luciferase reporter assay (FIGS. 10E, 10G). The 8×GTIIC-luciferase reporter monitors YAP-TEAD transcriptional activity. The luciferase assay for YAP/TEAD transcriptional activity was tested in HCT116 and ΔEcad cells. To measure the impact of Merlin knockout on YAP activity, YAP/TEAD activity in HCT116 shNT and shMerlin cells was measured using the 8×GTIIC-luciferase reporter. Low cell density (FIG. 10C), loss of Ecad (FIG. 10D-10E), or RNAi targeting Merlin (FIGS. 10F-10G) all increased YAP activity as measured using this luciferase reporter (FIGS. 10E, 10G), and also upregulated transcription of two canonical YAP targets CTGF and CYR61 (FIGS. 10C-10D and 10F).

Ablation of Hippo Signaling Enhances Ferroptosis Independently of Cell Proliferation.

The involvement of the Hippo signaling in the regulation of ferroptosis by cell-cell contact was also assessed. Cells depleted of Ecad, Merlin, or Lats were generated using shRNA technology. HCT116 cells were infected by lentiviruses expressing shRNA sequences targeting Ecad (shEcad), Merlin (shMerlin), or Lats1/2 (shLats1/2), as indicated. Knockdown efficiency was confirmed by Western blot (FIG. 2B). These cells were then cultivated as confluent cultures (seeded at 4×10⁵/well) for 24 h and subjected to cystine starvation for another 30 h (FIG. 2C). Dead cells were stained by PI. The cells were also treated with normal media, cystine starvation alone, or cystine starvation plus 204 ferrostatin-1 (Fer-1) (FIG. 2D). Cell death was measured at 30 h using SYTOX Green staining followed by flow cytometry. Lipid ROS was measured at 24 hours using C11-BODIPY staining coupled with flow cytometry. HCT116 cells expressing shNT, shEcad, shMerlin, or shLats1/2 as indicated were treated with 504 RSL3 with or without 204 Fer-1. Cell death was measured at 18 h and lipid ROS was measured at 14 h (FIG. 11A). FIGS. 2B-D and FIG. 11A show that effective knockdown of Ecad, Merlin, and Lats1/2 all sensitized HCT116 cells to ferroptosis and increased lipid ROS accumulation upon cystine starvation or RSL3 treatment. The shLats1/2#2 failed to sensitize ferroptosis (FIG. 2D) because it did not knockdown Lats2 (FIG. 2B).

RNAi of these Hippo signaling components also enhanced erastin-induced cell death in tumor spheroids of HCT116 cells (FIGS. 2E-2F). Tumor spheroids generated from shEcad, shMerlin and shLats1/2 cells were treated with 15 μM erastin with or without 2 μM Fer-1 for 30 h, and dead cells were stained green by SYTOX Green. Cell viability within the tumor spheroids was measured by determining ATP levels. As shown in FIGS. 2D, 2F and FIG. 11A, Fer-1 blocked cell death in these experiments.

The proliferation of Ecad, Merlin, and Lats1/2 knockdown cells was assessed to ensure that the observed increased ferroptosis was not due to reduced cell confluence. The cumulative cell growth curve of control, shEcad, shMerlin and shLats1/2 HCT116 cells grown with or without cystine starvation was analyzed. Cumulative cell growth curves were expressed as the total cell count of shEcad, shMerlin and shLats1/2 HCT116 cells. As shown in FIG. 11B, knockdown of Ecad, Merlin, and Lats1/2 did not affect cell proliferation, ruling out the possibility that increased ferroptosis was due to reduced cell confluence.

PAK, a Negative Regulator of Merlin, Regulates Density-Dependent Control of Ferroptosis.

Merlin is regulated by p21-activated kinase (PAK), which can phosphorylate and inactivate Merlin. To test whether PAK can regulate ferroptosis in this context, wild type or inactive mutant (K298R) PAK fused to a C-terminal CAAX prenylation motif were transfected in HCT116 cells. Expression and phosphorylation of Merlin was assayed by Western blot (FIG. 12A). As shown in FIGS. 12A-12B, PAK-CAAX, which is constitutively active strongly induced Merlin phosphorylation (FIG. 12A) and increased YAP activity (FIG. 12B). PAK-CAAX also increased ferroptosis following cystine starvation (FIG. 12C) or RSL3 treatment (FIG. 12D). However, the inactive mutant PAKK298R-CAAX had no effect on Merlin phosphorylation, YAP activity or ferroptosis following cystine starvation or RSL3 treatment (FIGS. 12A-12D).

Collectively, these results demonstrate that Ecad and Hippo signaling negatively regulate ferroptosis. Accordingly, these results demonstrate that the methods of the present technology are useful for selecting a cancer patient for treatment with a ferroptosis-inducing therapy.

### Example 4: Merlin Mediates Cell Density-Dependent Inhibition of Ferroptosis in Mesothelioma Cells

Restoration of Wild Type Merlin Subjects Merlin-Defective Cells to Density-dependent regulation of ferroptosis.

Heterozygous deletion and loss-of-function mutations of the Merlin-encoding gene NF2 have been detected with high frequency in malignant mesothelioma (MM), an aggressive cancer arising primarily from mesothelial cells of the pleura, underscoring the tumor suppressive nature of Merlin. Mechanistically, Merlin has been shown to activate the Hippo signaling pathway by inhibiting (through direct interaction) CRL4^{DCAF1}, an ubiquitin ligase that promotes proteasomal degradation of Lats1/2. Therefore, the status of Merlin and ferroptosis sensitivity in a cohort of human malignant mesothelioma cell lines was assessed. Cell lysates from a panel of human mesothelioma cell lines cultured with high confluence were probed for expression of Ecad, Merlin, and β-actin by Western blot. Expression of Lats1/2 and pan-cadherin were also tested in indicated mesothelioma cell lines by Western blot. Of 10 patient-derived mesothelioma cell lines that were examined, 4 were Merlin-wild type (wt) and 6 were Merlin-defective (FIG. 3A). All Merlin-wt cells were responsive to cell density, and they also all expressed cadherin proteins (FIG. 13A) that were not necessarily Ecad (FIG. 3A). Merlin-wt cells also all expressed either Lats1 or 2 (FIG. 13A).

The ferroptosis sensitivity of this cohort of human malignant mesothelioma cell lines was also assessed. Merlin-wt (left) or Merlin-mutant (right) mesothelioma cells were seeded at indicated densities (FIG. 3B). 24 hours later, cells were treated with cystine starvation for an additional 24 h, at which point they were stained with SYTOX Green followed by flow cytometry for cell death measurement. Percentage of mesothelioma cell lines with strong or weak density-dependent (DD) regulation of ferroptosis was also assessed (FIG. 3C). Several Merlin-mutant cell lines underwent potent ferroptosis even at the highest tested cell confluence (FIGS. 3B-3C).

The ferroptosis sensitivity of this cohort of human malignant mesothelioma cell lines was also assessed in its in vivo context. Spheroids generated from indicated mesothelioma cells were treated with 10 μM erastin for 24 h. SYTOX Green staining identified dead cells within the spheroids (FIG. 3D). Cell viability within tumor spheroids was assayed by measuring cellular ATP levels (FIG. 3E). In the 3D tumor spheroid model, Merlin-mutant mesothelioma cells were sensitive to erastin-induced ferroptosis (FIGS. 3D bottom; 3E), whereas Merlin-wt cells were largely resistant (FIG. 3D (Top); 3E).

To confirm the ferroptosis-regulating role of Merlin in mesothelioma cells, the effect of Merlin knockdown on ferroptosis sensitivity was assessed. MSTO-211H cells were infected with lentiviruses expressing shMerlin. RNAi knockdown efficiency was confirmed by Western blot (FIG. 3F). Then, shNT or shMerlin MSTO-211H cells were cultured to high confluence, at which point they were treated with media containing or lacking cystine, with or without Fer-1 for 24 h. Cell death was measured by SYTOX Green staining followed by flow cytometry (FIG. 3G). To measure lipid reactive species, shNT or shMerlin MSTO-211H cells were treated for 18 h, at which point they were stained with 2 μM C11-BODIPY for lipid ROS measurement (FIG. 311). Confluent MSTO-211H cells expressing shNT or shMerlin were also treated with 1 μM RSL3 with or without 204 Fer-1. Cell death (left, 24 h after treatment) and lipid ROS production (right, 16 h) were measured (FIG. 13B). Following RNAi knockdown of Merlin, highly confluent Merlin-wt MSTO-211H mesothelioma cells became sensitive to cystine starvation (FIG. 3G) or RSL3 (FIG. 13B), with enhanced lipid ROS generation and ferroptosis (FIG. 311 and FIG. 13B).

Conversely, Merlin-mutant Meso33 cells were reconstituted with wild type Merlin. Expression of Merlin was confirmed by Western blot and localization of YAP (green) under sparse or confluent conditions was determined by immunofluorescence (FIG. 13C). As shown in FIG. 13C, Merlin reconstitution in highly confluent Merlin-defective Meso33 cells resulted in decreased nuclear localization of YAP. Inhibition of ferroptosis and lipid ROS accumulation was assayed in reconstituted cells. Meso33 cells expressing wild type Merlin were cultured under sparse or confluent conditions and stimulated with cystine-free media. Cell death was measured by SYTOX Green staining coupled with flow cytometry after 24 h of treatment. Lipid ROS was measured after 16 h of cystine starvation. As shown in FIGS. 13D-13E, Merlin reconstitution in highly confluent Merlin-defective Meso33 cells resulted in decreased the inhibition of ferroptosis and lipid ROS accumulation in reconstituted cells.

In addition, a doxycycline (Dox)-inducible system to express Merlin in Meso33 cells was generated. Meso33 cells were transduced with a Dox-inducible Merlin construct. Cells were treated with 1 μg/mL Dox for 48 h and Merlin expression was measured by Western blot (FIG. 31). The sensitivity of Meso33 cells expressing Dox-inducible Merlin to ferroptosis was also tested by treating the cells with cystine-free media for 12 h, in the presence or absence of Dox, followed by cell death measurement (FIG. 3J). Moreover, Spheroids were generated from Meso33 cells expressing Dox-inducible Merlin, and tumor spheroids were grown in the presence or absence of Dox for 72 h, at which point 10 μM erastin was added. 24 h, spheroids were stained with SYTOX Green (FIG. 3K), and cell viability was measured by ATP assay (FIG. 3L). Consistently, Dox-induced Merlin restoration inhibited ferroptosis at high density and in a spheroid model (FIG. 3J-3L).

N-Cadherin Suppresses Ferroptosis in MSTO-211H Cells in a Density Dependent Manner.

Among the 10 mesothelioma cell lines tested, only H-meso cells (Merlin-wt) and H2052 cells (Merlin-mutant) had a detectable level of Ecad (FIG. 3A). Therefore, the cell density dependence of ferroptosis is not limited to epithelial cells expressing Ecad. As shown in FIG. 3A, MSTO-211H mesothelioma cells do not express Ecad. To assess the expression of N-cadherin in MSTO-211H mesothelioma cells, the cells were cultivated at different cell densities, and the levels of Ncad, p-YAP and total YAP were analyzed by Western blot. As shown in FIG. 14A, MSTO-211H mesothelioma cells expressed N-cadherin (Ncad) in a cell density-dependent manner. Therefore, the potential role of Ncad in ferroptosis was tested. MSTO-211H cells were infected with lentiviruses expressing Ncad shRNA (shNcad) and selected with puromycin. RNAi knockdown efficiency of Ncad was confirmed by Western blot (FIG. 14B). shNT or shNcad MSTO-211H cells, cultured in sparse or confluent density as indicated, were subjected to cystine starvation for 24 h, at which point cell death was measured by SYTOX Green staining followed by flow cytometry (FIG. 14C), and flow cytometric data were quantified to demonstrate cell death (FIG. 14D). shNT or shNcad MSTO-211H cells, cultured in sparse or confluent density as indicated, were also treated with 1 μM RSL3 for 16 h, at which point cell death was measured by SYTOX Green staining followed by flow cytometry (FIG. 14E). Ncad RNAi sensitized these cells to ferroptosis triggered by cystine starvation (FIG. 14C-14D) or RSL3 (FIG. 14E), when cultured at high confluence (FIGS. 14B-14E).

As shown in FIGS. 14F-14G, Ncad RNAi also sensitized 211H cells to ferroptosis in the 3D tumor spheroid model. Spheroids generated from shNT and shNcad MSTO-211H cells were treated with 10 μM erastin for 24 h. Cell death within the spheroids was determined by SYTOX Green staining (FIG. 14F), and cell viability of spheroids was assayed by measuring cellular ATP levels (FIG. 14G).

As Merlin is required for suppressing ferroptosis in 211H cells at high cell density (FIGS. 3G-3H and FIG. 13B), Ncad probably signals through Merlin-YAP pathway to regulate ferroptosis, as Ecad does in epithelial cells. To test this possibility, the activity of YAP following Ncad knockdown was examined. shNT or shNcad MSTO-211H cells were plated at high density and YAP localization was assessed by immunofluorescence (FIG. 1411). Luciferase assay for YAP/TEAD activity in shNT or shNcad MSTO-211H cells was also measured by 8×GTIIC-luciferase reporter assay; and by assessing the transcription levels of CTGF (FIG. 14I) and CYR61 were measured by qPCR (FIG. 14J). As shown in FIG. 1411, YAP nuclear localization was increased following Ncad knockdown. YAP activity, monitored by both the luciferase reporter assay and qPCR analysis for the expression of its target genes, was also increased following Ncad knockdown (FIG. 14I-14J). Together, these data suggest that Ncad can regulate ferroptosis via Hippo-YAP signaling.

Cell Density and Merlin Regulate Ferroptosis in Fibroblasts.

Merlin-regulated ferroptosis in cell density dependent manner was also observed in MEFs, which are not of epithelial origin. Cell death of MEFs with different cell density induced by cystine starvation for 12 h was measured by PI staining (FIG. 15A), and flow cytometry (FIG. 15B). The effect of cystine starvation on lipid ROS production in MEFs cells with different cell density (6 h treatment) is also shown in FIG. 15B. MEFs cultured at different densities following treatment with 1 μM erastin also underwent ferroptosis-mediated cell death (12 h) and enhanced production of lipid ROS (8 h) (FIG. 15C). RSL3-induced ferroptosis (8 h) and lipid ROS production (5 h) was also observed in MEFs cultured at different densities following treatment with 1 μM RSL3 (FIG. 15D). As shown in FIGS. 15A-15D, increased cell density mitigated both cell death and lipid ROS generation induced by cystine starvation, erastin and RSL3 in MEFs.

MEFs also regulated the activity of YAP at high density in a Merlin-dependent manner. MEFs seeded at increasing density were probed for YAP localization by Immunofluorescence (FIG. 15E). The effect of Merlin was assessed by infecting MEFs with shMerlin lentivirus and selected with puromycin. Merlin knockdown increased the nuclear accumulation of YAP as revealed by immunofluorescence (FIG. 15E); and knockdown efficiency was confirmed by Western blot. As shown in FIGS. 15E-15F, with increasing cell confluence, there was an increase of YAP nuclear exclusion in MEFs, which was attenuated by Merlin knockdown. Moreover, Merlin knockdown also enhanced ferroptosis and lipid ROS generation in MEFs (FIG. 15G). In particular, RNAi depletion of Merlin in confluent MEFs led to increased cell death and lipid ROS production upon cystine starvation, erastin (1 μM, 12 h) or RSL3 (1 μM, 8 h) treatment, which was blocked by Ferrostatin-1 (2 μM).

Accordingly, these results demonstrate that the methods of the present technology are useful for selecting a cancer patient for treatment with a ferroptosis-inducing therapy.

### Example 5: The Transcriptional Regulatory Activity of YAP Promotes Ferroptosis

The pro-oncogenic transcription co-activator YAP is one of the downstream effectors suppressed by Merlin-Hippo signaling. The above experiments demonstrate correlation between YAP activity (i.e., nuclear localization) and Ecad/Merlin-dependent regulation of ferroptosis. To determine whether YAP promotes ferroptosis, the series of functional experiments were performed.

Constitutive Activation of the YAP-TEAD Interaction Supports Ferroptosis in 211H Mesothelioma Cells.

First, the constitutively active YAP mutant, S127A (serine-127 mutated to alanine) was tested. Lats1/2 cannot phosphorylate the YAP at the S127 residue and thus YAP^{S127A} is retained in the nucleus to exert its transcriptional co-regulatory activity. To further confirm this observation, the luciferase reporter assay and qPCR analysis were used. Transcriptional levels of CTGF and CYR61 were measured by qPCR in HCT116 cells expressing YAP^{S127A} (FIG. 16A). YAP/TEAD activity in HCT116 cells expressing YAP^{S127A} was measured by 8×GTIIC-luciferase reporter assay (FIG. 16B). As shown in FIGS. 16A-16B, the transcriptional levels of CTGF and CYR61 and the luciferase activity were elevated in HCT116 cells expressing YAP^{S127A}.

To determine the effect of the constitutively active YAP^{S127A} mutant on ferroptosis, HCT116 cells were infected with retroviruses encoding Flag-YAP^{S127A}. Levels of Flag, YAP, and p-YAP were analyzed by Western blot (FIG. 4A) and localization of YAP (green) in HCT116 cells transduced with Flag-YAP^{S127A} was determined by immunofluorescence (FIG. 4B). Parental cells and YAP^{S127A}-overexpressing cells were then cultured under sparse or confluent conditions and ferroptosis was stimulated with cystine-free media. Cell death was measured after 24 h (FIG. 4C), and lipid ROS was measured by C11-BODIPY staining followed by flow cytometry after 16 h of cystine starvation (FIG. 4D). Compared with the parental control, HCT116 cells ectopically expressing the YAP^{S127A} mutant were unable to exclude the YAP mutant from the nucleus, even when cultured at a high density (FIGS. 4A-4B). These cells were markedly more sensitive to ferroptosis (FIGS. 4C-4D).

The effects of the constitutively active YAP^{S127A} mutant on ferroptosis were also tested in in vivo context. Tumor spheroids generated from parental HCT116 cells and YAP^{S127A}-overexpressing cells were treated with 15 μM erastin for 30 h. Cell death (left) and cell viability (right) within the spheroids were subsequently examined. The YAP^{S127A} mutant also sensitized ferroptosis in 3D tumor spheroids derived from HCT116 cells (FIG. 4E).

The results obtained in HCT116 cells were also observed with 211H mesothelioma cells (FIGS. 16C-16H). MSTO-211H cells were infected with retroviral vectors encoding the Flag-YAP^{S127A} mutant. Levels of Flag, YAP, and p-YAP were analyzed by Western blot (FIG. 16C). The localization of YAP (green) was determined by immunofluorescence in MSTO-211H cells expressing constitutively active YAP (FIG. 16D). Parental cells and YAP^{S127A}-overexpressing cells were cultured under sparse or confluent conditions and cell death was induced by cystine starvation. Cell death was measured after 24 h of treatment (FIG. 16E), and lipid ROS was measured after 16 h of cystine starvation (FIG. 16F). To determine the effect of constitutively active YAP^{S127A} mutant in in vivo context, tumor spheroids were generated from parental MSTO-211H cells and YAP^{S127A}-overexpression cells and were treated with 10 μM erastin for 24 h. Cell death was measured by SYTOX staining (FIG. 16G), and cell viability within spheroids was measured by cellular ATP levels (FIG. 16G).

Next, CRISPR-Cas9 technology was used to knock out YAP in HCT116 cells. YAP was knocked out by CRISPR-Cas9 approach in shMerlin HCT116 cells. YAP knockout was confirmed by Western blot (FIG. 4F). Cystine deprivation induced cell death (left, 24 h), and cystine deprivation induced lipid ROS production (right, 18 h) were then assessed in shMerlin cells and YAP knockout HCT116 cells. As shown in FIGS. 4F-4G, cells lacking YAP were no longer sensitized to ferroptosis following Merlin RNAi. These results suggest that Merlin suppresses ferroptosis by inhibiting the downstream YAP function.

To further confirm this conclusion, a pharmacological analysis was performed. As a transcriptional coactivator, YAP regulates the expression of multiple target genes through its interaction with the TEAD family of transcriptional factors. Verteporfin (VP), a pharmacological agent that can inhibit the interaction of YAP with the TEAD family members was used to determine the effect of Merlin on ferroptosis. The inhibition of YAP-TEAD interaction by verteporfin (0.5 μM) reduced cells sensitivity to ferroptosis after 24 h of cystine starvation in shMerlin HCT116 or 211H cells (FIG. 16I). YAP^{S127A}-expressing cells were also treated with verteporfin (0.5 μM) after 24 h of cystine starvation in shMerlin HCT116 or 211H cells. In both HCT116 cells and 211H cells cultured at high density, veterporfin ablated the effect of Merlin knockdown and YAP^{S127A} overexpression on restoring cystine starvation-induced ferroptosis (FIG. 16I-16J).

Overexpression of TFRC and ACSL4 Sensitize Dense Cells to Ferroptosis.

These results suggest that the transcriptional regulation mediated by the YAP-TEAD interaction is responsible for ferroptosis promotion. To test this possibility, a battery of putative YAP-TEAD gene targets that are known ferroptosis regulators were examined. Putative YAP-TEAD gene targets were selected based on publicly available ENCODE TEAD4 ChIP-seq datasets (GSM1010875 and GSM1010868). Among these genes, the transferrin receptor 1 (TFRC) and acyl-CoA synthetase long chain family member 4 (ACSL4) were validated. Both are critical mediators of ferroptosis, true targets of the YAP-TEAD complex, and are regulated by Ecad/Merlin/YAP signaling.

Expression of ACSL4 and TFRC in MSTO-211H cells with increased cell density or in confluent HCT116 cells and MSTO-211H cells upon Ecad depletion, Merlin knockdown, or YAP^{S127A} overexpression was probed by western blot. As shown in FIG. 411, the expression of TFRC and ACSL4 decreased along with increasing cell density, and TFRC and ACSL4 were upregulated upon Ecad depletion, Merlin knockdown, or YAP^{S127A} overexpression.

TEAD4 binding to ACSL4 and TFRC promoters in MSTO-211H cells was assessed by ChIP analysis using control immunoglobulin G (IgG) and TEAD4 antibody, as detailed in Example 1. Values are expressed as percent of input. TEAD4 binding peak regions in the promoters of ACSL4 and TFRC were amplified by qPCR. The β-actin (ACTB) promoter region was amplified as a negative control. ChIP assay was also used to monitor the occupancy of TEAD4 on ACSL4 and TFRC promoters of parental or YAP^{S127A}-overexpressing MSTO-211H cells. The enrichment was calculated based upon qPCR relative to the IgG control. As shown in a chromatin immunoprecipitation (ChIP) assay of FIG. 41, TEAD4 was found to bind to the promoter regions of TFRC and ACSL4 genes. The qPCR primers for the ChIP assay were designed based on TEAD4 binding peak regions depicted in the ENCODE TEAD4 ChIP-seq datasets. As shown in FIG. 4J, the binding of TEAD4 to the promoters of TFRC and ACSL4 can be stimulated by YAP^{S127A} overexpression.

To test whether TFRC and ACSL4 can directly enhance ferroptosis in confluent cells, TFRC and ACSL4 separately or in combination were overexpressed. HCT116 cells were transduced with retroviral particles containing mCherry-ACSL4, transfected with TFRC, or both. Expression was assayed by Western blot. Two bands were detected for mCherry-ACSL4, representing the full-length mCherry-ACSL4 and that with mCherry tag truncated (FIG. 17A). Additionally, cells were plated at the indicated density and treated with 2 μM RSL3 for 24 h. Cell death was measured by SYTOX Green staining coupled with flow cytometry. As shown in FIG. 17B, confluent HCT116 were partially sensitized to ferroptosis following expression of either TFRC or ACSL4. Co-expression of both further enhanced cell death (FIGS. 17A-17B).

Conversely, the effect of TFRC knockdown or CRISPR-Cas9-mediated ACSL4 knockout was assessed. HCT116 cells containing Merlin shRNA were transduced with hairpins targeting TFRC. HCT116 ΔEcad cells were transduced with hairpins targeting TFRC; and knockdown efficiency was assayed by Western blot (FIG. 4K and FIG. 17C). These cells were also treated with media containing or lacking cystine for 30 h. Cell death was measured by SYTOX Green staining (FIG. 4L and FIG. 17C). In addition, sgRNAs targeting ACSL4 were used to generate HCT116 ACSL4 knockouts. Knockout or parental cells were then transduced with Merlin shRNAs, and Merlin and ACSL4 expressions were confirmed by Western blot (FIG. 4M). As shown in FIGS. 4K-4N and FIGS. 17C-17D, TFRC knockdown or CRISPR-Cas9-mediated ACSL4 knockout mitigated ferroptosis in sensitized cells. Together, these data indicate that upregulation of TFRC and ACSL4 by the YAP-TEAD complex contributes to the function of YAP to promote ferroptosis. Notably, co-overexpression of TFRC and ACSL4 failed to restore ferroptosis in confluent cells to the level of that in sparse cells, even when the ectopic ACSL4 level was higher than that in sparse cells (FIGS. 17A-17B). Therefore, there might be additional YAP target genes that also contribute to YAP-promoted ferroptosis.

These results demonstrate that the methods of the present technology are useful for selecting a cancer patient for treatment with a ferroptosis-inducing therapy.

### Example 6: Merlin Dictates the Effect of GPX4 Inhibition in Murine Models of Mesothelioma

Inhibition of Merlin Increases Tumor Proliferation and Invasive Capability but Renders Tumor Sensitive to GPX4 Inhibition.

The loss of Merlin function is a frequent driving event in mesothelioma. In vivo mouse xenograft experiments were performed to examine whether the status of Merlin could be indicative of sensitivity to ferroptosis in mesothelioma. A doxycyline-inducible, CRISPR/Cas9-mediated GPX4 knockout (GPX4-iKO) MSTO-211H cells, harboring non-targeting shRNA (shNT) or Merlin shRNA (shMerlin) were first generated. MSTO-211H cells were infected with lentiviral particles expressing Gpx4 sgRNA and Dox-inducible Cas9 protein. Dox-inducible Cas9 expression and GPX4 knockout was confirmed by Western blot, with or without the treatment of 1 μg/ml Dox for 5 days as indicated (FIG. 5A, top). The Gpx4-iKO cells were subsequently infected with lentiviruses harboring non-targeting shRNA (shNT) or Merlin shRNA (shMerlin). Knockdown efficiency of Merlin in Gpx4-iKO cells was confirmed by Western blot (FIG. 5A, bottom). These system was then assessed in the 3D tumor spheroid system. Tumor spheroids formed by shNT-GPX4-iKO or shMerlin-GPX4-iKO 211H cells were treated with or without Doxycycline for 5 days. Cell death and cell viability within the spheroids were determined by SYTOX staining (top) and cellular ATP level measurement (bottom), respectively (FIG. 18A). In the 3D tumor spheroid system, shMerlin cells were more sensitive than shNT cells to GPX4 knockout-induced ferroptosis (FIG. 18A).

shNT-GPX4-iKO cells and shMerlin-GPX4-iKO cells were the used to produce subcutaneous xenograft tumors in athymic nude mice. shNT-GPX4-iKO cells and shMerlin-GPX4-iKO cells were subcutaneously injected into nude mice. The effect of Merlin knockdown on xenografted tumors was validated by immunohistochemical (IHC) staining of Merlin, ACSL4, TFRC, and YAP, all counter-stained with haematoxylin (blue), on sections of tumors bearing shNT and shMerlin as indicated. As shown in FIG. 18B, Merlin knockdown led to increased levels of TFRC and ACSL4 in tumors, as well as increased nuclear accumulation of YAP. To determine the levels of GPX4 following induction by doxycycline, shNT-GPX4-iKO cells and shMerlin-GPX4-iKO cells were subcutaneously injected into nude mice and fed with Dox diet or normal diet (n=8 per group). Representative images of haematoxylin and eosin (H&E) staining and IHC staining for GPX4, PTGS2, and Ki67, all counter-stained with haematoxylin (blue), were taken from sections of xenografted tumors, bearing shNT or shMerlin and with or without Dox diet (to induce GPX4 knockout) as indicated (FIG. 18C). After mice were fed food containing Dox to induce GPX4 knockout in the xenografted tumor, levels of GPX4 expression were indeed greatly reduced in tumor tissues (FIG. 18C).

Growth curves of subcutaneously injected shNT-GPX4-iKO cells and shMerlin-GPX4-iKO cells in nude mice fed with doxycycline diet or normal diet (n=8 per group) were then obtained (FIG. 5B), and images of resected subcutaneous tumors (scale bar=1 cm) analyzed. Upon Dox feeding, shMerlin tumors receded whereas shNT tumors only showed a decrease in growth (FIG. 5B and FIG. 18D), suggesting more potent ferroptosis in shMerlin tumors. Consistently, the expression of PTGS2, a marker of oxidative stress and ferroptosis, was elevated in tumor tissues upon Dox feeding, whereas Ki67 expression, indicative of cell proliferation, was reduced (FIG. 18C).

Development of an Orthotopic Intrapleural Mouse Model of Mesothelioma.

An orthotopic intrapleural mouse model of mesothelioma was also developed, by orthotopically implanting shMerlin-GPX4-iKO cells or shNT-GPX4-iKO cells harboring a retroviral TK-GFP-Luciferase (TGL) reporter. Representative bioluminescent imaging (BLI) showing the tumor growth of indicated cells in an orthotopic model of mesothelioma in NOD/SCID mice were taken (FIG. 5C). Dox treatment started when average total flux reached 108 photons per second (time-point 0). Dot plot of relative BLI signal (photons per second) percentage change versus time-point 0 was also generated (FIG. 5D). The median values were indicated with short lines. n=6 or 7 for each group. After imaging bioluminescence, region of interest (ROI) analysis was performed using the Living Image software version 4.3.1, targeting the mesothelium (FIG. 18E). The BLI signal was reported as total flux (photons per second) in each group (n=6 or 7 for each group). As shown in FIGS. 5C-5D and FIG. 18D, measurement of signal intensity indicated that shMerlin-GPX4-iKO cells grew more aggressively than shNT-GPX4-iKO cells in mice. These results were consistent with the tumor suppressive nature of Merlin in mesothelioma. Upon Dox feeding, the growth of shMerlin-GPX4-iKO xenografts was greatly reduced, whereas shNT-GPX4-iKO tumor growth was not inhibited, when compared with groups receiving a normal diet (FIGS. 5C-5D and FIG. 18E).

To assess cancer metastasis, mice were sacrificed at the endpoint, and various organs were excised for bioluminescence imaging. Bioluminescence imaging of the tumor-loaded organs were taken in mouse bodies before and after organs excision (FIG. 5E). The number of mice in each group with metastases burden in excised organs was quantified (FIG. 5F). The letters denoted the following organs: heart (H), lung (L), peritoneum (P). I, intestines/mesenteric lymph nodes (I), liver (Li), spleen (S), kidneys (K). As shown in FIGS. 5E-5F, shNT-GPX4-iKO tumors grew within the pleural cavity, attaching to the aortic arch, lung or thoracic muscles, whereas shMerlin-GPX4-iKO tumors tended to metastasize to pericardium, peritoneum, and abdominal organs including liver, intestine and distal lymph nodes. These observations were consistent with previous reports that loss of Merlin function enhances the metastasis of mesothelioma.

To further support the observation that metastasis was enhanced in the absence of Merlin, tumor spheroids of 211H cells expressing shNT or shMerlin were grown in Matrigel, and cell invasion was monitored (FIG. 18F). In the representative images, arrows show protrusions extruded from the main body of spheroids. The tumor spheroid analysis revealed that Merlin knockdown rendered tumor cells to extend more finger-like protrusions from the spheroid body into Matrigel (FIG. 18F). Importantly, the metastatic capability of shMerlin tumors was mitigated by Dox-induced GPX4 knockout (FIGS. 5E-5F). These results indicate that Merlin status in mesothelioma tumor tissues might be useful as a biomarker to predict tumor metastasis and responsiveness to the induction of ferroptosis cell death.

Inhibition of the Hippo Pathway Sensitizes Tumors to Imidazole Ketone Erastin (IKE).

To determine whether Lats1/2, the kinases that directly phosphorylate and inactivate YAP, can have similar effects in vivo as Merlin, subcutaneous xenograft tumors were formed from HCT116 cells harboring hairpins targeting Lats1/2. In particular, HCT116 cells containing non-targeting hairpins or hairpins targeting both Lats1 and 2 were injected subcutaneously into nude mice (n=6 per group). Tumors were grown to a volume of 90 mm³, at which point they were injected intratumorally with 50 mg/kg imidazole ketone erastin (IKE) once a day for 12 days. Tumor volume was measured daily by caliper. Images of resected tumors and their masses were also quantified (FIGS. 19B-19C). Imidazole ketone erastin (IKE) is an erastin analog that is amenable for in vivo use because of the increased solubility and stability. As shown in FIGS. 19A-19C, HCT116-derived xenograft tumors grew slowly or receded in response to IKE only when Lats1/2 were inhibited. This result was consistent with the effect of Lats1/2 knockdown shown in FIG. 2B, and further demonstrated that inhibition of the Hippo pathway rendered tumors more sensitive to GPX4 inhibition.

These results demonstrate that the methods of the present technology are useful for selecting a cancer patient for treatment with a ferroptosis-inducing therapy.

### Example 7: The Cadherin-Hippo-YAP Signaling Axis Regulates Sensitivity to Sorafenib-Induced Ferroptosis

Sorafenib is an orally administered drug used for the treatment of hepatocellular carcinoma and renal cell carcinoma. Sorafenib inhibits multiple kinases including VEGF, PDGF, and Raf family kinases, and has more recently been found to also be able to induce ferroptosis by inhibiting the system xc− antiporter. The potential for sorafenib as a therapy for malignant mesothelioma has been tested in clinical trials, and the results suggest that sorafenib can stabilize, but not necessarily treat mesothelioma. However, these trials did not examine the genetic status of the Merlin-Hippo pathway.

The effect of sorafenib on cell density-dependent ferroptosis was determined. First, HCT116 cells were seeded at a density of 0.5×105 cells/3.5 cm2 well (sparse) or 4×105 cells/3.5 cm2 well (confluent) and grown for 24 h. Cells were then treated with DMSO, 10 μM sorafenib, or 10 μM sorafenib and 2 μM Fer-1 as indicated. FIG. 20A confirmed that sorafenib could induce cell density-dependent ferroptosis. Second, ΔEcad or the parental HCT116 cells were seeded at 4×105 cells/3.5 cm2 well and grown for 24 h. Cells were then treated with 10 μM sorafenib and 2 μM Fer-1 as indicated. Third, HCT116 shNT or shMerlin cells, MSTO-211H shNT or shMerlin cells, HCT116 cells expressing YAP^{S127A} or the parental cells, MSTO-211H cells expressing YAP^{S127A} or the parental cells, and HCT116 shNT or shLats1/2 cells were seeded at high density and treated with 10 μM sorafenib and 2 μM Fer-1 as indicated. The results demonstrated that loss of Ecad (FIG. 20B) or Lats1/2 (FIG. 20G) in HCT116 cells, and inhibition of Merlin (FIG. 20C-20D) or activation of YAP (FIG. 20E-20F) in both HCT116 cells and 211H mesothelioma cells, could sensitize these cells to sorafenib-induced ferroptosis at high cell density.

Taken together, these results demonstrate that cells become resistant to ferroptosis when growing confluent. It has been reported previously that a high enough population of cells can sustain antioxidant defenses through de novo cysteine production via the transsulfuration pathway. However, RSL3-induced ferroptosis, which is independent of cellular cysteine or glutathione levels due to the direct inhibition of downstream GPX4, is also subjected to such regulation. These results demonstrate that when cells contact with each other, they become more resistant to ferroptosis because of cadherin-mediated cell adhesion. This intercellular interaction signals through Merlin-Hippo pathway to suppress transcription co-activator YAP, which can function as a positive regulator of ferroptosis by upregulating the expression of multiple ferroptosis factors. In particular, YAP upregulates ACSL4 and TFRC to shape cellular lipid and iron metabolism, respectively, rendering the cell more susceptible to ferroptosis.

Ferroptosis employs cell-intrinsic machinery to execute the death process. However, for ferroptosis, neighboring cells can have a significant impact on decision making, via the cadherin-Merlin-Hippo-YAP signaling axis. Such intercellular communication appears to be mutually beneficial, as it increases the resistance to ferroptosis of all involved cells. Notably, the consequence of this intercellular communication is in stark contrast to that of death receptor-mediated apoptosis, in which one cell (bearing the death receptor ligand) induces apoptotic death of the other (bearing the death receptor). Considering that multicellular organisms are under frequent insult of oxidative stress, this intercellular anti-ferroptotic mechanism might represent another layer of crucial defense to protect themselves from ferroptosis, a devastating and terminal consequence of oxidative stress.

These results demonstrate that the methods of the present technology are useful for selecting a cancer patient for treatment with a ferroptosis-inducing therapy.

### Example 8: Induction of an EMT-Like State in MMTV-Neu NF639 Breast Cancer Cells by TGFβ Mitigates Density-Dependent Control of Ferroptosis

The potential link between epithelial-mesenchymal transition (EMT) and ferroptosis was examined using cells generated from mammary tumors of MMTV-neu mice. NF639 cells, derived from mouse mammary tumors containing MMTV-neu, were treated with various concentrations of TGF-β for 48 h to induce EMT. TGF-β can enhance EMT in cells bearing overactive HER2/neu signaling. Expression of a panel of EMT-related genes was assayed by qPCR as indicated. As shown in FIG. 21A, TGF-β upregulated the expression of multiple EMT-related genes.

To determine the effect of TGF-β on cystine deprivation induced ferroptosis, NF639 cells were treated with or without 6 ng/μL TGF-β for 48 h, at which point they were plated at low density (0.8 cells×10⁵/3.5 cm² well), grown overnight, and treated with media containing or lacking cystine, or lacking cystine with 1 μM ferrostatin-1 for 12 h. NF639 cells were also plated at a high density of 3.2×10⁵ cells/3.5 cm² well, grown overnight and treated with media containing or lacking cystine, or lacking cystine with 1 μM ferrostatin-1 for 12 h. Cell death was measured by Sytox green staining coupled with flow cytometry. Cells treated with TGF-β died under cystine starvation at high cell density, while untreated cells were highly resistant (FIGS. 21B-21C).

In epithelial cancer cells, decreased Ecad or Merlin expression, reduced Hippo pathway activity, and enhanced YAP activation can promote EMT and metastasis. Strikingly, alteration of these factors, while promoting EMT and mesenchymal properties of cancer cells, can also enhance the sensitivity of cancer cells to ferroptosis, as demonstrated herein. Importantly, the role of Merlin-Hippo-YAP signaling in ferroptosis is not limited to epithelial cells or EMT. For example, MEFs, which are derived from fibroblasts rather than epithelial tissue, and multiple mesothelioma cell lines that do not express Ecad, were also regulated by such cell density-dependent mechanism. In these contexts, cell-cell contact might be mediated by other forms of cadherins, and ferroptosis is similarly modulated by the Merlin-YAP signaling. Therefore, the involvement of Merlin-Hippo-YAP signaling in the regulation of ferroptosis is widely applicable, and not limited to epithelial cells or tumor cells.

Two potential players that, at least in part, explain how ferroptosis is regulated by Hippo signaling were identified: TFRC and ACSL4. TFRC is critical for the maintenance of iron homeostasis within cells, and transferrin has been identified as an important iron source for the Fenton reaction that amplifies lipid peroxide species during ferroptosis. ACSL4 is a fatty acid-CoA ligase that is essential for ferroptosis due to its preferential ability to synthesize long polyunsaturated fatty acids that are highly sensitive to peroxidation. Accordingly, these results demonstrate that the methods of the present technology are useful for selecting a cancer patient for treatment with a ferroptosis-inducing therapy.

### EQUIVALENTS

The present technology is not to be limited in terms of the particular embodiments described in this application, which are intended as single illustrations of individual aspects of the present technology. Many modifications and variations of this present technology can be made without departing from its spirit and scope, as will be apparent to those skilled in the art. Functionally equivalent methods and apparatuses within the scope of the present technology, in addition to those enumerated herein, will be apparent to those skilled in the art from the foregoing descriptions. Such modifications and variations are intended to fall within the scope of the present technology. It is to be understood that this present technology is not limited to particular methods, reagents, compounds compositions or biological systems, which can, of course, vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

All patents, patent applications, provisional applications, and publications referred to or cited herein are incorporated by reference in their entirety, including all figures and tables, to the extent they are not inconsistent with the explicit teachings of this specification.

1. A method for selecting a cancer patient for treatment with a ferroptosis-inducing therapy comprising
(a) detecting the presence of a mutation in at least one polynucleotide encoding one or more proteins selected from the group consisting of E-cadherin, N-cadherin, Merlin, Mst1, Mst2, Lats1, and Lats2 in a biological sample obtained from the cancer patient, wherein the mutation is a frameshift mutation, a missense mutation, a deletion, an insertion, a nonsense mutation, an inversion, or a translocation; and
(b) administering to the cancer patient an effective amount of a ferroptosis-inducing agent.

2. The method of claim 1, wherein the mutation is detected via next-generation sequencing, PCR, real-time quantitative PCR (qPCR), digital PCR (dPCR), Southern blotting, Reverse transcriptase-PCR (RT-PCR), Northern blotting, microarray, dot or slot blots, in situ hybridization, or fluorescent in situ hybridization (FISH).

3. The method of claim 1, wherein the biological sample comprises genomic DNA, cDNA, RNA, and/or mRNA.

4. A method for treating a therapy-resistant, metastasis-prone cancer in a patient in need thereof comprising
administering to the cancer patient an effective amount of a ferroptosis-inducing agent, wherein mRNA or polypeptide expression and/or activity levels of one or more of E-cadherin, N-cadherin, Merlin, Mst1, Mst2, Lats1, and Lats2 in a biological sample obtained from the patient are reduced compared to that observed in a control sample obtained from a healthy subject or a predetermined threshold.

5. A method for treating a therapy-resistant, metastasis-prone cancer in a patient in need thereof comprising
administering to the cancer patient an effective amount of a ferroptosis-inducing agent, wherein mRNA or polypeptide expression and/or activity levels of one or more of YAP, TAZ, TFRC, ACSL4, and TGF-β are elevated compared to that observed in a control sample obtained from a healthy subject or a predetermined threshold, optionally wherein
mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH) or
polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

6. The method of claim 4, wherein mRNA expression levels are detected via real-time quantitative PCR (qPCR), digital PCR (dPCR), Reverse transcriptase-PCR (RT-PCR), Northern blotting, microarray, dot or slot blots, in situ hybridization, or fluorescent in situ hybridization (FISH).

7. The method of claim 5, wherein TFRC mRNA expression levels are detected using a forward primer comprising the sequence of SEQ ID NO: 36 and a reverse primer comprising the sequence of SEQ ID NO: 37.

8. The method of claim 5, wherein TFRC mRNA expression levels are detected using a probe comprising the sequence of SEQ ID NO: 15, SEQ ID NO: 16, SEQ ID NO: 36, SEQ ID NO: 37, or any complement thereof.

9. The method of claim 5, wherein ACSL4 mRNA expression levels are detected using a forward primer comprising the sequence of SEQ ID NO: 34 and a reverse primer comprising the sequence of SEQ ID NO: 35.

10. The method of claim 5, wherein ACSL4 mRNA expression levels are detected using a probe comprising the sequence of SEQ ID NO: 34, SEQ ID NO: 35, or any complement thereof.

11. The method of claim 4, wherein Merlin mRNA expression levels are detected using a forward primer comprising the sequence of SEQ ID NO: 1 and a reverse primer comprising the sequence of SEQ ID NO: 2.

12. The method of claim 4, wherein Merlin mRNA expression levels are detected using a probe comprising the sequence of SEQ ID NO: 1, SEQ ID NO: 2, SEQ ID NO: 7, SEQ ID NO: 8, SEQ ID NO: 9, SEQ ID NO: 10, or any complement thereof.

13. The method of claim 4, wherein E-cadherin or N-cadherin mRNA expression levels are detected using a probe comprising the sequence of SEQ ID NO: 3, SEQ ID NO: 4, SEQ ID NO: 5, SEQ ID NO: 6, or any complement thereof.

14. The method of claim 4, wherein Lats1 or Lats2 mRNA expression levels are detected using a probe comprising the sequence of SEQ ID NO: 11, SEQ ID NO: 12, SEQ ID NO: 13, SEQ ID NO: 14, or any complement thereof.

15. The method of claim 4, wherein polypeptide expression levels are detected via Western blotting, enzyme-linked immunosorbent assays (ELISA), dot blotting, immunohistochemistry, immunofluorescence, immunoprecipitation, immunoelectrophoresis, or mass-spectrometry.

16. The method of claim 4, wherein the metastasis-prone cancer is resistant to chemotherapy or radiation therapy.

17. The method of claim 1, wherein the patient suffers from a cancer selected from the group consisting of mesothelioma, lung cancer, liver cancer, colon cancer, rectal cancer, and breast cancer.

18. The method of claim 1, wherein the ferroptosis-inducing agent is a class 1 ferroptosis inducer (system X_{c}⁻ inhibitor) or a class 2 ferroptosis inducer (glutathione peroxidase 4 (GPx4) inhibitor).

19. The method of claim 1, wherein the ferroptosis-inducing agent is selected from the group consisting of erastin, erastin derivatives (e.g., MEII, PE, AE, imidazole ketone erastin (IKE)), DPI2, BSO, SAS, lanperisone, SRS13-45, SRS13-60, RSL3, DPI7, DPI10, DPI12, DPI13, DPI17, DPI18, DPI19, ML160, sorafenib, and artemisinin derivatives.

20. (canceled)


SEQUENCE LISTING (49 sequences)

SEQ ID NO: 1 - DNA, 35 bp, Artificial Sequence
  Description of Artificial Sequence Synthetic primer
actgttaattaacatggactacaaagacgatgacg

SEQ ID NO: 2 - DNA, 33 bp, Artificial Sequence
  Description of Artificial Sequence Synthetic primer
atgagagaattccaagcttctgcaggtcgactc

SEQ ID NO: 3 - DNA, 21 bp, Artificial Sequence
  Description of Artificial Sequence Synthetic oligonucleotide
gcagaaattattgggctcttt

SEQ ID NO: 4 - DNA, 21 bp, Artificial Sequence
  Description of Artificial Sequence Synthetic oligonucleotide
ccagtgaacaacgatggcatt

SEQ ID NO: 5 - DNA, 21 bp, Artificial Sequence
  Description of Artificial Sequence Synthetic oligonucleotide
ccagtgactattaagagaaat

SEQ ID NO: 6 - DNA, 21 bp, Artificial Sequence
  Description of Artificial Sequence Synthetic oligonucleotide
cgcattatgcaagactggatt

SEQ ID NO: 7 - DNA, 21 bp, Artificial Sequence
  Description of Artificial Sequence Synthetic oligonucleotide
gaagcaacccaagacgttcac

SEQ ID NO: 8 - DNA, 21 bp, Artificial Sequence
  Description of Artificial Sequence Synthetic oligonucleotide
tagttctctgacctgagtctt

SEQ ID NO: 9 - DNA, 21 bp, Artificial Sequence
  Description of Artificial Sequence Synthetic oligonucleotide
cgagcgtacaagagatgagtt

SEQ ID NO: 10 - DNA, 21 bp, Artificial Sequence
  Description of Artificial Sequence Synthetic oligonucleotide
gcagcaagcataataccatta

SEQ ID NO: 11 - DNA, 21 bp, Artificial Sequence
  Description of Artificial Sequence Synthetic oligonucleotide
caagtcagaaatccacccaaa

SEQ ID NO: 12 - DNA, 21 bp, Artificial Sequence
  Description of Artificial Sequence Synthetic oligonucleotide
gattacaacttcacctattac

SEQ ID NO: 13 - DNA, 21 bp, Artificial Sequence
  Description of Artificial Sequence Synthetic oligonucleotide
ccgtcgattacttcacttgaa

SEQ ID NO: 14 - DNA, 21 bp, Artificial Sequence
  Description of Artificial Sequence Synthetic oligonucleotide
gccatgaagaccctaaggaaa

SEQ ID NO: 15 - DNA, 21 bp, Artificial Sequence
  Description of Artificial Sequence Synthetic oligonucleotide
cccaacagatactggaagttt

SEQ ID NO: 16 - DNA, 21 bp, Artificial Sequence
  Description of Artificial Sequence Synthetic oligonucleotide
gctggtcagttcgtgattaaa

SEQ ID NO: 17 - DNA, 20 bp, Artificial Sequence
  Description of Artificial Sequence Synthetic oligonucleotide
cacgcccgatacgctgagtg

SEQ ID NO: 18 - DNA, 19 bp, Artificial Sequence
  Description of Artificial Sequence Synthetic primer
ccaatgacaacgcctcctg

SEQ ID NO: 19 - DNA, 19 bp, Artificial Sequence
  Description of Artificial Sequence Synthetic primer
tggtgcagccagaaagctc

SEQ ID NO: 20 - DNA, 21 bp, Artificial Sequence
  Description of Artificial Sequence Synthetic primer
agcctcgcatcctatacaacc

SEQ ID NO: 21 - DNA, 21 bp, Artificial Sequence
  Description of Artificial Sequence Synthetic primer
ttctttcacaaggcggcactc

SEQ ID NO: 22 - DNA, 21 bp, Artificial Sequence
  Description of Artificial Sequence Synthetic primer
agatggcaagctgcagctatg

SEQ ID NO: 23 - DNA, 24 bp, Artificial Sequence
  Description of Artificial Sequence Synthetic primer
gactgtccattttctccttctctg

SEQ ID NO: 24 - DNA, 21 bp, Artificial Sequence
  Description of Artificial Sequence Synthetic primer
aggtgtaagcgcagaaagcag

SEQ ID NO: 25 - DNA, 21 bp, Artificial Sequence
  Description of Artificial Sequence Synthetic primer
tggagtttgtcttcatcatcg

SEQ ID NO: 26 - DNA, 20 bp, Artificial Sequence
  Description of Artificial Sequence Synthetic primer
cgcttgacatcgctgaagga

SEQ ID NO: 27 - DNA, 20 bp, Artificial Sequence
  Description of Artificial Sequence Synthetic primer
cttgccacactccgtgcact

SEQ ID NO: 28 - DNA, 20 bp, Artificial Sequence
  Description of Artificial Sequence Synthetic primer
ctttctcttgccctcactgc

SEQ ID NO: 29 - DNA, 20 bp, Artificial Sequence
  Description of Artificial Sequence Synthetic primer
acagcagccagactcctcat

SEQ ID NO: 30 - DNA, 20 bp, Artificial Sequence
  Description of Artificial Sequence Synthetic primer
catccgagtgggtttggagg

SEQ ID NO: 31 - DNA, 19 bp, Artificial Sequence
  Description of Artificial Sequence Synthetic primer
cttctctaggccctggctg

SEQ ID NO: 32 - DNA, 20 bp, Artificial Sequence
  Description of Artificial Sequence Synthetic primer
ccatgttcgtcatgggtgtg

SEQ ID NO: 33 - DNA, 20 bp, Artificial Sequence
  Description of Artificial Sequence Synthetic primer
caggggtgctaagcagttgg

SEQ ID NO: 34 - DNA, 20 bp, Artificial Sequence
  Description of Artificial Sequence Synthetic primer
gtgctgtacctaaatgcaac

SEQ ID NO: 35 - DNA, 20 bp, Artificial Sequence
  Description of Artificial Sequence Synthetic primer
acattgccacaagagtacaa

SEQ ID NO: 36 - DNA, 20 bp, Artificial Sequence
  Description of Artificial Sequence Synthetic primer
acgagggtcggtgtagttct

SEQ ID NO: 37 - DNA, 20 bp, Artificial Sequence
  Description of Artificial Sequence Synthetic primer
ctcaggaagtgacgcacagc

SEQ ID NO: 38 - DNA, 4811 bp, Homo sapiens
agtggcgtcggaactgcaaagcacctgtgagcttgcggaagtcagttcagactccagcccgctccagcccggcccgacccgaccgcacccggcgcctgccctcgctcggcgtccccggccagccatgggcccttggagccgcagcctctcggcgctgctgctgctgctgcaggtctcctcttggctctgccaggagccggagccctgccaccctggctttgacgccgagagctacacgttcacggtgccccggcgccacctggagagaggccgcgtcctgggcagagtgaattttgaagattgcaccggtcgacaaaggacagcctatttttccctcgacacccgattcaaagtgggcacagatggtgtgattacagtcaaaaggcctctacggtttcataacccacagatccatttcttggtctacgcctgggactccacctacagaaagttttccaccaaagtcacgctgaatacagtggggcaccaccaccgccccccgccccatcaggcctccgtttctggaatccaagcagaattgctcacatttcccaactcctctcctggcctcagaagacagaagagagactgggttattcctcccatcagctgcccagaaaatgaaaaaggcccatttcctaaaaacctggttcagatcaaatccaacaaagacaaagaaggcaaggttttctacagcatcactggccaaggagctgacacaccccctgttggtgtctttattattgaaagagaaacaggatggctgaaggtgacagagcctctggatagagaacgcattgccacatacactctcttctctcacgctgtgtcatccaacgggaatgcagttgaggatccaatggagattttgatcacggtaaccgatcagaatgacaacaagcccgaattcacccaggaggtctttaaggggtctgtcatggaaggtgctcttccaggaacctctgtgatggaggtcacagccacagacgcggacgatgatgtgaacacctacaatgccgccatcgcttacaccatcctcagccaagatcctgagctccctgacaaaaatatgttcaccattaacaggaacacaggagtcatcagtgtggtcaccactgggctggaccgagagagtttccctacgtataccctggtggttcaagctgctgaccttcaaggtgaggggttaagcacaacagcaacagctgtgatcacagtcactgacaccaacgataatcctccgatcttcaatcccaccacgtacaagggtcaggtgcctgagaacgaggctaacgtcgtaatcaccacactgaaagtgactgatgctgatgcccccaataccccagcgtgggaggctgtatacaccatattgaatgatgatggtggacaatttgtcgtcaccacaaatccagtgaacaacgatggcattttgaaaacagcaaagggcttggattttgaggccaagcagcagtacattctacacgtagcagtgacgaatgtggtaccttttgaggtctctctcaccacctccacagccaccgtcaccgtggatgtgctggatgtgaatgaagcccccatctttgtgcctcctgaaaagagagtggaagtgtccgaggactttggcgtgggccaggaaatcacatcctacactgcccaggagccagacacatttatggaacagaaaataacatatcggatttggagagacactgccaactggctggagattaatccggacactggtgccatttccactcgggctgagctggacagggaggattttgagcacgtgaagaacagcacgtacacagccctaatcatagctacagacaatggttctccagttgctactggaacagggacacttctgctgatcctgtctgatgtgaatgacaacgcccccataccagaacctcgaactatattcttctgtgagaggaatccaaagcctcaggtcataaacatcattgatgcagaccttcctcccaatacatctcccttcacagcagaactaacacacggggcgagtgccaactggaccattcagtacaacgacccaacccaagaatctatcattttgaagccaaagatggccttagaggtgggtgactacaaaatcaatctcaagctcatggataaccagaataaagaccaagtgaccaccttagaggtcagcgtgtgtgactgtgaaggggccgctggcgtctgtaggaaggcacagcctgtcgaagcaggattgcaaattcctgccattctggggattcttggaggaattcttgctttgctaattctgattctgctgctcttgctgtttcttcggaggagagcggtggtcaaagagcccttactgcccccagaggatgacacccgggacaacgtttattactatgatgaagaaggaggcggagaagaggaccaggactttgacttgagccagctgcacaggggcctggacgctcggcctgaagtgactcgtaacgacgttgcaccaaccctcatgagtgtcccccggtatcttccccgccctgccaatcccgatgaaattggaaattttattgatgaaaatctgaaagcggctgatactgaccccacagccccgccttatgattctctgctcgtgtttgactatgaaggaagcggttccgaagctgctagtctgagctccctgaactcctcagagtcagacaaagaccaggactatgactacttgaacgaatggggcaatcgcttcaagaagctggctgacatgtacggaggcggcgaggacgactaggggactcgagagaggcgggccccagacccatgtgctgggaaatgcagaaatcacgttgctggtggtttttcagctcccttcccttgagatgagtttctggggaaaaaaaagagactggttagtgatgcagttagtatagctttatactctctccactttatagctctaataagtttgtgttagaaaagtttcgacttatttcttaaagcttttttttttttcccatcactctttacatggtggtgatgtccaaaagatacccaaattttaatattccagaagaacaactttagcatcagaaggttcacccagcaccttgcagattttcttaaggaattttgtctcacttttaaaaagaaggggagaagtcagctactctagttctgttgttttgtgtatataattttttaaaaaaaatttgtgtgcttctgctcattactacactggtgtgtccctctgccttttttttttttttaagacagggtctcattctatcggccaggctggagtgcagtggtgcaatcacagctcactgcagccttgtcctcccaggctcaagctatccttgcacctcagcctcccaagtagctgggaccacaggcatgcaccactacgcatgactaattttttaaatatttgagacggggtctccctgtgttacccaggctggtctcaaactcctgggctcaagtgatcctcccatcttggcctcccagagtattgggattacagacatgagccactgcacctgcccagctccccaactccctgccattttttaagagacagtttcgctccatcgcccaggcctgggatgcagtgatgtgatcatagctcactgtaacctcaaactctggggctcaagcagttctcccaccagcctcctttttatttttttgtacagatggggtcttgctatgttgcccaagctggtcttaaactcctggcctcaagcaatccttctgccttggccccccaaagtgctgggattgtgggcatgagctgctgtgcccagcctccatgttttaatatcaactctcactcctgaattcagttgctttgcccaagataggagttctctgatgcagaaattattgggctcttttagggtaagaagtttgtgtctttgtctggccacatcttgactaggtattgtctactctgaagacctttaatggcttccctctttcatctcctgagtatgtaacttgcaatgggcagctatccagtgacttgttctgagtaagtgtgttcattaatgtttatttagctctgaagcaagagtgatatactccaggacttagaatagtgcctaaagtgctgcagccaaagacagagcggaactatgaaaagtgggcttggagatggcaggagagcttgtcattgagcctggcaatttagcaaactgatgctgaggatgattgaggtgggtctacctcatctctgaaaattctggaaggaatggaggagtctcaacatgtgtttctgacacaagatccgtggtttgtactcaaagcccagaatccccaagtgcctgcttttgatgatgtctacagaaaatgctggctgagctgaacacatttgcccaattccaggtgtgcacagaaaaccgagaatattcaaaattccaaatttttttcttaggagcaagaagaaaatgtggccctaaagggggttagttgaggggtagggggtagtgaggatcttgatttggatctctttttatttaaatgtgaatttcaacttttgacaatcaaagaaaagacttttgttgaaatagctttactgtttctcaagtgttttggagaaaaaaatcaaccctgcaatcactttttggaattgtcttgatttttcggcagttcaagctatatcgaatatagttctgtgtagagaatgtcactgtagttttgagtgtatacatgtgtgggtgctgataattgtgtattttctttgggggtggaaaaggaaaacaattcaagctgagaaaagtattctcaaagatgcatttttataaattttattaaacaattttgttaaa

SEQ ID NO: 39 - DNA, 4016 bp, Homo sapiens
gccgtttctccgcgccgctgttggtgctgccgctgcctcctcctcctccgccgccgccgccgccgccgccgcctcctccggctcttcgctcggcccctctccgcctccatgtgccggatagcgggagcgctgcggaccctgctgccgctgctggcggccctgcttcaggcgtctgtagaggcttctggtgaaatcgcattatgcaagactggatttcctgaagatgtttacagtgcagtcttatcgaaggatgtgcatgaaggacagcctcttctcaatgtgaagtttagcaactgcaatggaaaaagaaaagtacaatatgagagcagtgagcctgcagattttaaggtggatgaagatggcatggtgtatgccgtgagaagctttccactctcttctgagcatgccaagttcctgatatatgcccaagacaaagagacccaggaaaagtggcaagtggcagtaaaattgagcctgaagccaaccttaactgaggagtcagtgaaggagtcagcagaagttgaagaaatagtgttcccaagacaattcagtaagcacagtggccacctacaaaggcagaagagagactgggtcatccctccaatcaacttgccagaaaactccaggggaccttttcctcaagagcttgtcaggatcaggtctgatagagataaaaacctttcactgcggtacagtgtaactgggccaggagctgaccagcctccaactggtatcttcattatcaaccccatctcgggtcagctgtcggtgacaaagcccctggatcgcgagcagatagcccggtttcatttgagggcacatgcagtagatattaatggaaatcaagtggagaaccccattgacattgtcatcaatgttattgacatgaatgacaacagacctgagttcttacaccaggtttggaatgggacagttcctgagggatcaaagcctggaacatatgtgatgaccgtaacagcaattgatgctgacgatcccaatgccctcaatgggatgttgaggtacagaatcgtgtctcaggctccaagcaccccttcacccaacatgtttacaatcaacaatgagactggtgacatcatcacagtggcagctggacttgatcgagaaaaagtgcaacagtatacgttaataattcaagctacagacatggaaggcaatcccacatatggcctttcaaacacagccacggccgtcatcacagtgacagatgtcaatgacaatcctccagagtttactgccatgacgttttatggtgaagttcctgagaacagggtagacatcatagtagctaatctaactgtgaccgataaggatcaaccccatacaccagcctggaacgcagtgtacagaatcagtggcggagatcctactggacggttcgccatccagaccgacccaaacagcaacgacgggttagtcaccgtggtcaaaccaatcgactttgaaacaaataggatgtttgtccttactgttgctgcagaaaatcaagtgccattagccaagggaattcagcacccccctcagtcaactgcaaccgtgtctgttacagttattgacgtaaatgaaaacccttattttgcccccaatcctaagatcattcgccaagaagaagggcttcatgccggtaccatgttgacaacattcactgctcaggacccagatcgatatatgcagcaaaatattagatacactaaattatctgatcctgccaattggctaaaaatagatcctgtgaatggacaaataactacaattgctgttttggaccgagaatcaccaaatgtgaaaaacaatatatataatgctactttccttgcttctgacaatggaattcctcctatgagtggaacaggaacgctgcagatctatttacttgatattaatgacaatgcccctcaagtgttacctcaagaggcagagacttgcgaaactccagaccccaattcaattaatattacagcacttgattatgacattgatccaaatgctggaccatttgcttttgatcttcctttatctccagtgactattaagagaaattggaccatcactcggcttaatggtgattttgctcagcttaatttaaagataaaatttcttgaagctggtatctatgaagttcccatcataatcacagattcgggtaatcctcccaaatcaaatatttccatcctgcgtgtgaaggtttgccagtgtgactccaacggggactgcacagatgtggacaggattgtgggtgcggggcttggcaccggtgccatcattgccatcctgctctgcatcatcatcctgcttatccttgtgctgatgtttgtggtatggatgaaacgccgggataaagaacgccaggccaaacaacttttaattgatccagaagatgatgtaagagataatattttaaaatatgatgaagaaggtggaggagaagaagaccaggactatgacttgagccagctgcagcagcctgacactgtggagcctgatgccatcaagcctgtgggaatccgacgaatggatgaaagacccatccacgccgagccccagtatccggtccgatctgcagccccacaccctggagacattggggacttcattaatgagggccttaaagcggctgacaatgaccccacagctccaccatatgactccctgttagtgtttgactatgaaggcagtggctccactgctgggtccttgagctcccttaattcctcaagtagtggtggtgagcaggactatgattacctgaacgactgggggccacggttcaagaaacttgctgacatgtatggtggaggtgatgactgaacttcagggtgaacttggtttttggacaagtacaaacaatttcaactgatattcccaaaaagcattcagaagctaggctttaactttgtagtctactagcacagtgcttgctggaggctttggcataggctgcaaaccaatttgggctcagagggaatatcagtgatccatactgtttggaaaaacactgagctcagttacacttgaattttacagtacagaagcactgggattttatgtgcctttttgtacctttttcagattggaattagttttctgtttaaggctttaatggtactgatttctgaaacgataagtaaaagacaaaatattttgtggtgggagcagtaagttaaaccatgatatgcttcaacacgcttttgttacattgcatttgcttttattaaaatacaaaattaaacaaacaaaaaaactcatggagcgattttattatcttgggggatgagaccatgagattggaaaatgtacattacttctagttttagactttagtttgtttttttttttttcactaaaatcttaaaacttactcagctggttgcaaataaagggagttttcatatcaccaatttgtagcaaaattgaattttttcataaactagaatgttagacacattttggtcttaatccatgtacacttttttatttctgtatttttccacttcactgtaaaaatagtatgtgtacataatgttttattggcatagtctatggagaagtgcagaaacttcagaacatgtgtatgtattatttggactatggattcaggttttttgcatgtttatatctttcgttatggataaagtatttacaaaacagtgacatttgattcaattgttgagctgtagttagaatactcaatttttaatttttttaatttttttattttttattttctttttggtttggggagggagaaaagttcttagcacaaatgttttacataatttgtaccaaaaaaaaaaaaaaaggaaaggaaagaaaggggtggcctgacactggtggcactactaagtgtgtgtttttttaaaaaaaaaatggaaaaaaaaaagcttttaaactggagagacttctgacaacagctttgcctctgtattgtgtaccagaatataaatgatacacctctgaccccagcgttctgaataaaatgctaattttggatctgg

SEQ ID NO: 40 - DNA, 6046 bp, Homo sapiens
tgcgcttcccgcgggcgcgcggagtgaggacggtgacagccacgcgcgcgcgtacgcgcccgatgcagcgcggccccgtgaccctagtcggccgctgagaggcgcgcggagtctgggccgctgccgtctaggggtcccgtcccgaggcgtccccggcatctccggcccgaatcccggagtgccgggtcgcgcctgcaccgaaggtcccggctcctgtgccctccctgcagccgtcagggcccgtcccccaactcccctttccgctcaggcagggtcctcgcggcccatgctggccgctggggacccgcgcagcccagaccgttcccgggccgggcagccggccaccatggtggccctgaggcctgtgcagcaactccaggggggctaaagggctcagagtgcaggccgtggggcgcgagggtcccgggcctgagccccgcgccatggccggggccatcgcttcccgcatgagcttcagctctctcaagaggaagcaacccaagacgttcaccgtgaggatcgtcaccatggacgccgagatggagttcaattgcgagatgaagtggaaagggaaggacctctttgatttggtgtgccggactctggggctccgagaaacctggttctttggactgcagtacacaatcaaggacacagtggcctggctcaaaatggacaagaaggtactggatcatgatgtttcaaaggaagaaccagtcacctttcacttcttggccaaattttatcctgagaatgctgaagaggagctggttcaggagatcacacaacatttattcttcttacaggtaaagaagcagattttagatgaaaagatctactgccctcctgaggcttctgtgctcctggcttcttacgccgtccaggccaagtatggtgactacgaccccagtgttcacaagcggggatttttggcccaagaggaattgcttccaaaaagggtaataaatctgtatcagatgactccggaaatgtgggaggagagaattactgcttggtacgcagagcaccgaggccgagccagggatgaagctgaaatggaatatctgaagatagctcaggacctggagatgtacggtgtgaactactttgcaatccggaataaaaagggcacagagctgctgcttggagtggatgccctggggcttcacatttatgaccctgagaacagactgacccccaagatctccttcccgtggaatgaaatccgaaacatctcgtacagtgacaaggagtttactattaaaccactggataagaaaattgatgtcttcaagtttaactcctcaaagcttcgtgttaataagctgattctccagctatgtatcgggaaccatgatctatttatgaggagaaggaaagccgattctttggaagttcagcagatgaaagcccaggccagggaggagaaggctagaaagcagatggagcggcagcgcctcgctcgagagaagcagatgagggaggaggctgaacgcacgagggatgagttggagaggaggctgctgcagatgaaagaagaagcaacaatggccaacgaagcactgatgcggtctgaggagacagctgacctgttggctgaaaaggcccagatcaccgaggaggaggcaaaacttctggcccagaaggccgcagaggctgagcaggaaatgcagcgcatcaaggccacagcgattcgcacggaggaggagaagcgcctgatggagcagaaggtgctggaagccgaggtgctggcactgaagatggctgaggagtcagagaggagggccaaagaggcagatcagctgaagcaggacctgcaggaagcacgcgaggcggagcgaagagccaagcagaagctcctggagattgccaccaagcccacgtacccgcccatgaacccaattccagcaccgttgcctcctgacataccaagcttcaacctcattggtgacagcctgtctttcgacttcaaagatactgacatgaagcggctttccatggagatagagaaagaaaaagtggaatacatggaaaagagcaagcatctgcaggagcagctcaatgaactcaagacagaaatcgaggccttgaaactgaaagagagggagacagctctggatattctgcacaatgagaactccgacaggggtggcagcagcaagcacaataccattaaaaagctcaccttgcagagcgccaagtcccgagtggccttctttgaagagctctagcaggtgacccagccaccccaggacctgccacttctcctgctaccgggaccgcgggatggaccagatatcaagagagccatccatagggagctggctgggggtttccgtgggagctccagaactttccccagctgagtgaagagcccagcccctcttatgtgcaattgccttgaactacgaccctgtagagatttctctcatggcgttctagttctctgacctgagtctttgttttaagaagtatttgtcttcctttgtctaatgtgggattcctgactcccttcgtccaaggcaccggtgtgtgtgtgtcttgcactccagagctgacctccaccgcccagcctgggaagtcattgtagggagtgagacactgaagccctgagaagccagtgccatcatccccaccccgcccagggttccggaacattcattcccccaccggtgaggacctggcatgcagcgaagcagcccagcccggcggatcccaggccagcacgcctgccggcttctcatcgtcagggagcccgcccagagctcgtgacgagcaagtgctgggtccccgccaggcaccccgaggcggcgctctggctggcagctggtggggaataggcagggcagctgtggctggggagagactttaggcagaagctgtgatgcaggctgactgccagccgaggggctgggtagtgccgtgcgggagctgatggtacagggcactcgctgtccccctccggccaccctagaccagggtccgagaggcaggcaggagccactcatgtcttccccattgcccgacgcccatagacgctccttcctgtgtggggctggggtactccctggtcgtactgcagtcagcacccgtaacccggctatgaccagggatctgtaagccctgtggctccacaggtgctgcttctcactggcccagactctgagctccaccggcccagtctgcacggcccatctgcttcaccttccctcccagccacgtgccagtggccacagcccacttcccaacccactgttgtacccaggcctcactttgctgttgcccttgtccctcttcgggccctgaattttctgttccctgggggccagccagggccctttgtgcccctcccagcacaggcctgatgcaggtgtccactcacaggtggcgctcacctaggctgtcacaggacccacctccatgccaggcaacagagggccacagaaccacccccacggctcactccttggtctggggccaccttcttgccctttctttttttttttttttttttttttccgagatggagtctctctctgtcacccaggtgggagtgtggtggcacaatctcggctcactgcaacctccacctcctgagttcaagcaattctcctgcctcagcctcccaagtagctgggactacaggcatgcaccaccacacttggccaatgttctgtatttttaatagggacggggttttgccatgttagctaggctggtctcaaactcacacctgggattacaggcatgagccactgcacccagccccttcttgccctttcttttctccatggctgatgctgctgtggccagccagggcccttgagatccttccagtttggctgttatgcaaagcaggtgatttgtcttaatcagataaaagatagaggctatgggggcctcaagatttttggagagcagaggtggtctctggcaattccatctggttttgagaaacttagcagctcacagagcacagagatcctgccttcttcctactatcaggctgacctaatggggttgggctgctcggcaactgcttgggtcaccttgccccaaggaaaccagccctgggtgccacccagccacttagggtctacagggtgggactccagacctagagcgtaagtatggatgttgtggccctgtgtcttcctagtgtgacccagccaggagcggaagcttcaggcgtttgtaaagtgaggtctggctctgcctcctccgttttttttttttttctgtttctgtttctgttttttttttgagatggagtctcgctctgtcgcccaggctggagtgcagtgtcacgatctcagctcactgcaacctccgcctcccaggtacaagagattctcctgcctcagcctcccgagtagctgggactacaggcgtgtgccaccatgcctggctaatttttgtatttttagtagagatggggtttcgccatgttagccagactggtctcgaactcctgacctcaggtgatcctcccaccccggcttcccaaagttctgggattataggcgtgagccaccatgcccggtctcttctcagtcttgaagcccatccctggatttccaccaggagttactttcctcctgacctgtaaatttgttctttaacaatggctgcaggtgggagcatatggtggtttataaaaacgctgtcgggctttgtttccttctttagctgccgtgtctacttctgaagtctgggaagtgccaagccacgcggcctcaagggagctggctggtgtttgagctgtggcagaagcacctggggctccagggagcaggctgggaactgcaggaccttgctcagccaggagcacttccccctccttgaggcaggaatactgaggtgcctccccacagatggagaaggtggagaggaggatgggcctcaggagcatctcaagccccagtagcaggagaaagaaagaaagagatgcctggttttcacagactggttcctgtggctgggatgactgcatccttttttttttttttttgagacggagtttttgcctttgtcgcccaggctggagtgcaatggcgtgatctcggctcaccgcaacctccgcctcccggattcaagcaattctcctgcctcagcctcccgagtagctgggattacaggcacgcacctccacgtccggctaattttgtatttttagtggagacggggtttctccatgtcggtcaggctggtctcgaactcccgacctcaggtgatctgcccacctcggcctcccaaagtgctgggattacaggcatgagccaccgcgcttggccagactgcgtcctttttaagcgaacattttagggcctgggagtttgtcaagtaaggaagtctcaagcccaaagagcagcgtcctgaccatggtggtttcattacgagcccttctgctggctctcaggcagaagccccacagcaccgggaccattcatgaggtcactgcccagctcatgatgtccgtgaggctgtccttttggccagtagccgtgtgcagctgtgtggcacagatggcttcgttcatcctgatcaaggccccacctcagccacagcagtccccccaacctgtgttgtccaccctattattcatgtacctgccaggccctgctagatagcaccccgtggcattacataacacttcatgagtggctgtgtcttgtaattttggggacaggtttctctctttccctctcttttttttgtcaaaagcccagagactgacaaccagctgcagtgtctaagtgttcctcactgacagggtggggcctcaccacccctggagggagcagcgttggcagggagacagcctggcccagtgaccctgggcccaagccagcccctccagggctttcagggaagcgccatccattttcaaagatgtcaaacgtcacttcttcctgtagggcccgagtcctgcctcctatcagggccagatcatagaaggctattttctattctggggaacgattataacttaaatgattgttttaataaaaattctaagctggaaaataaaaaaaaaaaaaaaaaa

SEQ ID NO: 41 - DNA, 2348 bp, Homo sapiens
cagcaggccctgagctgacgtgtggagccagagccacccaatcccgtagggacaggtttcacaacttcccggatggggctgtggtgggtcacagtgcagcctccagccagaaggatggggtggctcccactcctgctgcttctgactcaatgcttaggggtccctgggcagcgctcgccattgaatgacttccaagtgctccggggcacagagctacagcacctgctacatgcggtggtgcccgggccttggcaggaggatgtggcagatgctgaagagtgtgctggtcgctgtgggcccttaatggactgccgggccttccactacaacgtgagcagccatggttgccaactgctgccatggactcaacactcgccccacacgaggctgcggcgttctgggcgctgtgacctcttccagaagaaagactacgtacggacctgcatcatgaacaatggggttgggtaccggggcaccatggccacgaccgtgggtggcctgccctgccaggcttggagccacaagttcccaaatgatcacaagtacacgcccactctccggaatggcctggaagagaacttctgccgtaaccctgatggcgaccccggaggtccttggtgctacacaacagaccctgctgtgcgcttccagagctgcggcatcaaatcctgccgggaggccgcgtgtgtctggtgcaatggcgaggaataccgcggcgcggtagaccgcacggagtcagggcgcgagtgccagcgctgggatcttcagcacccgcaccagcaccccttcgagccgggcaagttcctcgaccaaggtctggacgacaactattgccggaatcctgacggctccgagcggccatggtgctacactacggatccgcagatcgagcgagagttctgtgacctcccccgctgcgggtccgaggcacagccccgccaagaggccacaactgtcagctgcttccgcgggaagggtgagggctaccggggcacagccaataccaccactgcgggcgtaccttgccagcgttgggacgcgcaaatcccgcatcagcaccgatttacgccagaaaaatacgcgtgcaaagaccttcgggagaacttctgccggaaccccgacggctcagaggcgccctggtgcttcacactgcggcccggcatgcgcgcggccttttgctaccagatccggcgttgtacagacgacgtgcggccccaggactgctaccacggcgcaggggagcagtaccgcggcacggtcagcaagacccgcaagggtgtccagtgccagcgctggtccgctgagacgccgcacaagccgcagttcacgtttacctccgaaccgcatgcacaactggaggagaacttctgccggaacccagatggggatagccatgggccctggtgctacacgatggacccaaggaccccattcgactactgtgccctgcgacgctgcgctgatgaccagccgccatcaatcctggaccccccagaccaggtgcagtttgagaagtgtggcaagagggtggatcggctggatcagcggcgttccaagctgcgcgtggttgggggccatccgggcaactcaccctggacagtcagcttgcggaatcggcagggccagcatttctgcggggggtctctagtgaaggagcagtggatactgactgcccggcagtgcttctcctcctgccatatgcctctcacgggctatgaggtatggttgggcaccctgttccagaacccacagcatggagagccaagcctacagcgggtcccagtagccaagatggtgtgtgggccctcaggctcccagcttgtcctgctcaagctggagagatctgtgaccctgaaccagcgtgtggccctgatctgcctgccccctgaatggtatgtggtgcctccagggaccaagtgtgagattgcaggctggggtgagaccaaaggtacgggtaatgacacagtcctaaatgtggccttgctgaatgtcatctccaaccaggagtgtaacatcaagcaccgaggacgtgtgcgggagagtgagatgtgcactgagggactgttggcccctgtgggggcctgtgagggtgactacgggggcccacttgcctgctttacccacaactgctgggtcctggaaggaattataatccccaaccgagtatgcgcaaggtcccgctggccagctgtcttcacgcgtgtctctgtgtttgtggactggattcacaaggtcatgagactgggttaggcccagccttgatgccatatgccttggggaggacaaaacttcttgtcagacataaagccatgtttcctctttatgcctgtaaaaaaaaaaaaaaaaaa

SEQ ID NO: 42 - DNA, 2833 bp, Homo sapiens
agatccgccgcggagttacgggaaagttggtccgagttcccagagtttccctctgtggtgccctaggctcggccggccggtgccccggctcctttcctcctttcggccttcgccgtccaccaggtccctctctctgtccccggccgccatggagcagccgccggcgcctaagagtaaactaaaaaagctgagtgaagacagtttgactaagcagcctgaagaagtttttgatgtattagagaagcttggagaagggtcttatggaagtgtatttaaagcaatacacaaggaatccggtcaagttgtcgcaattaaacaagtacctgttgaatcagatcttcaggaaataatcaaagaaatttccataatgcagcaatgtgacagcccatatgttgtaaagtactatggcagttattttaagaatacagacctctggattgttatggagtactgtggcgctggctctgtctcagacataattagattacgaaacaagacattaatagaagatgaaattgcaaccattcttaaatctacattgaaaggactagaatatttgcactttatgagaaaaatacacagagatataaaagctggaaatattctcctcaatacagaaggacatgcaaaattggcagattttggagtggctggtcagttaacagatacaatggcaaaacgcaatactgtaataggaactccattttggatggctcctgaggtgattcaagaaataggctataactgtgtggccgacatctggtcccttggcattacttctatagaaatggctgaaggaaaacctccttatgctgatatacatccaatgagggctatttttatgattcccacaaatccaccaccaacattcagaaagccagaactttggtccgatgatttcaccgattttgttaaaaagtgtttggtgaagaatcctgagcagagagctactgcaacacaacttttacagcatccttttatcaagaatgccaaacctgtatcaatattaagagacctgatcacagaagctatggagatcaaagctaaaagacatgaggaacagcaacgagaattggaagaggaagaagaaaattcggatgaagatgagctggattcccacaccatggtgaagactagtgtggagagtgtgggcaccatgcgggccacaagcacgatgagtgaaggggcccagaccatgattgaacataatagcacgatgttggaatccgacttggggaccatggtgataaacagtgaggatgaggaagaagaagatggaactatgaaaagaaatgcaacctcaccacaagtacaaagaccatctttcatggactactttgataagcaagacttcaagaataagagtcacgaaaactgtaatcagaacatgcatgaacccttccctatgtccaaaaacgtttttcctgataactggaaagttcctcaagatggagactttgactttttgaaaaatctaagtttagaagaactacagatgcggttaaaagcactggaccccatgatggaacgggagatagaagaacttcgtcagagatacactgcgaaaagacagcccattctggatgcgatggatgcaaagaaaagaaggcagcaaaacttttgagtctaatttcctctctgtttttaactattctggagaccaagaaaccactaggaattgaaggaatatttggatatttttaatcctaagattttgccctacaattaggcagaggtcaaaaagtgacaatggtacatgcccaggtaaattcccaaaaggcagaattgacagttgtatctgctgtgcattcactctaagatgaggagaacaaaagaagtgtattctcttgttctgtcagctgcataccagtaataaaactgttatgaaatggattttcaaggtctctaaaccttgaaaatccaaagctattgttgcattgtacagcactgaagggctttatgttacaatattctttattcctatctagtatactaggctatttattgtatccccttaggtaaacttatttatttatgctattttgctttgtttcattttttaaggacaagatcaggatagctttggtgaaggtagggtcatattaatatgatgataatgtgcaaccaatttatactttctgcagggagctatggggtacattccttgatttccaggatagtttttcaaataggaaagcaataatggcagtagttctcaaatgggctaggccttttttatattgaagcaataattccatttttaccctttgaaattttgtttttttgatttttgatgtttggtacaaatagaactatatatatttaggtaaaatagatctatcgtgtttaaaaccaaagaaatcaatggaacccttgcacaaaaaagtgtgataaatatttttaaataaaaacttaatacaaatgtaatttgttaatattgtttcatgttttatgtgtagatctaatagctgaactgattcaaactgtaataagctcatcaatttcatttctatgaaaatgtgctctgttgtcacaggatgtttctgttgattttattcatttcctgggaattggtaaacatcatgttcctgatgataacccagtagcaaaaacatttgtactgagtggtacaagccttggggactgaaaaaaaaaagattaaaaccattaaaaagaaactcatttttacgctgaatgaacatttatatgattgcattgggaccagtcatttcctaagctacatatggccatcttgacagtgttttttcttttgtgtgtttaattattatgtgtaaatcataaagacaaataaatttcactgtgccacccagca

SEQ ID NO: 43 - DNA, 7362 bp, Homo sapiens
agcggagtgcggcggcggcgacactgagtggaaggcaaaatggcggcggcggcggcggtggcctggtgttaaggggagagccaggtcctcacgacccctgggacgggccgcgctggcccgcggcagcccccccgttcgtctccccgctctgccccaccagggatacttggggttgctgggacggactctggccgcctcagcgtccgccctcaggcccgtggccgctgtccaggagctctgctctcccctccagagttaattatttatattgtaaagaattttaacagtcctggggacttccttgaaggatcattttcacttttgctcagaagaaagctctggatctatcaaataaagaagtccttcgtgtgggctacatatatagatgttttcatgaagaggagtgaaaagccagaaggatatagacaaatgaggcctaagacctttcctgccagtaactatactgtcagtagccggcaaatgttacaagaaattcgggaatcccttaggaatttatctaaaccatctgatgctgctaaggctgagcataacatgagtaaaatgtcaaccgaagatcctcgacaagtcagaaatccacccaaatttgggacgcatcataaagccttgcaggaaattcgaaactctctgcttccatttgcaaatgaaacaaattcttctcggagtacttcagaagttaatccacaaatgcttcaagacttgcaagctgctggatttgatgaggatatggttatacaagctcttcagaaaactaacaacagaagtatagaagcagcaattgaattcattagtaaaatgagttaccaagatcctcgacgagagcagatggctgcagcagctgccagacctattaatgccagcatgaaaccagggaatgtgcagcaatcagttaaccgcaaacagagctggaaaggttctaaagaatccttagttcctcagaggcatggcccgccactaggagaaagtgtggcctatcattctgagagtcccaactcacagacagatgtaggaagacctttgtctggatctggtatatcagcatttgttcaagctcaccctagcaacggacagagagtgaaccccccaccaccacctcaagtaaggagtgttactcctccaccacctccaagaggccagactccccctccaagaggtacaactccacctcccccttcatgggaaccaaactctcaaacaaagcgctattctggaaacatggaatacgtaatctcccgaatctctcctgtcccacctggggcatggcaagagggctatcctccaccacctctcaacacttcccccatgaatcctcctaatcaaggacagagaggcattagttctgttcctgttggcagacaaccaatcatcatgcagagttctagcaaatttaactttccatcagggagacctggaatgcagaatggtactggacaaactgatttcatgatacaccaaaatgttgtccctgctggcactgtgaatcggcagccaccacctccatatcctctgacagcagctaatggacaaagcccttctgctttacaaacagggggatctgctgctccttcgtcatatacaaatggaagtattcctcagtctatgatggtgccaaacagaaatagtcataacatggaactatataacattagtgtacctggactgcaaacaaattggcctcagtcatcttctgctccagcccagtcatccccgagcagtgggcatgaaatccctacatggcaacctaacataccagtgaggtcaaattcttttaataacccattaggaaatagagcaagtcactctgctaattctcagccttctgctacaacagtcactgcaattacaccagctcctattcaacagcctgtgaaaagtatgcgtgtattaaaaccagagctacagactgctttagcacctacacacccttcttggataccacagccaattcaaactgttcaacccagtccttttcctgagggaaccgcttcaaatgtgactgtgatgccacctgttgctgaagctccaaactatcaaggaccaccaccaccctacccaaaacatctgctgcaccaaaacccatctgttcctccatacgagtcaatcagtaagcctagcaaagaggatcagccaagcttgcccaaggaagatgagagtgaaaagagttatgaaaatgttgatagtggggataaagaaaagaaacagattacaacttcacctattactgttaggaaaaacaagaaagatgaagagcgaagggaatctcgtattcaaagttattctcctcaagcatttaaattctttatggagcaacatgtagaaaatgtactcaaatctcatcagcagcgtctacatcgtaaaaaacaattagagaatgaaatgatgcgggttggattatctcaagatgcccaggatcaaatgagaaagatgctttgccaaaaagaatctaattacatccgtcttaaaagggctaaaatggacaagtctatgtttgtgaagataaagacactaggaataggagcatttggtgaagtctgtctagcaagaaaagtagatactaaggctttgtatgcaacaaaaactcttcgaaagaaagatgttcttcttcgaaatcaagtcgctcatgttaaggctgagagagatatcctggctgaagctgacaatgaatgggtagttcgtctatattattcattccaagataaggacaatttatactttgtaatggactacattcctgggggtgatatgatgagcctattaattagaatgggcatctttccagaaagtctggcacgattctacatagcagaacttacctgtgcagttgaaagtgttcataaaatgggttttattcatagagatattaaacctgataatattttgattgatcgtgatggtcatattaaattgactgactttggcctctgcactggcttcagatggacacacgattctaagtactatcagagtggtgaccatccacggcaagatagcatggatttcagtaatgaatggggggatccctcaagctgtcgatgtggagacagactgaagccattagagcggagagctgcacgccagcaccagcgatgtctagcacattctttggttgggactcccaattatattgcacctgaagtgttgctacgaacaggatacacacagttgtgtgattggtggagtgttggtgttattctttttgaaatgttggtgggacaacctcctttcttggcacaaacaccattagaaacacaaatgaaggttatcaactggcaaacatctcttcacattccaccacaagctaaactcagtcctgaagcttctgatcttattattaaactttgccgaggacccgaagatcgcttaggcaagaatggtgctgatgaaataaaagctcatccattttttaaaacaattgacttctccagtgacctgagacagcagtctgcttcatacattcctaaaatcacacacccaacagatacatcaaattttgatcctgttgatcctgataaattatggagtgatgataacgaggaagaaaatgtaaatgacactctcaatggatggtataaaaatggaaagcatcctgaacatgcattctatgaatttaccttccgaaggttttttgatgacaatggctacccatataattatccgaagcctattgaatatgaatacattaattcacaaggctcagagcagcagtcggatgaagatgatcaaaacacaggctcagagattaaaaatcgcgatctagtatatgtttaacacactagtaaataaatgtaatgaggatttgtaaaagggcctgaaatgcgaggtgttttgaggttctgagagtaaaattatgcaaatatgacagagctatatatgtgtgctctgtgtacaatattttattttcctaaattatgggaaatccttttaaaatgttaatttattccagccgtttaaatcagtatttagaaaaaaattgttataaggaaagtaaattatgaactgaatattatagtcagttcttggtacttaaagtacttaaaataagtagtgctttgtttaaaaggagaaacctggtatctatttgtatatatgctaaataattttaaaatacaagagtttttgaaatttttttgaaagacagttttagttttatcttgctttaaccaaatatgaaacataccccctattttacagagctcttttttcccctcataaccttgtttttggtagaaaataagctagagaaattaagccatcgtgttggtgagtgttcctaggctaatgataatctgtataattcacatcctgaaactaaggaatacagggttgaaaaaatattaatatgtttgtcagaaggaaaaataatgcatttatcttcccccccaccccccgccccatggaatatttaatctatttaatcttcttgcatttatttctcaagaattactggctttaaaagaagccaaagcactactagctttttttccatattggtatttttgatgctgcttccaattttaaaagggaacaaagctgccataaatcgaaatgttcaatactaaaagctaaaatatttctcaccatcctaagcagataattattttaattttcatatacttttcctgtatagtaactattttgattatatcatcaatgttacctgtttcctctttcagaacagtgctgcatatacagattgttattggcaaaggaaaatctggctatctggcaatattttacctaagcgcagattaattggtgaaaaaattaactcttaagatggccattaataattaggaaagtttacagagtggtcttagtagaaaattcaagtcctcctaatttatttaaggttcaataatgcgttcaacatgcctgttatgtataacgcttaggttctaaggaagattaaggtttcataccaaaatacatgtagcttatcttttaggaaggggaaaaaggctccattttgaccatagtaaaatttgtgttgtgttttatttccttttcttaagctccactgataagggattgtttttatcaaaagttactatttgtagattggaggcataattttagtgattttcatacttttagctttcttcgcataaaagctaattgaaaccgtatatgtagtaaaattaaaggcagagctgttgcagttgaattggagagttagggcaaagaacacttattagcccacacttcccacctttctacaggtggtcctttcagagctcagcctgaaaacccactactgtgttatcgtgcgtcttttggggttagtggttcttttgagaatctgaaggaagctgtggactcttcctagaaaaaaaaaccacacatacacatacaatgttgcatgcagtttcaagggattttggacatattgaaacctatcacaggctgtaggttatggacctctgtgccatgagaaaattgatacattaaactaagaactttgtttttaacttaccaatcactactcagcacatcttatataagctgataatttgtgatggaaaaggtctgtagcatgtgatataaggtgaccttatgaatgcctctcttgctggtacattaagttgttttaatatatcatttggaggggactgaaatgttaggctcattacaagcttgatacagaaatatttctgaaggatttctaatcagaattgtaaaacaatgtgctatcatgaaatcgcagtcttcacctcatggttcatggaacatttggttagtcccataaaatcctatgcaaaacaaagtagttcaagaatttttaggtgggtagtcacatttataaggtattcctcttactctttgggctttttcagtctgatttatttaaattttcatttagttgttttacttttggactaaggtgcaatacagtagaagataactttgttacatttatgttgtaggaaaactaaggtgctgtctcctcccccttcccttcccacaaaatctgtattccccctattgctgaaatgtaacagacactacaaattttgtattctttttttgttttttgttttgagacagggtctcactctgtcacccaggctggagggcagtggcgcttcacagctcactgcatcctcaaccttgggggctcacgcagtcctcccgcctcagcctcccaagtagctgggcatgcgccaccaagcccagctaatttttgtatctttagtagagatggggtttcgccatgttgcccaggttggtgtggaattcctgggctccagttatatgcccacctcagcctcccaaagtgctgggattacagacgtgacccaccgcgcctggcgcaaatatgtattcttttaaaatttcctctgatactataagctttttgcatttatctgaagcagtatacatgcctttggtatcagcaattttaacagtttggatatacttatcagctatcttattccaaaactacatctacttcttccagtatagaatctggtgcttcctgaccaaaaagatgagaaaaacaatgttaaaaatatagatgctttccattgaaatggagtgaaaacattggttctatatgttttcttttaaaataattttcttattaaaaacttgctgtctttattatacttaccctttttatgcatatcaatagtatttataagatgtgttctataattatgtaattgtagatactgttatgcattgtccagtgacatcataaggcaggccctactgctgtatcttttctaccttcttatttgtaatagaaactatagaatgtatgactaaaaagtcactttgagattgacttttttaaaaagttattaccttctgctgttgcaaagtgcaaaactgtgagtggaattgttttattctgacttaatgtgttagaaattagagaatacagtgggaggatttttagacattgctgctgctgttacccaaggtattttagataaaaaatttttaataaacatccctttggtatttaaagtggaacatttagcctgttcattttaatctaaagcaaaaagtaatttgggtcaaaatattggtatatttgtaaagcgccttaatatatccctttgtggaaggcactacacagtttacttttatattgtattgtgtatataagtattttgtattaaaattgaatcagtggcaacattaaagttttataaaatcatgctttgttagaaaaagaattacagctttgcaatataactaattgtttcgcataattctgaatgtaatagatatgaataatcagcctgtgtttttaatgaacttatttgtattttcccaatcattttctctagtgtaatgtttgctgggataataaaaaaaattcaaatctttcaa

SEQ ID NO: 44 - DNA, 5546 bp, Homo sapiens
gacgcccgtggaatgccaacaatgtagcgaatgtcccacttgggtctgcgctttggaaccgcggcgtgagcgccccgggaagatggagcagtcgccgtccacgccaccgccgccgcccggggctcccccgtccctgcggggccagcagcagctccagccaccagtgcccggtctcccggcgcgagaggcccgggagccgccggccaggacgcccccgagggtgtagaccgcgcccctggagagagtgataatcttcaaaatgaagactttggaaaattttaggttctctataggaactacaaaaatggaaggaaagaacattttcaaaaggaaattattttgaaagtatgtttacaacaaactgatactattgacagtttttttttttaaataataaaacactttaagaagattgtatttatggtaaaaggaaactggactaacaatgaggccaaagacttttcctgccacgacttattctggaaatagccggcagcgactgcaagagattcgtgaggggttaaaacagccatccaagtcttcggttcaggggctacccgcaggaccaaacagtgacacttccctggatgccaaagtcctggggagcaaagatgccaccaggcagcagcagcagatgagagccaccccaaagttcggaccttatcagaaagccttgagggaaatcagatattccttgttgccttttgctaatgaatcgggcacctctgcagctgcagaagtgaaccggcaaatgctgcaggaactggtgaacgcaggatgcgaccaggagatggctggccgagctctcaagcagactggcagcaggagcatcgaggccgccctggagtacatcagcaagatgggctacctggacccgaggaatgagcagattgtgcgggtcattaagcagacctccccaggaaaggggctcatgccaaccccagtgacgcggaggcccagcttcgaaggaaccggcgattcgtttgcgtcctaccaccagctgagcggtaccccctacgagggcccaagcttcggcgctgacggccccacggcgctggaggagatgccgcggccgtacgtggactaccttttccccggagtcggcccccacgggcccggccaccagcaccagcacccacccaagggctacggtgccagcgtagaggcagcaggggcacacttcccgctgcagggcgcgcactacgggcggccgcacctgctggtgcctggggaacccctgggctacggagtgcagcgcagcccctccttccagagcaagacgccgccggagaccgggggttacgccagcctgcccacgaagggccagggaggaccgccaggcgccggcctcgctttcccaccccctgccgccgggctctacgtgccgcacccacaccacaagcaggccggtcccgcggcccaccagctgcatgtgctgggctcccgcagccaggtgttcgccagcgacagccccccgcagagcctgctcactccctcgcggaacagcctcaacgtggacctgtatgaattgggcagcacctccgtccagcagtggccggctgccaccctggcccgccgggactccctgcagaagccgggcctggaggcgccgccgcgcgcgcacgtggccttccggcctgactgcccagtgcccagcaggaccaactccttcaacagccaccagccgcggcccggtccgcctggcaaggccgagccctccctgcccgcccccaacaccgtgacggctgtcacggccgcgcacatcttgcacccggtgaagagcgtgcgtgtgctgaggccggagccgcagacggctgtggggccctcgcaccccgcctgggtgcccgcgcctgccccggcccccgcccccgcccccgccccggctgcggagggcttggacgccaaggaggagcatgccctggcgctgggcggcgcaggcgccttcccgctggacgtggagtacggaggcccagaccggaggtgcccgcctccgccctacccgaagcacctgctgctgcgcagcaagtcggagcagtacgacctggacagcctgtgcgcaggcatggagcagagcctccgtgcgggccccaacgagcccgagggcggcgacaagagccgcaaaagcgccaagggggacaaaggcggaaaggataaaaagcagattcagacctctcccgttcccgtccgcaaaaacagcagagacgaagagaagagagagtcacgcatcaagagctactcgccatacgcctttaagttcttcatggagcagcacgtggagaatgtcatcaaaacctaccagcagaaggttaaccggaggctgcagctggagcaagaaatggccaaagctggactctgtgaagctgagcaggagcagatgcggaagatcctctaccagaaagagtctaattacaacaggttaaagagggccaagatggacaagtctatgtttgtcaagatcaaaaccctggggatcggtgcctttggagaagtgtgccttgcttgtaaggtggacactcacgccctgtacgccatgaagaccctaaggaaaaaggatgtcctgaaccggaatcaggtggcccacgtcaaggccgagagggacatcctggccgaggcagacaatgagtgggtggtcaaactctactactccttccaagacaaagacagcctgtactttgtgatggactacatccctggtggggacatgatgagcctgctgatccggatggaggtcttccctgagcacctggcccggttctacatcgcagagctgactttggccattgagagtgtccacaagatgggcttcatccaccgagacatcaagcctgataacattttgatagatctggatggtcacattaaactcacagatttcggcctctgcactgggttcaggtggactcacaattccaaatattaccagaaagggagccatgtcagacaggacagcatggagcccagcgacctctgggatgatgtgtctaactgtcggtgtggggacaggctgaagaccctagagcagagggcgcggaagcagcaccagaggtgcctggcacattcactggtggggactccaaactacatcgcacccgaggtgctcctccgcaaagggtacactcaactctgtgactggtggagtgttggagtgattctcttcgagatgctggtggggcagccgccctttttggcacctactcccacagaaacccagctgaaggtgatcaactgggagaacacgctccacattccagcccaggtgaagctgagccctgaggccagggacctcatcaccaagctgtgctgctccgcagaccaccgcctggggcggaatggggccgatgacctgaaggcccaccccttcttcagcgccattgacttctccagtgacatccggaagcagccagccccctacgttcccaccatcagccaccccatggacacctcgaatttcgaccccgtagatgaagaaagcccttggaacgatgccagcgaaggtagcaccaaggcctgggacacactcacctcgcccaataacaagcatcctgagcacgcattttacgaattcaccttccgaaggttctttgatgacaatggctacccctttcgatgcccaaagccttcaggagcagaagcttcacaggctgagagctcagatttagaaagctctgatctggtggatcagactgaaggctgccagcctgtgtacgtgtagatgggggccaggcacccccaccactcgctgcctcccaggtcagggtcccggagccggtgccctcacaggccaatagggaagccgagggctgttttgttttaaattagtccgtcgattacttcacttgaaattctgctcttcaccaagaaaacccaaacaggacacttttgaaaacaggactcagcatcgctttcaataggcttttcaggaccttcactgcattaaaacaatatttttgaaaatttagtacagtttagaaagagcacttattttgtttatatccattttttcttactaaattatagggattaactttgacaaatcatgctgctgttattttctacatttgtattttatccatagcacttattcacatttaggaaaagacataaaaactgaagaacattgatgagaaatctctgtgcaataatgtaaaaaaaaaaaaagataacactctgctcaatgtcacggagaccattttatccacacaatggtttttgttttttattttttcccatgtttcaaaattgtgatataatgatataatgttaaaagctgctttttttggctttttgcatatctagtataataggaagtgtgagcaaggtgatgatgtggctgtgatttccgacgtctggtgtgtggagagtactgcatgagcagagttcttctattataaaattaccatatcttgccattcacagcaggtcctgtgaatacgtttttactgagtgtctttaaatgaggtgttctagacagtgtgctgataatgtattgtgcgggtgacctcttcgctatgattgtatctcttactgttttgttaaagaaatgcagatgtgtaactgagaagtgatttgtgtgtgtgtcttggttgtgattggattctttggggggggggaactgaaacatttgtcatatactgaacttatatacatcaaaagggattaatacagcgatgccaaaaagtttaatcacggacacatgtccgtttctgtagtccgtatgctctttcattcttggtagagctggtatgtggaatgccatacctctgaccctactacttacctttttactgacagactgcccacactgaaagcttcagtgaatgttcttagtcctgttttcttctgttactgtcaggaaactgagtgatctaatggttctctcactttttttttgttcttttagtgtactttgaagtatcaaatcttaacttggtttaaacaatacatattcctaacctttgtaaaaaagcaaagattcttcaaaatgacattgaaataaaaagtaagccatacgtattttcttagaagtatagatgtatgtgcgtgtatacacacacacacacacacacagagataaacacaatattccttatttcaaattagtatgattcctatttaaagtgatttatatttgagtaaaaagttcaattcttttttgctttttaaaaaatctgatgcttcataattttcattatattattccacatatttttccttgaagttcttagcataatgtatccattacttagtatatatctaggcaacaacacttagaagtttatcagtgtttaaactaaaaaaataaagattcctgtgtactggtttacatttgtgtgagtggcatactcaagtctgctgtgcctgtcgtcgtgactgtcagtattctcgctattttatagtcgtgccatgttgttactcacagcgctctgacatactttcatgtggtaggttctttctcaggaactcagtttaactattatttattgatatatcattacctttgaaaagcttctactggcacaatttattattaaaattttgaatccaaa

SEQ ID NO: 45 - DNA, 5408 bp, Homo sapiens
gccgccgccagggaaaagaaagggaggaaggaaggaacaagaaaaggaaataaagagaaaggggaggcggggaaaggcaacgagctgtccggcctccgtcaagggagttggagggaaaaagttctcaggcgccgcaggtccgagtgcctcgcagcccctcccgaggcgcagccgccagaccagtggagccggggcgcagggcgggggcggaggcgccggggcgggggatgcggggccgcggcgcagccccccggccctgagagcgaggacagcgccgcccggcccgcagccgtcgccgcttctccacctcggcccgtggagccggggcgtccgggcgtagccctcgctcgcctgggtcagggggtgcgcgtcgggggaggcagaagccatggatcccgggcagcagccgccgcctcaaccggccccccagggccaagggcagccgccttcgcagcccccgcaggggcagggcccgccgtccggacccgggcaaccggcacccgcggcgacccaggcggcgccgcaggcaccccccgccgggcatcagatcgtgcacgtccgcggggactcggagaccgacctggaggcgctcttcaacgccgtcatgaaccccaagacggccaacgtgccccagaccgtgcccatgaggctccggaagctgcccgactccttcttcaagccgccggagcccaaatcccactcccgacaggccagtactgatgcaggcactgcaggagccctgactccacagcatgttcgagctcattcctctccagcttctctgcagttgggagctgtttctcctgggacactgacccccactggagtagtctctggcccagcagctacacccacagctcagcatcttcgacagtcttcttttgagatacctgatgatgtacctctgccagcaggttgggagatggcaaagacatcttctggtcagagatacttcttaaatcacatcgatcagacaacaacatggcaggaccccaggaaggccatgctgtcccagatgaacgtcacagcccccaccagtccaccagtgcagcagaatatgatgaactcggcttcaggtcctcttcctgatggatgggaacaagccatgactcaggatggagaaatttactatataaaccataagaacaagaccacctcttggctagacccaaggcttgaccctcgttttgccatgaaccagagaatcagtcagagtgctccagtgaaacagccaccacccctggctccccagagcccacagggaggcgtcatgggtggcagcaactccaaccagcagcaacagatgcgactgcagcaactgcagatggagaaggagaggctgcggctgaaacagcaagaactgcttcggcaggtgaggccacaggcaatgcggaatatcaatcccagcacagcaaattctccaaaatgtcaggagttagccctgcgtagccagttaccaacactggagcaggatggtgggactcaaaatccagtgtcttctcccgggatgtctcaggaattgagaacaatgacgaccaatagctcagatcctttccttaacagtggcacctatcactctcgagatgagagtacagacagtggactaagcatgagcagctacagtgtccctcgaaccccagatgacttcctgaacagtgtggatgagatggatacaggtgatactatcaaccaaagcaccctgccctcacagcagaaccgtttcccagactaccttgaagccattcctgggacaaatgtggaccttggaacactggaaggagatggaatgaacatagaaggagaggagctgatgccaagtctgcaggaagctttgagttctgacatccttaatgacatggagtctgttttggctgccaccaagctagataaagaaagctttcttacatggttatagagccctcaggcagactgaattctaaatctgtgaaggatctaaggagacacatgcaccggaaatttccataagccagttgcagttttcaggctaatacagaaaaagatgaacaaacgtccagcaagatactttaatcctctattttgctcttccttgtccattgctgctgttaatgtattgctgacctctttcacagttggctctaaagaatcaaaagaaaaaaactttttatttcttttgctattaaaactactgttcattttgggggctgggggaagtgagcctgtttggatgatggatgccattccttttgcccagttaaatgttcaccaatcattttaactaaatactcagacttagaagtcagatgcttcatgtcacagcatttagtttgttcaacagttgtttcttcagcttcctttgtccagtggaaaaacatgatttactggtctgacaagccaaaaatgttatatctgatattaaatacttaatgctgatttgaagagatagctgaaaccaaggctgaagactgttttactttcagtattttcttttcctcctagtgctatcattagtcacataatgaccttgattttattttaggagcttataaggcatgagacaatttccatataaatatattaattattgccacatactctaatatagattttggtggataattttgtgggtgtgcattttgttctgttttgttgggttttttgttttttttgtttttggcagggtcggtgggggggttggttggttggttggttttgtcggaacctaggcaaatgaccatattagtgaatctgttaatagttgtagcttgggatggttattgtagttgttttggtaaaatcttcatttcctggttttttttaccaccttatttaaatctcgattatctgctctctcttttatatacatacacacacccaaacataacatttataatagtgtggtagtggaatgtatccttttttaggtttccctgctttccagttaatttttaaaatggtagcgctttgtatgcatttagaatacatgactagtagtttatatttcactggtagtttaaatctggttggggcagtctgcagatgtttgaagtagtttagtgttctagaaagagctattactgtggatagtgcctaggggagtgctccacgccctctgggcatacggtagatattatctgatgaattggaaaggagcaaaccagaaatggctttattttctcccttggactaatttttaagtctcgattggaattcagtgagtaggttcataatgtgcatgacagaaataagctttatagtggtttaccttcatttagctttggaagttttctttgccttagttttggaagtaaattctagtttgtagttctcatttgtaatgaacacattaacgactagattaaaatattgccttcaagattgttcttacttacaagacttgctcctacttctatgctgaaaattgaccctggatagaatactataaggttttgagttagctggaaaagtgatcagattaataaatgtatattggtagttgaatttagcaaagaaatagagataatcatgattatacctttatttttacaggaagagatgatgtaactagagtatgtgtctacaggagtaataatggtttccaaagagtattttttaaaggaacaaaacgagcatgaattaactcttcaatataagctatgaagtaatagttggttgtgaattaaagtggcaccagctagcacctctgtgttttaagggtctttcaatgtttctagaataagcccttattttcaagggttcataacaggcataaaatctcttctcctggcaaaagctgctatgaaaagcctcagcttgggaagatagatttttttccccccaattacaaaatctaagtattttggcccttcaatttggaggagggcaaaagttggaagtaagaagttttattttaagtactttcagtgctcaaaaaaatgcaatcactgtgttgtatataatagttcataggttgatcactcataataattgactctaaggcttttattaagaaaacagcagaaagattaaatcttgaattaagtctggggggaaatggccactgcagatggagttttagagtagtaatgaaattctacctagaatgcaaaattgggtatatgaattacatagcatgttgttgggattttttttaatgtgcagaagatcaaagctacttggaaggagtgcctataatttgccagtagccacagattaagattatatcttatatatcagcagattagctttagcttagggggagggtgggaaagtttggggggggggttgtgaagatttagggggaccttgatagagaactttataaacttctttctctttaataaagacttgtcttacaccgtgctgccattaaaggcagctgttctagagtttcagtcacctaagtacacccacaaaacaatatgaatatggagatcttcctttacccctcaactttaatttgcccagttatacctcagtgttgtagcagtactgtgatacctggcacagtgctttgatcttacgatgccctctgtactgacctgaaggagacctaagagtcctttccctttttgagtttgaatcatagccttgatgtggtctcttgttttatgtccttgttcctaatgtaaaagtgcttaactgcttcttggttgtattgggtagcattgggataagattttaactgggtattcttgaattgcttttacaataaaccaattttataatctttaaatttatcaactttttacatttgtgttattttcagtcagggcttcttagatctacttatggttgatggagcacattgatttggagtttcagatcttccaaagcactatttgttgtaataacttttctaaatgtagtgcctttaaaggaaaaatgaacacagggaagtgactttgctacaaataatgttgctgtgttaagtattcatattaaatacatgccttctatatggaacatggcagaaagactgaaaaataacagtaattaattgtgtaattcagaattcataccaatcagtgttgaaactcaaacattgcaaaagtgggtggcaatattcagtgcttaacacttttctagcgttggtacatctgagaaatgagtgctcaggtggattttatcctcgcaagcatgttgttataagaattgtgggtgtgcctatcataacaattgttttctgtatcttgaaaaagtattctccacattttaaatgttttatattagagaattctttaatgcacacttgtcaaatatatatatatagtaccaatgttacctttttattttttgttttagatgtaagagcatgctcatatgttaggtacttacataaattgttacattattttttcttatgtaatacctttttgtttgtttatgtggttcaaatatattctttccttaaactcttaaaaaaaaaa

SEQ ID NO: 46 - DNA, 5234 bp, Homo sapiens
agagcgtcgggatatcgggtggcggctcgggacggaggacgcgctagtgtgagtgcgggcttctagaactacaccgaccctcgtgtcctcccttcatcctgcggggctggctggagcggccgctccggtgctgtccagcagccatagggagccgcacggggagcgggaaagcggtcgcggccccaggcggggcggccgggatggagcggggccgcgagcctgtggggaaggggctgtggcggcgcctcgagcggctgcaggttcttctgtgtggcagttcagaatgatggatcaagctagatcagcattctctaacttgtttggtggagaaccattgtcatatacccggttcagcctggctcggcaagtagatggcgataacagtcatgtggagatgaaacttgctgtagatgaagaagaaaatgctgacaataacacaaaggccaatgtcacaaaaccaaaaaggtgtagtggaagtatctgctatgggactattgctgtgatcgtctttttcttgattggatttatgattggctacttgggctattgtaaaggggtagaaccaaaaactgagtgtgagagactggcaggaaccgagtctccagtgagggaggagccaggagaggacttccctgcagcacgtcgcttatattgggatgacctgaagagaaagttgtcggagaaactggacagcacagacttcaccggcaccatcaagctgctgaatgaaaattcatatgtccctcgtgaggctggatctcaaaaagatgaaaatcttgcgttgtatgttgaaaatcaatttcgtgaatttaaactcagcaaagtctggcgtgatcaacattttgttaagattcaggtcaaagacagcgctcaaaactcggtgatcatagttgataagaacggtagacttgtttacctggtggagaatcctgggggttatgtggcgtatagtaaggctgcaacagttactggtaaactggtccatgctaattttggtactaaaaaagattttgaggatttatacactcctgtgaatggatctatagtgattgtcagagcagggaaaatcacctttgcagaaaaggttgcaaatgctgaaagcttaaatgcaattggtgtgttgatatacatggaccagactaaatttcccattgttaacgcagaactttcattctttggacatgctcatctggggacaggtgacccttacacacctggattcccttccttcaatcacactcagtttccaccatctcggtcatcaggattgcctaatatacctgtccagacaatctccagagctgctgcagaaaagctgtttgggaatatggaaggagactgtccctctgactggaaaacagactctacatgtaggatggtaacctcagaaagcaagaatgtgaagctcactgtgagcaatgtgctgaaagagataaaaattcttaacatctttggagttattaaaggctttgtagaaccagatcactatgttgtagttggggcccagagagatgcatggggccctggagctgcaaaatccggtgtaggcacagctctcctattgaaacttgcccagatgttctcagatatggtcttaaaagatgggtttcagcccagcagaagcattatctttgccagttggagtgctggagactttggatcggttggtgccactgaatggctagagggatacctttcgtccctgcatttaaaggctttcacttatattaatctggataaagcggttcttggtaccagcaacttcaaggtttctgccagcccactgttgtatacgcttattgagaaaacaatgcaaaatgtgaagcatccggttactgggcaatttctatatcaggacagcaactgggccagcaaagttgagaaactcactttagacaatgctgctttccctttccttgcatattctggaatcccagcagtttctttctgtttttgcgaggacacagattatccttatttgggtaccaccatggacacctataaggaactgattgagaggattcctgagttgaacaaagtggcacgagcagctgcagaggtcgctggtcagttcgtgattaaactaacccatgatgttgaattgaacctggactatgagaggtacaacagccaactgctttcatttgtgagggatctgaaccaatacagagcagacataaaggaaatgggcctgagtttacagtggctgtattctgctcgtggagacttcttccgtgctacttccagactaacaacagatttcgggaatgctgagaaaacagacagatttgtcatgaagaaactcaatgatcgtgtcatgagagtggagtatcacttcctctctccctacgtatctccaaaagagtctcctttccgacatgtcttctggggctccggctctcacacgctgccagctttactggagaacttgaaactgcgtaaacaaaataacggtgcttttaatgaaacgctgttcagaaaccagttggctctagctacttggactattcagggagctgcaaatgccctctctggtgacgtttgggacattgacaatgagttttaaatgtgatacccatagcttccatgagaacagcagggtagtctggtttctagacttgtgctgatcgtgctaaattttcagtagggctacaaaacctgatgttaaaattccatcccatcatcttggtactactagatgtctttaggcagcagcttttaatacagggtagataacctgtacttcaagttaaagtgaataaccacttaaaaaatgtccatgatggaatattcccctatctctagaattttaagtgctttgtaatgggaactgcctctttcctgttgttgttaatgaaaatgtcagaaaccagttatgtgaatgatctctctgaatcctaagggctggtctctgctgaaggttgtaagtggtcgcttactttgagtgatcctccaacttcatttgatgctaaataggagataccaggttgaaagaccttctccaaatgagatctaagcctttccataaggaatgtagctggtttcctcattcctgaaagaaacagttaactttcagaagagatgggcttgttttcttgccaatgaggtctgaaatggaggtccttctgctggataaaatgaggttcaactgttgattgcaggaataaggccttaatatgttaacctcagtgtcatttatgaaaagaggggaccagaagccaaagacttagtatattttcttttcctctgtcccttcccccataagcctccatttagttctttgttatttttgtttcttccaaagcacattgaaagagaaccagtttcaggtgtttagttgcagactcagtttgtcagactttaaagaataatatgctgccaaattttggccaaagtgttaatcttaggggagagctttctgtccttttggcactgagatatttattgtttatttatcagtgacagagttcactataaatggtgtttttttaatagaatataattatcggaagcagtgccttccataattatgacagttatactgtcggttttttttaaataaaagcagcatctgctaataaaacccaacagatactggaagttttgcatttatggtcaacacttaagggttttagaaaacagccgtcagccaaatgtaattgaataaagttgaagctaagatttagagatgaattaaatttaattaggggttgctaagaagcgagcactgaccagataagaatgctggttttcctaaatgcagtgaattgtgaccaagttataaatcaatgtcacttaaaggctgtggtagtactcctgcaaaattttatagctcagtttatccaaggtgtaactctaattcccattttgcaaaatttccagtacctttgtcacaatcctaacacattatcgggagcagtgtcttccataatgtataaagaacaaggtagtttttacctaccacagtgtctgtatcggagacagtgatctccatatgttacactaagggtgtaagtaattatcgggaacagtgtttcccataattttcttcatgcaatgacatcttcaaagcttgaagatcgttagtatctaacatgtatcccaactcctataattccctatcttttagttttagttgcagaaacattttgtggtcattaagcattgggtgggtaaattcaaccactgtaaaatgaaattactacaaaatttgaaatttagcttgggtttttgttacctttatggtttctccaggtcctctacttaatgagatagtagcatacatttataatgtttgctattgacaagtcattttaactttatcacattatttgcatgttacctcctataaacttagtgcggacaagttttaatccagaattgaccttttgacttaaagcagagggactttgtatagaaggtttgggggctgtggggaaggagagtcccctgaaggtctgacacgtctgcctacccattcgtggtgatcaattaaatgtaggtatgaataagttcgaagctccgtgagtgaaccatcattataaacgtgatgatcagctgtttgtcatagggcagttggaaacggcctcctagggaaaagttcatagggtctcttcaggttcttagtgtcacttacctagatttacagcctcacttgaatgtgtcactactcacagtctctttaatcttcagttttatctttaatctcctcttttatcttggactgacatttagcgtagctaagtgaaaaggtcatagctgagattcctggttcgggtgttacgcacacgtacttaaatgaaagcatgtggcatgttcatcgtataacacaatatgaatacagggcatgcattttgcagcagtgagtctcttcagaaaacccttttctacagttagggttgagttacttcctatcaagccagtacgtgctaacaggctcaatattcctgaatgaaatatcagactagtgacaagctcctggtcttgagatgtcttctcgttaaggagatgggccttttggaggtaaaggataaaatgaatgagttctgtcatgattcactattctagaacttgcatgacctttactgtgttagctctttgaatgttcttgaaattttagactttctttgtaaacaaatgatatgtccttatcattgtataaaagctgttatgtgcaacagtgtggagattccttgtctgatttaataaaatacttaaacactgaaaaaaaaaaa

SEQ ID NO: 47 - DNA, 5039 bp, Homo sapiens
gcgattcggctggctctgccacaccaccgcgcgcccccgctccgcccgcccctccgggcgcgtcttttccgggctcgcgctgagtcccgcctccgccggctgtccgggtgcgcgcgcgccgctgcggctttttctctggcctccgccgcgcgctcctcctcgtcccagcgctagcgggcacgcggttcctttttgcgagctttccgagtgccaggcgccggccggctgcgaagacgcggtgggccgcccctccgattgaaatcacagaagatattcgtgttcttcttaagagaaaaagaggacatttaaaaacgctatggcaaagagaataaaagctaagcccacttcagacaaacctggaagtccatatcgctctgtcacacacttcgactcactagctgtaatagacatccctggagcagatactctggataaattatttgaccatgctgtatccaagtttgggaagaaggacagccttgggaccagggaaatcctaagtgaagaaaatgaaatgcagccaaatggaaaagtttttaagaagttaattcttgggaattataaatggatgaactatcttgaagtgaatcgcagagtgaataactttggtagtggactcactgcactgggactaaaaccaaagaacaccattgccatcttctgtgagaccagggccgaatggatgattgcagcacagacctgctttaagtacaactttcctcttgtgactttatatgccacacttggcaaagaagcagtagttcatgggctaaatgaatctgaggcttcctatctgattaccagtgttgaacttctggaaagtaaacttaagactgcattgttagatatcagttgtgttaaacatatcatttatgtggacaataaggctatcaataaagcagagtaccctgaaggatttgagattcacagcatgcaatcagtagaagagttgggatctaacccagaaaacttgggcattcctccaagtagaccaacgccttcagacatggccattgttatgtatactagtggttctactggccgacctaagggagtgatgatgcatcatagcaatttgatagctggaatgacaggccagtgtgaaagaatacctggactgggaccgaaggacacatatattggctacttgcctttggctcatgtgctagaactgacagcagagatatcttgctttacctatggctgcaggattggatattcttctccgcttacactctctgaccagtccagcaaaattaaaaaaggaagcaaaggagactgtactgtactgaagcccacacttatggctgctgttccggaaatcatggatagaatttataagaatgttatgagcaaagtccaagagatgaattatattcagaaaactctgttcaagatagggtatgattacaaattggaacagatcaaaaagggatatgatgcacctctttgcaatctgttactgtttaaaaaggtcaaggccctgctgggagggaatgtccgcatgatgctgtctggaggggccccgctatctcctcagacacaccgattcatgaatgtctgcttctgctgcccaattggccagggttatggactgacagaatcatgtggtgctgggacagttactgaagtaactgactatactactggcagagttggagcacctcttatttgctgtgaaattaagctaaaagactggcaagaaggcggttatacaattaatgacaagccaaaccccagaggtgaaatcgtaattggtggacagaacatctccatgggatattttaaaaatgaagagaaaacagcagaagattattctgtggatgaaaatggacaaaggtggttttgcactggtgatattggagaattccatcccgatggatgtttacagattatagatcgtaagaaagatctagtgaagttacaagcaggagagtatgtatctcttgggaaagtagaagctgcactgaagaattgtccacttattgacaacatctgtgcttttgccaaaagtgatcagtcctatgtgatcagttttgtggttcctaaccagaaaaggttgacacttttggcacaacagaaaggggtagaaggaacttgggttgatatctgcaataatcctgctatggaagctgaaatactgaaagaaattcgagaagctgcaaatgccatgaaattggagcgatttgaaattccaatcaaggttcgattaagcccagagccatggacccctgaaactggtttggtaactgatgctttcaaactgaaaaggaaggagctgaggaaccattacctcaaagacattgaacgaatgtatgggggcaaataaaatgttgttgtcttattgacagttgtgcaggaggtagcctggtggttttcaacctctagaattttaagcctttgttgaactgttagaatgtaaggtatatcattctaaagatagagtaaaaagaaaacaaaaccaaaagttattaaaattgttgtccggtttactttaacttagttttgcatagttctagtgcagctgaaattgaaaagttatttccctttagctgtgttattatagagcagaaattctgtttttaaaaattagcctaagatatacttgtttttgtaaagaaaaatatttaatgttgaacaaaataaattggagttggagtagaatgtagtttgaggaaatttgcagcttccaatgcctcttgtcttcctatttcagaagtttaaatattaagcatgacagaaaatatgtattaacactactcaaagcaaaagtgctgcagggctttaaaattctcttccaaccatttatcttgaaggaaaaattcaatagtaatataatacacaaaatcaaataataccttagaaggtattaagattataattgttgcataggttagatatagagtcattgtaatgttgtgaataattacagtgcctaaaataagaatagaacaacatatacaacaccaaaaaatatctagtaatatatttaaagggaaattgagctgctttttttgaaactttgagatctaaaaataactgtaattatttgaatgactaagaggaaagtacattttttgaaatgctgaaaattgcctttctgtgtttattcaaactgaaaagctgagaccaagagcaaggaaggtaaaaagttaacaggcaaacattttctcttagaaaaggtgataaaatcataagtatttggaattagaacccttgcacagcactgaacctgggaaagagatttaaactctgaatttatctttgataacagggattgattttaaaatgtacatgtattaaattacatttgtaatttaaggtctgtttgctgttgctgattttattcttgatcagtagtttgcatttcagaaagcctttcattttgctttaattttagcaaagcgggttataatgaatgacttccccaatatcttgcttgaacttacaggtgattaacttggatgagttttgggaagttaaagggaagaaaacactgttatcattttttcctgtttgggaagagcttagaaactggaaatactagatttgggagaagggcagagttacttgataagggactgatgtttgtgcagtaacttgggagtgtggtttctttttgaatctttaattaaaacctgggattatatatccctgataaatattcacacttgaaccatagttactgtaaaatgcaaaaaatcttaatactgttattctttgcactttttcttaatcattttttatatatatgcatatatatatgtgtgtgtgtgtgttgcttatgttgttttgtacagatgtgggccaccattgcaacaaaatacattctttttgctctaaaatatttatgaagaaaatacttaaatgttatgtatatggtggtaataagggaaaaatcaagtattataaacaagaatgaaggtttttgtaaagatttctgttcagcgttttgcaaggtaaaattttaggcaagttttccctgaagttatgtgtatgtgagtattctcattcttcccaacttgcctttgaagagtgaaataccattattatcaagtagactactgttcagcttttattcctgccctgctgtttatcccttaagaatgagtttcttagacttttccaatatgtgattttttttcccatttagaatggtgattttaaatgtgtgagtgcatgtactatcttatctcagatatttgcacccccaatctgcccccaactcccaaaagctagaacactgccaactgatctgttataggtcctttagaaacacataattaacacttaaggttgggtgctgctaattctttgcaaaaatccaaatattgttaagggaccagggagatgccactaccccttgattttccatctaaaaatatacatgtttatgtaaacaaatctttccatatccatagtgacttttcaagtatttaagcctaaagattttgatctcacatttttatacctgtttaaattgctcacagttattacatacacatcagccatcaactaaagttgtactttaaaaatttactacaatatgtacatttctaagtcaaacacttgtgacttttgctttaattccatgaatgttcctgcctccttgatatttgtatttattctttttttctctagagtagaggtataattgtgtgatatttcagaaatacagataaatgattcaaaaagtcacagttaaggagaatcatgtttctttgatcatgaataactgattagtaagtcttgcctatattttcctgatagcatatgacaaatgtttctaaggtaacaagatgagaacagataaagattgtgtggtgttttggatttggagagaaatattttaatttttaaatgcagttacaaattataatgtattcatatttgtactttctgttaaaatgcatgattgcagaattgtttagattttgtgtttattcttgatgaaaagctttgtttgttcttgtttttaagtttgcactcaaatcttaagaaataaatccacccatgttatcaaaaaaaaaaaaaaaaaa

SEQ ID NO: 48 - DNA, 2741 bp, Homo sapiens
acctccctccgcggagcagccagacagcgagggccccggccgggggcaggggggacgccccgtccggggcacccccccggctctgagccgcccgcggggccggcctcggcccggagcggaggaaggagtcgccgaggagcagcctgaggccccagagtctgagacgagccgccgccgcccccgccactgcggggaggagggggaggaggagcgggaggagggacgagctggtcgggagaagaggaaaaaaacttttgagacttttccgttgccgctgggagccggaggcgcggggacctcttggcgcgacgctgccccgcgaggaggcaggacttggggaccccagaccgcctccctttgccgccggggacgcttgctccctccctgccccctacacggcgtccctcaggcgcccccattccggaccagccctcgggagtcgccgacccggcctcccgcaaagacttttccccagacctcgggcgcaccccctgcacgccgccttcatccccggcctgtctcctgagcccccgcgcatcctagaccctttctcctccaggagacggatctctctccgacctgccacagatcccctattcaagaccacccaccttctggtaccagatcgcgcccatctaggttatttccgtgggatactgagacacccccggtccaagcctcccctccaccactgcgcccttctccctgaggacctcagctttccctcgaggccctcctaccttttgccgggagacccccagcccctgcaggggcggggcctccccaccacaccagccctgttcgcgctctcggcagtgccggggggcgccgcctcccccatgccgccctccgggctgcggctgctgccgctgctgctaccgctgctgtggctactggtgctgacgcctggccggccggccgcgggactatccacctgcaagactatcgacatggagctggtgaagcggaagcgcatcgaggccatccgcggccagatcctgtccaagctgcggctcgccagccccccgagccagggggaggtgccgcccggcccgctgcccgaggccgtgctcgccctgtacaacagcacccgcgaccgggtggccggggagagtgcagaaccggagcccgagcctgaggccgactactacgccaaggaggtcacccgcgtgctaatggtggaaacccacaacgaaatctatgacaagttcaagcagagtacacacagcatatatatgttcttcaacacatcagagctccgagaagcggtacctgaacccgtgttgctctcccgggcagagctgcgtctgctgaggctcaagttaaaagtggagcagcacgtggagctgtaccagaaatacagcaacaattcctggcgatacctcagcaaccggctgctggcacccagcgactcgccagagtggttatcttttgatgtcaccggagttgtgcggcagtggttgagccgtggaggggaaattgagggctttcgccttagcgcccactgctcctgtgacagcagggataacacactgcaagtggacatcaacgggttcactaccggccgccgaggtgacctggccaccattcatggcatgaaccggcctttcctgcttctcatggccaccccgctggagagggcccagcatctgcaaagctcccggcaccgccgagccctggacaccaactattgcttcagctccacggagaagaactgctgcgtgcggcagctgtacattgacttccgcaaggacctcggctggaagtggatccacgagcccaagggctaccatgccaacttctgcctcgggccctgcccctacatttggagcctggacacgcagtacagcaaggtcctggccctgtacaaccagcataacccgggcgcctcggcggcgccgtgctgcgtgccgcaggcgctggagccgctgcccatcgtgtactacgtgggccgcaagcccaaggtggagcagctgtccaacatgatcgtgcgctcctgcaagtgcagctgaggtcccgccccgccccgccccgccccggcaggcccggccccaccccgccccgcccccgctgccttgcccatgggggctgtatttaaggacacccgtgccccaagcccacctggggccccattaaagatggagagaggactgcggatctctgtgtcattgggcgcctgcctggggtctccatccctgacgttcccccactcccactccctctctctccctctctgcctcctcctgcctgtctgcactattcctttgcccggcatcaaggcacaggggaccagtggggaacactactgtagttagatctatttattgagcaccttgggcactgttgaagtgccttacattaatgaactcattcagtcaccatagcaacactctgagatgcagggactctgataacacccattttaaaggtgaggaaacaagcccagagaggttaagggaggagttcctgcccaccaggaacctgctttagtgggggatagtgaagaagacaataaaagatagtagttcaggccaggcggggtggctcacgcctgtaatcctagcacttttgggaggcagagatgggaggattacttgaatccaggcatttgagaccagcctgggtaacatagtgagaccctatctctacaaaacacttttaaaaaatgtacacctgtggtcccagctactctggaggctaaggtgggaggatcacttgatcctgggaggtcaaggctgcag

SEQ ID NO: 49 - DNA, 1906 bp, Homo sapiens
gctttccggcggttgcaccgggccggggtgccagcgcccgccttcccgtttcctcccgttccgcagcgcgcccacggcctgtgaccccggcgaccgctccccagtgacgagagagcggggccgggcgctgctccggcctgacctgcgaagggacctcggtccagtcccctgttgcgccgcgcccccgtccgtccgtgcgcgggccagtcaggggccagtgtctcgagcggtcgaggtcgcagacctagaggcgccccacaggccggcccggggcgctgggagcgccggccgcgggccgggtggggatgcctctgcacgtgaagtggccgttccccgcggtgccgccgctcacctggaccctggccagcagcgtcgtcatgggcttggtgggcacctacagctgcttctggaccaagtacatgaaccacctgaccgtgcacaacagggaggtgctgtacgagctcatcgagaagcgaggcccggccacgcccctcatcaccgtgtccaatcaccagtcctgcatggacgaccctcatctctgggggatcctgaaactccgccacatctggaacctgaagttgatgcgttggacccctgcagctgcagacatctgcttcaccaaggagctacactcccacttcttcagcttgggcaagtgtgtgcctgtgtgccgaggagcagaatttttccaagcagagaatgaggggaaaggtgttctagacacaggcaggcacatgccaggtgctggaaaaagaagagagaaaggagatggcgtctaccagaaggggatggacttcattttggagaagctcaaccatggggactgggtgcatatcttcccagaagggaaagtgaacatgagttccgaattcctgcgtttcaagtggggaatcgggcgcctgattgctgagtgtcatctcaaccccatcatcctgcccctgtggcatgtcggaatgaatgacgtccttcctaacagtccgccctacttcccccgctttggacagaaaatcactgtgctgatcgggaagcccttcagtgccctgcctgtactcgagcggctccgggcggagaacaagtcggctgtggagatgcggaaagccctgacggacttcattcaagaggaattccagcatctgaagactcaggcagagcagctccacaaccacctccagcctgggagataggccttgcttgctgccttctggattcttggcccgcacagagctggggctgagggatggactgatgcttttagctcaaacgtggcttttagacagatttgttcatagaccctctcaagtgccctctccgagctggtaggcattccagctcctccgtgcttcctcagttacacaaaggacctcagctgcttctcccacttggccaagcagggaggaagaagcttaggcagggctctctttccttcttgccttcagatgttctctcccaggggctggcttcaggagggagcatagaaggcaggtgagcaaccagttggctaggggagcagggggcccaccagagctgtggagaggggaccctaagactcctcggcctggctcctacccaccgcccttgccgaaccaggagctgctcactacctcctcagggatggccgttggccacgtcttccttctgcctgagcttcccccccaccacaggccctttcctcaggcaaggtctggcctcaggtgggccgcaggcgggaaaagcagcccttggccagaagtcaagcccagccacgtggagcctagagtgagggcctgaggtctggctgcttgcccccatgctggcgccaacaacttctccatcctttctgcctctcaacatcacttgaatcctagggcctgggttttcatgtttttgaaacagaaccataaagcatatgtgttggcttgttgtaaaa